(19) United States
(12) Patent Application Publication
(10) Pub. No.: US 2024/0409645 A1
(43) Pub. Date: Dec. 12, 2024

(54) Binding Molecules Targeting IL-2 Receptor

(71) Applicant: DUALYX NV, Keerbergen (BE)

(72) Inventors: Luc Van ROMPAEY, Keerbergen (BE); Valentina LYKHOPIY, Zwijnaarde, Gent (BE); Susan Mariola SCHLENNER, Leuven (BE); Tom Lander VAN BELLE, Destelbergen (BE); Christophe Frederic Jerome BLANCHETOT, Destelbergen (BE); Tugsan TEZIL, Elsene (BE); Giel TANGHE, De Pinte (BE)

(21) Appl. No.: 18/703,456

(22) PCT Filed: Oct. 21, 2022
(86) PCT No.: PCT/EP2022/079483

§ 371 Date: Apr. 22, 2024

(30) Foreign Application Priority Data
Oct. 21, 2021 (GB) 2115122.0

(51) Int. Cl. C07K 16/28; A61K 39/00; A61P 37/06; G01N 33/53; G01N 33/58; G01N 33/68

(52) U.S. Cl. CPC C07K 16/2866; A61P 37/06; G01N 33/53; G01N 33/58; G01N 33/6869; A61K 2039/505; C07K 2317/31; C07K 2317/52; C07K 2317/565; C07K 2317/569; C07K 2317/74; C07K 2317/76; C07K 2317/92; G01N 2333/7155

(57) ABSTRACT

The present invention provides binding molecules, particularly antibodies, or antigen-binding portions thereof, which bind to the IL-2 receptor and selectively activate regulatory T cells. The invention further relates to compositions and therapeutic methods for use of these binding molecules for the treatment and/or prevention of autoimmune diseases, and/or other conditions responsive to therapy that is effective to a selective increase in numbers and activation of regulatory T cells over effector T cells.

### FIELD OF INVENTION

The present invention relates to binding molecules that bind to one or more of the polypeptide chains of interleukin-2 receptor, hence that bind to one or more of the interleukin-2 receptor α-chain (IL-2Rα, encoded by IL2RA; also known as CD25), interleukin 2 receptor β-chain (IL-2Rβ, encoded by IL2RB; also known as CD122) and the common cytokine receptor γ-chain (γc, encoded by IL2RG; also known as IL-2Rγ or CD132). The present invention further relates to the use of such binding molecules to target interleukin receptors (IL-2R), particularly the high affinity IL-2Rα/IL-2Rβ/γc, as well as the binding molecules for use in methods of treatment and diagnosis. Preferred binding molecules are antibodies, with particularly preferred binding molecules comprising, or consisting of, VHH domain antibodies.

### BACKGROUND OF INVENTION

IL-2 is a pleiotropic cytokine involved in immune system function, playing a role in immune responses and also immune tolerance. The main cells that release IL-2 are activated CD4⁺ T cells. IL-2 influences the differentiation, proliferation, survival, and activity of a wide range of immune cells. It can act on multiple cell types, including T regulatory cells (Tregs), type 2 innate lymphoid cells (ILC2), Natural Killer (NK) cells, T memory (Tmem) cells, effector T cells (Teffs), activated B cells, and monocytes. Stimulation by IL-2 is often key to the survival and proliferation of such immune system cells.

The functional receptor for IL-2, IL-2R, exists as a two-chain heterodimeric form with medium affinity for IL-2 and a high affinity three chain heterotrimeric form. In particular, three different chains can be present in a receptor for IL-2, those being the interleukin-2 receptor α-chain (IL-2Rα; CD25), interleukin 2 receptor β-chain (IL-2Rβ; CD122) and common cytokine receptor γ-chain (γc; IL-2Rγ; CD132). IL-2Rβ and γc can form an IL-2R complex with medium affinity for IL-2. IL-2Rα, IL-2Rβ and γc can together form a heterotrimeric IL-2R complex with high affinity for IL-2. The medium affinity two chain IL-2Rβ/γc receptor complex may be an intermediate in the formation of the higher affinity IL-2Rα/IL-2Rβ/γc receptor complex. IL-2Rα on its own has low affinity for IL-2, but binding of IL-2 to IL-2Rα may also play a part in the formation of IL-2Rα/IL-2Rβ/γc receptor complexes.

As IL-2Rα is the polypeptide chain which is unique to the trimeric IL-2Rα/IL-2Rβ/γc receptor complex, the higher expression of IL-2Rα on Treg cells means that they are most responsive to IL-2. Low dose IL-2 therapy has been used as a way to attempt to preferentially stimulate Tregs, whilst higher dose IL-2 has been used to try and stimulate T effector cells. Lower dose IL-2 has been investigated for treating autoimmune disorders. However, low dose therapy has poor specificity, short in vivo half-life, and the potential for immunogenicity. Higher dose IL-2 has been used in cancer therapy, but has undesirable side-effects, such as vascular leak syndrome (VLS), and different patients show different levels of responsiveness to the treatment. Thus, whilst offering promise, IL-2 therapy has been slow to enter the clinic for the benefit of many patients. Mutant forms of IL-2, IL-2 muteins, have also been developed with increased specificity for the high affinity IL-2Rα/IL-2Rβ/γc receptor complex, but which showed off-target binding to CD25+ cells.

Given the importance of IL-2 in the immune system, a real need remains to provide further and improved ways to target IL-2 and IL-2R complexes, particularly the higher affinity IL-2Rα/IL-2Rβ/γc receptor complex to modulate Tregs.

### SUMMARY OF THE INVENTION

The present invention provides binding molecules against the polypeptide chains of the interleukin-2 receptor (IL-2R). The binding molecules are, or comprise, single domain binding regions, particularly comprising heavy chain only antibodies, and especially VHH domain antibodies. The present invention provides binding molecules comprising, or consisting of, single domain binding regions that are able to bind at least one of the IL-2Rα, IL-2Rβ and γc. Preferred single domain binding regions are heavy chain only antibodies. Especially preferred single domain binding regions are VHH domain antibodies. Hence, in a particularly preferred embodiment, the binding molecule may be, or may comprise a VHH domain antibody or antibodies.

Typically, the binding molecules of the present invention do not comprise IL-2 or mutant forms of IL-2. One advantage of the binding molecules of the present invention is therefore that, unlike the mutant forms of IL-2 being developed in the art as therapeutics, they do not run the risk of inducing antibodies against a mutant IL-2 that will cross-react with endogenous IL-2.

In a particularly preferred embodiment, the binding molecules are able to bind all three of IL-2Rα, IL-2Rβ, and γc, and preferably bind the IL-2Rα/IL-2Rβ/γc receptor complex. Hence, the invention allows for the targeting of the IL-2Rα/IL-2Rβ/γc receptor complex and so of Tregs. Such binding molecules are able to preferentially target Treg cells that express high levels of the interleukin-2 receptor α chain associated with stable FoxP3 expression and immunosuppressive properties, hence in one preferred embodiment, the binding molecules may be used to target Treg cells, for instance to preferentially activate Treg cells. The ability to stimulate Treg cells means that one preferred use of the binding molecules of the present invention is in methods to treat or prevent autoimmune disorders.

In one particularly preferred embodiment, the binding molecules of the present invention are, or comprise, VHH domain antibodies. The present inventors have taken advantage of the versatility of VHH domains, and their single polypeptide chain nature, to generate various monospecific, bispecific, trispecific and multispecific binding molecules for targeting IL-2 receptors. In a preferred embodiment, the present invention provides a binding molecule comprising VHH domains that mean that the binding molecule is able to bind at least one of the interleukin-2 receptor α-chain, β-chain, and common cytokine receptor γ-chain. In a particularly preferred embodiment, the binding molecule comprises at least three VHH domains, with at least one VH domain specific against each of the interleukin-2 receptor α chain, β chain, and common γ chain.

The present invention provides a trispecific binding molecule comprising:
at least one single domain binding region specific for the IL-2α receptor α-chain;
at least one single domain binding region specific for the IL-2 receptor β-chain; and
at least one single domain binding region specific for the γc.

The present invention further provides a trispecific binding molecule of the present invention for use as a medicament.

The present invention further provides a trispecific binding molecule of the present invention for use in a method of treating or preventing an autoimmune disorder, or an inflammatory disorder, preferably wherein:
(a) the disorder is graft versus host disease (GvHD), preferably where the antibody is for use in a method where it is administered prior to, at the same time, or after a transplant of a cell, tissue, or organ;
(b) the disorder is one involving dysfunction or unwanted proliferation of leukocytes, preferably of T cells, more preferably of Teff cells; such disorders may present with an imbalance of Tregs compared to Teff cells;
(c) the disorder is selected from inflammatory bowel disease (IBD) (such as ulcerative colitis (UC), Crohn's disease, pouchitis or celiac disease), systemic lupus erythematosus (SLE), multiple sclerosis, type 1 diabetes, myasthenia gravis, pemphigus vulgaris, and bullous pemphigoid; or
(d) the disorder is selected from SLE, cGvHD, psoriasis, autoimmune hepatitis, ulcerative colitis, eczema.

The present invention further provides a method of stimulating cell proliferation comprising contacting a target cell expressing the IL-2Rα/IL-2Rβ/γc receptor complex with a trispecific binding molecule of the present invention.

The present invention also provides a pharmaceutical composition comprising a trispecific binding of the present invention and a pharmaceutically acceptable carrier.

The present invention further provides a method of detecting the IL-2Rα/IL-2Rβ/γc receptor complex comprising contacting a test sample with a binding molecule of the present invention and detecting binding of the binding molecule to the IL-2Rα/IL-2Rβ/γc receptor complex, preferably wherein the binding molecule is labelled and the binding of the antibody to the IL-2Rα/IL-2Rβ/γc receptor complex is detected via the label.

### BRIEF DESCRIPTION OF THE FIGURES

FIG. 1A shows the binding of selected VHH antibodies specific for the IL-2Rα-, β-, or γ-chain to HEK-Blue cells expressing all three of the IL-2R α-, β-, or γ-chain as measured by FACS analysis.

FIG. 1B shows the dose-response binding curves of selected monospecific monovalent IL-2Rα VHH antibodies to HEK cells expressing the trimeric IL-2Rα/β/γ as measured by FACS analysis. The format of a monospecific bivalent IL-2R subunit antibody used to obtain the results is shown above the graph.

FIG. 2 shows the ability of selected VHH antibodies specific for the IL-2Rα-, β-, or γ-chain to block binding of IL-2 to HEK-Blue cells expressing all three IL-2R chains as measured by FACS analysis. The higher the column in the graph, the greater the inhibition of binding of IL-2 to the HEK-blue cells by the antibody.

FIG. 3A shows results from testing the monospecific bi- and mono-valent VHH-hFc molecules specific for IL-2Rα/CD25 in a binding ELISA on human and cynomolgus monkey IL-2Rα/CD25 subunits. The structure of the monospecific monovalent and monospecific bivalent antibodies are shown below the graph.

FIG. 3B shows results from testing the monospecific bi- and mono-valent VHH-hFc molecules specific for IL-2Rβ/CD122 in a binding ELISA on human and cynomolgus monkey IL-2Rβ/CD122 subunits. The structure of the monospecific monovalent and monospecific bivalent antibodies assessed was the same as that shown below the graph in FIG. 3A.

FIG. 3C shows results from testing the monospecific bi- and mono-valent VHH-hFc molecules specific for IL-2Rγ/CD132 in a binding ELISA on human and cynomolgus monkey IL-2Rγ/CD132 subunits. The structure of the monospecific monovalent and monospecific bivalent antibodies assessed was the same as that shown below the graph in FIG. 3A

FIG. 4 summarises the VHH clones specific for the IL-2R α-, β-, or γ-chain employed to generate monospecific, bispecific, and trispecific antibodies, with the structure of the antibodies generated shown below the Table. The structures shown are monospecific, bispecific, or trispecific in the sense of whether they bind just IL-2R α alone (the structure on the left), bind IL-2R α and β (bivalent, bispecific structure in the middle), or bind all three of IL-2R α-, β-, and γ (the structure on the right). The structures are referred to as monovalent or bivalent in the sense of how many binding sites that they have individually for each of the IL-2R α-, β-, and γ chains present, even though the overall valency is higher (the structure on the left has an overall valency of two, that in the middle an overall valency of four, whist that on the right has an overall valency of three).

FIG. 5 shows hydrophobic interaction chromatography (HIC) chromatograms for two parental monospecific anti-IL-2Rα and bispecific anti-IL-2Rβ/γc antibodies and the heterodimeric trispecific anti-IL-2Rα/IL-2Rβ/γc antibody. The large peak in each chromatogram represents the main species of the antibody in the sample. The purity obtained for selected trispecific antibodies is indicated in the Table below the HIC chromatograms.

FIG. 6 shows the ability of particular monospecific, bispecific and trispecific antibodies to: bind HEK cells expressing IL-2Rα/IL-2Rβ/γc (top panel); inhibit binding of IL-2 to HEK cells expressing IL-2Rα/IL-2Rβ/γc (middle panel); and activate IL-2R signalling as measured by STAT5 phosphorylation.

FIG. 7A shows the dose-response binding curves of selected bispecific anti-IL-2Rβ/γ VHH antibodies to HEK cells expressing the trimeric IL-2Rα/β/γ as measured by FACS analysis. The antibodies assessed had a valency of two against each of the IL-2Rβ and γ chains and hence an overall valency of four.

FIG. 7B shows the dose-response binding curves of selected trispecific anti-IL-2Rα/β/γc VHH antibodies to HEK cells expressing the trimeric IL-2Rα/β/γ as measured by FACS analysis. The antibodies assessed had a valency of one against each of the IL-2R α β and γ chains and hence an overall valency of three.

FIG. 8 shows the ability of particular bispecific IL-2Rβ/γc and trispecific IL-2Rα/IL-2Rβ/γc antibodies to activate IL-2R signalling as measured by pSTAT5 phosphorylation in HEK cells expressing IL-2Rα/IL-2Rβ/γc, with the Table displaying EC50 (nM) and maximum signal values (% of live cells positive for phosphorylated STAT5).

FIG. 9A shows the pSTAT5 dose-response curves of selected bispecific monovalent anti-IL-2Rβ/γ VHH antibodies in HEK cells expressing the trimeric IL-2Rα/β/γ as measured by FACS analysis.

FIG. 9B shows the pSTAT5 dose-response curves of selected trispecific (monovalent) anti-IL-2Rα/β/γ VHH antibodies in HEK cells expressing the trimeric IL-2Rα/β/γ as measured by FACS analysis.

FIG. 9C shows the pSTAT5 dose-response curves of selected trispecific monovalent anti-IL-2Rα/β/γ VHH antibodies (cfr. FIG. 12C) in HEK cells expressing the trimeric IL-2Rα/β/γ as measured by FACS analysis.

FIG. 9D shows the pSTAT5 dose-response curves of tsVHH48 variants, in particular selected trispecific symmetrical, bivalent anti-IL-2α/β/γ (DC00011, DC00015, DC00019, DC00020, DC00021; cfr. FIG. 12A) and corresponding bispecific mono-(one armed, OA) and bivalent anti-IL-2β/γ VHH antibodies in HEK cells expressing the trimeric IL-2R α/β/γ as measured by FACS analysis.

FIG. 10A shows the ability of particular bispecific IL-2Rβ/γc and trispecific IL-2Rα/IL-2Rβ/γc antibodies to induce dose-dependent pSTAT5 activation of human PBMCs, with trispecific antibodies demonstrating enhanced selectivity and potency for human Tregs.

FIG. 10B shows the ability of monoparatopic tsVHH-48 geometry variants to induce dose-dependent pSTAT5 activation of human PBMCs, with particular trispecific IL-2Rα/IL-2Rβ/γc antibodies demonstrating enhanced selectivity and potency versus IL-2 or the parental tsVHH-48 for human Tregs over NK cells. Graphs represent data from one experiment with one PBMC donor.

FIG. 10C shows the ability of anti-CD25-biparatopic variants of ts VHH48 to induce dose-dependent pSTAT5 activation of human PBMCs, with particular trispecific biparatopic IL-2Rα/IL-2Rβ/γc antibodies demonstrating enhanced selectivity and potency versus the parental tsVHH48 for human Tregs over NK cells. Graphs represent data from one experiment with two PBMC donors.

FIG. 11 shows the ability of trispecific IL-2Rα/IL-2Rβ/γc antibodies to preferentially expand Tregs in human PBMC culture.

FIG. 12A shows illustrative examples of antibody formats that may be employed in the present invention.

FIG. 12B shows further illustrative examples of antibody formats that may be employed in the present invention.

FIG. 12C shows further illustrative examples of antibody formats that may be employed in the present invention. The upper panel illustrates antibodies employing the same VHH building blocks as used for tsVHH48, but with a different order and/or number of VHHs in one or both arms of the antibody thereby resulting in different overall geometries and/or valencies. The lower panel illustrates the use of tsVHH48 building blocks fused to one Fc tail, paired with CD25-binding VHHs belonging to different CDR3 families fused to a second Fc tail (the binding molecules therefore being biparatopic for CD25 and overall being tsVHHs in the sense of having at least one specificity for each of IL-2 α-, β-, and γ-).

FIG. 13 shows the result for treatment of aGvHD mice with tsVHH48.

FIG. 13A shows the survival and disease activity score of xenogeneic graft-versus-host-diseased mice treated with tsVHH48. The mice from the control group were injected intraperitoneally with 100 μg PBS (n=6 mice). Survival rates (A), weight loss (B) and the disease activity (C) are represented from one experiment. Data under B. and C. are represented as mean±SEM. P-value. Paired t-test (* p<0.05; ** p<0.005, *** p<0.0005).

FIG. 13B shows the frequency and proliferation of immune cells in blood over time. Blood was individually collected on a weekly basis and stained for flow cytometry analysis. Percentage of human CD45 engraftment (A), T lymphocytes (B), NK T cells (C), NK cells (D), CD8 T cells (E), effector CD8 T cells (F), proliferating CD8 T cells (G), CD4 T cells (H), effector CD4 T cells (I) and proliferating CD4 T cells (J) are respectively represented. Data are plotted as mean±SEM and represent one experiment.

FIG. 13C shows the frequency and proliferation of Tregs in blood over time. Blood was individually collected on a weekly basis and stained for flow cytometry analysis. Frequency (A) of Tregs (Foxp3+CD127− of the total CD4+ cells) and proliferating Tregs (B) are represented. Data are plotted as mean±SEM and represent one experiment.

### DETAILED DESCRIPTION

### Binding Molecules, Antibodies and IL-2 Receptors

The present invention provides binding molecules that are able to bind one or more of the polypeptide chains of the IL-2R. For example, the present invention provides a binding molecule that can bind all three of the polypeptide chains of the IL-2R, preferably which can bind all three at the same time.

The binding molecules provided are typically characterised as being, or comprising, single domain binding regions. A single domain binding region consists of a single domain able to bind a target. In one embodiment, the single domain binding region is characterised by not including an antibody light chain. In one embodiment, the binding molecule as a whole does not include an antibody light chain. An advantage of employing single domain binding regions is that it is easier to join together permutations of different single binding domains. In one particularly preferred embodiment the binding molecule is, or comprises, antibody-based sequences. In an alternative embodiment it does not. In one embodiment of the invention, a binding molecule does not comprise Fab binding regions.

In embodiments where a binding molecule of the present invention comprises antibody-based sequences, the binding molecule may be simply referred to as an antibody. Reference to an antibody may be used to refer to the overall structure, even if all of the constituents of the overall structure are not antibody based, the overall structure is not a naturally occurring antibody, or the overall structure includes non-antibody-based sequences. Reference to an “antibody” herein specifically encompasses an individual VHH molecule, as well as an antibody that comprises a VHH molecule as part of the overall structure. Hence, reference to an antibody is not limited to a four polypeptide IgG structure with two light and two heavy chain polypeptides, but also antibody structures where the overall structure is not a naturally occurring one, but the antibody still includes antibody-based sequences. For instance, whilst VHH heavy chain only antibodies are naturally occurring structures, antibodies that comprise more than one VHH molecule or domain are not naturally occurring, but they are still specifically part of the present invention and represent an “antibody” as defined herein. Reference to an antibody herein also includes antibodies that themselves therefore comprise antibodies as one of their constituent parts.

Reference to the “geometry” of a binding molecule and in particular an antibody refers in particular to the number, order, and what the antigen binding sites present bind for a given binding molecule. In one embodiment, the overall structure of the antibody is referred to the “format” of an antibody, with reference to an antibody format though not preferably being limiting to specific sequences.

In a preferred embodiment, the binding molecules of the present invention are, or comprise, heavy chain only antibodies (HCAb). Reference to a heavy chain only antibody includes molecules that represent the heavy chain of an antibody, but lack the CH1 domain, and which are able to bind antigen without needing an accompanying light chain. Reference to a heavy chain only antibody also include VHH domain antibodies, for instance from camelids and VNAR antibodies, for example from cartilaginous fish. In an especially preferred embodiment, a heavy chain antibody employed in the present invention is, or comprises, a VHH domain antibody. However, other types of HCAb may be employed such as human, rat or mouse HCAbs. In another embodiment, other single domain binding regions may be employed which are not antibody based. So, for instance, in one embodiment, the single domain binding regions employed are non-Ig engineered protein scaffolds such as darpins, affibodies, adnectins, anticalin proteins, or peptides and the like. So wherever reference to a VHH domain is used herein, as an alternative embodiment any HCAbs in general may be employed, as well as non-antibody based single domain binding regions, including any of those referred to herein. Further, wherever reference to a single binding domain is made herein, instead a heavy chain only antibody may be employed, with the term heavy chain only antibody encompassing both single binding domains, such as VHH, but also heavy chain only antibodies that are heavy chains able to bind antigen without a light chain, for instance heavy chains lacking a CH1 region.

In a preferred embodiment of the present invention, an antigen binding site present in a binding molecule of the present invention is a VHH domain. In one preferred embodiment, all of the antigen binding sites are provided by VHH domains. In one embodiment, a binding molecule, consisting of a VHH domain as set out herein is provided. In another embodiment, a binding molecule comprising at least one VHH domain as set out herein is provided. In one embodiment, a binding molecule comprising a VHH domain as a sole antigen binding site is provided. In a preferred embodiment a binding molecule of the present invention comprises at least two VHH domains as set out herein. In one embodiment, a binding molecule of the present invention comprises two different VHH domain as set out herein. The present invention provides a bispecific binding molecule comprising two different VHH domains as set out herein. In one preferred embodiment, a binding molecule of the present invention comprises at least three different VHH domains as set out herein. In one embodiment a binding molecule of the present invention is a trispecific comprising three different VHH molecules as set out herein. In a preferred embodiment, the VHH domain or VHH domains will all be specific for an IL-2R polypeptide chain.

VHH antibodies comprise three CDRs, CDR1, CDR2, and CDR3. Reference to a “set of CDRs” in relation to a VHH domain antibody refers to the CDR1, CDR2, and CDR3 of that VHH domain. So, for instance, TABLE 3 identifies preferred VHH domain antibodies which are individually provided, but which may also be used as constituents for a binding molecule of the present invention. So the present invention provides a binding molecule comprising a VHH domain as set out in TABLE 3.

TABLE 4 of the present application sets out the CDR sequences of the VHH domains from TABLE 3. The present invention also provides a binding molecule comprising a “set” of CDRs, so CDR1, CDR2, and CDR3, from

TABLE 4, so from one of the VHH domain antibodies in TABLE 3.

TABLE 7 of the present application provides the VHH domain sequences and CDR sequences for further VHH domain antibodies specific for the IL-2Rα polypeptide, with the invention providing such VHH antibodies, as well as a binding molecule comprising one of the VHH domain antibodies from TABLE 7, and also a binding molecule comprising a set of CDRs from one of the VHH domain antibodies from TABLE 7.

TABLE 8 of the present application provides the VHH domain sequences and CDR sequences for VHH domain antibodies specific for the IL-2Rβ polypeptide, with the invention providing such VHH antibodies, as well as a binding molecule comprising one of the VHH domain antibodies from

TABLE 8, and also a binding molecule comprising a set of CDRs from one of the VHH domain antibodies from TABLE 8. TABLE 9 of the present application provides the VHH domain sequences and CDR sequences for VHH domain antibodies specific for the IL-2Rγ polypeptide, with the invention providing such VHH antibodies, as well as a binding molecule comprising one of the VHH domain antibodies from TABLE 9, and also a binding molecule comprising a set of CDRs from one of the VHH domain antibodies from TABLE 9. The present invention also provides a VHH domain antibody, or a binding molecule comprising such a VHH domain, which comprises a set of CDR sequences from one of TABLES 3, 6, 7, or 8, but with different, or at least modified, framework sequences. As discussed herein variant sequences are also provided, so anywhere herein reference to a specific sequence is made, a variant sequence may also be employed, particularly a variant that retains ability to bind to the specific IL-2R polypeptide chain. In another embodiment, a variant may be one that has one or more CDRs with sequence modifications present, for instance a CDR may comprise one, two, three, or four sequence changes compared to the specific ones set out, with one, two, or three CDRs each having such a level of sequence change. In one embodiment, the sequence changes are conservative sequence changes. Variant sequences will typically retain binding activity, for instance having substantially the same binding activity for the target.

The binding molecule provided by the invention bind to one or more of the interleukin-2 receptor α-chain (IL-2Rα; CD25), interleukin 2 receptor β-chain (IL-2Rβ; CD122) and common cytokine receptor γ-chain (γc; IL-2Rγ; CD132). In a particularly preferred embodiment, the IL-2R polypeptide chain bound by a binding molecule of the present invention is human. The sequences of the human IL-2R polypeptide chains are provided as follows:
SEQ ID NO: 2036 provides the sequence of the human interleukin-2 receptor α-chain and an antibody of the invention may specifically bind that sequence:

MDSYLLMWGLLTFIMVPGCQAELCDDDPPEIPHATFKAMAYKEGT
MLNCECKRGFRRIKSGSLYMLCTGNSSHSSWDNQCQCTSSATRNT
TKQVTPQPEEQKERKTTEMQSPMQPVDQASLPGHCREPPPWENEA
TERIYHFVVGQMVYYQCVQGYRALHRGPAESVCKMTHGKTRWTQP
QLICTGEMETSQFPGEEKPQASPEGRPESETSCLVTTTDFQIQTE
MAATMETSIFTTEYQVAVAGCVFLLISVLLLSGLTWQRRQRKSRR
TI
(SEQ ID NO: 1865->NP 000408.1 interleukin-2
receptor subunit alpha
isoform 1 precursor [Homo sapiens])

SEQ ID NO: 2037 provides the sequence of the human interleukin-2 receptor β-chain and an antibody of the invention may specifically bind that sequence:

MAAPALSWRLPLLILLLPLATSWASAAVNGTSQFTCFYNSRANIS
CVWSQDGALQDTSCQVHAWPDRRRWNQTCELLPVSQASWACNLIL
GAPDSQKLTTVDIVTLRVLCREGVRWRVMAIQDFKPFENLRLMAP
ISLQVVHVETHRCNISWEISQASHYFERHLEFEARTLSPGHTWEE
APLLTLKQKQEWICLETLTPDTQYEFQVRVKPLQGEFTTWSPWSQ
PLAFRTKPAALGKDTIPWLGHLLVGLSGAFGFIILVYLLINCRNT
GPWLKKVLKCNTPDPSKFFSQLSSEHGGDVQKWLSSPFPSSSFSP
GGLAPEISPLEVLERDKVTQLLLQQDKVPEPASLSSNHSLTSCFT
NQGYFFFHLPDALEIEACQVYFTYDPYSEEDPDEGVAGAPTGSSP
QPLQPLSGEDDAYCTFPSRDDLLLFSPSLLGGPSPPSTAPGGSGA
GEERMPPSLQERVPRDWDPQPLGPPTPGVPDLVDFQPPPELVLRE
AGEEVPDAGPREGVSFPWSRPPGQGEFRALNARLPLNTDAYLSLQ
ELQGQDPTHLV
(SEQ ID NO: 1866->NP 000869.1
interleukin-2 receptor subunit beta
precursor [Homo sapiens])

SEQ ID NO: 2038 provides the sequence of the human interleukin-2 receptor γ-chain and an antibody of the invention may specifically bind that sequence:

MLKPSLPFTSLLFLQLPLLGVGLNTTILTPNGNEDTTADFFLTTM
PTDSLSVSTLPLPEVQCFVENVEYMNCTWNSSSEPQPTNLTLHYW
YKNSDNDKVQKCSHYLFSEEITSGCQLQKKEIHLYQTFVVQLQDP
REPRRQATQMLKLQNLVIPWAPENLTLHKLSESQLELNWNNRFLN
HCLEHLVQYRTDWDHSWTEQSVDYRHKFSLPSVDGQKRYTFRVRS
RFNPLCGSAQHWSEWSHPIHWGSNTSKENPFLFALEAVVISVGSM
GLIISLLCVYFWLERTMPRIPTLKNLEDLVTEYHGNFSAWSGVSK
GLAESLQPDYSERLCLVSEIPPKGGALGEGPGASPCNQHSPYWAP
PCYTLKPET
(SEQ ID NO: 1867->NP_000197.1 cytokine
receptor common subunit gamma precursor
[Homo sapiens])

In other embodiments of the present invention the binding molecule may bind to IL-2R polypeptide chains from any of the species mentioned herein. In one preferred embodiment, a binding molecule of the present invention may bind to both the human IL-2R polypeptide and the corresponding mouse polypeptide. In another embodiment, a binding molecule may bind the human polypeptide, but not bind the mouse polypeptide.

In one embodiment, a binding molecule of the present invention will bind to a cell expressing an IL-2Rα/IL-2Rβ/γc complex. In another embodiment, a binding molecule of the present invention will bind to a cell expressing an IL-2Rβ/γc complex. In a preferred embodiment, a binding molecule of the present invention will bind preferentially to (for instance it may be specific for, or specifically interact with, or specifically bind) cells expressing IL-2Rα/IL-2Rβ/γc complex over cells expressing IL-2Rβ/γc complex. In one embodiment, a binding molecule may bind both an IL-2Rα/IL-2Rβ/γc complex and an IL-2Rβ/γc complex.

In one preferred embodiment, a binding molecule of the present invention will bind to an IL-2Rα/IL-2Rβ/γc receptor complex and stimulate IL-2R signalling. IL-2R complexes are thought to signal through a pathway involving the tyrosine kinases Jak1 and Jak3 which are associated respectively with IL-2Rβ and γc. Phosphorylation of IL-2Rβ leads to activation of the MAPK, PI-3K and predominately the Stat5 transcription factor. In one embodiment, a binding molecule of the invention may act as an agonist of the IL-2R complex, for instance increased phosphorylation of STAT5 may be seen in the target cell when contacted with a binding molecule of the invention. In one embodiment MAPK, PI-3K, and/or STAT5 may be activated, for instance all three may be activated, or at least STAT5. In one embodiment, downstream members of the STAT5 signalling pathway may be activated. In another embodiment, a binding molecule of the present invention may act as an antagonist of IL-2R activation. In one preferred embodiment, a binding molecule of the present invention blocks or inhibits the binding of IL-2 to an IL-2R, so for instance decreased STAT5 phosphorylation may be seen when a cell expressing IL-2Rα/IL-2B/γc complex is incubated with the binding molecule and IL-2 compared to when the cell is incubated with only IL-2. In another embodiment, a binding molecule of the present invention binds to the receptor, but does not also prevent IL-2 binding to the receptor as well.

The specificity of a binding molecule, in particular of an antibody, denotes what epitope/antigen it binds. In a particularly preferred embodiment, it will be used to denote how many different antigens a binding molecule binds. Thus a monospecific antibody binds one antigen. A bispecific antibody binds two antigens. A trispecific antibody binds three antigens. In relation to IL-2Rα, IL-2Rβ, and γc, a monospecific antibody will be said to bind one of those chains, a bispecific two, and a trispecific three. Hence, a trispecific antibody is one that has at least one binding site for each of IL-2Rα, IL-2Rβ, and γc. If an antibody has binding sites for more than one epitope on one of IL-2Rα, IL-2Rβ, and γc that will not change whether the antibody is said to be monospecific, bispecific, or trispecific in relation to IL-2Rα, IL-2Rβ, and γc, but will be instead denoted using biparatopic, triparatopic and so on nomenclature. Thus, an antibody with two different binding sites for IL-2Rα which each bind a different epitope of IL-2Rα will be referred to herein as a biparatopic antibody in relation to IL-2Rα. An antibody with three different binding sites each recognising a different epitope of IL-2Rα will be referred to as tri-paratopic in relation to IL-2Rα. Such nomenclature may also be used in relation to other antigens including IL-2Rβ, and γc.

The valency of a binding molecule, in particular an antibody, denotes the number of antigen-binding sites it has. A binding molecule of the present invention will have a valency of at least one. For instance, a binding molecule of the invention may have a valency of one. It may have a valency of two. It may have a valency of three. It may have a valency of four. In one embodiment, an antibody may have a valency of five. In another embodiment, it may have a valency of six. In another embodiment, it may have a valency of seven. In a further embodiment, it may have a valency of eight. In one embodiment, a binding molecule of the invention has at least those values as a valency. In one embodiment, a binding molecule of the invention has a valency of those values for IL-2R polypeptides. In one embodiment, reference to a valency may indicate how many binding sites are present for a given antigen. Hence, for example, a molecule may be referred to as bivalent for IL-2Rα to denote the number of binding sites for IL-2Rα, even though the overall number of binding sites for different antigens, and hence the overall valency is greater.

In one particularly preferred embodiment, a binding molecule is biparatopic for at least one of IL-2R α β, and γc. Preferably it is biparatopic for at least IL-2R α. In one particularly preferred embodiment, a binding molecule is trispecific in respect of IL-2R α, β, and γc, so having binding sites for all three, and is at least biparatopic for at least one of IL-2R α β, and γc. In a further particularly preferred embodiment, a binding molecule is trispecific in respect of IL-2R α, β, and γc, so having binding sites for all three, and is at least biparatopic for IL-2R α. In one preferred embodiment, a binding molecule, particularly an antibody, of the present invention is biparatopic for IL-2R α, but is monoparatopic for the other IL-2R chain or chains. In one preferred embodiment, a binding molecule, in particular an antibody, is trispecific for IL-2R α, β, and γc, biparatopic for IL-2R α, and is monoparatopic for β, and γc. In another preferred embodiment, a binding molecule, in particular an antibody, of the invention has more binding sites for IL-2R α, than for either of IL-2R β, and γc.

The strength of binding of an individual binding site to an IL-2R polypeptide may be referred to as the affinity of the binding site for its target, the IL-2R polypeptide. Whilst the overall strength of binding of a binding molecule is often also referred to as the affinity of the binding molecule, where the binding molecule has more than one binding site, the strength of binding may be referred to using the term avidity, which reflects the overall strength of binding when all of the binding sites of the binding molecule are taken into account.

As well as the preferred tri-specific binding molecules set out herein, all of the specific and variant sets of CDRs, VHH domains and polypeptides are also provided in the context of binding molecules that just bind one IL-2R α, β, and γc, as well as versions that bind two of IL-2R α, β, and γc. Hence, the binding sites set out herein may be provided as well as monovalent molecules binding the relevant one of IL-2R α, β, and γc. They are also provided where binding sites for two of IL-2R α, β, and γc are present, but not for all three. For example, a binding molecule of the present invention may also be provided which binds β, and γc, but not IL-2R α.

In one preferred embodiment, a binding molecule of the present invention will bind an IL-2Rα/IL-2Rβ/γc complex preferentially compared to an IL-2Rβ/γc complex. For instance, the strength of binding for the former compared to the latter may be at least 2, 10, 50, 100, 500, 1000 or more times higher. In one embodiment, the strength of binding may be at least 10,000, or at least 100,000 times greater. So, for instance, the avidity of the binding molecule for the IL-2Rα/IL-2Rβ/γc complex may be greater than that for the IL-2Rβ/γc complex. In one embodiment, a binding molecule of the present invention may be selective for the IL-2Rα/IL-2Rβ/γc complex over the IL-2Rβ/γc complex, in the sense that it specifically binds the trimeric receptor complex, but not the dimeric complex, or does not significantly bind it.

In another embodiment, a binding molecule of the present invention will bind both an IL-2Rα/IL-2Rβ/γc complex and an IL-2Rβ/γc complex. In one embodiment, the binding molecule may bind both IL-2Rα/IL-2Rβ/γc and IL-2Rβ/γc complexes, but bind the former with greater strength because extra binding site or sites are binding IL-2Rα as well as IL-2Rβ and γc. For example, in the case of a trispecific binding molecule it may be that it binds the IL-2Rα/IL-2Rβ/γc complex with greater strength because three binding sites are binding that complex, rather than the two that bind an IL-2Rβ/γc complex. In another embodiment, the binding molecule may preferentially bind the IL-2Rα/IL-2Rβ/γc complex because the binding molecule comprises more binding sites for IL-2Rα than the number of binding sites it has individually for either of IL-2Rβ and γc. In another embodiment, the binding molecule may preferentially bind the IL-2Rα/IL-2Rβ/γc complex because the binding site or sites for IL-2Rα are individually of higher affinity than those for either of IL-2Rβ and γc. In a further embodiment, the binding molecule may have a higher avidity for the IL-2Rα/IL-2Rβ/γc complex because of a combination of those factors.

In one embodiment, an antigen binding domain of a binding molecule of the invention for its target IL-2R polypeptide may have a K_{D} which is about 400 nM or smaller, 200 nM or smaller such as about 100 nM, 50 nM, 20 nM, 10 nM, 1 nM, 500 pM, 250 pM, 200 pM, 100 pM or smaller. In one embodiment, the K_{D} is 50 pM or smaller. In one embodiment, the KD of an individual antigen-binding site of a binding molecule of the present invention may be less than 1 μM, less than 750 nM, less than 500 nM, less than 250 nM, less than 200 nM, less than 150 nM, less than 100 nM, less than 75 nM, less than 50 nM, less than 10 nM, less than 1 nM, less than 0.1 nM, less than 10 pM, less than 1 pM, or less than 0.1 pM. In some embodiments, the K_{D} is from about 0.1 pM to about 1 μM. It may be an individual antigen-binding domain has such K_{D}. It may be that such a K_{D} is displayed by the overall binding molecule of the invention for the IL-2R polypeptide. It may be that such a K_{D} is displayed for IL-2Rα/IL-2Rβ/γc complexes.

In one embodiment, an antigen binding domain of a binding molecule of the invention for its target IL-2R polypeptide may have an EC50 which is about 400 nM or smaller, 200 nM or smaller such as about 100 nM, 50 nM, 20 nM, 10 nM, 1 nM, 500 pM, 250 pM, 200 pM, 100 pM or smaller. In one embodiment, the EC50 is 50 pM or smaller. In one embodiment, the EC50 of an individual antigen-binding site of a binding molecule of the present invention may be less than 1 μM, less than 750 nM, less than 500 nM, less than 250 nM, less than 200 nM, less than 150 nM, less than 100 nM, less than 75 nM, less than 50 nM, less than 10 nM, less than 1 nM, less than 0.1 nM, less than 10 pM, less than 1 pM, or less than 0.1 pM. In some embodiments, the EC50 is from about 0.1 pM to about 1 μM. It may be an individual antigen-binding domain has such EC50. It may be that such a EC50 is displayed by the overall binding molecule of the invention for the IL-2R polypeptide. It may be that such a EC50 is displayed for IL-2Rα/IL-2Rβ/γc complexes.

Binding, including the presence or absence of binding, can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). Binding to the IL-2Rα/IL-2Rβ/γc and IL-2Rβ/γc complexes may be, for instance, measured using cells expressing such complexes, preferably where such complexes are human. In one embodiment, HEK cells expressing the trimeric IL-2 receptor are used to measure binding, for instance via FACS.

In one particularly preferred embodiment, a binding molecule of the invention may have greater potency for targeting cells that express IL-2Rα/IL-2Rβ/γc versus that displayed by IL-2Rβ/γc alone. For instance, a binding molecule of the invention may preferentially activate cells expressing IL-2Rα/IL-2Rβ/γc versus those expressing the IL-2Rβ/γc alone. In one embodiment, a binding molecule of the present invention may be used to preferentially target Treg cells because of their higher level of expression of the IL-2Rα/IL-2Rβ/ye receptor and hence to preferentially activate Treg cells versus other cell types, including Teff cells. In one embodiment, a binding molecule of the present invention activates Tregs by a factor of at least 5, ten, 50, 100, or 1000 fold more than it does other cells, for instance Teff cells. In one embodiment, employing a binding molecule of the present invention shifts the balance of an immune response from one characterised by Teff cells to Treg cells.

### Constant Regions

In one embodiment, a binding molecule, in particular an antibody, of the present invention does not comprise a constant region. However, in one preferred embodiment of the present invention the binding molecule of the present invention is an antibody that comprises a constant region. For instance, in one embodiment an antibody of the present invention comprises a polypeptide comprising a VHH domain and an Fc region. The constant region, if present, can be from any class of antibody, for instance can be a gamma, mu, alpha, delta, or epsilon constant region, or a part thereof. In a particularly preferred embodiment, the constant region is an IgG constant region. For instance, it may be an IgG1, IgG2, IgG3, or IgG4 constant region. The IgG1 constant region, or part thereof, is particularly preferred. In a particularly preferred embodiment, the constant region is an Fc region and so comprises the CH2 and CH3 domains, but does not comprise a CH1 domain. Hence, reference herein to a constant region or a heavy chain constant region encompasses such a constant region lacking a CH1 region. Where the antibody comprises two polypeptides that combine to form an Fc region, it may be that the individual polypeptides comprise charge and/or shape modifications that lead preferentially to the formation of heterodimers and so bring two polypeptides carrying VHH domains for different specificities together, rather than identical polypeptides with VHH domains for the same specificity. Additionally, or alternatively, the constant regions may comprise such modifications that allow the separation of heterodimers from homodimers. In one preferred embodiment, a binding molecule, and in particular an antibody, of the present invention does not comprise a light chain.

Fc domain as employed herein generally refers to —(CH2CH3)₂, unless the context clearly indicates otherwise, where CH2 is the heavy chain CH2 domain, CH3 is the heavy chain CH3 domain, and there are two CH2CH3 with one from each heavy chain.

In one preferred embodiment, a binding molecule, and in particular an antibody, of the present invention does not bind Fc receptors and in particular does not bind to Fc gamma receptors (FcγR). In one preferred embodiment, the binding molecule, and in particular antibody, does not bind to Fc receptors, either because it does not comprise a constant region or alternatively because its Fc region is modified so that it does not bind Fc receptors. In one embodiment, a binding molecule, and in particular an antibody, of the present invention binds to an FcγR, but to a substantially decreased extent relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured). In a particularly preferred embodiment though the binding molecule, and in particular the antibody, has no detectable binding to an FcγR at all.

In one embodiment, where an Fc region is present in an antibody of the present invention, the Fc region employed is mutated, in particular comprising a mutation described herein. In one embodiment the mutation is to remove binding to Fe receptors and in particular FcγR. In one preferred embodiment the antibody has been mutated so that it does not bind Fc receptors. In one embodiment, an antibody may comprise an aglycosylated Fc region, for example to bring about reduced Fc function and in particular a nearly Fc-null phenotype. In one embodiment, an antibody has a modification at N297 and in particular N297A. In one embodiment an antibody has modifications at F243 and/or F244 of the constant region, in particular ones that mean that the antibody comprises a glycosylated constant region. In one embodiment, an antibody may comprise the F243A and/or F244A heavy chain modifications. In another embodiment, one or more of F241, F243, V262 and V264 may be modified and particularly to amino acids that influence glycosylation. In one embodiment, an antibody may have modifications at F241A, F243A, and/or V262E. In one embodiment, it may have the modification V264E. Such modifications are discussed in Yu et al. (2013) Journal of the American Chemical Society, 135 (26): 9723-9732, which is incorporated by reference in its entirety, particularly in relation to the modifications discussed therein. In one particular preferred embodiment, an antibody of the present invention may comprise the LALA modification, Leu234Ala/Leu235Ala. In another particularly preferred embodiment, an antibody of the present invention may comprise the LFLEPS modification, Leu234Phe/Leu235Glu/Pro331/Ser. Further, a binding molecule, in particular an antibody, of the present invention may be produced in a cell type that influences glycosylation as a further approach for sugar engineering. In one embodiment, the fucosylation, sialylation, galactosylation, and/or mannosylation may be altered either by sequence modifications and/or via the type of cell used to produce the binding molecule, and in particular antibody.

In one embodiment, an antibody has modifications at position 297 and/or 299. For example, in one embodiment, an antibody of the present invention comprises a N297A modification in its heavy chains, preferably N297Q or mutation of Ser or Thr at 299 to other residues. In one embodiment it has both those modifications. In one embodiment, an antibody comprises two different heavy chain constant regions where the heavy chain constant regions comprise modifications that allow the different heavy chains to preferentially associate compared to heavy chains associating with identical heavy chains. In one embodiment, the two different heavy chains comprise knob-in-hole mutations. In certain embodiments, the knob-into-hole mutations are a T366W mutation in one heavy chain constant region and a T366S, L368A, and a Y407V mutation in the other domain. In certain embodiments, the modifications comprise charge-pair mutations. In certain embodiments, the charge-pair mutations are a T366K mutation in one of the heavy chain constant regions and a corresponding L351D mutation in the other domain. In an alternative embodiment, rather than have modifications that result in preferential pairing of different heavy chain constant regions the heavy chain constant regions comprise modifications that mean a heterodimer comprising the two heavy chain constant regions can be purified preferentially from the homodimers only comprising one type of heavy chain constant region. For example, the modifications may alter affinity for Protein A, with one heavy chain constant region still able to bind Protein A, whilst the modified heavy chain constant region does not do so, meaning that heterodimers of the two different heavy chains can be purified based on their affinity for Protein A.

In other embodiments, a binding molecule, in particular an antibody, may comprise a modification that changes whether or not a disulfide bridge is formed.

In one embodiment, binding molecules, and in particular antibodies, of the present invention may comprise modifications that alter serum half-life. Hence, in another embodiment, an antibody of the present invention has Fc region modification(s) that alter the half-life of the antibody. Such modifications may be present as well as those that alter Fc functions. In one particularly preferred embodiment, a binding molecule, and in particular an antibody, of the present invention has modification(s) that alter its serum half-life compared to in the absence of such modifications. In one embodiment, the modifications result in increased serum half-life. In another embodiment, they result in decreased serum half-life. In another preferred embodiment, an antibody comprises one or more modifications that collectively both silence the Fc region and decrease the serum half-life of the antibody compared to an antibody lacking such modifications.

Illustrative examples of constant region modifications that may be included in particular embodiments of the invention include:
N297A-Asn297Ala which confers Fc silencing;
LALA-Leu234Ala/Leu235Ala which decreases binding to Fc receptors;
LFLEPS-Leu234Phe/Leu235Glu/Pro331/Ser which decreases binding to Fc receptors;
PG-Pro329Gly which decreases binding to Clq;
LALA-PG-Leu234Ala/Leu235Ala/Pro329Gly which decreases binding to Fc receptors and Clq;
TM-Pro331Ser/Leu234Glu/Leu235Phe-which decreases binding to Fc receptors and Clq;
DA-Asp265Ala-which decreases binding to Fc receptors;
GRLR-Gly236Arg/Leu328Arg-which decreases binding to Fc receptors; and
cFAE-K409R/F405L which promote heterodimer formation.

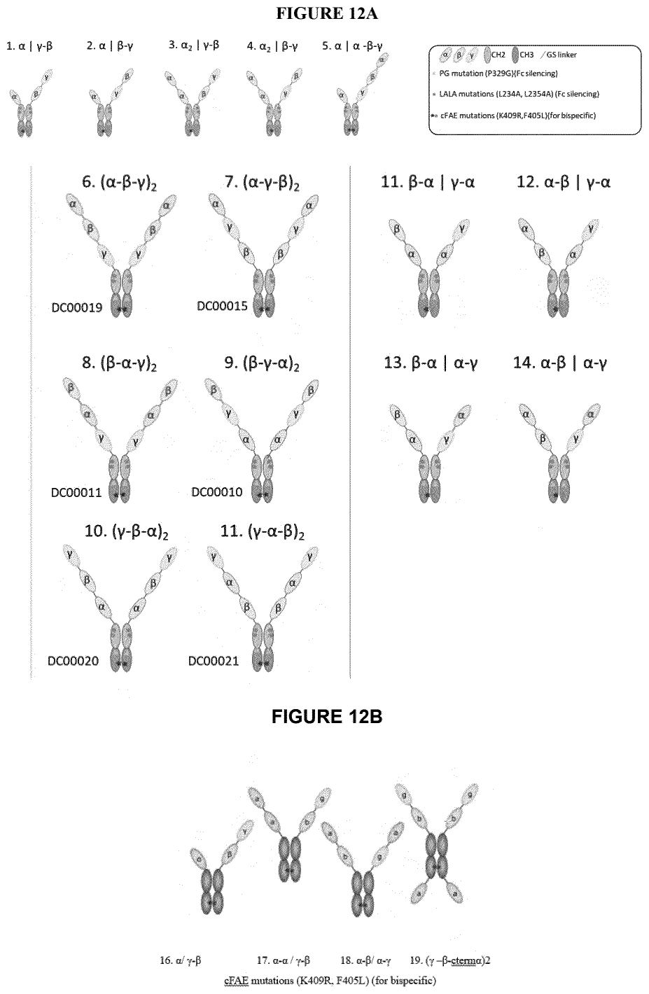

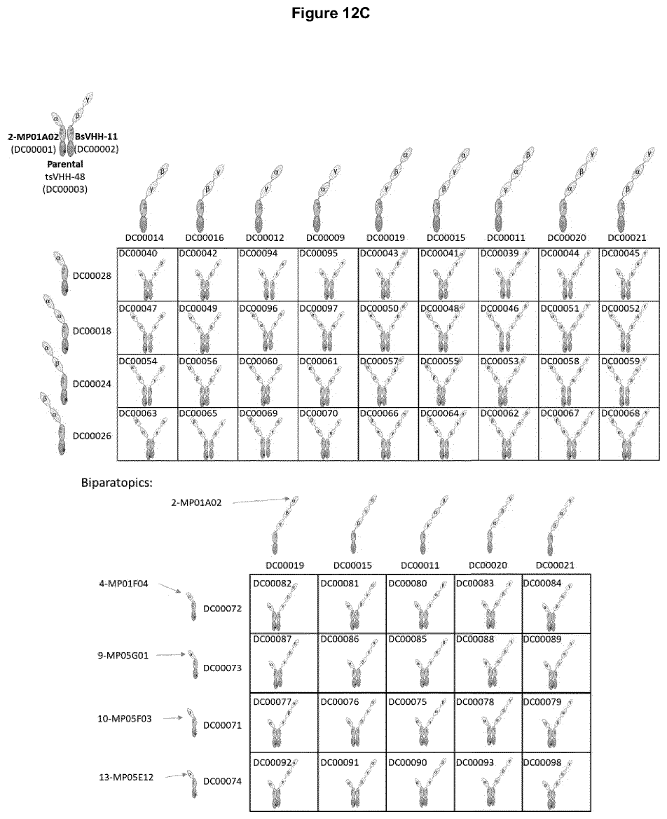

The LALA-PG, and cFAE modifications are particularly preferred, for instance in one embodiment the constant regions will include all of those modifications. In one preferred embodiment, the LALA modifications are present. FIG. 12A also shows examples of modifications that may be employed individually or together with each other in antibodies.

In another embodiment, a binding molecule, particularly an antibody, may lack one of the constant region modifications set out herein.

### Monospecific Binding Molecules and Binding Molecules Comprising VHH Domains

In one embodiment, a binding molecule, particularly an antibody, of the invention is monospecific and recognises just one of the IL-2R polypeptide chains. For example, the present invention provides VHH domains and in one embodiment the binding molecule provided is simply a VHH domain, such as one of those detailed herein. Hence, in one embodiment, the binding molecule provided is an antibody which is monospecific and monovalent, particularly being a VHH domain. In one embodiment, a binding molecule, in particular an antibody, of the present invention may comprise other sequences to the VHH domain, but only includes the VHH domain as a single antigen-binding site.

In one embodiment, the present invention provides a monospecific antibody, comprising, or consisting of, one of the VHH domain antibodies set out in TABLE 3 of the present application or a variant of such a VHH domain antibody. In another embodiment, the present invention provides a monospecific antibody comprising, a set of three CDRs from one of the VHH domain antibodies of TABLE 4 or variants of such CDRs. In another embodiment, the present invention provides a monospecific antibody comprising a VHH domain selected from one of those identified in TABLES 6 to 8 of the present application. In another embodiment, a monospecific antibody is provided comprising a set of three CDRs from one of the VHH domain antibodies identified in Tables 3 and 6 to 8 or a variant set of CDRs. In one embodiment, the invention also provides variants of such antibodies, for instance, where a CDR comprises one, two, three, or four sequence changes compared to the specific sequence set out. In one embodiment, one, two or three CDRs may have such a level of sequence changes.

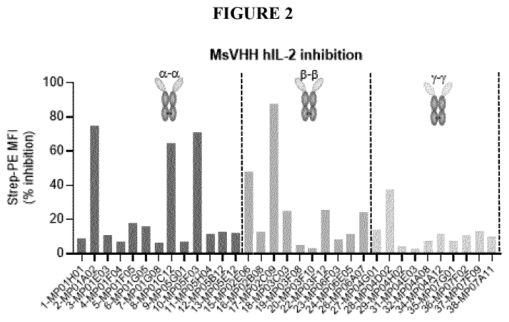

As well as providing the VHH domains individually, the present invention also provides antibodies comprising one or more of the VHH domains as discussed further below. Thus, also provided is an antibody comprising at least one of the VHH domains set out in TABLES 2 and 6 to 8. Further provided is an antibody comprising a CDR set comprising the CDR1, CDR2, and CDR3 of one of the VHH domain antibodies set out in FIGS. 2 and 6 to 8. Antibodies which comprise variants of those VHH domains or CDR sets are further provided.

### Linkers

In a preferred embodiment, binding molecules, in particular antibodies, of the invention comprise more than one antigen-binding site on the same polypeptide. For example, in one embodiment, an antibody of the present invention comprises at least two VHH domains present in the same polypeptide. In one embodiment, an antibody of the present invention comprises two VHH domains on the same polypeptide. In another embodiment, an antibody of the present invention comprises at least three, and preferably three, VHH domains as part of the same polypeptide. In one embodiment, a binding molecule, in particular an antibody, of the present invention may comprise one or more linkers. For instance, a linker may be a non-antibody sequence used to join together different VHH domains and hence aid in providing a polypeptide with several active VHH domains. Any suitable linker may be employed, for instance linkers that are employed in the Examples of the present application or a variant linker sequence.

### Bispecific Binding Molecules

In one embodiment, a binding molecule of the present invention is bispecific binding two different antigens. In one preferred embodiment an antibody of the present invention is a bispecific antibody. For instance, the present invention provides a bispecific binding molecule, in particular a bispecific antibody, that recognises two of the chains of the IL-2R, particularly the IL-2Rβ and γc. In a preferred embodiment, a bispecific antibody provided by the present invention comprises one of the VHH domain antibodies set out in TABLES 2, 6, 7, and 8, more preferably two such VHH domain antibodies. In another preferred embodiment, a bispecific antibody provided by the present invention comprises a set of CDRs from one of the VHH domain antibodies set out in TABLES 3, 6, 7, and 8, more preferably two sets of CDRs from those VHH domain antibodies. The antibody may comprise a variant of those specific sequences, for instance one with one, two, three, or more amino acid sequence changes. In one embodiment, such sequence variations may be in the framework regions, in another they may be in the CDRs themselves.

In one preferred embodiment, one VHH domain, or set of CDRs, is from those in TABLES 2, 3 and 6 and is specific for IL-2Rα. In another preferred embodiment, one VHH domain, or set of CDRs, is from those in TABLES 3 and 7 and is specific for IL-2Rβ. In another embodiment, one VHH domain, or set of CDRs, is from those in TABLES 2, 3 and 8 and is specific for IL-2Rγ. In one embodiment, one VHH domain, or set of CDRs, is from those in TABLES 2, 3 and 7 and is specific for IL-2Rβ and one VHH domain, or set of CDRs, is from those in TABLES 2, 3 and 8 and is specific for IL-2Rγ.

### Trispecific and Multi-Specific Binding Molecules

In one preferred embodiment, a binding molecule, in particular an antibody, of the present invention is multi-specific and so has at least two specificities. In a further preferred embodiment, a binding molecule, in particular an antibody, of the present invention has at least three specificities. In an especially preferred embodiment, a binding molecule, in particular an antibody, of the present invention is trispecific. In particularly preferred embodiment, a binding molecule, in particular an antibody, of the present invention is trispecific with a specificity for each of the three polypeptide chains of the IL-2R, so for the IL-2Rα-, β-, and γ-chains. In another embodiment, the binding molecule, in particular an antibody, has those specificities, plus at least one other specificity as well. For instance, in one embodiment the other specificity is for serum albumin.

Any suitable trispecific format may be used for a trispecific antibody of the present invention and in particular any suitable trispecific antibody format. In one embodiment, the antibody is a single polypeptide chain comprising three VHH domains, with each domain specific for a different IL-2R polypeptide chain, so IL-2Rα, IL-2Rβ, and γc. In one embodiment, the polypeptide also comprises a constant domain, for instance comprising a CH2-CH3 region, and in another a CH1-CH2-CH3. In one instance, the polypeptide also comprises linkers joining together the different VHH domains and optionally to the constant region. The constant region may, for example, include modifications to prevent association with other constant regions to maintain the antibody as a single polypeptide chain. In another embodiment, the antibody does not comprise any constant region and is a single chain polypeptide.

In one particularly preferred embodiment, a binding molecule, in particular an antibody, of the present invention comprises two polypeptides. For instance, an antibody may comprise two polypeptide chains with a constant region to allow the two polypeptide chains to associate. In one preferred embodiment, such a two-polypeptide antibody is a trispecific antibody or is trispecific for the three different IL-2R polypeptide chains and may also comprise other specificities. Any combination of antigen binding sites giving the required trispecificity may be employed. For example, in a preferred embodiment, one polypeptide chain comprises a VHH domain specific for one of the IL-2R receptor polypeptides, with the other polypeptide chain comprising two VHH domains for the specificities of the other two IL-2R polypeptide chains. So, for example, an antibody may have the format a/b-g where “a” denotes a VHH with specificity for IL-2Rα, “b” denotes a VHH with specificity for IL-2Rβ, “g” denotes a VHH with specificity for γc, and “/” denotes the changeover from the first to second polypeptide being defined, where within a polypeptide the VHH domains are defined in N to C-terminal order. Where a polypeptide chain has a binding domain or binding domains at the C terminus of a constant region in a polypeptide, the binding domain or domains may be denoted by cterm-a, cterm-a-a and so on. The “-” may be a linker or simply denote joining of the VHH domains to each other. Examples of possible formats that may be employed which include one VHH on one polypeptide and two VHHs on the other include: a/b-g; a/g-b; b/a-g; b/g-a; g/a-b; and g/b-a. In another embodiment, an antibody of the present invention comprises two polypeptides where each polypeptide comprises two VHH domains, with collectively the two polypeptides comprising at least one VHH specific for each IL-2R chain. That may mean, for example, that collectively for one of the IL-2R polypeptides there are two VHH domains present in that antibody that are specific for that IL-2R polypeptide chain. So, examples of possible formats that may be include, using the numbering system discussed above: a-a/b-g; a-a/g-b; b-b/a-g; b-b/g-a; g-g/a-b; and g-g/b-a. Some of the Figures of the present application use the Greek symbols α β γ but the structures may also be set out using the equivalent “a”, “b”, and “c” format, or using the equivalent “alpha”, “beta”, and “gamma” format, or using the equivalent “CD25”, “CD122”, and “CD132” format as well.

In a further embodiment, an antibody of the present invention may comprise two polypeptides where:
one polypeptide chain comprises at least two VHH domains, where the at least two VHH domains include two VHHs specific for different IL-2R polypeptides; and
the other polypeptide chain comprises at least one VHH,
where collectively the two polypeptides have at least one VHH for each of the three IL-2R polypeptides. In one embodiment, each polypeptide comprises three VHH domains.

In one embodiment of the present invention has a valency of one, two, or three for one of the IL-2R polypeptides, where the antibody also has binding sites for each of the other two IL-2R polypeptides. In one embodiment, all of the antigen binding sites on one polypeptide have the same specificity, with the other polypeptide providing the antigen binding sites specific for the other two IL-2R polypeptides.

FIG. 12A and FIG. 12B and FIG. 12C of the present application provides illustrative examples of possible trispecific antibody formats comprising VHH domains, where collectively there is at least one VHH specific for each IL-2R polypeptide. An antibody of the present invention may be provided in any of those formats.

In any of the above discussed formats, at least one additional VHH may be present which is specific for something other than an IL-2R polypeptide, for instance, a VHH specific for serum albumin may be present. Any of the different antibody formats discussed herein may be employed with any of the heavy chain Fc region modifications discussed herein, examples of preferred modifications which may be present include those shown in FIG. 12A to 12C.

### Illustrative Antibodies and VHH Domains of the Present Invention

TABLE 3 provides examples of particularly preferred VHH domain antibodies of the present invention, with TABLE 4 providing the CDR sequences for those VHH domains. Those VHH domains may be, for instance, employed in any of the antibody formats discussed herein, as may be CDR sets from those VHHs, and variants of either.

TABLE 7 provides examples of further preferred VHH domains specific for IL-2Rα polypeptide and CDR sets from them that may be employed in any of the antibody formats discussed herein, as may be variants of them. TABLE 8 provides examples of further preferred VHH domains specific for IL-2Rβ polypeptide and CDR sets from them that may be employed in any of the antibody formats discussed herein, as may be variants of them. TABLE 9 provides examples of further preferred VHH domains specific for γc polypeptide and CDR sets from them that may be employed in any of the antibody formats discussed herein, as may be variants of them.

Hence, the present invention provides an antibody comprising any of those VHH domains. It also provides an antibody comprising any of those CDR sets. Also provided is an antibody comprising a variant of those. In one embodiment, an antibody is provided comprising one or at least one of those VHH domains/CDR sets/or variants thereof, in another an antibody comprising at least three of those. In a particularly preferred embodiment, an antibody comprising three of those VHH domains/CDR sets/or variants thereof is provided.

TABLE 5 provides examples of particularly preferred multi-specific antibodies and those form preferred embodiments of the invention, as do variants of them.

### Further Examples of Preferred Binding Molecules

In one preferred embodiment, a binding molecule of the present invention consists of, or comprises, a VHH domain against IL-2Rα selected from the group consisting of that of SEQ ID NOs: 2, 3, 4, 8 and 10. In one embodiment, rather than comprising the whole VHH, a binding molecule of the invention comprises a set of CDR1, CDR2, and CDR3 from one of those VHHs. In one embodiment, the employed sequence is a variant of any of those sequences which is still able to bind IL-2Rα.

In one preferred embodiment, a binding molecule of the present invention consists of, or comprises, a VHH domain against IL-2Rβ selected from the group consisting of that of SEQ ID NOs: 16, 18, 19, 22 and 26. In one embodiment, rather than comprising the whole VHH, a binding molecule of the invention comprises a set of CDR1, CDR2, and CDR3 from one of those VHHs. In one embodiment, the employed sequence is a variant of any of those sequences which is still able to bind IL-2Rβ.

In one preferred embodiment, a binding molecule of the present invention consists of, or comprises, a VHH domain against γc selected from the group consisting of that of SEQ ID NOs: 27, 31, 32, 35 and 36. In one embodiment, rather than comprising the whole VHH, a binding molecule of the invention comprises a set of CDR1, CDR2, and CDR3 from one of those VHHs. In one embodiment, the employed sequence is a variant of any of those sequences which is still able to bind γc.

In one particularly preferred embodiment, a binding molecule of the present invention comprises the three VHH domains of SEQ IDs 2, 19, and 27. In another preferred embodiment, the binding molecule comprises the CDRs sets of each of SEQ IDs 2, 19, and 27. In one preferred embodiment, the binding molecule has the structure a2/g27-b19 where a2, g27, and b19 represent respectively SEQ ID NOs 2, 27 and 19. In other embodiments, the antibody has the structure g27/a2-b19. In another embodiment, it has the structure g27/b19-a2. In another embodiment, it has the structure b19/a2-g27. In another embodiment, it has the structure b19/g27-a2. Variants of such sequences may also be employed. For example,

In one preferred embodiment, a binding domain of the present invention comprises the three VHH domains of SEQ IDs 3, 22, and 36. In another preferred embodiment, the binding molecule comprises the CDRs sets of each of SEQ IDs 3, 22, and 36. In one preferred embodiment, the binding molecule has the structure a3/g36-b22 where a3, g36, and b22 represent respectively SEQ ID NOs 3, 36 and 22. In other embodiments, the antibody has the structure g36/a3-b22. In another embodiment, it has the structure g36/b22-a3. In another embodiment, it has the structure b22/a3-g36. In another embodiment, it has the structure b22/g36-a3. Variants of such sequences may also be employed.

In one preferred embodiment, a binding domain of the present invention comprises the three VHH domains of SEQ IDs 4, 16, and 36. In another preferred embodiment, the binding molecule comprises the CDRs sets of each of SEQ IDs 4, 16, and 36. In one preferred embodiment, the binding molecule has the structure a4/g36-b16 where a4, g36, and b16 represent respectively SEQ ID NOs 4, 36 and 16. In other embodiments, the antibody has the structure g36/a4-b16. In another embodiment, it has the structure g36/b16-a4. In another embodiment, it has the structure b16/a4-g36. In another embodiment, it has the structure b16/g36-a4. Variants of such sequences may also be employed.

In one preferred embodiment, a binding domain of the present invention comprises the three VHH domains of SEQ IDs 3, 18, and 27. In another preferred embodiment, the binding molecule comprises the CDRs sets of each of SEQ IDs 3, 18, and 27. In one preferred embodiment, the binding molecule has the structure a3/g27-b18 where a3, g27, and b18 represent respectively SEQ ID NOs 3, 27, and 18. In other embodiments, the antibody has the structure g27/a3-b18. In another embodiment, it has the structure g27/b18-a3. In another embodiment, it has the structure b18/a3-g27. In another embodiment, it has the structure b18/g27-a3. Variants of such sequences may also be employed.

In one embodiment, a binding molecule of the present invention comprises one of the SEQ ID Nos set out in TABLE 5. In one embodiment the binding molecule comprises the VHH sequences of SEQ ID Nos: 32 and 16. In another, those of SEQ ID NOs: 35 and 16. In another, those of SEQ ID NOs: 36 and 16. In another, those of SEQ ID NOs: 27 and 18. In another, those of SEQ ID NOs: 31 and 18. In another, those of SEQ ID NOs: 32 and 18. In another, those of SEQ ID NOs: 35 and 18. In an alternative embodiment, rather than comprise those VHHs it may comprise the two CDR sets from them. It may also be a variant of such sequences. In one embodiment, the binding molecule may also comprise one of SEQ ID NOs 1 to 38 which is not those mentioned above as an additional VHH or it may comprise a CDR set from such VHH.

In one embodiment, a binding molecule of the present invention employs one of the VHHs or combination of VHHs employed in the Examples of this application. In another embodiment, it employs a CDR set or sets from those employed in the Examples of this application. Any of the other features set out in here may also be employed in addition to the VHHs employed in the Examples.

FIG. 12A and FIG. 12B provide examples of preferred formats for a trispecific antibodies which each bind all three IL-2R chains and those formats are discussed further below in the following number preferred embodiments where “a” denotes a binding domain with specificity of the IL-2a, “b” for the IL-2Rβ, and “g” for the γc, “/” denotes the swap over from one polypeptide to the next, “-” denotes the binding domains being joined by a linker or simply joined, “cterm” denotes the presence of binding domains at the C terminus of the constant region, and each polypeptide comprises one of the two polypeptide sequences so the polypeptides can associate to form an Fc region, so typically CH2CH3. For each of the further embodiments set out below, preferably, the antibody has the LALA mutations, N297A mutation, and/or the cFAE mutations in the Fc region. More preferably the antibody has all of the LALA mutations, PG mutation, and the cFAE mutations. In another embodiment, the binding molecule has the LALA mutations, PG mutation, and knob-in-hole modifications (such as any set out herein). The further numbered embodiments of trispecific antibodies binding all three IL-2R polypeptide chains being:
1. A trispecific antibody having the format a/g-b, which comprises an Fc region. Preferably, where the antibody has the structure of structure 1 in FIG. 12A.
2. A trispecific antibody having the format a/b-g, which comprises an Fc region.
Preferably, where the antibody has the structure of structure 2 in FIG. 12A.
3. A trispecific antibody having the format a-a/g-b, which comprises an Fc region. Preferably, where the antibody has the structure of structure 3 in FIG. 12A.
4. A trispecific antibody having the format a-a/b-g, which comprises an Fc region. Preferably, where the antibody has the structure of structure 4 in FIG. 12A.
5. A trispecific antibody having the format a/a-b-g, which comprises an Fc region. Preferably, where the antibody has the structure of structure 5 in FIG. 12A.
6. A trispecific antibody having the format a-b-g/a-b-g, which comprises an Fc region. Preferably, where the antibody has the structure of structure 6 in FIG. 12B.
7. A trispecific antibody having the format a-g-b/a-g-b, which comprises an Fc region. Preferably, where the antibody has the structure of structure 7 in FIG. 12B.
8. A trispecific antibody having the format b-a-g/b-a-g, which comprises an Fc region. Preferably, where the antibody has the structure of structure 8 in FIG. 12B.
9. A trispecific antibody having the format b-g-a/b-g-a, which comprises an Fc region. Preferably, where the antibody has the structure of structure 9 in FIG. 12B.
10. A trispecific antibody having the format g-b-a/g-b-a, which comprises an Fc region. Preferably, where the antibody has the structure of structure 10 in FIG. 12B.
11. A trispecific antibody having the format g-a-b/g-a-b, which comprises an Fc region. Preferably, where the antibody has the structure of structure 11 in FIG. 12B.
12. A trispecific antibody having the format b-a/g-a, which comprises an Fc region. Preferably, where the antibody has the structure of structure 12 in FIG. 12B.
13. A trispecific antibody having the format a-b/g-a, which comprises an Fc region. Preferably, where the antibody has the structure of structure 13 in FIG. 12B.
14. A trispecific antibody having the format b-a/a-g, which comprises an Fc region. Preferably, where the antibody has the structure of structure 14 in FIG. 12B.
15. A trispecific antibody having the format a-b/a-g, which comprises an Fc region. Preferably, where the antibody has the structure of structure 15 in FIG. 12B.
16. A trispecific antibody having the format g-b-cterm-a/g-b-cterm-a, which comprises an Fc region. Preferably, where the antibody has the structure of structure 16 in FIG. 12B.
17. An antibody having one of the formats shown in FIG. 12C. Preferably, wherein the antibody has one of the structures shown in FIG. 12C with optionally Fc mutations also present.

In one embodiment any of the formats discussed above in further embodiments 1 to 17 are provided comprising one of the specific VHH domains against IL-2Rα described herein. In one embodiment, all of the VHH domains against IL-2Rα present are that specific VHH domain. In one embodiment any of the formats discussed above in further embodiments 1 to 17 are provided comprising one of the specific VHH domains against IL-2Rβ described herein. In one embodiment, all of the VHH domains against IL-2Rβ present are that specific VHH domain. In one embodiment any of the formats discussed above in further embodiments 1 to 17 are provided comprising one of the specific VHH domains against IL-2Ry described herein. In one embodiment, any of the formats discussed above in further embodiments 1 to 17 are provided comprising a combination of specific VHH domains set out herein, for instance in the sense that all of the VHH domain(s) against IL-2Rα, IL-2Rβ, and ye are those used as a combination of VHH domains set out herein. Also provided are trispecific antibodies of the formats set out in numbered embodiments 1 to 17, where the combination of VHH domains providing specificities for IL-2Rβ, IL-2Rβ, and γc is one of the combinations set out herein, even where set out for an antibody of a different format. In one embodiment a combination of VHH domains or CDR sets used in the Examples of the present application is employed in a format as set out in one of numbered embodiments 1 to 17 set out above.

FIG. 12C, top Table shows examples of particularly preferred formats. The a, b, and g VHH subunits respectively correspond to those present in the parental tsVHH-48, but in different permutations as depicted in the Table. Hence, the VHH corresponding to VHH “a” is that of SEQ ID NO: 2. The VHH corresponding to VHH “b” is that of SEQ ID NO: 19. The VHH corresponding to VHH “g” is that of SEQ ID NO: 27.

Hence, in one embodiment the antibody has the format a/b-g. A preferred such antibody is DC00040 or a variant thereof. In another embodiment, the antibody is in the format a/g-b. A preferred such antibody is DC00042 or a variant thereof. In another embodiment, the antibody is in the format a/a-g. A preferred such antibody is DC00094. In another embodiment, the antibody is in the format a/g-a. A preferred such antibody is DC00095. In another embodiment, the antibody is in the format a/a-b-g. A preferred such antibody is DC00043. In another embodiment, the antibody is in the format a/a-g-b. A preferred such antibody is DC00041. In another embodiment, the antibody is in the format a/b-a-g. A preferred such antibody is DC00039. In another embodiment, the antibody is in the format a/g-b-a. A preferred such antibody is DC00044. In another embodiment, the antibody is in the format a/g-a-b. A preferred such antibody is DC00045.

In another embodiment, the antibody is in the format a-a/b-g. A preferred such antibody is DC00047. In another embodiment, the antibody is in the format a-a/g-b. A preferred such antibody is DC00049. In another embodiment, the antibody is in the format a-a/a-g. A preferred such antibody is DC00096. In another embodiment, the antibody is in the format a-a/g-a. A preferred such antibody is DC00097. In another embodiment, the antibody is in the format a-a/a-b-g. A preferred such antibody is DC00050. In another embodiment, the antibody is in the format a-a/a-g-b. A preferred such antibody is DC00048. In another embodiment, the antibody is in the format a-a/b-a-g. A preferred such antibody is DC00046. In another embodiment, the antibody is in the format a-a/g-b-a. A preferred such antibody is DC00051. In another embodiment, the antibody is in the format a-a/g-a-b. A preferred such antibody is DC00052.

In another embodiment, the antibody is in the format a-b/b-g. A preferred such antibody is DC00054. In another embodiment, the antibody is in the format a-b/g-b. A preferred such antibody is DC00056. In another embodiment, the antibody is in the format a-b/a-g. A preferred such antibody is DC00060. In another embodiment, the antibody is in the format a-b/g-a. A preferred such antibody is DC00061. In another embodiment, the antibody is in the format a-b/a-b-g. A preferred such antibody is DC00057. In another embodiment, the antibody is in the format a-b/a-g-b. A preferred such antibody is DC00055. In another embodiment, the antibody is in the format a-b/b-a-g. A preferred such antibody is DC00053. In another embodiment, the antibody is in the format a-b/g-b-a. A preferred such antibody is DC00058. In another embodiment, the antibody is in the format a-b/g-a-b. A preferred such antibody is DC00059.

In another embodiment, the antibody is in the format b-a/b-g. A preferred such antibody is DC00063. In another embodiment, the antibody is in the format b-a/g-b. A preferred such antibody is DC00065. In another embodiment, the antibody is in the format b-a/a-g. A preferred such antibody is DC00069. In another embodiment, the antibody is in the format b-a/g-a. A preferred such antibody is DC00070. In another embodiment, the antibody is in the format b-a/a-b-g. A preferred such antibody is DC00066. In another embodiment, the antibody is in the format b-a/a-g-b. A preferred such antibody is DC00064. In another embodiment, the antibody is in the format b-a/b-a-g. A preferred such antibody is DC00062. In another embodiment, the antibody is in the format b-a/g-b-a. A preferred such antibody is DC00067. In another embodiment, the antibody is in the format b-a/g-a-b. A preferred such antibody is DC00068.

FIG. 12C, bottom Table shows further examples of particularly preferred formats, with the formats depicted being biparatopic with respect to IL-2Rα. One of the VHHs against IL-2Rα is that from tsVHH-48 so that of SEQ ID NO: 2, with the top of the Table showing the polypeptides including that VHH. The other polypeptide making up the overall antibody is shown on the left hand side, with it being that of SEQ ID NO: 4. SEQ ID NO: 9, SEQ ID NO: 10, or SEQ ID NO: 13. Hence preferred binding molecules include a binding molecule with a geometry set out in the bottom Table with the VHH domains indicated. Thus a preferred binding molecule is one with the VHH domains and geometry of one of DC00082, DC00081, DC00080, DC00083, DC00084. Further preferred binding molecules include one with the VHH domains and geometry of one of DC00087, DC00086, DC00085, DC00088, or DC00089. Further preferred binding molecules include one with the VHH domains and geometry of one of DC00077, DC00076, DC00075, DC00078, or DC00079. Further preferred binding molecules include one with the VHH domains and geometry of one of DC00092, DC00091, DC00090, DC00093, or DC00098.

The present invention further provides variants of the binding molecules discussed above for FIG. 12C provided that the variants remain functional.

Further preferred embodiments include the following:
1. A binding molecule comprising a polypeptide comprising an amino acid sequence selected from SEQ ID NO: 2043, 2045, 2039, 2047, 2044, 2041, 2048, 2049 and a variant of any thereof which still retains the ability to bind to the relevant IL-2 α, β or γc chain or chains.
2. A binding molecule comprising: (i) a polypeptide comprising an amino acid sequence selected from SEQ ID NO: 2052 and a variant of any thereof which still retains the ability to bind to IL-2 a chain; and (ii) a polypeptide comprising an amino acid sequence selected from SEQ ID NO: 243, 2045, 2039, 2047, 2044, 2041, 2048, 2049 and a variant of any thereof which still retains the ability to bind to the relevant IL-2 α, β or γc chain or chains.
3. A binding molecule comprising: (i) a polypeptide comprising an amino acid sequence selected from SEQ ID NO: 2046 and a variant of any thereof which still retains the ability to bind to IL-2 a chain; and (ii) a polypeptide comprising an amino acid sequence selected from SEQ ID NO: 243, 2045, 2039, 2047, 2044, 2041, 2048, 2049 or a variant of any thereof which still retains the ability to bind to the relevant IL-2 α, β or γc chain or chains.
4. A binding molecule comprising: (i) a polypeptide comprising an amino acid sequence selected from SEQ ID NO: 2050 and a variant of any thereof which still retains the ability to bind to IL-2 a chain; and (ii) a polypeptide comprising an amino acid sequence selected from SEQ ID NO: 243, 2045, 2039, 2047, 2044, 2041, 2048, 2049 and a variant of any thereof which still retains the ability to bind to the relevant IL-2 α, β or γc chain or chains.
5. A binding molecule comprising: (i) a polypeptide comprising an amino acid sequence selected from SEQ ID NO: 2051 and a variant of any thereof which still retains the ability to bind to IL-2 a chain; and (ii) a polypeptide comprising an amino acid sequence selected from SEQ ID NO: 243, 2045, 2039, 2047, 2044, 2041, 2048, 2049 and a variant of any thereof which still retains the ability to bind to the relevant IL-2 α, β or γc chain or chains.
6. A binding molecule comprising (i) a polypeptide comprising an amino acid sequence selected from SEQ ID NO: 2052, 2046, 2050, 2051, and a variant of any thereof which still retains the ability to bind to IL-2 a chain; and (ii) a polypeptide comprising an amino acid sequence selected from SEQ ID NO: 243 and a variant of any thereof which still retains the ability to bind to the relevant IL-2 a, B or Yc chain or chains.
7. A binding molecule comprising: (i) a polypeptide comprising an amino acid sequence selected from SEQ ID NO: 2052, 2046, 2050, 2051 and a variant of any thereof which still retains the ability to bind to IL-2 a chain; and (ii) a polypeptide comprising an amino acid sequence selected from SEQ ID NO: 2045 and a variant of any thereof which still retains the ability to bind to the relevant IL-2 α, β or γc chain or chains.
8. A binding molecule comprising: (i) a polypeptide comprising an amino acid sequence selected from SEQ ID NO: 2052, 2046, 2050, 2051 and a variant of any thereof which still retains the ability to bind to IL-2 a chain; and (ii) a polypeptide comprising an amino acid sequence selected from SEQ ID NO: 2042 and a variant of any thereof which still retains the ability to bind to the relevant IL-2 α, β or γc chain or chains.
9. A binding molecule comprising: (i) a polypeptide comprising an amino acid sequence selected from SEQ ID NO: 2052, 2046, 2050, 2051 and a variant of any thereof which still retains the ability to bind to IL-2 a chain; and (ii) a polypeptide comprising an amino acid sequence selected from SEQ ID NO: 2039 and a variant of any thereof which still retains the ability to bind to the relevant IL-2 α, β or γc chain or chains.
10. A binding molecule comprising: (i) a polypeptide comprising an amino acid sequence selected from SEQ ID NO: 2052, 2046, 2050, 2051 and a variant of any thereof which still retains the ability to bind to IL-2 a chain; and (ii) a polypeptide comprising an amino acid sequence selected from SEQ ID NO: 2047 and a variant of any thereof which still retains the ability to bind to the relevant IL-2 α, β or γc chain or chains.
11. A binding molecule comprising: (i) a polypeptide comprising an amino acid sequence selected from SEQ ID NO: 2052, 2046, 2050, 2051 and a variant of any thereof which still retains the ability to bind to IL-2 a chain; and (ii) a polypeptide comprising an amino acid sequence selected from SEQ ID NO: 2044 and a variant of any thereof which still retains the ability to bind to the relevant IL-2 α, β or γc chain or chains.
12. A binding molecule comprising: (i) a polypeptide comprising an amino acid sequence selected from SEQ ID NO: 2052, 2046, 2050, 2051 and a variant of any thereof which still retains the ability to bind to IL-2 a chain; and (ii) a polypeptide comprising an amino acid sequence selected from SEQ ID NO: 2048, and a variant of any thereof which still retains the ability to bind to the relevant IL-2 α, β or γc chain or chains.
13. A binding molecule comprising: (i) a polypeptide comprising an amino acid sequence selected from SEQ ID NO: 2052, 2046, 2050, 2051 and a variant of any thereof which still retains the ability to bind to IL-2 α chain; and (ii) a polypeptide comprising an amino acid sequence selected from SEQ ID NO: 2041 and a variant of any thereof which still retains the ability to bind to the relevant IL-2 α, β or γc chain or chains.
14. A binding molecule comprising: (i) a polypeptide comprising an amino acid sequence selected from SEQ ID NO: 2052, 2046, 2050, 2051 and a variant of any thereof which still retains the ability to bind to IL-2 α chain; and (ii) a polypeptide comprising an amino acid sequence selected from SEQ ID NO: 2048 and a variant of any thereof which still retains the ability to bind to the relevant IL-2 α, β or γc chain or chains.
15. A binding molecule comprising: (i) a polypeptide comprising an amino acid sequence selected from SEQ ID NO: 2052, 2046, 2050, 2051 and a variant of any thereof which still retains the ability to bind to IL-2 α chain; and (ii) a polypeptide comprising an amino acid sequence selected from SEQ ID NO: 2049 and a variant of any thereof which still retains the ability to bind to the relevant IL-2 α, β or γc chain or chains.
16. A binding molecule comprising a polypeptide comprising an amino acid sequence selected from SEQ ID NO: 2052, 2046, 2050, 2051, and a variant of any thereof which still retains the ability to bind to the relevant IL-2 α, β or γc chain or chains.

The following represent further preferred embodiments:
(a) A binding molecule comprising a polypeptide comprising an amino acid sequence selected from SEQ ID NO: 2054, 2055, 2053, 2056, and a variant of any thereof which still retains the ability to bind IL-2 α.
(b) A binding molecule comprising a polypeptide comprising an amino acid sequence selected from SEQ ID NO: 2047, 2044, 2041, 2048, 2049, and a variant of any thereof which still retains the ability to bind IL-2 α, β or γc chain or chains.
(c) A binding molecule binding molecule comprising: (i) a polypeptide comprising an amino acid sequence selected from SEQ ID NO: 2054, 2055, 2053, 2056, and a variant of any thereof which still retains the ability to bind to the IL-2 α chain; and (ii) a polypeptide comprising an amino acid sequence selected from SEQ ID NO: 2047, and a variant of any thereof which still retains the ability to bind to the relevant IL-2 α, β or γc chain or chains.
(d) A binding molecule binding molecule comprising: (i) a polypeptide comprising an amino acid sequence selected from SEQ ID NO: 2054, 2055, 2053, 2056, and a variant of any thereof which still retains the ability to bind to the IL-2 α chain; and (ii) a polypeptide comprising an amino acid sequence selected from SEQ ID NO: 2044, and a variant of any thereof which still retains the ability to bind to the relevant IL-2 α, B or Yc chain or chains.
(e) A binding molecule binding molecule comprising: (i) a polypeptide comprising an amino acid sequence selected from SEQ ID NO: 2054, 2055, 2053, 2056, and a variant of any thereof which still retains the ability to bind to the IL-2 α chain; and (ii) a polypeptide comprising an amino acid sequence selected from SEQ ID NO: 2041, and a variant of any thereof which still retains the ability to bind to the relevant IL-2 α, β or γc chain or chains.
(f) A binding molecule binding molecule comprising: (i) a polypeptide comprising an amino acid sequence selected from SEQ ID NO: 2054, 2055, 2053, 2056, and a variant of any thereof which still retains the ability to bind to the IL-2 α chain; and (ii) a polypeptide comprising an amino acid sequence selected from SEQ ID NO: 2048, and a variant of any thereof which still retains the ability to bind to the relevant IL-2 α, β or γc chain or chains.
(g) A binding molecule binding molecule comprising: (i) a polypeptide comprising an amino acid sequence selected from SEQ ID NO: 2054, 2055, 2053, 2056, and a variant of any thereof which still retains the ability to bind to the IL-2 α chain; and (ii) a polypeptide comprising an amino acid sequence selected from SEQ ID NO: 2049, and a variant of any thereof which still retains the ability to bind to the relevant IL-2 α, β or γc chain or chains.
(h) A binding molecule binding molecule comprising: (i) a polypeptide comprising an amino acid sequence selected from SEQ ID NO: 2054, and a variant of any thereof which still retains the ability to bind to the IL-2 α chain; and (ii) a polypeptide comprising an amino acid sequence selected from SEQ ID NO: 2047, 2044, 2041, 2048, 2049, and a variant of any thereof which still retains the ability to bind to the relevant IL-2 α, β or γc chain or chains.
(i) A binding molecule binding molecule comprising: (i) a polypeptide comprising an amino acid sequence selected from SEQ ID NO: 2055, and a variant of any thereof which still retains the ability to bind to the IL-2 α chain; and (ii) a polypeptide comprising an amino acid sequence selected from SEQ ID NO: 2047, 2044, 2041, 2048, 2049, and a variant of any thereof which still retains the ability to bind to the relevant IL-2 α, β or γc chain or chains.
(j) A binding molecule binding molecule comprising: (i) a polypeptide comprising an amino acid sequence selected from SEQ ID NO: 2053, and a variant of any thereof which still retains the ability to bind to the IL-2 α chain; and (ii) a polypeptide comprising an amino acid sequence selected from SEQ ID NO: 2047, 2044, 2041, 2048, 2049, and a variant of any thereof which still retains the ability to bind to the relevant IL-2 α, β or γc chain or chains.
(k) A binding molecule binding molecule comprising: (i) a polypeptide comprising an amino acid sequence selected from SEQ ID NO: 2056, and a variant of any thereof which still retains the ability to bind to the IL-2 α chain; and (ii) a polypeptide comprising an amino acid sequence selected from SEQ ID NO: 2047, 2044, 2041, 2048, 2049, and a variant of any thereof which still retains the ability to bind to the relevant IL-2 α, β or γc chain or chains.

Examples of other preferred embodiments include the following pairwise combinations of polypeptides: (1) SEQ ID NO 2052 and SEQ ID NO: 2043; (2) SEQ ID NO 2052 and SEQ ID NO: 2045; (3) SEQ ID NO 2052 and SEQ ID NO: 2042; (4) SEQ ID NO 2052 and SEQ ID NO: 2039; (5) SEQ ID NO 2052 and SEQ ID NO: 2047; (6) SEQ ID NO 2052 and SEQ ID NO: 2044; (7) SEQ ID NO 2052 and SEQ ID NO: 2041; (8) SEQ ID NO 2052 and SEQ ID NO: 2048; SEQ ID NO 2052 and SEQ ID NO: 2049; (10) a variant of any of (1) to (9).

Examples of other preferred embodiments include the following pairwise combinations of polypeptides: (1) SEQ ID NO 2046 and SEQ ID NO: 2043; (2) SEQ ID NO 2046 and SEQ ID NO: 2045; (3) SEQ ID NO 2046 and SEQ ID NO: 2042; (4) SEQ ID NO 2046 and SEQ ID NO: 2039; (5) SEQ ID NO 2046 and SEQ ID NO: 2047; (6) SEQ ID NO 2046 and SEQ ID NO: 2044; (7) SEQ ID NO 2046 and SEQ ID NO: 2041; (8) SEQ ID NO 2046 and SEQ ID NO: 2048; SEQ ID NO 2046 and SEQ ID NO: 2049; (10) a variant of any of (1) to (9).

Examples of other preferred embodiments include the following pairwise combinations of polypeptides: (1) SEQ ID NO 2050 and SEQ ID NO: 2043; (2) SEQ ID NO 2050 and SEQ ID NO: 2045; (3) SEQ ID NO 2050 and SEQ ID NO: 2042; (4) SEQ ID NO 2050 and SEQ ID NO: 2039; (5) SEQ ID NO 2050 and SEQ ID NO: 2047; (6) SEQ ID NO 2050 and SEQ ID NO: 2044; (7) SEQ ID NO 2050 and SEQ ID NO: 2041; (8) SEQ ID NO 2050 and SEQ ID NO: 2048; SEQ ID NO 2050 and SEQ ID NO: 2049; (10) a variant of any of (1) to (9).

Examples of other preferred embodiments include the following pairwise combinations of polypeptides: (1) SEQ ID NO 2051 and SEQ ID NO: 2043; (2) SEQ ID NO 2051 and SEQ ID NO: 2045; (3) SEQ ID NO 2051 and SEQ ID NO: 2042; (4) SEQ ID NO 2051 and SEQ ID NO: 2039; (5) SEQ ID NO 2051 and SEQ ID NO: 2047; (6) SEQ ID NO 2051 and SEQ ID NO: 2044; (7) SEQ ID NO 2051 and SEQ ID NO: 2041; (8) SEQ ID NO 2051 and SEQ ID NO: 2048; SEQ ID NO 2051 and SEQ ID NO: 2049; (10) a variant of any of (1) to (9).

Examples of other preferred embodiments include the following pairwise combinations of polypeptides: (1) SEQ ID NO 2054 and SEQ ID NO: 2047; (2) SEQ ID NO 2054 and SEQ ID NO: 2044; (3) SEQ ID NO 2054 and SEQ ID NO: 2041; (4) SEQ ID NO 2054 and SEQ ID NO: 2048; (5) SEQ ID NO 2054 and SEQ ID NO: 2049; (6) a variant of any of (1) to (3).

Examples of other preferred embodiments include the following pairwise combinations of polypeptides: (1) SEQ ID NO 2055 and SEQ ID NO: 2047; (2) SEQ ID NO 2055 and SEQ ID NO: 2044; (3) SEQ ID NO 2055 and SEQ ID NO: 2041; (4) SEQ ID NO 2055 and SEQ ID NO: 2048; (5) SEQ ID NO 2055 and SEQ ID NO: 2049; (6) a variant of any of (1) to (5).

Examples of other preferred embodiments include the following pairwise combinations of polypeptides: (1) SEQ ID NO 2053 and SEQ ID NO: 2047; (2) SEQ ID NO 2053 and SEQ ID NO: 2044; (3) SEQ ID NO 2053 and SEQ ID NO: 2041; (4) SEQ ID NO 2053 and SEQ ID NO: 2048; (5) SEQ ID NO 2053 and SEQ ID NO: 2049; (6) a variant of any of (1) to (5).

Examples of other preferred embodiments include the following pairwise combinations of polypeptides: (1) SEQ ID NO 2056 and SEQ ID NO: 2047; (2) SEQ ID NO 2056 and SEQ ID NO: 2044; (3) SEQ ID NO 2056 and SEQ ID NO: 2041; (4) SEQ ID NO 2056 and SEQ ID NO: 2048; (5) SEQ ID NO 2056 and SEQ ID NO: 2049; (6) a variant of any of (1) to (5).

In a further preferred embodiment, a binding molecule of the present invention comprises the CDR sets or variant versions thereof or the tsVHH-48 antibody shown in FIG. 12C. In another embodiment, it comprises the VHH domains of the tsVHH-48 antibody or variant versions thereof. In one embodiment, a binding molecule comprises a polypeptide which is monovalent selected from SEQ ID NOs: 2052, 2053, 2054, 2055 or 2056, or a variant thereof. In another embodiment, a binding molecule comprises a polypeptide comprising the sequence of SEQ ID NO: 2028 or a variant thereof. In another embodiment, a binding molecule comprise: (i) a polypeptide comprising the sequence of any one of SEQ ID NOs: 2052, 2053, 2054, 2055 or a variant thereof; and (ii) a polypeptide comprising the sequence of SEQ ID NO: 2028 or a variant thereof. For example, a binding molecule may comprise (i) a polypeptide comprising the sequence of SEQ ID NO: 2052 or a variant thereof; and (ii) a polypeptide comprising the sequence of SEQ ID NO: 2028 or a variant thereof. For example, a binding molecule may comprise: (i) a polypeptide comprising the sequence of SEQ ID NO: 2053 or a variant thereof; and (ii) a polypeptide comprising the sequence of SEQ ID NO: 2028 or a variant thereof. For example, a binding molecule may (i) a polypeptide comprising the sequence of SEQ ID NO: 2054 or a variant thereof; and (ii) a polypeptide comprising the sequence of SEQ ID NO: 2028 or a variant thereof. For example, a binding molecule may (i) a polypeptide comprising the sequence of SEQ ID NO: 2055 or a variant thereof; and (ii) a polypeptide comprising the sequence of SEQ ID NO: 2028 or a variant thereof. Also provided are binding molecules comprising the CDRs sets or variant versions thereof of such binding molecules. Also provided are binding molecules comprising the VHH domains or variant versions thereof of such binding molecules.

In a particularly preferred embodiment, the binding molecules set out are tri-specific. In one embodiment, a variant comprises the VHH regions set out herein, but the other sequences may be different. In another embodiment, a variant sequence will have the CDRs of a binding molecule set out herein, but the other sequences may vary. In one embodiment, the CDRs may have from 1 to 10 amino acid modifications in total, provided that the binding molecule retains functionality. In one embodiment, the modifications will be conservative amino acid modifications. Variants are explained in more detail elsewhere herein and any such degree or type of variation may apply to the specific binding molecules set out herein.

Also provided are binding molecules which are humanised versions of any of those set out herein. Further provided are binding molecules which have the same, or variant versions, of the CDRs for one of the binding molecules set out herein and in the same format, but the non-CDR sequences are different. Also provided are binding molecules with the same VHHs as a binding molecule set out herein, or variant VHH sequences, where the binding molecule is in the same format, but the non-VHH sequences are different. For any of the specific binding molecules set out herein variant versions are also provided where the constant region modifications and mutations are rather those present others set out herein.

The present invention also provides a trispecific binding molecule of the present invention wherein the binding molecule is an antibody comprising two heavy chains wherein the antibody has four antigen-binding regions. In one embodiment, the antibody has four antigen-binding regions, with two antigen-binding regions on each heavy chain polypeptide. In another embodiment, the antibody has four antigen binding sites, with one antigen-binding region on one heavy chain polypeptide and three antigen-binding regions on the other heavy chain polypeptide.

In one embodiment, an antibody of the present invention has six antigen-binding regions. In one embodiment, the antibody has six antigen-binding regions, with three antigen binding regions present on each heavy chain polypeptide. In another embodiment the antibody is symmetrical in the sense that each of the two heavy chain polypeptides is the same.

In one preferred embodiment, an antibody of the present invention is symmetrical in the sense that each of the two heavy chain polypeptides is the same, with each heavy chain comprising two antigen binding regions. In one embodiment, the antibody is symmetrical in the sense that each of the two heavy chain polypeptides is the same, with each heavy chain comprising three antigen binding regions. In another embodiment, the antibody comprises two different single domain binding regions that each bind a different epitope of the same IL-2R chain polypeptide.

In one particularly preferred embodiment, a binding molecule, particularly an antibody, does not comprise an antibody light chain. In another preferred embodiment, a binding molecule, particularly an antibody, of the present invention does not comprise a Fab region.

In one embodiment, a binding molecule of the present invention may be at least as good or improved for a particular parameter in comparison to IL-2. For instance, in one embodiment, the fold EC50 NK/Treg value of a binding molecule of the present invention may be at least as good or better than the value for IL-2. In another embodiment the fold maximal percent pSTAT5 signalling Treg/NK may be at least as good or better as for IL-2. In one preferred embodiment, the method used to measure such values is that employed in the Examples of the present application.

### Variant, Cross-Blocking, and Competing Antibodies

Also provided are variant binding molecules, in particular antibodies, derived from the specific molecules set out herein. Further provided, are binding molecules, in particular antibodies, that are able to cross-block the specific binding molecules set herein. Further provided are binding molecules, in particular antibodies, that are able to compete for binding with the specific molecules set out herein. In embodiments where an antibody of the present invention is a multi-specific antibody, it may be that just one antigen-binding specificity is defined in terms of being a variant of one of the specific antigen-binding sites set out herein, or able to compete, or cross-block with one of the specific antigen-binding sites set out here. As in a preferred embodiment the antigen-binding sites of an antibody of the present invention are based on VHH sequences, the individual VHH sequences set out herein may be used to define other VHH sequences that are able to compete or cross-block the specific VHH molecules set out herein.

Cross-blocking binding molecules, in particular antibodies, can be identified using any suitable method in the art, for example by using competition ELISA or BIAcore assays where binding of the cross-blocking binding molecule to antigen prevents the binding of a binding molecule of the present invention or vice versa. Such cross-blocking assays may use cells expressing IL-2R as a target. In one embodiment, flow cytometry is used to assess binding to cells expressing IL-2R. In another embodiment, the ability to compete or cross-block binding to an individual chain of the IL-2R is measured. A technique such as ELISA may be used. A technique such as surface plasmon resonance may be employed. In one embodiment, cross-blocking may be studied for each specificity individually. In one embodiment, that may be done by looking at the ability of individual VHHs to cross-block.

In one embodiment, the degree of cross-blocking may be, for instance, at least 75%, at least 80% or at least 90%. In another embodiment, it may be at least 95%. In another embodiment, it may be at least 99%. Such levels of cross-blocking may be in relation to the overall molecule.

Variant binding molecules, and in particular antibodies, may be employed where they still retain the desired properties of binding molecules of the present invention, particularly in relation to binding IL-2R. For instance, a variant binding molecule, in particular a variant antibody, or an antigen binding site of the variant, may be defined in terms of still being able to bind the same IL-2R chain as the original binding molecule, in particular antibody. Hence, binding molecules and antibodies with degrees of sequence identity to specific ones set out herein are also provided. The sequence identity may be over the entire length of a sequence, such as over the entire length of a VHH domain, or just over the CDR sequences. Sequence identity may also be defined in terms of over the entire length of the polypeptide in question. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656). The present invention also extends to novel polypeptide sequences disclosed herein and sequences at least 80% similar or identical thereto, for example 85% or greater, 90% or greater, in particular 95%, 96%, 97%, 98% or 99% or greater similarity or identity. In one embodiment a sequence may have at least 99% sequence identity to at least one of the specific sequences provided herein. “Identity”, as used herein, indicates that at any particular position in the aligned sequences, the amino acid residue is identical between the sequences. In one embodiment, similarity or identity is measured in relation to the entire length of the shortest sequence of the two being compared. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

In one embodiment, a variant may have from one to ten, such as one, two, three, four, five or up to those values of amino acid sequence changes or at least those values, or up to those values, so long as the variant is still able to specifically bind the desired IL-2R chain. In another embodiment, a variant of the present invention may have at least five, six, seven, eight, nine, ten, eleven or twelve amino acid sequence changes compared to the CDRs of one of the specific antibodies set out herein, for example is may have that number of sequence changes in a set of CDRs making up a VHH domain. An antibody of the present invention may have that number of sequence changes in the CDRs compared to the specific antibody set out herein. It may have up to that number of sequence changes. It may have at least that number of amino acid sequence changes. In one embodiment, a variant sequence may have one, two, three, four, five, or more amino acid sequence changes compared to one of the specific binding molecules set out herein. In one embodiment, it may have from five to ten, ten to fifteen, or fifteen to twenty amino acid sequence changes compared to a specific binding molecule set out herein. It may be that a binding molecule has that number of sequence changes in the overall VHH domain. It may have that number of sequence changes overall in the CDRs of a VHH domain. It may have such a number of sequence changes in the individual CDR. Such variant antibody molecules will typically retain the ability to specifically bind IL-2R or in the case of a VHH domain the IL-2R polypeptide it is specific for. They may also retain one of the other functions set out herein. Typically a variant will retain the ability to bind the IL-2R or individual IL-2R polypeptide. It will be appreciated that this aspect of the invention also extends to variants of the specific binding molecules and antibodies, and in particular antibodies, including humanised versions and modified versions, including those in which amino acids have been mutated in the CDRs to remove one or more isomerisation, deamidation, glycosylation site or cysteine residue.

In one embodiment, the binding molecules, an in particular antibodies, of the present invention are mutated to provide improved affinity for IL-2R polypeptides. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al J. Mol. Biol., 250, 359-368, 1996), DNA shuffling (Patten et al Curr. Opin. Biotechnol., 8, 724-733, 1997), phage display (Thompson et al., J. Mol. Biol., 256, 77-88, 1996), and sexual PCR (Crameri et al Nature, 391, 288-291, 1998). Vaughan et al discusses these methods of affinity maturation (Vaughan et al., Nat. Biotech., 16, 535-539, 1998). Where not specifically for VHH domains such approaches may be adapted for them. Improving the affinity of binding of individual binding sites will typically also improve the overall avidity for the target where the binding molecule has more than one binding site.

The skilled person may generate antibodies for use in the antibodies of the invention using any suitable method known in the art. Antigen polypeptides, for use in generating antibodies for example for use to immunize a host or for use in panning, such as in phage display, may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The antigen polypeptide may in some instances be part of a larger protein such as a fusion protein for example fused to an affinity tag or similar. In one embodiment, the host may be immunised with a cell expressing an IL-2R or an IL-2R polypeptide. In a particularly preferred embodiment, a VHH domain of the present invention is obtained by immunising a camelid and in particular a llama.

In one example, the antigen-binding sites, and in particular the variable regions, of the antibodies according to the invention are humanised. Humanised (which include CDR-grafted antibodies) as employed herein refers to molecules having one or more complementarity determining regions (CDRs) from a non-human species and a framework region from a human immunoglobulin molecule (see, e.g., U.S. Pat. No. 5,585,089; WO91/09967 which are incorporated by reference). It will be appreciated that it may only be necessary to transfer the specificity determining residues of the CDRs rather than the entire CDR (see for example, Kashmiri et al., 2005, Methods, 36, 25-34). In a preferred embodiment though, the whole CDR or CDRs is/are transplanted. Humanised antibodies may optionally further comprise one or more framework residues derived from the non-human species from which the CDRs were derived. As used herein, the term “humanised antibody molecule” refers to an antibody molecule wherein one or more CDRs (including, if desired, one or more modified CDRs) from a donor antibody (e.g., a murine monoclonal antibody) are grafted into a framework of an acceptor antibody (e.g., a human antibody). For a review, see Vaughan et al, Nature Biotechnology, 16, 535-539, 1998. In one embodiment, rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment, only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the humanised antibody according to the present invention has a variable domain comprising human acceptor framework regions as well as one or more of the CDRs provided herein. Examples of human frameworks which can be used in the present invention are KOL, NEWM, REI, EU, TUR, TEI, LAY and POM. For example, KOL and NEWM can be used for the heavy chain, REI can be used for the light chain and EU, LAY and POM can be used for both the heavy chain and the light chain. Alternatively, human germline sequences may be used; these are available at: http://www2.mrc-lmb.cam.ac.uk/vbase/list2.php.

In a humanised antibody molecule of the present invention, the acceptor framework does not necessarily need to be derived from the same antibody and may, if desired, comprise composite chains having framework regions derived from different chains. The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al 1998, Nature, 332, 323-324). Such changes should be kept to the minimum necessary to recover the affinity of the donor antibody. A protocol for selecting residues in the acceptor framework regions which may need to be changed is set forth in WO 91/09967. Derivatives of frameworks may have 1, 2, 3, 4, 5, 6, 7, 8, 9, 10 or more amino acids replaced with an alternative amino acid, for example with a donor residue. Donor residues are residues from the donor antibody, i.e., the antibody from which the CDRs were originally derived, in particular the residue in a corresponding location from the donor sequence is adopted. Donor residues may be replaced by a suitable residue derived from a human receptor framework (acceptor residues).

The Kabat et al numbering system is referred to herein. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated. The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard Kabat numbered sequence. The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A.M. J. Mol. Biol., 196, 901-917 (1987)), the loop equivalent to CDR-H1 extends from residue 26 to residue 32. Thus, unless indicated otherwise “CDR-H1” as employed herein is intended to refer to residues 26 to 35, as described by a combination of the Kabat numbering system and Chothia's topological loop definition. The CDRs of the light chain variable domain are located at residues 24-34 (CDR-L1), residues 50-56 (CDR-L2) and residues 89-97 (CDR-L3) according to the Kabat numbering system.

The skilled person is able to test variants of CDRs or humanised sequences in any suitable assay such as those described herein to confirm activity is maintained.

A preferred variant binding molecule will retain the ability to act as a trispecific binding molecule in the sense of binding all three of IL-2 α, β, and γ.

In one embodiment, variant antibodies may be identified by identifying such antibodies that are able to cross-block specific antibodies set out herein. Cross-blocking binding molecules, in particular antibodies, can be identified using any suitable method in the art, for example by using competition ELISA or BIAcore assays where binding of the cross-blocking antibody to antigen prevents the binding of an antibody of the present invention or vice versa. Such cross-blocking assays may use cells expressing IL-2Rα/IL-2Rβ/γc as a target. In one embodiment, flow cytometry is used to assess binding to cells expressing IL-2Rα/IL-2Rβ/γc.

Further provided, are binding molecules that bind the same epitope on one of the IL-2R polypeptide chains as one of the specific antibodies set out herein. For instance, the binding molecule may be an antibody that binds to the same epitope. It may be an antibody that belongs to the same “epitope bin” as one of those set out in the Examples of the present application. In one embodiment, the binding molecule may bind to all three of the epitopes recognised in the three IL-2R polypeptide chains.

In one preferred embodiment, a variant CDR has one of the levels of sequence identity recited herein. In another it has one of the levels of sequence identity. For instance, in one embodiment, a variant binding molecule may have at least 90% sequence identity to all of the relevant CDRs of the binding molecule it is being compared to. In another embodiment, the CDRs have at least 95% sequence identity over the CDRs they are being compared to. In another embodiment a variants may have VHH domains with at least 90% sequence identity to the VHH domains of the specific binding molecule it is being compared to. In another embodiment, the VHH domains have at least 95% sequence identity. In another embodiment, a variant CDR may show one, two, or three amino acid sequence changes compared to the specific CDR. A set of variants may be one where each CDR shows that level of variation compared to the specific sequence CDRs. It may be that level of variation is shown cumulatively over the whole CDRs compared to those of the specific binding molecules. A variant will typically retain functionality compared to the specific binding molecule. For example, a variant will typically still be able to bind all of the IL-2Rα, IL-2Rβ and γc chains.

For any of the specific CDRs, VHH domains, polypeptides, and binding molecules set out herein, the present invention also provides variant versions as set out herein.

### Conjugates, Fusion Proteins, Effector Molecules, and Labels

In a preferred embodiment, a binding molecule, particularly an antibody, of the present invention may exert its effect by binding the IL-2R without any need for a further effector molecule or label. In some embodiments though it may be conjugated to an effector molecule or label. Hence, a binding molecule, particularly an antibody, for use in the present invention may be conjugated to one or more effector or label molecule(s). Where it is desired to obtain a binding molecule, particularly an antibody, according to the present invention linked to an effector molecule or label, this may be prepared by standard chemical or recombinant DNA procedures in which the binding molecule is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector or label molecule is a protein or polypeptide the linkage may be achieved using recombinant DNA procedures, for example as described in WO 86/01533 and EP0392745.

Effector and label molecules which may be employed include, for example, drugs, toxins, biologically active proteins, for example enzymes, antibody or antibody fragments, synthetic or naturally occurring polymers, nucleic acids and fragments thereof, e.g., DNA, RNA, and fragments thereof, radionuclides, particularly radioiodide, radioisotopes, chelated metals, nanoparticles and reporter groups such as fluorescent compounds or compounds which may be detected by NMR or ESR spectroscopy. Antibodies of the present invention may comprise a detectable substance for use as a label. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, betagalactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another embodiment, the effector molecule may increase or decrease the half-life of the binding molecule, in particular antibody, in vivo, and/or reduce immunogenicity and/or enhance delivery across an epithelial barrier to the immune system. Examples of suitable effector molecules of this type include polymers, albumin, albumin-binding proteins or albumin-binding compounds such as those described in WO 05/117984. Where the effector molecule is a polymer, it may, in general, be a synthetic or a naturally occurring polymer, for example an optionally substituted straight or branched chain polyalkylene, polyalkenylene or polyoxyalkylene polymer or a branched or unbranched polysaccharide, e.g., a homo-or heteropolysaccharide. Specific optional substituents which may be present on the above-mentioned synthetic polymers include one or more hydroxy, methyl or methoxy groups. Specific examples of synthetic polymers include optionally substituted straight or branched chain poly (ethyleneglycol), poly (propyleneglycol) poly (vinylalcohol) or derivatives thereof, especially optionally substituted poly (ethyleneglycol) such as methoxypoly (ethyleneglycol) or derivatives thereof.

A binding molecule, particularly an antibody, of the present invention may be conjugated to a molecule that modulates or alters serum half-life. A binding molecule, particularly antibody, of the invention may bind to albumin, for example in order to modulate the serum half-life. Hence, in one embodiment a binding molecule, particularly an antibody, of the present invention includes a binding site for albumin, for instance it may include a VHH domain specific for albumin in addition to the other antigen-binding sites of the antibody. In another embodiment, a binding molecule, particularly an antibody, of the invention may include a peptide linker which is an albumin binding peptide. Examples of albumin binding peptides are included in WO 2015/197772 and WO 2007/106120 the entirety of which are incorporated by reference. In one embodiment, an antibody of the present invention may comprise an Fc tail, serum albumin, and/or a moiety which is a binder of serum albumin, and PEG.

In another embodiment, a binding molecule, particularly an antibody, of the invention is not conjugated to an effector molecule. In one embodiment, a binding molecule, particularly an antibody, of the invention is not conjugated to a toxin. In another embodiment, a binding molecule, particularly an antibody, of the invention is not conjugated to a radioisotope. In another embodiment, it is not conjugated to an agent for imaging.

### Assays

In one embodiment, a functional assay may be employed to determine if a binding molecule, particularly an antibody, of the present invention, or an individual component of it, has a particular property or properties, for instance such as any of those mentioned herein. In one embodiment, one or more of the assays described in the Examples of the present application may be employed to assess a particular binding molecule, particularly an antibody, and whether it has a desired property or properties.

A binding molecule, particularly an antibody molecule, of the present invention is able to bind at least one polypeptide chain of IL-2R, so at least one of IL-2Rα, IL-2Rβ, and the γc polypeptide chains. Preferably, it will be able to bind at least two of those polypeptide chains. Particularly preferably, it will be able to bind all three of the IL-2Rα, IL-2Rβ, and the γc polypeptide chains. The ability of antibody binding molecule, or individual VHH, of the present invention, or a candidate, to bind may be assessed in a variety of ways. For example, in one embodiment the ability to bind a given IL-2R polypeptide chain is assessed by employing the polypeptide, such as by using techniques like surface plasmon resonance using the polypeptide chain, or a portion thereof, bound to a chip. Any suitable method for measuring binding may be employed, such as any of the methods discussed herein. In a particularly preferred embodiment, the ability to bind IL-2Rα, IL-2Rβ, and the γc will be assessed using a cell expressing the high affinity IL-2Rα/IL-2Rβ/γc receptor complex on its surface. In one embodiment, candidate molecules are labelled and then screened for their ability to bind cells expressing the receptor, using techniques such as ELISA or flow cytometry. In another embodiment, candidate molecules may be incubated with cells expressing the receptor and then bound candidate molecules detected using secondary agents such as a labelled antibody specific for the species of the candidate molecules. In one embodiment, an antibody, or VHH domain, of present invention is labelled, for example using luciferase-tagged (e.g., Gaussia princeps luciferase (GpL)) variants of an antibody, an in particular antibody or the fusion proteins, for example as described in Kums et al., MAbs. 2017 April; 9 (3): 506-520). Such tagged antibodies may also be used in competitive binding assays.

In one embodiment, a binding molecule, particularly an antibody, of the present invention is able to act as an agonist of the IL-2Rα/IL-2Rβ/γc complex. The present invention provides a method comprising: (a) contacting a cell expressing the IL-2Rα/IL-2Rβ/γc complex with the candidate; and (b) measuring STAT5 phosphorylation, where if the candidate triggers STAT5 phosphorylation it is selected. Such methods may further comprise comparison to a positive control known to activate signalling and hence STAT5 phosphorylation. In one embodiment, the positive control is IL-2. In another embodiment, the positive control is one of the specific binding molecules disclosed herein known to activate the receptor. For instance, a desired variant may be one that gives at least 50% of the level of the phosphorylation as the control. In one embodiment, it gives at least 75% of the level of STAT5 phosphorylation in comparison to the control.

In another embodiment, a binding molecule, particularly an antibody, of the present invention is able to act as an antagonist of the receptor. For instance, in one embodiment, it prevents the binding of IL-2 to the receptor, but does not activate the receptor itself. In one embodiment, a method is provided comprising: (a) contacting a cell expressing the IL-2Rα/IL-2Rβ/γc complex with the candidate; and labelled IL-2; (b) measuring the amount of labelled IL-2 bound to the cell; and (c) comparing the level of IL-2 bound to that seen in the absence of the candidate. If the candidate results in a drop in the amount of labelled IL-2 binding to the cell it is said to have antagonistic activity. In one embodiment, a binding molecule, particularly an antibody, of the invention will reduce IL-2 binding by at least 10%, preferably at least 25%, and more preferably by at least 50%.

In one preferred embodiment, a binding molecule, particularly an antibody, of the present invention does not bind FcγR. In one preferred embodiment, a binding molecule, particularly a candidate antibody, is assessed both for its ability to bind IL-2R, but also for its ability not to bind to and activate FcγR. In one embodiment, the ability of a binding molecule, particularly an antibody, of the present invention to bind Fc receptors and in particular FcγR is assessed. The lack of binding to Fc receptors may be assessed, for instance to determine whether or not CDC, ADCP or ADCC activity is displayed and preferably neither will be by an antibody of the present invention.

In another embodiment, the ability of a binding molecule, particularly an antibody, of the present invention to stimulate activation and/or expansion of cells will be assessed, for example to stimulate particular immune cells in that way, as a binding molecule, particularly an antibody, of the present invention will be typically able to bring about activation and/or expansion of cells such as T cells. In one embodiment, ability to stimulate Treg cells and Treg subsets such as CD25bright Tregs, from PBMC is assessed. In one embodiment, ability to expand Tregs is assessed by a method comprising: isolating PBMC and then culturing the PBMC; harvesting the cells and then seeding the PBMC; incubating the cells with a candidate binding molecule, particularly antibody; and performing analysis to determine the number of cells. In a preferred embodiment, a negative control is performed where the cells are cultured without contacting with the candidate. The cells may be assessed using flow cytometry in particular staining for CD4+CD25+CD127−FoxP3+ cells. In a preferred embodiment the number of CD25+CD127−FoxP3+ cells within the CD3+CD4+ cell population is measured. The cells may also be stained with antibodies specific for CD3 and/or CD8. In one preferred embodiment, a binding molecule, particularly an antibody, of the present invention will give higher numbers of CD4+CD25+CD127−FoxP3+ cells compared to incubation without the binding molecule/antibody. In another embodiment, a candidate may also be compared to a specific binding molecule of the present invention, for example to assess whether a variant antibody is also able to expand Tregs to the same or greater degree than the specific binding molecule of the present invention.

In another preferred embodiment, FoxP3.Luci mice are employed to study Treg cell expansion as the mice express luciferase under the control of the mouse FoxP3 promoter, which acts as a marker for Treg cells. For example, such mice may be injected with a candidate then bioluminescence imaging is used to image Treg cells. A positive control with a known ability to stimulate the proliferation of Tregs cell may be performed, as may be a negative control. In one embodiment a variant or candidate will be compared to a known antibody of the present invention set out herein and if it results in an equivalent or greater level of Tregs as assessed by the bioluminescence imaging in a preferred embodiment it itself is also classified as binding molecule, in particular an antibody, of the present invention. Such assessment may also be combined with ex vivo assessment, for example by subsequently sacrificing the animal, isolating cells, and then analyzing Treg numbers.

In another preferred embodiment, for testing of human-specific molecules, transgenic mice expressing one or more human IL-2R chains are employed to study Treg levels and in particular expansion. Such transgenic mice can be crossbred with FoxP3.Luci transgenic mice for in vivo imaging of Treg expansion. Upon sacrifice, separate tissues can also be processed via imaging for changed levels of Tregs, versus negative control animals. Treg expansion and Treg/Teff ratios can also be quantitated using flow cytometry, sourcing splenocytes, leukocytes in blood or other tissues. Alternatively, immunodeficient mice such as NSG mice can be injected with human PBMCs, human CD34+ cells or human Tregs and the expansion of Tregs determined via flow cytometry.

The efficacy of binding molecule, in particular of an antibody, may be assessed in an in vivo system such as in animal models. For example, various models of graft versus host disease (GvHD) may be employed, with a candidate antibody given to such an animal model and then compared to a control animal which is the same animal model for GvHD but which has not been given the antibody. In one embodiment, an antibody of the present invention will present or reduce the GvHD in the animal model. One preferred model for GvHD employs NOD/Scid/IL2Rg−/−(NSG) mice into which human T cells are transferred, for example by the transfer of human PBMCs into the mice. In one preferred embodiment, the model employed is the NOD/Scid/IL2Rg−/− model used in the Examples of the present application. Other animal models may be used in the same way, for example models of conditions such as inflammatory bowel disease, lupus, multiple sclerosis, and type 1 diabetes.

### Pharmaceutical Compositions

In one embodiment, the present invention provides a pharmaceutical composition comprising: (a) a binding molecule of the present invention; and (b) a pharmaceutically acceptable carrier, diluent, and/or excipient. The particularly preferred binding molecule for any of the pharmaceutical compositions of the present invention is an antibody. In one embodiment, a pharmaceutical composition of the present invention comprises binding molecule of the present invention as well as a carrier, a stabilizer, an excipient, a diluent, a solubilizer, a surfactant, an emulsifier, a preservative and/or adjuvant. In one embodiment, a pharmaceutical composition of the present invention is in solid or liquid form. In one embodiment, the pharmaceutical composition may be in the form of a powder, a tablet, a solution or an aerosol. In one embodiment, a pharmaceutical composition of the present invention is provided in a frozen form. In one embodiment, a pharmaceutical composition of the present invention is provided in lyophilized form.

A pharmaceutical composition of the present invention will usually be supplied as a sterile, pharmaceutical composition. A pharmaceutical composition of the present invention may additionally comprise a pharmaceutically acceptable adjuvant. In another embodiment, no such adjuvant is present in a pharmaceutical composition of the present invention. The present invention also provides a process for preparation of a pharmaceutical or medicament composition comprising adding and mixing binding molecule of the present invention together with one or more of a pharmaceutically acceptable excipient, diluent or carrier.

Pharmaceutically acceptable carriers in therapeutic compositions may additionally contain liquids such as water, saline, glycerol and ethanol. Such carriers may be used, for example, so that the pharmaceutical compositions to be formulated as tablets, pills, dragées, capsules, liquids, gels, syrups, slurries and suspensions, for ingestion by the patient. The term “pharmaceutically acceptable excipient” as used herein typically refers to a pharmaceutically acceptable formulation carrier, solution or additive to enhance the desired characteristics of the compositions of the present invention. Excipients are well known in the art and include buffers (e.g., citrate buffer, phosphate buffer, acetate buffer, and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. Suitable carriers may be large, slowly metabolised macromolecules such as proteins, polypeptides, liposomes, polysaccharides, polylactic acids, polyglycolic acids, polymeric amino acids, amino acid copolymers and inactive virus particles. Pharmaceutically acceptable salts can be used, for example mineral acid salts, such as hydrochlorides, hydrobromides, phosphates and sulphates, or salts of organic acids, such as acetates, propionates, malonates, and benzoates.

In certain embodiments, the pharmaceutical composition may contain formulation materials for the purpose of modifying, maintaining or preserving certain characteristics of the composition such as the pH, osmolarity, viscosity, clarity, color, isotonicity, odour, sterility, stability, rate of dissolution or release, adsorption or penetration. A thorough discussion of pharmaceutically acceptable carriers is available in Remington's Pharmaceutical Sciences (Mack Publishing Company, N.J. 1991). Additional pharmaceutical compositions include formulations involving the antibody of the present invention in sustained or controlled delivery formulations. Techniques for formulating a variety of sustained-or controlled-delivery means are known to those skilled in the art. A binding molecule, in particular antibody, of the present invention may also be entrapped in microcapsules prepared, for example, by coacervation techniques or by interfacial polymerization, in colloidal drug delivery systems, or in macroemulsions. Such techniques are also disclosed in Remington's Pharmaceutical Sciences.

A subject will be typically administered a therapeutically effective amount of a pharmaceutical composition and hence of a binding molecule, in particular an antibody, of the present invention. The term “therapeutically effective amount” typically refers to an amount of a therapeutic agent needed to treat, ameliorate or prevent a targeted disease or condition, or to exhibit a detectable therapeutic or preventative effect. The precise therapeutically effective amount for a human subject will depend upon the severity of the disease state, the general health of the subject, the age, weight and gender of the subject, diet, time and frequency of administration, drug combination(s), reaction sensitivities, and tolerance/response to therapy. This amount can be determined by routine experimentation and is within the judgement of the clinician. For example, a low dose may be used initially and then increased if needed to be based on the response seen. Generally, a therapeutically effective amount will be from 0.01 mg/kg to 50 mg/kg, for example 0.1 mg/kg to 20 mg/kg per day. Alternatively, the dose may be 1 to 500 mg per day, such as 10 to 100, 200, 300 or 400 mg per day. In one embodiment, the amount in a given dose is at least enough to bring about a particular function.

In one embodiment, a binding molecule, in particular an antibody, of the present invention may be given in combination with another treatment for the condition being treated. For example, a binding molecule, in particular an antibody, of the present invention may be provided simultaneously, sequentially, or separately with such a further agent. In another embodiment, an antibody of the present invention may be provided in the same pharmaceutical composition as a second therapeutic agent.

In one preferred embodiment, the therapeutic agent of the invention, when in a pharmaceutical preparation, may be present in unit dose forms. Suitable doses may be calculated for patients according to their weight, for example suitable doses may be in the range of 0.01 to 20 mg/kg, for example 0.1 to 20 mg/kg, for example 1 to 20 mg/kg, for example 10 to 20 mg/kg or for example 1 to 15 mg/kg, for example 10 to 15 mg/kg. To effectively treat conditions of use in the present invention in a human, suitable doses may be within the range of 0.001 to 10 mg, 0.01 to 1000 mg, for example 0.1 to 1000 mg, for example 0.1 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of a dual targeting protein of this invention, which may be administered parenterally, for example subcutaneously, intravenously or intramuscularly. Such a dose may be, if necessary, repeated at appropriate time intervals selected as appropriate by a physician. A binding molecule, and in particular an antibody, of the present invention may be, for instance, lyophilized for storage and reconstituted in a suitable carrier prior to use. Lyophilization and reconstitution techniques can be employed.

The binding molecules, in particular antibodies, and pharmaceutical compositions of this invention may be administered by any number of routes including, but not limited to, oral, intravenous, intramuscular, intra-arterial, intramedullary, intrathecal, intraventricular, transdermal, transcutaneous (for example, see WO 98/20734), subcutaneous, intraperitoneal, intranasal, enteral, topical, sublingual, intravaginal or rectal routes. Hyposprays may also be used to administer the pharmaceutical compositions of the invention. Direct delivery of the compositions will generally be accomplished by injection, subcutaneously, intraperitoneally, intravenously or intramuscularly, or delivered to the interstitial space of a tissue. In one preferred embodiment, administration is via intravenous administration. In another preferred embodiment, administration is via subcutaneous administration, for example via subcutaneous injection. The compositions can also be administered into a specific tissue of interest. In some embodiments, administration is via site-specific or targeted local delivery techniques. Examples of site-specific or targeted local delivery techniques include various implantable depot sources of the antibody molecule or local delivery catheters, such as infusion catheters, indwelling catheters, or needle catheters, synthetic grafts, adventitial wraps, shunts and stents or other implantable devices, site specific carriers, direct injection, or direct application.

Dosage treatment may be a single dose schedule or a multiple dose schedule. Where the product is for injection or infusion, it may take the form of a suspension, solution or emulsion in an oily or aqueous vehicle and it may contain formulary agents, such as suspending, preservative, stabilising and/or dispersing agents. Alternatively, the pharmaceutical may be in dry form, for reconstitution before use with an appropriate sterile liquid. In one embodiment, a pharmaceutical composition comprising an antibody of the present invention is provided in lyophilised form. If a composition is to be administered by a route using the gastrointestinal tract, the composition will typically need to contain agents which protect the binding molecule, in particular antibody, from degradation but which release the binding molecule once it has been absorbed from the gastrointestinal tract. In another embodiment, a nebulisable formulation according to the present invention may be provided, for example, as single dose units (e.g., sealed plastic containers or vials) packed in foil envelopes. Each vial contains a unit dose in a volume, e.g., 2 ml, of solvent/solution buffer.

A pharmaceutical composition of the present invention may be provided in a receptacle that provides means for administration to a subject. In one embodiment, a pharmaceutical composition of the present invention may be provided in a prefilled syringe. The present invention therefore provides such a loaded syringe. It also provides an auto-injector loaded with a pharmaceutical composition of the present invention.

In one embodiment the formulation is provided as a formulation for topical administrations including inhalation. Suitable inhalable preparations include inhalable powders, metering aerosols containing propellant gases or inhalable solutions free from propellant gases. Inhalable powders according to the invention containing the active substance may consist solely of the abovementioned active substances or of a mixture of the abovementioned active substances with physiologically acceptable excipient. These inhalable powders may include monosaccharides (e.g., glucose or arabinose), disaccharides (e.g., lactose, saccharose, maltose), oligo- and polysaccharides (e.g., dextranes), polyalcohols (e.g., sorbitol, mannitol, xylitol), salts (e.g., sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 1 to 5 μm. The particle size of the active ingredient such as the antibody or fragment is of primary importance. The propellant gases which can be used to prepare the inhalable aerosols are known in the art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The above mentioned propellent gases may be used on their own or in mixtures thereof. Particularly suitable propellent gases are halogenated alkane derivatives selected from among TG 11, TG 12, TG 134a and TG227. Of the above-mentioned halogenated hydrocarbons, TG134a (1,1,1,2-tetrafluoroethane) and TG227 (1,1,1,2,3,3,3-heptafluoropropane) and mixtures thereof are particularly suitable. The propellent-gas-containing inhalable aerosols may also contain other ingredients such as cosolvents, stabilisers, surface-active agents (surfactants), antioxidants, lubricants and means for adjusting the pH. All these ingredients are known in the art. The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active ingredient.

Alternatively topical administrations to the lung may also be by administration of a liquid solution or suspension formulation, for example employing a device such as a nebulizer, for example, a nebulizer connected to a compressor (e.g., the Pari LC-Jet Plus (R) nebulizer connected to a Pari Master (R) compressor manufactured by Pari Respiratory Equipment, Inc., Richmond, Va.).

Nebulisable formulation according to the present invention may be provided, for example, as single dose units (e.g., sealed plastic containers or vials) packed in foil envelopes. Each vial contains a unit dose in a volume, e.g., 2 mL, of solvent/solution buffer. The present invention also provides a syringe loaded with a composition comprising an antibody of the invention. In one embodiment, a pre-filled syringe loaded with a unit dose of an antibody is provided. In another embodiment, an autoinjector loaded with a binding molecule, in particular an antibody, of the invention is provided. In a further embodiment, an IV bag loaded with a pharmaceutical composition of the invention is provided.

It is also envisaged that an antibody of the present invention may be administered by use of gene therapy. In order to achieve this, DNA sequences encoding the binding molecule, in particular antibody, under the control of appropriate DNA components are introduced into a patient such that the binding molecule, in particular antibody chains and so antibody, are expressed from the DNA sequences and assembled in situ.

Once formulated, the compositions of the invention can be administered directly to the subject. By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on. In a preferred embodiment, the subject to be treated is a mammal. The subjects to be treated can be animals. However, in one or more embodiments the compositions are adapted for administration to humans. In a particularly preferred embodiment, the subject is human.

### Kits

The present invention also extends to a kit comprising a binding molecule, in particular an antibody, of the invention, optionally with instructions for administration. In yet another embodiment, the kit further comprises one or more reagents for performing one or more functional assays. In another embodiment, a kit containing single-chambered or multi-chambered pre-filled syringes is provided which is pre-filled with a pharmaceutical composition of the invention. The invention also provides a kit for a single-dose administration unit which comprises a pharmaceutical composition of the invention. In another embodiment, the kit comprises packaging.

### Pathological Conditions, Medical, and Diagnostic Uses

Also provided is a binding molecule, in particular an antibody, of the present invention for use as a medicament. In another embodiment a binding molecule, in particular an antibody, of the present invention is provided for use in a method of therapy of the human or animal body. Please note that, in the various therapeutic uses set out herein where reference is made to a binding molecule or an antibody of the present invention, a pharmaceutical composition comprising it may be also employed and vice versa unless stated otherwise, as may be a composition encoding an antibody of the invention. A binding molecule, in particular an antibody, of the present invention may also be used in diagnosis, including in both in vivo diagnosis and also in vitro diagnosis, for example such diagnosis performed on a sample from a subject.

As discussed further below, a binding molecule, in particular an antibody, of the present invention may be employed to treat a condition. As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The binding molecule, in particular antibody, of the invention may be used to treat any condition that would benefit. For instance, it may be used to treat an autoimmune condition. For instance, examples of conditions that may be treated include:
(multispecific conditions:) graft versus host disease (GvHD), systemic lupus erythematosus (SLE) rheumatoid arthritis (RA) (e.g., juvenile rheumatoid arthritis, polyarticular juvenile idiopathic arthritis, psoriatic arthritis, pediatric arthritis, osteoarthritis), type 1 diabetes (TID), autoimmune hepatitis, alopecia areata, polychondritis, ankylosing spondylitis,
(gastro-intestinal tract conditions:) Crohn's disease (CD), ulcerative colitis (UC), pouchitis, celiac disease,
(muscle conditions:) polymyalgia, polymyositis, idiopathic inflammatory myopathy (IIM), myasthenia gravis,
(skin conditions:) psoriasis, dermatitis, atopic dermatitis, eczema, scleroderma, pemphigus vulgaris, bullous pemphigoid,
(nerve conditions:) multiple sclerosis (MS), Guillain-Barré syndrome, amyotrophic lateral sclerosis (ALS), chronic inflammatory demyelinating polyneuropathy (CIDP),
(glandular conditions:) Sjögren's syndrome, Grave's disease, Hashimoto's thyroiditis, Addison's disease,
(blood conditions:) vasculitis, Behcet's disease, Takayasu's arteritis, granulomatosis with polyangitisis, antiphospholipid syndrome (APS), idiopathic thrombocytopeni purpura (ITP), and
(conditions affecting airways:) birch pollen allergy, asthma, respiratory-COVID19.

In one preferred embodiment, the invention may be used to treat or prevent graft versus host disease (GvHD). In one embodiment, the autoimmune disease is selected from type 1 diabetes (TID), multiple sclerosis (MS), Crohn's disease (CD), ulcerative colitis (UC), psoriasis, Guillain-Barré syndrome (GBS), systemic lupus erythematosus (SLE), rheumatoid arthritis (RA), chronic inflammatory demyelinating polyneuropathy (CIDP), Hashimoto's thyroiditis, celiac disease, Addison's disease, autoimmune hepatitis, antiphospholipid syndrome (APS), and Grave's disease. In another embodiment, the autoimmune disease is selected from diseases where the autoreactive T cell compartment, potentially in collaboration with autoreactive B cells, contributes significantly to disease pathology. Such diseases include, but are not limited to myasthenia gravis, pemphigus vulgaris, and bullous pemphigoid.

In one embodiment, the disease to be treated is selected from acute or chronic GvHD, SLE, autoimmune hepatitis, ulcerative colitis, and eczema. In another embodiment, the disease to be treated is selected from alopecia areata, type 1 diabetes, SLE, multiple sclerosis, birch pollen allergy, pemphigus vulgaris, bullous pemphigoid, amyotrophic lateral sclerosis (ALS), polymyalgia, Behcet's disease, polychondritis, idiopathic inflammatory myopathy (IIM), Crohn's disease, rheumatoid arthritis, psoriasis, dermatitis, respiratory-COVID19, vasculitis, idiopathic thrombocytopenia purpura (ITP), and polymyositis. In a further preferred embodiment, the disease to be treated is selected from Takayasu's arteritis, ankylosing spondylitis, granulomatosis with polyangiitis, and Sjögren's syndrome. Particularly preferred disorders to be treated are GvHD, atopic dermatitis, and psoriasis. Other preferred disorders to be treated are ulcerative colitis and SLE.

In one embodiment, a binding molecule, in particular an antibody, of the invention is used to treat or prevent an immune response against a transplant. Examples of organs and tissues that can be transplanted in a mammal that can be treated as described herein include, without limitation, skin, bone, blood, heart, liver, kidney, pancreas, intestine, stomach, testis, penis, cornea, bone marrow, and lung. A transplant can be an allogeneic transplant or an autologous transplant. In some cases, the materials and methods described herein also can be used to treat a mammal having a complication or disease associated with a transplant (e.g., GvHD). In one embodiment, the transplant reject is of an autologous transplant or an allogenic transplant.

In some cases, a binding molecule, in particular an antibody, of the present invention can be administered as a combination therapy with one or more additional treatments used to treat an autoimmune disease and/or one or more additional immunosuppressants. For example, a combination therapy used to treat an autoimmune disease can include administering to the subject a binding molecule, in particular an antibody, as described herein and one or more autoimmune disease treatments such as an adoptive cell (e.g., Treg) transfer, tolerogenic vaccination, an immune checkpoint agonist, and/or steroid administration. For example, a combination therapy used to enhance an immune response can include administering to the mammal an antibody as described herein and one or more immunosuppressants such as cyclosporine, rapamycin, methotrexate, azathioprine, chlorambucil, leflunomide, and/or mycophenolate mofetil.

As discussed further below, a binding molecule, in particular an antibody, of the present invention may be employed to treat a condition. As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In one particularly preferred embodiment, a binding molecule, in particular an antibody, of the present invention may be used to modulate the immune system. For example, it may be used to stimulate cells of the immune system, for instance activating particular cells of the immune system. In one embodiment the cells may be stimulated to proliferate. In one preferred embodiment, a binding molecule, in particular an antibody, of the present invention is used to activate cells expressing high affinity IL-2R on their surface. For example, the cells in question may be white blood cells and in particular T cells. In a particularly preferred embodiment, a binding molecule, in particular an antibody, of the present invention is used to activate Treg cells, in particular CD25bright Tregs. For example, a binding molecule, in particular an antibody, of the present invention may be used to stimulate Treg cells which in turn suppress, reduce, or prevent an immune response.

The ability of the present invention to modulate the immune system means that it represents a particular good way to target, for example, an autoimmune disorder, or an inflammatory disorder. Hence, the present invention provides for a binding molecule, in particular an antibody, or pharmaceutical composition of the present invention for use in a method of treating or preventing an autoimmune disorder, or an inflammatory disorder. The present invention provides a binding molecule, in particular an antibody, or pharmaceutical composition for use in such a method wherein:
(a) the disorder is graft versus host disease (GvHD), preferably where the antibody is for use in a method where it is administered prior to, at the same time, or after a transplant of a cell, tissue, or organ; or
(b) the disorder is one involving dysfunction or unwanted proliferation of leukocytes, preferably of T cells, more preferably of Teff cells; such disorders may present with an imbalance of Tregs compared to Teff cells, for example due to increased numbers or activity of Teff which is not balance the numbers and/or immunosuppressive properties of Tregs and in one embodiment the present invention promotes Tregs and in particular shifts the balance with effector cell to, or towards, normal.
(c) the disorder is selected from inflammatory bowel disease (such as ulcerative colitis, Crohn's disease, pouchitis, or celiac disease), multiple sclerosis, myasthenia gravis, skin autoimmune diseases such as pemphigus vulgaris or bullous pemphigoid, and type 1 diabetes.

The present invention may be used in treating graft versus host disease (GvHD). In one embodiment, the present invention is employed to promote Treg activity prior to a cell, tissue or organ transplant. For example, in one embodiment the present invention is used to promote Treg activity before transplantation of cells, in particular prior to transplantation of stem cells, and preferably before the transplantation of hematopoietic stem cells. In another embodiment, rather than stimulate Tregs in the recipient prior to transplantation, the invention is used to expand Tregs in a cell population, tissue, or organ that is to be transplanted to the host. In a further embodiment, they are used as part of the treatment for non-malignant hematopoietic diseases.

The present invention may be used to reduce, prevent or treat an immune response against a transplant, for example against transplanted cells, tissue or an organ. Hence, the invention may be used to reduce, prevent or treat graft versus host disease (GvHD). In one embodiment the GvHD is chronic (cGvHD). In one embodiment, the present invention may be used in that way where what is transplanted are cells such as a cell population. In one embodiment, the transplanted material is, or comprises, haematopoietic stem cells (HSCs). In another embodiment, the transplanted material may be an organ or tissue, such as the transplant of a heart, lung, kidney, cornea, or other organ. In another embodiment, the transplanted material may be a graft, such as a skin graft. In one embodiment, the present invention provides a method that comprises administering a binding molecule, in particular an antibody, of the present invention to treat, prevent, or ameliorate an unwanted immune response against transplanted cells, tissues or organs. In one embodiment, the method may actually further comprise performing the transplant. In another embodiment, the binding molecule, in particular antibody, of the present invention is given to the subject before, during, and/or after the transplant. In a further embodiment, rather than administration of the binding molecule, in particular antibody, to the subject the method comprises treating the material to be transplanted ex vivo with a binding molecule before it is transplanted. In one embodiment, a binding molecule of the present invention may be used to expand Treg cells prior to transplantation into a subject and may also activate the Treg cells. In one embodiment, the invention provides a way to expand and activate Tregs ex vivo.

In one embodiment, rather than treat, prevent, or ameliorate the disease itself, the invention is employed to help ensure that the treatment for the disease, namely the transplanted cells, tissue, or organ, is effective by preventing or reducing the severity of GvHD. Hence, the present invention may be employed in a variety of embodiments where a disease is treated by transplanting cells, tissue or organ. In one preferred embodiment, the condition may be one treated via a stem cell transplant, for example a hematopoietic stem cell (HSC) transplant. In some embodiments, the subject has or is otherwise affected by a metabolic storage disorder which is to be treated by a transplant. The subject may suffer or otherwise be affected by a metabolic disorder selected from the group consisting of glycogen storage diseases, mucopolysaccharidoses, Gaucher's disease, Hurler's disease, sphingolipidoses, metachromatic leukodystrophy, or any other diseases or disorders which may benefit from the treatments and therapies disclosed herein and including, without limitation, severe combined immunodeficiency (SCID), Wiskott-Aldrich syndrome, hyper immunoglobulin M (IgM) syndrome, Chediak-Higashi disease (CHS), hereditary lymphohistiocytosis, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. In one embodiment, where the invention concerns transplantation, it may be that the transfer is of allogenic cells, tissues, or organs. In one embodiment, the transferred cells may be cells expressing a chimeric antigen receptor (CAR). In some embodiments, the subject is in need of chimeric antigen receptor T-cell (CART) therapy. Such T cells can be Teff, but also Treg cells. For instance, such therapy may form part of a method of the present invention. In another preferred embodiment, the invention provides a method of promoting the engraftment of a cell population, tissue, or organ in a subject by treating, reducing, or preventing an immune response against said population, tissue, or organ.

The ability of a binding molecule, in particular an antibody, of the present invention to modulate the immune system also makes it a particularly valuable approach for targeting autoimmune disease. Hence, in another embodiment, the subject to be treated has an autoimmune disorder. In one particularly preferred embodiment, the autoimmune disorder is multiple sclerosis. In a further particular preferred embodiment, the subject has ulcerative colitis. In another particularly preferred embodiment, the condition is scleroderma. In one embodiment, the condition to be treated is lupus. Further examples of autoimmune diseases include scleroderma, Crohn's disease, type 1 diabetes, or another autoimmune pathology described herein. In one embodiment, the autoimmune disease to be treated is selected from ulcerative colitis, Crohn's disease, celiac disease, inflammatory bowel disease, multiple sclerosis, lupus, Graves' disease and type 1 Diabetes. In one embodiment, the subject has type 1 Diabetes and that is treated.

In one preferred embodiment, the condition treated is a condition involving unwanted inflammation. In one preferred embodiment, the condition is arthritis. For example, the present invention may be used to treat rheumatoid or osteo-arthritis. Non-limiting types of Examples of diseases which may be treated include rheumatoid arthritis, polyarticular juvenile idiopathic arthritis, psoriatic arthritis, and paediatric arthritis. In another preferred embodiment, the condition to be treated is selected from multiple sclerosis, ankylosing spondylitis, Crohn's disease, and ulcerative colitis.

In one embodiment, the ability of the invention to stimulate Treg cells is employed as a way to treat allergy. In another embodiment, the ability to stimulate Treg cells may be employed as a way to treat asthma.

The invention may also be used to treat aging, in particular age related inflammation. For example, individuals may display chronic, senescence associated inflammation as a function of older age which can be reduced by promoting Tregs using the binding molecule of the present invention.

In one embodiment, a binding molecule of the present invention is used to preferentially activate Treg cells, for example as compared to Teff cells. In one embodiment, a binding molecule of the invention is used to activate Treg cells and hence to downregulate an immune response, for instance as a way of treating one of the conditions mentioned herein. In one embodiment, the invention may be used to treat a disease that can be treated or ameliorated by expansion of Tregs. In another embodiment, a binding molecule of the present invention is used to treat one of the disorders mentioned herein by expanding the number of Tregs in an individual, in particular by expanding Treg numbers and activating those Tregs.

### Detection and Diagnosis

A binding molecule, in particular an antibody, of the present invention may be used to detect any of the chains of the IL-2R that it is specific for. For example, the present invention provides a method comprising contacting a binding molecule, in particular an antibody, of the present invention with a test sample and detecting any binding of a binding molecule. A binding molecule of the present invention may be labelled or linked to an enzyme which allows the detection of the binding molecule and hence that the binding molecule has bound. In one embodiment, such detections methods may be, for instance, ELISA assays or flow cytometry as a way to detect whether or not cells in a test sample express IL-2R on their surface. A binding molecule, in particular antibody, of the present invention may be used in in vitro detection, it may also be used in detection of IL-2R in vivo.

In one embodiment, the present invention provides an in vivo method for detecting IL-2R that comprises administering a labelled binding molecule, in particular antibody, of the present invention and then detecting the location of the binding molecule in the body of a subject. In another embodiment, an antibody of the present invention may be used in the diagnosis of a condition, for example in identifying a reduction of cells expressing IL-2R. In one preferred embodiment, the present invention provides a method of patient stratification comprising subdividing patients on the basis of the level of IL-2R expression.

The present invention also provides a kit for detecting IL-2R comprising a binding molecule, in particular an antibody, of the present invention and optionally instructions for employing the antibody in a method of detecting IL-2R.

In one embodiment, the present invention provides a binding molecule, in particular an antibody, of the present invention as a companion diagnostic, for instance to determine whether or not to administer a drug to a subject based on detection of IL-2R, such as levels of IL-2R, for instance the number of particular cell types expressing IL-2R or their location.

In one embodiment, a monovalent binding molecule of the present invention may be used in diagnosis that binds just one of the α, β, γc polypeptides. Such monovalent molecules may be used to detect the individual polypeptide. In a further embodiment, bivalent binding molecules of the invention may be used to detect two of the α, β, γc polypeptides. In one preferred embodiment, the bivalent molecule binds both the β and γc polypeptides. Hence, the detection methods outlined herein can be used for detecting one chain, two chain, or all three chains.

All documents referred to herein are incorporated by reference. Reference herein to the singular, using terms such as “a” and “an” also encompasses the plural unless specifically stated otherwise. Where something is referred to herein as “comprising” in another embodiment what the invention may “consist essentially of” what is set out. In another embodiment, it may “consist of” what is set out. Unless defined otherwise, all technical and scientific terms used herein have the meaning commonly understood by a person skilled in the art to which this invention belongs.

### EXAMPLES

The invention will be further understood with reference to the following non-limiting Examples.

### Example 1: Generation of Monoclonal Antibodies Targeting IL-2Rα, IL-2Rβ and IL-2Rγ

### A. Llama Immunization and Library Construction

Two llamas were immunized with a mix of three pUNO1 plasmids encoding the human IL-2Rα (pUNO1-hIL02RA), IL-2Rβ and IL-2Rγ proteins under control of constitutively active EF-1α/HTLV promoter (Invivogen) in a 2:1:2 mass ratio. Intramuscular DNA injections were repeated a total of 6 times with 2-week intervals. Blood samples of 10 mL were collected pre- and post-immunization to investigate immune response. Four days after the last immunization, 400 mL blood from each immunized llama was collected to isolate PBMCs using Ficoll-Paque gradient and used for RNA extraction. Total RNA was then converted into random primed cDNA using reverse transcriptase, and gene sequences encoding for VHH regions of llama heavy chain-only antibodies were amplified by PCR and subcloned into a phagemid vector.

Specific immune responses to human IL-2Rα, IL-2Rβ and IL-2Rγ were measured by ELISA on coated recombinant proteins. Both immunized llamas showed a strong immune response against human IL-2Rα, IL-2Rβ and IL-2Rγ when pre- and post-immune sera were compared.

### B. Selection of VHH Binding to IL-2Rα, IL-2Rβ and IL-2Rγ

Llama VHH phage display libraries in pDCL1 vector were generated and used for selections against the different subunits of the human IL-2R. The VHH-pDCL1 phage display libraries passed the QC criteria of size above 1.0E+08 and showed 100% VHH insert percentage.

Specific VHH antibody fragments were identified by selecting and screening using recombinant human and mouse IL-2Rα, IL-2Rβ and IL-2Rγ proteins as antigens. Two parallel phage display selection strategies were used to identify antibodies binding to the different subunits of the IL-2 receptor: either in-solution selections on pre-captured antigens or panning on antigens coated on a plate. In-solution selections were performed using the KingFisher™ Flex system. In the first round the human proteins were used, in rounds two and three both human and mouse IL-2R proteins were used.

Very high phage enrichments were observed for selections performed on both human and mouse IL-2Rα, IL-2Rβ and IL-2Rγ for one of the animals; outputs on mouse receptor subunits indicated presence of cross-reactive binders. Selections using library from the other animal resulted in high enrichment only for human IL-2Rβ.

### C. VHH Screening and Characterisation

Individual clones were isolated and periplasmic extracts (P.E.) containing soluble VHH fragments were produced and screened in a binding ELISA and by surface plasmon resonance (SPR).

In a binding ELISA, human or mouse IL-2Rα, IL-2Rβ and IL-2Rγ proteins were coated directly on maxisorp microtiter plates overnight at 4° C. Free binding sites were blocked using 4% Marvel in PBS for 1 hour. Next, 1:5 dilution of P.E. in 1% Marvel/PBS were added to wells and incubated for 1 hour. After incubation and an extensive PBS washing step, VHH binding was revealed using mouse anti-c-myc IgG and anti-mouse IgG-HRP antibodies. Binding specificity was determined based on O.D. at 450 nm values compared to negative controls.

SPR was performed to determine dissociation rates using Biacore 3000 instrument (GE Healthcare). Briefly, human and mouse IL-2R proteins were immobilized on carboxylmethyl dextran sensor chip (CM5) at approximately 2500 RUs using amine coupling in acetate buffer (GE Healthcare). The VHH-containing P.E. were loaded with a flow rate of 30 uL/min and the off-rates were measured over a 120s period.

VHH clones that showed binding to IL-2R subunits were sequenced and divided into families based on the sequence of the VHH CD3 region. 85 IL-2Rα-, 153 IL-2Rβ-, 92 IL-2Rγ-specific clones with unique VHH sequences were identified, which resulted in 15, 38, and 7 VHH CDR3 families, respectively.

The binding characteristics and the VHH CD3 sequences of the selected clones are shown in TABLE 1.

### D. Monospecific Antibody Purification and Characterisation

For each IL-2R subunit, 10 clones displaying varying characteristics were reformatted as VHH-human Fc fusion molecules. For this purpose, the cDNA encoding the VHH of each clone was engineered into a mammalian expression vector comprising the cDNA encoding the CH2 and CH3 domains of human IgG1 and containing mutations that abrogate antibody effector functions mediated by the Fc receptor. Particularly, the molecules comprised the amino acid substitutions L234A, L235A and P329G (EU numbering) in the immunoglobulin heavy chains.

Antibody molecules were subsequently produced by transient transfection in HEK293E cells and purified from cell supernatant by protein A affinity chromatography. Finally, SDS-PAGE analysis was carried out to assess the purity and the integrity of the VHH-human Fc molecules. Produced proteins were highly pure and of correct size (around 78 kDa).

### Example 2: In vitro characterisation of monospecific antibodies targeting the IL-2Rα, IL-2Rβ and IL-2Rγ

### A. Epitope Mapping

Biacore 3000 system (GE Healthcare) was used to determine whether IL-2Rα-, IL-2Rβ- and IL-2Rγ-specific antibodies compete for the same epitope or bind a different one on their respective targets. A CM5 sensor chip was coated with human IL-2Rα, IL-2Rβ and IL-2Rγ proteins at approximately 100 RU using standard amine coupling. Antibodies were diluted in HBS-EP pH 7.4 buffer at a concentration of 100 nM. Antibodies binding to the same IL-2R subunit were injected pairwise using the Biacore COINJECT method and a flow rate of 30 μL/min. An increased signal observed after the injection of a second antibody indicates that it binds to another epitope on its target than the first antibody. IL-2Rα-, IL-2Rβ- and IL-2Rγ-specific antibodies bound to three, three and four distinct epitopes on their target, respectively (TABLE 1).

The same setup was used to test whether anti-IL-2Rα antibodies blocked human IL-2 binding to human IL-2Rα (CD25). Here, each antibody injection was followed by injection with 100 nM human IL-2. IL-2Rα-specific antibodies from one epitope bin (H) did not block the binding of human IL-2 to human IL-2Rα, while the antibodies from the other two epitope bins (I and J) were blocking (TABLE 1).

### B. Binding to HEK-Blue IL-2 Cells

The ability of monospecific anti-IL-2Rα, anti-IL-2Rβ and anti-cy antibodies to bind the human IL-2 receptor was analysed using HEK-Blue IL-2 recombinant cell line (Invivogen, #hkb-il2) overexpressing the three IL-2R subunits. Cell culture was performed following the manufacturer's protocol. Cells were seeded at 100 000 cells/well in a 96-well plate, washed with FACS buffer, and incubated with antibodies diluted in FACS buffer at the concentration of 10 nM for 1 hour at 4° C., washed again with FACS buffer and stained with anti-human IgG-PE detection antibody (eBioscience) for 1 hour at 4° C. Dead cells were excluded from the analysis by using a fixable viability dye (eFluor780, eBioscience). Stained cells were analyzed on a LSR Fortessa flow cytometer (BD Biosciences). Final analysis and graphic output were performed with FlowJo v10.7.1 software (BD Biosciences) and GraphPad Prism version 8 (GraphPad Software). The dose-response binding curves were fit to a nonlinear regression model (log (agonist) vs. response with a variable slope (four parameters)).

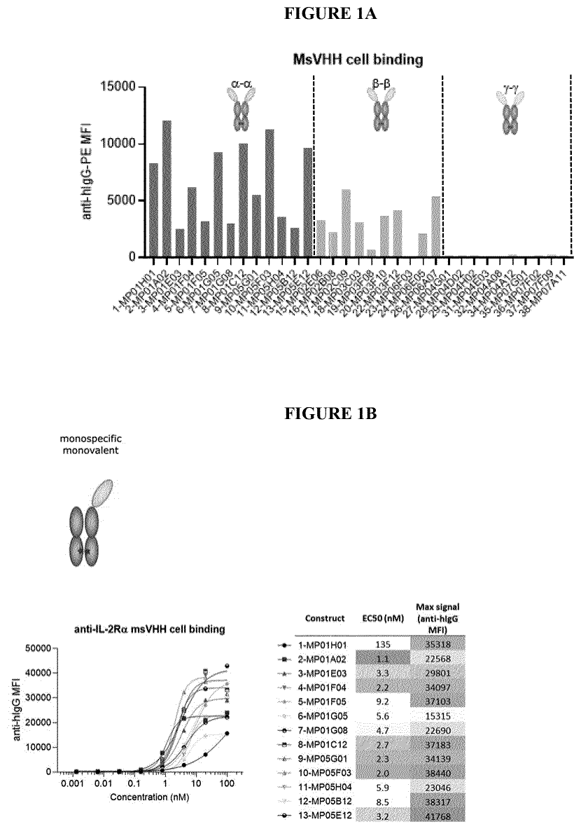

The cell binding properties of monospecific anti-IL-2Rα, anti-IL-2Rβ and anti-γc antibodies are shown in FIG. 1A. IL-2Rα and IL-2Rβ-specific clones display high and intermediate cell binding, respectively, while no binding is detected with anti-γc antibodies.

The monospecific monovalent anti-IL-2Rα antibodies were further tested at multiple concentrations; the dose-response curves for cell binding on HEK-Blue IL-2R cells are shown in FIG. 1B.

C. Interference with Human IL-2 Binding to its Receptor

The ability of monospecific anti-IL-2Rα, anti-IL-2Rβ and anti-IL-2Rγ antibodies to compete with the human IL-2 binding to its receptor was analysed using HEK-Blue IL-2R recombinant cell line (Invivogen, #hkb-il2) overexpressing the three IL-2R subunits. Cell culture was performed following the manufacturer's protocol. Cells were seeded at 100,000 cells/well in a 96-well plate, washed with FACS buffer, and preincubated with antibodies diluted in FACS buffer at the concentration of 10 nM for 20 min at 4° C., after which biotinylated human IL-2 (proteintech) at 2 nM was added for another 1 hour at 4° C. Cells were washed again with FACS buffer and stained with Streptavidin-PE (eBioscience) for 1 hour at 4° C. Dead cells were excluded from the analysis by using a fixable viability dye (cFluor780, eBioscience). Stained cells were analyzed on a LSR Fortessa flow cytometer (BD Biosciences). Final analysis and graphic output were performed with FlowJo v10.7.1 software (BD Biosciences) and GraphPad Prism version 8 (GraphPad Software).

The neutralizing potencies of monospecific anti-IL-2Rα, anti-IL-2Rβ and anti-γc antibodies are depicted in FIG. 2 Several clones greatly inhibited the human IL-2 binding to its receptor. Specifically, anti-IL-2Rα Abs 2, 8 and 10 and anti-IL-2Rβ Abs 15 and 17 potently blocked human IL-2 binding to its receptor. Interestingly, although no binding signal is detected with γc-specific clones, clone 28 moderately inhibits human IL-2 binding to its receptor.

D. Human-cynomolgous monkey IL2R subunit cross-reactivity testing

Monospecific monovalent and bivalent IL-2Rα-, IL-2Rβ- and IL-2Rγ-specific antibodies that were selected on human IL-2R subunits, were evaluated for their cross-reactivity in an ELISA. Human and cynomolgus monkey receptor subunits (Acrobiosystems, KactusBiosystems) were coated at 1 μg/mL in PBS (pH 7.4) in a Maxisorp plate (Nunc) and incubated overnight at 4° C. The plates were further washed with PBS-Tween pH 7.4 and incubated with 1% cascin/PBS-Tween blocking solution for 1 hour shaking at 400 rpm. Subsequently, the plate was washed three times with PBS-Tween pH 7.4, after which the test antibodies diluted in 0.1% casein/PBS-Tween were added to the plate and incubated for 1 hour. The plate was again washed three times, after which goat anti-human IgG Fc (HRP) detection antibody (abcam) was added to the plate and incubated for 30 min. The colouring reaction was performed with TMB (Sigma-Aldrich) and stopped with 0.5N H₂SO₄.

Absorbance was measured at 450 nm with the reference wavelength of 620 nm using spectrophotometer. Final analysis and graphic output were performed with GraphPad Prism version 8 (GraphPad Software). The dose-response binding curves were fit to a nonlinear regression model (log (agonist) vs. response with a variable slope (four parameters)).

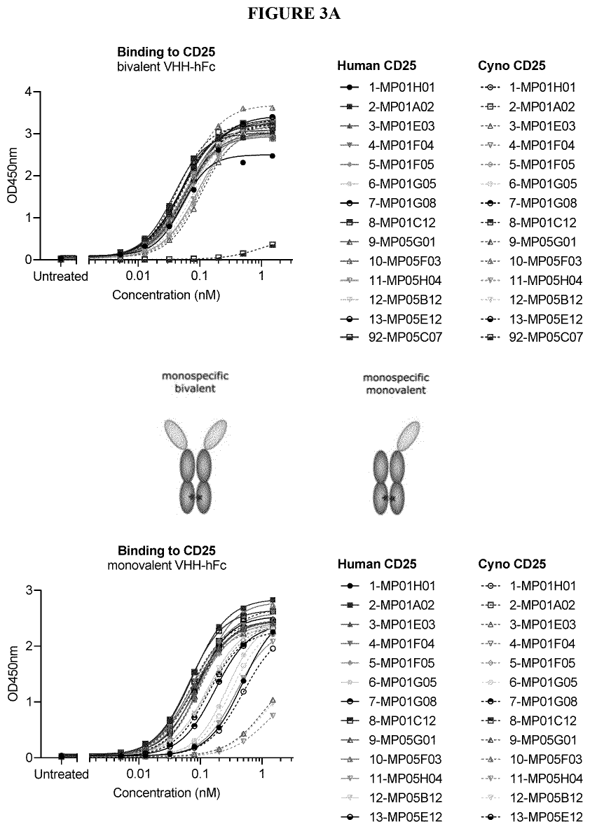

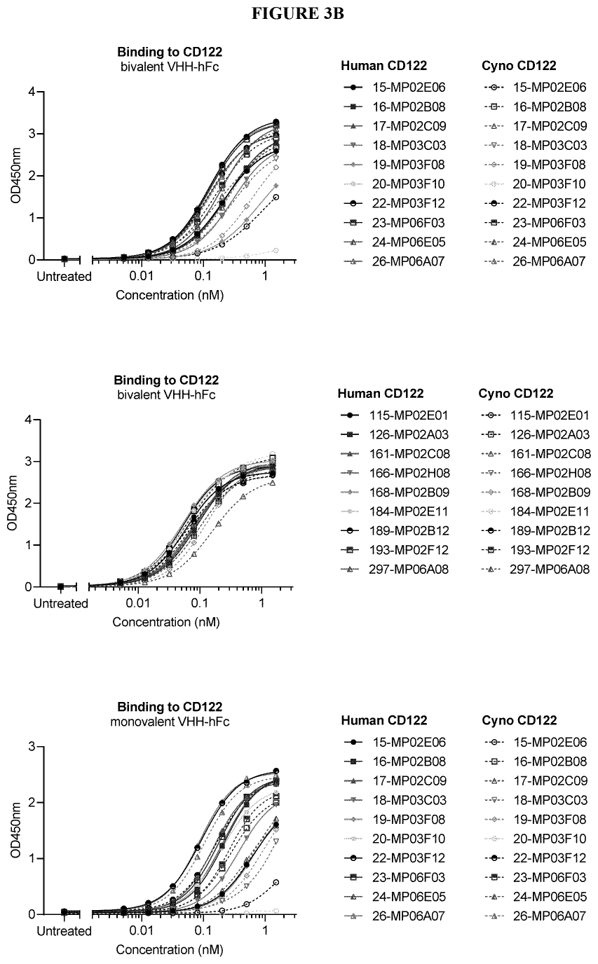

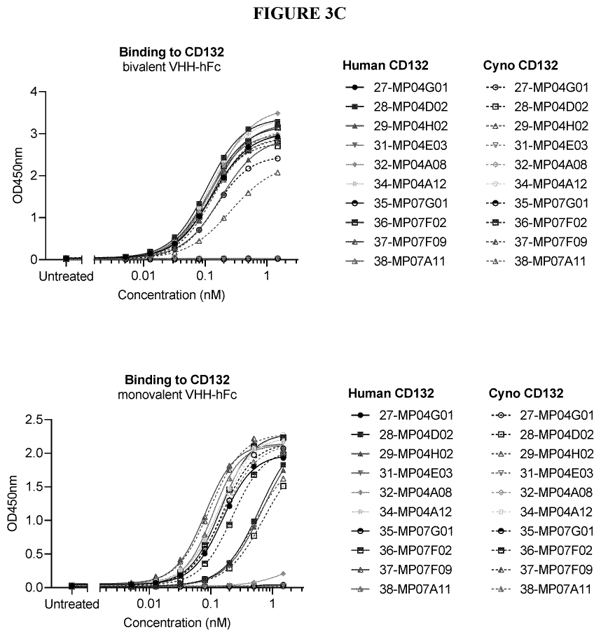

The binding properties of monospecific monovalent and bivalent anti-IL-2Rα, anti-IL-2Rβ and anti-γc antibodies are shown in FIG. 3A, FIG. 3B and FIG. 3C. The lower affinity of some antibody clones for the cynomolgus monkey IL-2R subunits is more pronounced in monovalent or one-armed format. In bivalent format, only several clones have significantly lower affinity for the cyno IL-2R subunits than for their human counterpart. The binding properties of the monospecific monovalent and bivalent VHH hFc clones to the human and cyno IL-2R subunits are shown in the TABLE 2. Fold-change of more than 1.5 between EC50 (nM) for binding to human and cyno receptor subunits indicates minor/no cross-reactivity.

### Example 3: Construction bispecific and trispecific antibodies co-targeting the IL-2Rα, IL-2Rβ and cγ

### A. Construction Bispecific Antibodies Targeting the IL-2Rβ and γc:

5 VHH clones specific for IL-2Rβ and 5 VHH clones specific for IL-2Rγ were selected and used to construct bispecific bivalent anti-IL-2Rβ/γc antibodies. Two VHH fragments were linked to a IgG1 backbone Fc region, while a (G₄S)₃ linker between the two VHH fragments and between the anti-IL-2Rβ VHH and the Fc region was used. The molecules comprised the amino acid substitutions L234A, L235A and P329G (LALA-PG) (EU numbering) in the immunoglobulin heavy chains, known to abrogate Fc-mediated effector functions. The Fc regions of the antibodies also included the mutations necessary for Fc domain heterodimerization by controlled Fab arm exchange (cFAE) (Labrijn et al. 2013. Proc Natl Acad Sci USA 110 (13): 5145-50; WO 2011/131746). In particular, the anti-IL-2Rβ/γc antibodies contained F405L CH3 domain mutation. Antibody molecules were produced by transient transfection in HEK293E cells and purified from cell supernatant by protein A affinity chromatography.

### B. Construction Trispecific Antibodies Targeting the IL-2Rα, IL-2β and IL-2Rγ:

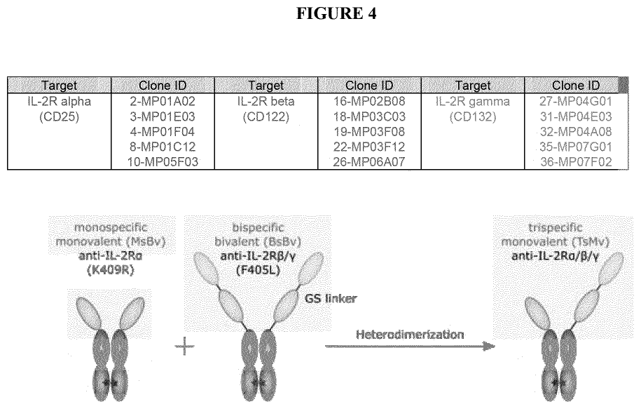

5 VHH clones specific for anti-IL-2Rα were produced as monospecific bivalent VHH-hFc fusion proteins (FIG. 4). The Fc portions of the antibodies comprised LALA-PG mutations and cFAE K409R CH3 domain mutation.

Trispecific anti-IL-2Rα/IL-2Rβ/γc antibodies were obtained using controlled Fab-arm exchange (cFAE) method described in Labrijn et al. 2013. Proc Natl Acad Sci USA 110 (13): 5145-5150 and WO 2011/131746. Monospecific bivalent anti-IL-2Rα antibodies containing K409R mutation and bispecific bivalent anti-IL-2Rβ/γc antibodies containing F405L mutation were mixed with a reducing agent at equimolar quantities. The resulting heterodimerisation of the Fc domains yielded trispecific monovalent anti-IL-2Rα/IL-2Rβ/γc antibodies (FIG. 4).

Protein integrity and heterodimerisation efficiency were analysed by an HPLC method using hydrophobic interaction chromatography (HIC) combined with ultraviolet spectrophotometry. HIC is a technique for separation of proteins based on their relative degree of hydrophobicity.

In HPLC-HIC the starting mobile phase contains a salting out agent. The high concentration of salt retains the protein by increasing hydrophobic interaction between solute and stationary phase. The bound proteins are eluted by decreasing the salt concentration. This is done using a gradient: starting with mobile phase A, high salt, gradually decreasing mobile phase A towards more mobile phase B, which contains very limited/no salt and if needed also organic solvent. The trispecific antibodies with asymmetric architecture are readily distinguished by this method: the retention time of the heterodimeric trispecific antibody is in between the parental homodimeric antibodies.

The HIC-HPLC was run through the MAbPac HIC-20 (ThermoFisher) column at a flow rate of 700 μL/min. The column temperature was kept at 30° C. and the sample temperature at 6° C. Stop time was set at 80 min. A sample having a total of 10 μg protein was run through HIC-HPLC. The antibodies were monitored by measuring their absorbance at 280 nm on the UV spectrum. The mobile phases included a Mobile Phase A and a Mobile Phase B. Mobile Phase A included 2.0 M ammonium sulphate and 100 mM sodium phosphate pH 7.1/H2O (75:25 (v/v)). Mobile Phase B included 100 mM sodium phosphate pH 7.0/H2O/isopropanol (60:20:20 (v/v/v)). The following gradient program was used:

| Time | A | B |
|---|---|---|
| min | % | % |
| 1.00 | 100.0 | 0.0 |
| 10.00 | 76.0 | 24.0 |
| 55.00 | 42.2 | 57.8 |
| 56.00 | 0.0 | 100.0 |
| 62.00 | 0.0 | 100.0 |
| 63.00 | 100.0 | 0.0 |
| 80.00 | 100.0 | 0.0 |

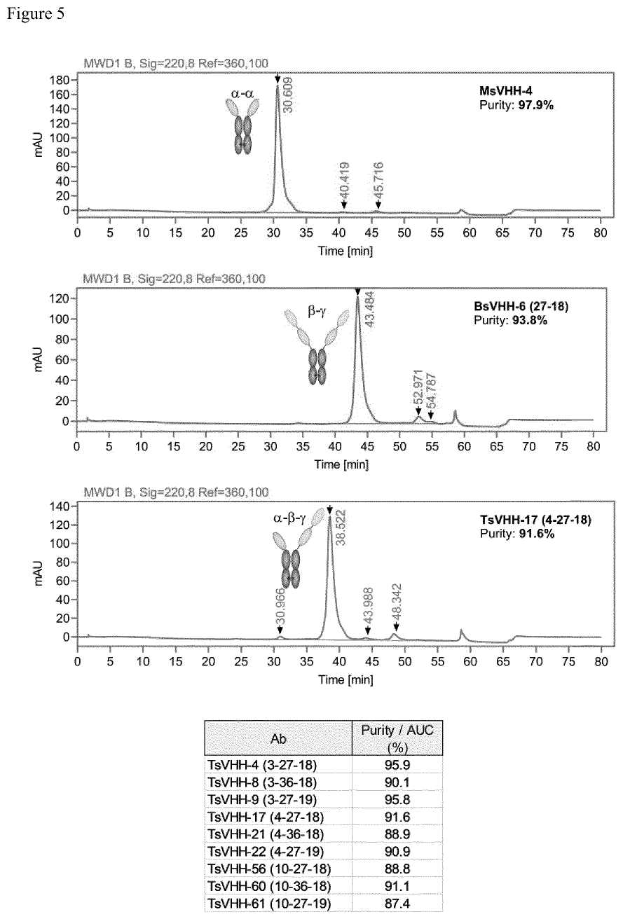

The trispecific antibodies with asymmetric architecture are readily distinguished by this method: the retention time of the heterodimeric trispecific antibody is in between the parental homodimeric antibodies. Heterodimerisation using cFAE method resulted in highly pure trispecific constructs. The purity and heterodimerisation efficiency were expressed as % main peak area; the results for the tested antibodies are summarised in (FIG. 5).

### Example 4: In vitro characterisation of antibodies targeting the IL-2Rα, IL-2Rβ and IL-2Rγ

Trispecific anti-IL-2Rα/IL-2Rβ/γc antibodies were tested for their ability to bind the human IL-2 receptor and to activate IL-2 signalling on human engineered cells expressing the three IL-2R subunits and on human PBMCs. Final analysis and graphic output were performed with FlowJo v10.7.1 software (BD Biosciences) and GraphPad Prism version 8 (GraphPad Software), respectively. The dose-response binding curves were fit to a nonlinear regression model (log (agonist) vs. response with a variable slope (four parameters)).

### A. Binding to HEK-Blue IL-2 Cells

The ability of trispecific anti-IL-2Rα/IL-2Rβ/γc antibodies to bind the human IL-2 receptor was analysed using the HEK-Blue IL-2 recombinant cell line (Invivogen, #hkb-il2) overexpressing the three IL-2R subunits. Cell culture was performed following the manufacturer's protocol. Cells were seeded at 100 000 cells/well in a 96-well plate, washed with FACS buffer, and incubated with antibodies diluted in FACS buffer at the concentration of 10 nM for 1 hour at 4° C., washed again with FACS buffer and stained with anti-human IgG-PE detection antibody (eBioscience) for 1 hour at 4° C. Dead cells were excluded from the analysis by using a fixable viability dye (eFluor780, eBioscience). Flow cytometric measurements were performed on a LSR Fortessa flow cytometer (BD Biosciences).

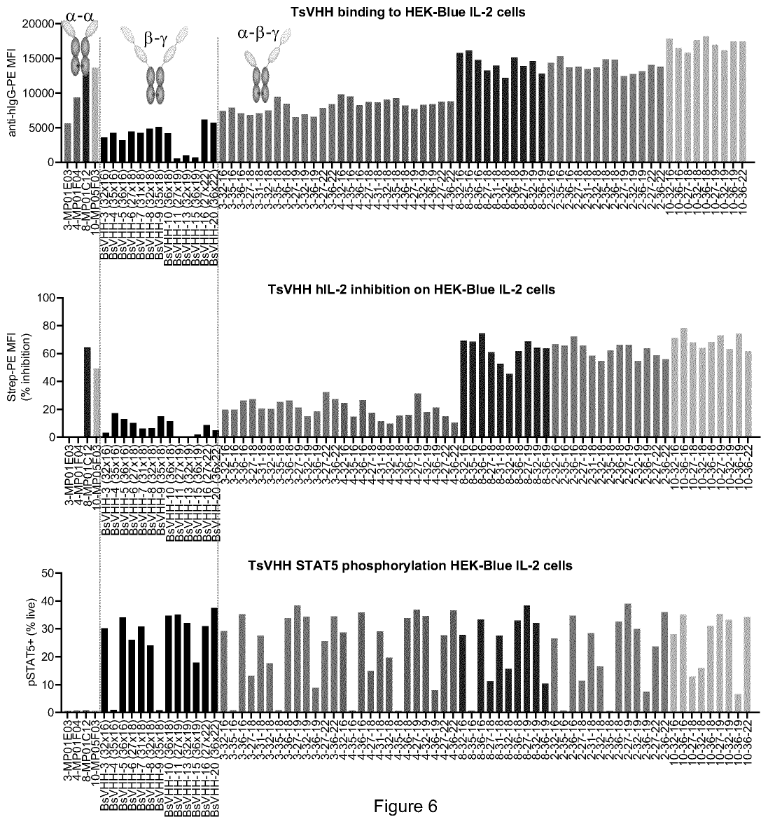

The cell binding properties of trispecific anti-IL-2Rα/IL-2Rβ/γc antibodies and their parental mono- and bispecific antibodies are shown in FIG. 6.

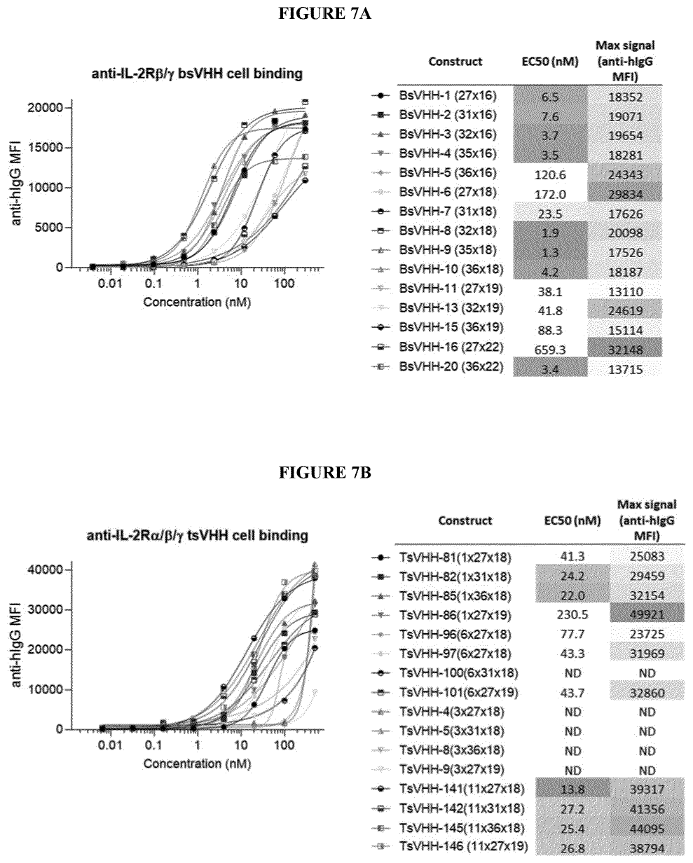

The cell binding properties of bispecific bivalent anti-IL-2Rβ/γc and monovalent trispecific anti-IL-2Rα/IL-2Rβ/γc antibodies were further assayed at multiple concentrations for cell binding on HEK-Blue IL-2 cells; the results are shown in FIG. 7A and FIG. 7B.

B. Interference with Human IL-2 Binding to its Receptor

The ability of trispecific anti-IL-2Rα/IL-2Rβ/γc antibodies to compete with the human IL-2 binding to its receptor was analysed using HEK-Blue IL-2 recombinant cell line (Invivogen, #hkb-il2) overexpressing the three IL-2R subunits. Cell culture was performed following the manufacturer's protocol. Cells were seeded at 100 000 cells/well in a 96-well plate, washed with FACS buffer, and preincubated with antibodies diluted in FACS buffer at the concentration of 10 nM for 20 min at 4° C., after which biotinylated human IL-2 (proteintech) at 2 nM was added for another 1 hour at 4° C. Cells were washed again with FACS buffer and stained with Streptavidin-PE (eBioscience) for 1 hour at 4° C. Dead cells were excluded from the analysis by using a fixable viability dye (eFluor780, eBioscience). Flow cytometric measurements were performed on a LSR Fortessa flow cytometer (BD Biosciences).

The neutralizing potencies of trispecific anti-IL-2Rα/IL-2Rβ/γc antibodies are depicted in FIG. 6. Several clones greatly inhibited the human IL-2 binding to its receptor. Specifically, trispecific antibodies that contain the strongest binding anti-IL-2Rα VHH 8 and 10 also induce the greatest inhibition of hIL-2 binding to its receptor.

C. Activation of pSTAT5 on HEK-Blue IL-2 cells

The potency of trispecific anti-IL-2Rα/IL-2Rβ/γc antibodies to induce IL-2 signalling was analysed by determining the level of STAT5 phosphorylated by HEK-Blue IL-2 recombinant cell line (Invivogen, #hkb-il2) in the presence of the antibodies. Cell culture was performed following the manufacturer's protocol. Cells were seeded at 200 000 cells/well in a 96-well plate in RPMI 0.1% BSA medium and treated with antibodies at a concentration of 50 nM for 1 hour at 4° C. Cells were further treated with IC Fixation buffer (eBioscience) for 15 min at room temperature, washed with FACS buffer, and incubated with BD Phosflow Perm Buffer III (BD Biosciences) for 30 min on ice. After washing with FACS buffer the cells were stained with anti-Stat5 (pY694)-PE detection antibody (BD Biosciences) overnight at 4° C. Dead cells were excluded from the analysis by using a fixable viability dye (eFluor780, eBioscience). Flow cytometric measurements were performed on a LSR Fortessa flow cytometer (BD Biosciences). The dose-response binding curves were fit to a nonlinear regression model (log (agonist) vs. response with a variable slope (four parameters)).

Results are depicted in FIG. 6. IL-2 signalling pathway is activated by the antibodies that contain both IL-2Rβ and IL-2Rγ VHH, but not by the monospecific anti-IL-2Rα antibodies. As such, pSTAT5 signal strength of trispecific anti-IL-2Rα/IL-2Rβ/γc clones depends mainly on the parental bispecific anti-IL-2Rα/β. These findings are expected because IL-2 signalling is proposed to occur via phosphorylation of IL-2Rβ and γc subunits, with IL-2Rα increasing the affinity of IL-2 for its receptor. Interestingly, pSTAT5 signal strength does not correlate with binding ability of the antibodies nor with their neutralization of human IL-2 potency.

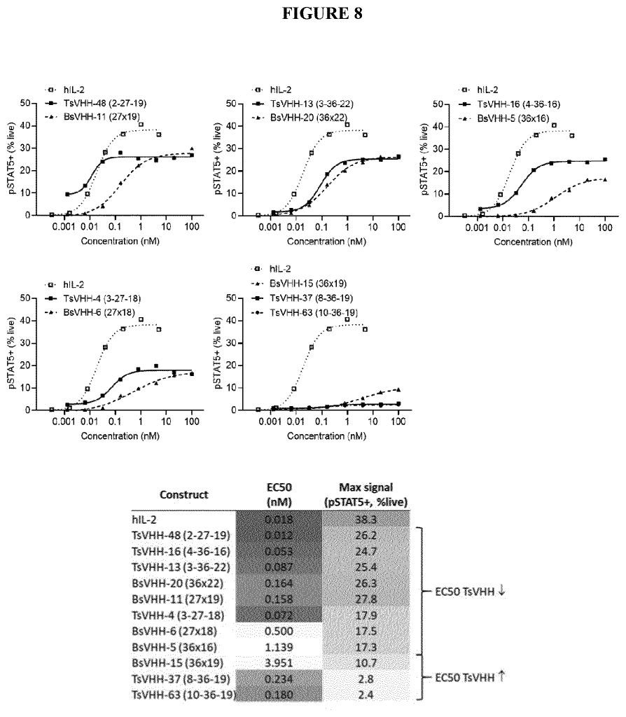

Several clones were further tested at multiple concentrations in order to assay the antibody concentration that gives half-maximal response (EC50). Bispecific bivalent anti-IL-2Rα/IL-2Rβ and trispecific anti-IL-2Rα/IL-2Rβ/γc antibodies induce dose-dependent pSTAT5 activation of HEK-Blue IL-2 cells. For some bispecific clones, addition of anti-IL-2Rα VHH decreases the EC50 value, suggesting improved CD25 targeting (FIG. 8).

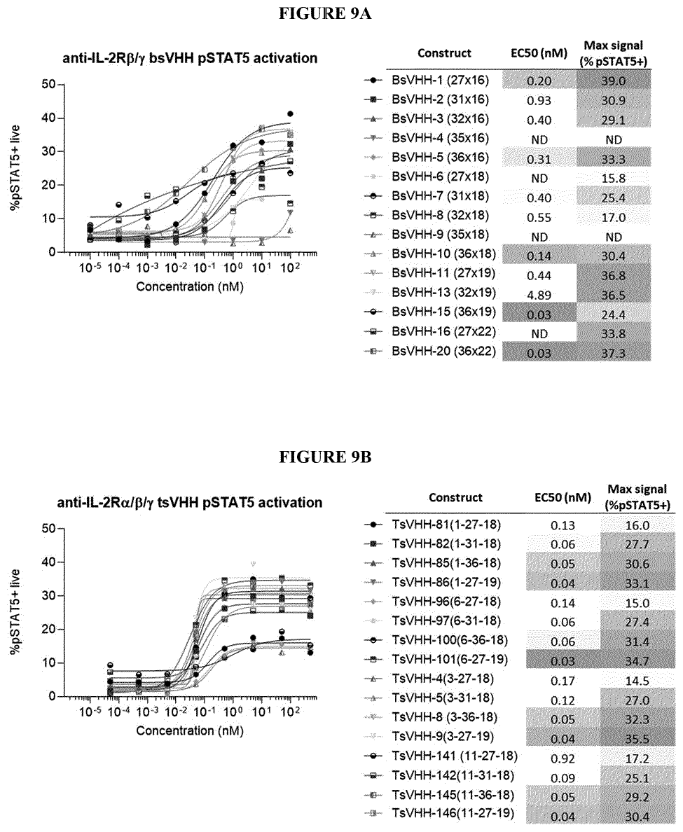

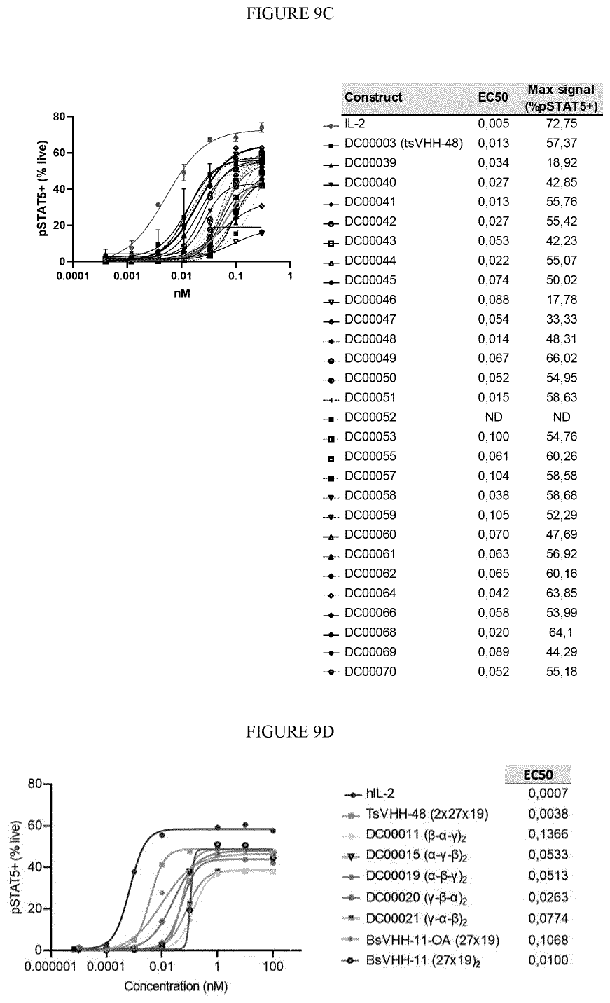

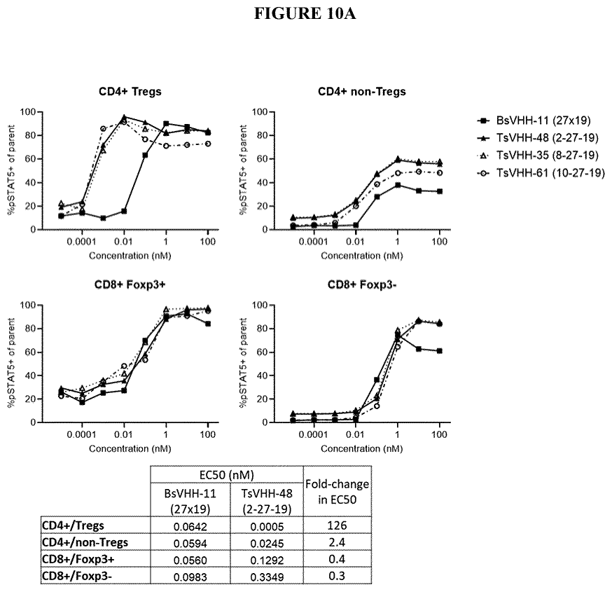

The potential of bispecific monovalent anti-IL-2Rβ/γc and trispecific anti-IL-2Rα/IL-2Rβ/γc antibodies to induce IL-2 signalling was further analysed at multiple concentrations using HEK-Blue IL-2 cells reading out STAT5 phosphorylation by flow cytometry. The results are shown in FIG. 9A and FIG. 9B. Several trispecific antibodies displayed a higher potency than the corresponding bispecific antibodies in this cell line assay, suggesting improved targeting of CD25 and the trimeric receptor.

The ability of bivalent trispecific anti-IL-2Rα/IL-2Rβ/γc antibody variants of tsVHH48 to induce pSTAT5 signalling via the human trimeric IL-2 receptor was analysed using a HEK-Blue cell line expressing the trimeric IL-2 receptor. The results are shown in FIG. 9E and the geometries of the antibodies are shown in FIG. 12.

D. Activation of pSTAT5 on Human PBMCs

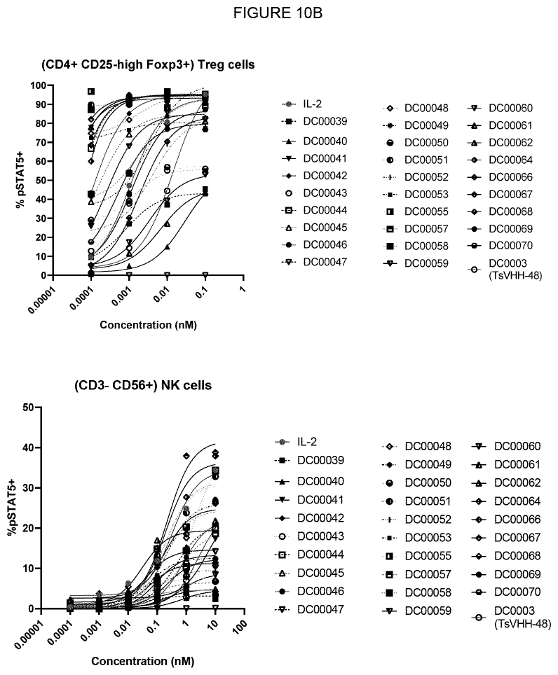

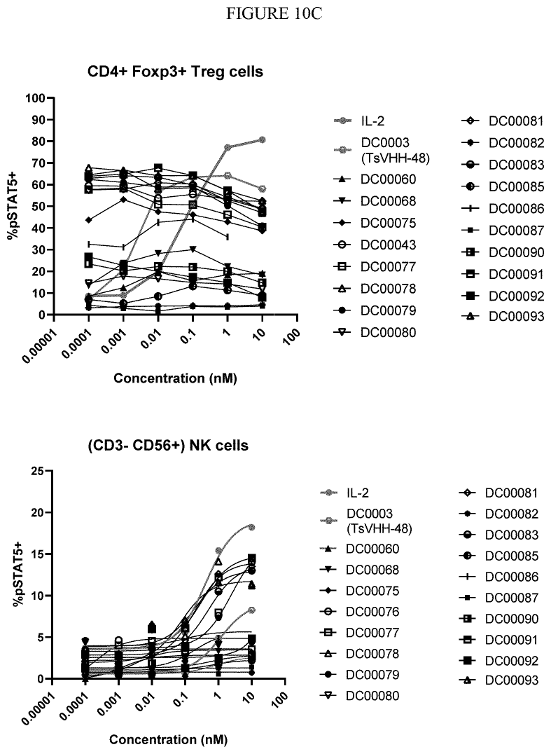

Peripheral blood mononuclear cells (PBMCs) were isolated from human healthy donor buffy coat donations (supplied by the Red Cross Flanders Blood Service, Belgium) using a Ficoll-Paque gradient. The cells were cultured for 2 days at high cell density using the protocol for resetting T cells to original reactivity (Wegner et al., Blood 2015). On the day of the experiment, the PBMCs were seeded at 10×10⁶ cells/well in a 96-well plate in RPMI 0.1% BSA medium and treated with antibodies for 1 hour at 4° C. Cells were further treated with IC Fixation buffer (eBioscience) for 15 min at room temperature, washed with FACS buffer, and incubated with BD Phosflow Perm Buffer III (BD Biosciences) for 30 min on ice. After washing with FACS buffer the cells were stained overnight at 4° C. with following detection antibodies: anti-human CD3 APC-eFluor780, CD4 PerCP-cFluor710, CD127 PE, Foxp3 cFluor660 (eBioscience), CD8 FITC, Stat5 (pY694) Pacific Blue (BD Biosciences). CD25 staining was performed either with anti-human CD25 PE-Cy7 clone 4E3 (eBioscience) or clone 2A3 (BD Biosciences), depending on which IL-2Rα-specific VHH was used for treatment. Dead cells were excluded from the analysis by using a fixable viability dye (cFluor506, eBioscience). Flow cytometric measurements were performed on a LSR Fortessa flow cytometer (BD Biosciences). Next, monospecific tsVHH-48 geometry variants and anti-CD25-biparatopic tsVHH-48 variants (FIG. 12C, upper & lower panels) were tested for their ability to more selectively activate pSTAT5 in CD4+ Tregs versus NK cells. Amongst non-Treg cell types, NK cells express the higher levels of CD122. Frozen PBMCs were seeded in 96-well plates and rested for 1 hour. Cells were then stimulated with varying doses of tsVHH variants for 40 minutes at 37° C. Stimulation was stopped by fixing cells with paraformaldehyde followed by methanol permeabilization of the cells. Cells were stained for 1 hour at room temperature with the following antibodies: anti-CD3 (clone UCHT1), anti-CD4 (clone SK3), anti-CD8 (clone SK1), anti-CD19 (HIB19), anti-CD56 (clone NCAM16.21), anti-CD127 (clone A019D5), anti-CD25 (clone M-A251), anti-Foxp3 (clone 259D/C7), anti-pSTAT5 (pY694), and dead cells were excluded using a fixable viability dye. Cells were analysed by flow cytometry. The pSTAT5 induction data is represented in FIGS. 10B and 10C and in TABLES 10 and 11. TsVHH-48 geometry variants were identified with increased potency for Tregs versus NK cells as compared to IL-2 or parental ts VHH-48 (TABLE 10). TsVHH-48 variants were also identified with decreased efficacy (max. % pSTAT5) on NK cells versus Tregs as compared to IL-2 and tsVHH-48. Furthermore, particular anti-CD25-biparatopic ts VHH variants show increased potency compared to parental tsVHH-48 or IL-2 (TABLE 11).

### E. Preferential Treg Expansion in Human PBMC Assay

Trispecific anti-IL-2Rα/IL-2Rβ/γc antibodies were assayed for their ability to preferentially expand CD25+ Tregs in human PBMC culture. Peripheral blood mononuclear cells (PBMCs) were isolated from human healthy donor buffy coat donations (supplied by the Red Cross Flanders Blood Service, Belgium) and cultured at high density for 2 days in order to restore the reactivity of T cells (Romer et al. 2011, Wegner et al. 2015 and US20110082091). Cells were seeded at 200 000 cells/well in 96-well U-bottom culture plates in RPMI-1640 culture medium (Gibco) supplemented with 10% FBS, 1% P/S, 2 mM L-Glutamine and freshly added 1:1000 B-mercaptoethanol. Cells were labelled with CFSE proliferation dye (Quah et al. 2007 Nature protocols) and stimulated with antibodies at different concentrations (100, 10, 1, 01 nM) for 4 days. Cells were stained with the following FACS antibodies: anti-human CD3 PerCP-VIO 700 (Miltenyi), CD4 BUV496, CD8 BUV805, CD56 BUV563 (BD Bioscience), FoxP3 APC, CD127 BV421, CD19 BV510, HLA-DR BV570 (BioLegend), CD69 PE-Cy7 (eBioscience). CD25 staining was performed either with anti-human CD25 PE-Cy7 clone 4E3 (eBioscience) or clone 2A3 (BD Biosciences), depending on which IL-2Rα-specific VHH was used for treatment. Dead cells were excluded from the analysis by using a fixable viability dye (eFluor780, eBioscience). Flow cytometric measurements were performed on a FACSymphony™ flow cytometer (BD Biosciences). Cell expansion was assessed by measuring CFSE proliferation profiles. TsVHH-48 demonstrates increased Treg selectivity and potency of inducing Treg proliferation versus wild-type IL-2 and the bsVHH-11 used to construct TsVHH-48.

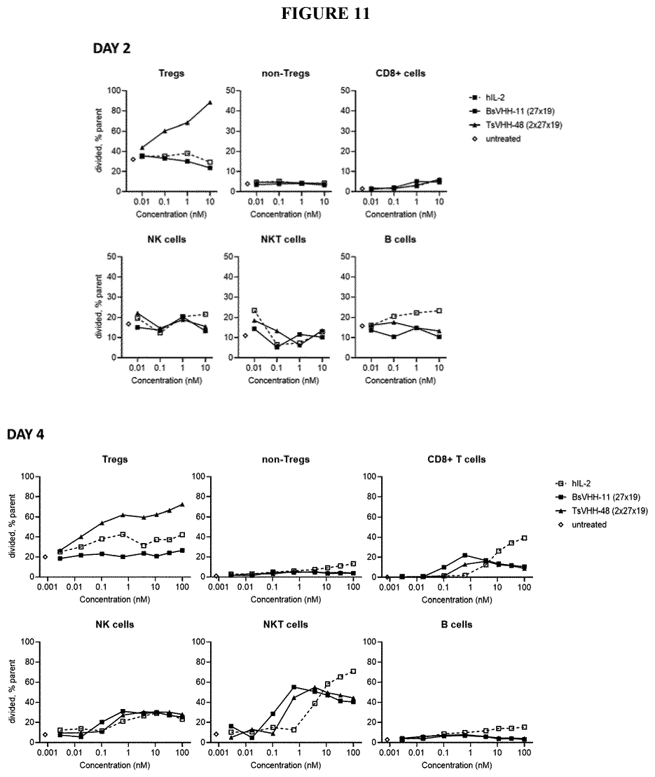

### Results are Depicted in FIG. 11.

Next to the monovalent trispecific geometry shown in FIG. 4, additional geometries were designed (FIG. 12A and FIG. 12B and FIG. 12C) to further modify the selectivity and/or potency of IL-2 signaling on Tregs, via increasing for example the number of epitopes for one of the IL-2R chains bound by the antibody from 1 to 2 and/or by increasing the number of CD25 binding moieties within the trispecific Abs. Varying geometries may also be tested for increased yield upon transfecting mammalian cells such as HEK293 cells as well as for case of production and purification. In particular, molecules with a symmetric architecture may be tested.

### Example 5: In Vivo Characterization of tsVHH48 in Acute Graft-Vs-Host-Disease Model

The agonistic anti-IL-2R antibodies were further evaluated for their ability to potentiate human Treg function in vivo. A model of xenogeneic graft-versus-host disease (GvHD) was used, which was induced by the infusion of human peripheral blood mononuclear cells (hPBMCs) into immuno-compromised NOD/Scid/IL2Rg−/−(NSG) mice. NSG mice have defective cytokine signaling and lack functional T, B and NK cells, allowing very efficient engraftment of human T cells upon i.v. injection of PBMCs. After hPBMC transfer, recipient mice develop xenogeneic GVHD, due to the activity of human cytotoxic T lymphocytes against murine tissues (Shultz, Nat Rev Immunol. 2012). Preferential Treg expansion would attenuate the disease. This model can thus be used to demonstrate the therapeutic efficacy of agonistic anti-Treg IL-2R trimer antibodies.

Male and female NSG mice between 6 to 10 weeks of age (bred and housed in specific pathogen-free facilities of the University of Leuven unless otherwise stated), were infused with 2×10E7 hPBMCs on day 0. These hPBMC were isolated from healthy blood donors' buffy coats (Belgian Red Cross) using density centrifugation (LSM MP Biomedicals, Germany). The GvH disease activity was evaluated by scoring the mice thrice per week. This score incorporated 6 clinical parameters, each one incrementing: 0 (no symptom), 1 (mild), or 2 (maximum). Parameters included are: weight loss (1 for >10% and 2 for >20%), posture (hunching), mobility, anemia, fur texture, and skin integrity. Mice reaching a disease activity score of 8 or those losing more than 20% of their initial weight were sacrificed in agreement with the KUL ethical committee procedure. All experimental procedures were approved by the Animal Care and Animal Experiments Ethical Committee of KU Leuven.

Mice were injected intraperitoneally with 1 μg, 0.3 μg or 0.1 μg ts VHH48 (100 μl, diluted in DPBS 1X), from day 2 and every 4 days for a total of 4 injections. As a control group, mice were intraperitoneally injected with 100 μl of PBS (Gibco) following the same scheme injection.

To evaluate the human leucocyte engraftment and the modulation of T and NK cell subsets over time, weekly immunophenotyping on blood was performed. Around 150 μl of blood was individually collected into 50 μl of heparin from day 7. Upon red blood cell lysis, each sample was stained with a live dead marker (live dead blue, thermofisher) for 20 min at 4° C. Then, the cells were blocked with Human BD Fc Block (BD) for 10 min at 4° C. and stained with the following antibodies: anti-mCD45 (clone 30-F11, BD), anti-hCD45 (clone HI30, Biolegend), anti-hCD3 (clone UCHT1, Biolegend), anti-hCD4 (clone OKT4, Sony), anti-hCD8 (clone SK1, Biolegend), anti-Ki67 (clone RUO, BD), anti-hCD127 (clone eBioRDR5, Ebioscience), anti-hFOXP3 (clone 206D, Biolegend), anti-hCD56 (clone 5.1H11, Sony), anti-hCD45RO (clone UCHL1, BD), anti-hCD45RA (clone GRT22, Invitrogen), anti-hCD25 (clone BC96, Sony), anti-hCCR4 (L291H4, Biolegend). The engraftment of the human cells was calculated using % hCD45/(% mCD45+% hCD45) from the total alive (Live dead blue negative population) cells. Flow cytometry was performed on the high parameter spectral SONY ID 7000 and analyzed on FCS express v7 (De Novo software). All the graphs and statistical analyses were performed using GraphPad Prism software.

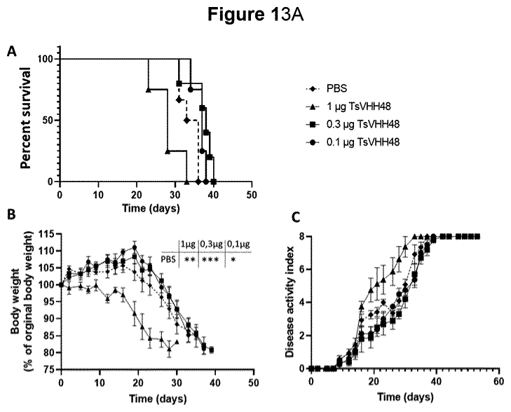

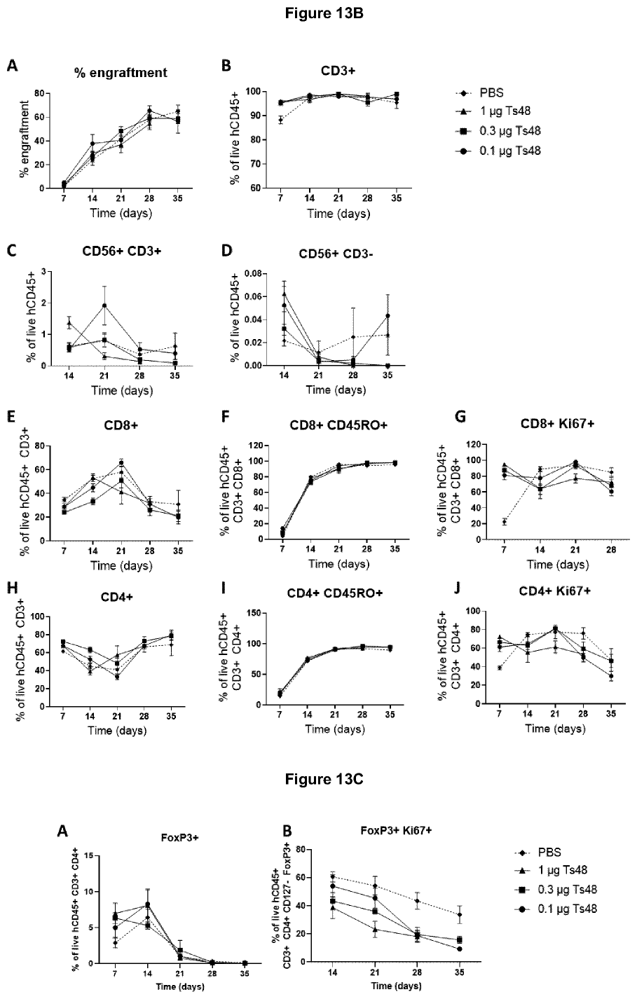

The survival and disease activity readouts are shown in FIG. 13A. The survival of the mice treated with tsVHH48 1 μg was lower than the PBS group (median survival 28 days and 34.5 days respectively) while survival increased for the doses 0.3 and 0.1 μg (median survival 38 and 37 days respectively). The weight loss (and disease activity readouts correlated with the survival trend: the 1 μg tsVHH48 group showed an earlier and faster decrease in weight while animals in the 0.3 μg and 0.1 μg groups kept their weight above animals from the PBS group. After day 14, disease activity for the 1 μg tsVHH48 group increased significantly above, (1 μg vs PBS, P=0.0024, Paired t-test) while the disease activity for the 0.3 μg and 0.1 μg groups were significantly below that of the PBS group (P=0.0002 and P=0.0146 respectively, Paired t-test).

To understand whether these differences were associated with a modulation of the effector or regulatory T cell population, we analyzed the frequency of these immune cell populations over time on blood (FIG. 13B). First, engraftment of human leukocytes (hCD45) was comparable between each group and the human CD45 population constituted mainly of T cells (>95% CD3+ cells,). Transient increases in NK T cell frequency was observed at early time points while frequencies of NK and NKT cells were comparable to or below the PBS group otherwise. A complete differentiation of T cells into effector memory T cells was observed for both CD4 and CD8 T cells without any major difference between the groups. With CD4 and CD8 T cells overall following the same pattern of expansion and attrition as the PBS group, CD4 T cells dominated the CD3+ T cells. The frequency of the Treg population (CD4+CD127-FOXP3+) was increased at day 7 and day 14 compared to the PBS group, with Treg numbers significantly higher for the 0.3 μg tsVHH48 dose group vs PBS group (p=0.0045). Hence the 0.3 μg and 0.1 μg dose groups seemed to increase the Treg frequency at the early timepoints (FIG. 13C).

TABLE 1
Binding characteristics of the selected clones.

| | | P.E. binding ELISA | | | | | | | | Sequence |
|---|---|---|---|---|---|---|---|---|---|---|
| | | (OD450 nm) | | P.E. Off-rate ranking SPR | | | | Competition | Epitope | analysis |
| | | human | mouse | huIL2R-a | | moIL2R-a | | SPR | binding SPR | CDR3 |
| Target | Clone ID | IL-2R | IL-2R | Rmax (Rus) | kd (1/s) | Rmax (Rus) | kd (1/s) | hIL-2:hIL-2RA | Epitope bin | Family ID |
| IL-2R | 12-MP05B12 | 2.136 | 0.936 | 585 | 2.22E−04 | 567 | 5.47E−04 | | H | 4 |
| alpha | 11-MP05H04 | 2.221 | 0.163 | 202 | 1.20E−03 | 533 | 1.63E−03 | no | H | 4 |
| (CD25) | 4-MP01F04 | 2.493 | 0.046 | 690 | 2.58E−04 | 18 | 2.56E−01 | no | H | 6 |
| | 1-MP01H01 | 1.705 | 0.048 | 127 | 2.29E−02 | 8 | N/A | no | H | 6 |
| | 9-MP05G01 | 2.353 | 0.047 | 570 | 1.69E−04 | 119 | 4.42E−02 | no | H | 7 |
| | 6-MP01G05 | 1.794 | 0.046 | 123 | 2.06E−02 | 10 | N/A | no | H | 10 |
| | 3-MP01E03 | 2.535 | 0.046 | 413 | 7.99E−04 | 9 | N/A | no | H | 14 |
| | 5-MP01F05 | 2.620 | 0.046 | 312 | 4.20E−04 | 9 | N/A | yes | I | 9 |
| | 2-MP01A02 | 2.615 | 0.046 | 847 | 1.53E−03 | 9 | N/A | yes | I | 2 |
| | 8-MP01C12 | 2.531 | 0.045 | 819 | 3.83E−04 | 8 | N/A | yes | I | 3 |
| | 10-MP05F03 | 2.465 | 0.054 | 780 | 6.39E−04 | 329 | 1.05E−01 | yes | J | 12 |
| IL-2R | 16-MP02B08 | 1.379 | 0.048 | 135 | 1.41E−04 | 3 | N/A | | A | 1 |
| beta | 20-MP03F10 | 0.497 | 0.048 | 52 | 5.95E−04 | 7 | N/A | | A | 3 |
| (CD122) | 24-MP06E05 | 0.355 | 0.046 | 315 | 4.71E−04 | 9 | N/A | | A | 5 |
| | 22-MP03F12 | 0.781 | 0.048 | 167 | 4.19E−04 | 4 | N/A | | A | 33 |
| | 15-MP02E06 | 0.581 | 0.048 | 88 | 1.69E−03 | 3 | N/A | | A | 38 |
| | 18-MP03C03 | 0.405 | 0.048 | 125 | 5.94E−04 | 14 | N/A | | ND | 34 |
| | 26-MP06A07 | 1.027 | 0.048 | 451 | 3.63E−04 | 46 | 8.17E−03 | | B | 20 |
| | 23-MP06F03 | 0.540 | 0.047 | 467 | 1.05E−03 | 29 | 1.93E−01 | | B | 20 |
| | 19-MP03F08 | 0.245 | 0.049 | 387 | 2.96E−04 | 5 | N/A | | C | 9 |
| | 17-MP02C09 | 1.205 | 0.047 | 100 | 8.48E−04 | −4 | N/A | | ND | 12 |
| IL-2R | 34-MP04A12 | 1.299 | 3.599 | 323.31 | 3.33E−04 | 342.33 | 8.09E−03 | | D | 1 |
| gamma | 28-MP04D02 | 0.782 | 0.054 | 166.11 | 8.80E−03 | 30.94 | 3.196−01 | | D | 1 |
| (CD132) | 38-MP07A11 | 1.307 | 0.173 | 59.76 | 2.35E−03 | 215.77 | 1.49E−02 | | D | 1 |
| | 36-MP07F02 | 0.896 | 0.060 | 63.96 | 3.31E−03 | 112.27 | 7.47E−02 | | D | 1 |
| | 37-MP07F09 | 1.806 | 1.487 | 97.14 | 1.26E−03 | 309.78 | 7.00E−03 | | D | 1 |
| | 31-MP04E03 | 0.706 | 0.495 | 169.31 | 8.43E−02 | 27.1 | 2.83E−01 | | F | 2 |
| | 32-MP04A08 | 0.407 | 0.050 | 256.29 | 6.16E−02 | 9.53 | N/A | | G | 3 |
| | 27-MP04G01 | 0.362 | 0.048 | 96.63 | 1.31E−03 | 9.52 | N/A | | E | 4 |
| | 35-MP07G01 | 0.134 | 0.045 | −1.24 | N/A | 5.31 | N/A | | E | 5 |
| | 29-MP04H02 | 0.157 | 0.050 | 104.01 | 6.25E−03 | 10.31 | N/A | | E | 7 |

TABLE 2
Cross-reactivity of the monospecific VHH-hFe clones binding the
three IL-2 receptor subunits CD25, CD122 and CD122.
Results from a binding ELISA on human and
cynomolgus IL-2R subunits.
NB: no binding; NC: not cross-reactive.

| | | | | BIVALENT | | | MONOVALENT | | |
|---|---|---|---|---|---|---|---|---|---|
| | | | | EC50 values (nM) | | | EC50 values (nM) | | |
| | | | CDR3 | Human | Cyno | | Human | Cyno | |
| IL-2R | | | family | IL-2R | IL-2R | Fold | IL-2R | IL-2R | Fold |
| subunit | Clone ID | CDR3 sequence | ID | subunit | subunit | change | subunit | subunit | change |
| CD25 | 1-MP01H01 | DNIPLSSDVAATATEYDY | 6 | 0.052 | 0.061 | 1.2 | 0.478 | 0.498 | 1.0 |
| | 2-MP01A02 | ATSYDSIRSGS | 2 | 0.038 | 0.043 | 1.1 | 0.074 | 0.096 | 1.3 |
| | 3-MP01E03 | TASSYSTYEANYNY | 14 | 0.057 | 0.106 | 1.9 | 0.086 | 1.149 | 13.4 |
| | 4-MP01F04 | DNIPLSSDMRPTATEYDY | 6 | 0.053 | 0.056 | 1.1 | 0.077 | 0.074 | 1.0 |
| | 5-MP01F05 | DRTGVGTNDYNY | 9 | 0.060 | 0.095 | 1.6 | 0.088 | 0.136 | 1.5 |
| | 6-MP01G05 | DSIPLSSDMSPTATEYGY | 10 | 0.056 | 0.050 | 0.9 | 0.270 | 0.317 | 1.2 |
| | 7-MP01G08 | DSIRLRSDVTRIPLEYDY | 11 | 0.072 | 0.067 | 0.9 | 0.173 | 0.133 | 0.8 |
| | 8-MP01C12 | ATSYTSIRGAP | 3 | 0.051 | 0.054 | 1.1 | 0.065 | 0.069 | 1.1 |
| | 9-MP05G01 | DPLSLTSDWRVDELSS | 7 | 0.065 | 0.062 | 1.0 | 0.078 | 0.074 | 1.0 |
| | 10-MP05F03 | RDGGVVAGSRSSAQYNY | 12 | 0.061 | 0.060 | 1.0 | 0.105 | 0.098 | 0.9 |
| | 11-MP05H04 | DRLGSQGRYASAWWRSGDMDL | 4 | 0.093 | 0.110 | 1.2 | 0.378 | 1.246 | 3.3 |
| | 12-MP05B12 | DRLGSRGAYVPIWWRSSDMDL | 4 | 0.074 | 0.084 | 1.1 | 0.130 | 1.100 | 8.5 |
| | 13-MP05E12 | ARERATWAYSEDDCDY | 1 | 0.049 | 0.051 | 1.0 | 0.089 | 0.095 | 1.1 |
| | 92-MP05C07 | YTYSGSFYSTVKTHHDEYRY | 15 | 0.047 | NB | NC | | | |
| CD122 | 15-MP02E06 | YSSSTYYPPTPARGRDY | 38 | 0.209 | 0.895 | 4.3 | 0.640 | HIGH | NC |
| | 16-MP02B08 | ALKTITRGQNDYSY | 1 | 0.245 | 0.230 | 0.9 | 0.215 | 0.280 | 1.3 |
| | 17-MP02C09 | DSWGGDDY | 12 | 0.124 | 0.184 | 1.5 | 0.148 | 0.495 | 3.3 |
| | 18-MP03C03 | VDAYGCSLVQPTTYDF | 34 | 0.294 | 0.300 | 1.0 | 0.373 | HIGH | NC |
| | 19-MP03F08 | DRRPMGSRSYFEPTEYDD | 9 | 0.794 | 0.603 | 0.8 | 0.670 | 0.953 | 1.4 |
| | 20-MP03F10 | ARGLPVTPLGDIIY | 3 | 0.218 | NB | NC | 0.197 | NB | NC |
| | 22-MP03F12 | TRAIGWTARWITTDFDF | 33 | 0.124 | 0.122 | 1.0 | 0.092 | 0.156 | 1.7 |
| | 23-MP06F03 | NTDYFQIKSLDANT | 20 | 0.125 | 0.157 | 1.3 | 0.174 | 0.260 | 1.5 |
| | 24-MP06E05 | DGPPYSGTYYRYDTYDY | 5 | 0.191 | 0.265 | 1.4 | 0.206 | 0.657 | 3.2 |
| | 26-MP06A07 | NTDYFQIRSLDLNT | 20 | 0.117 | 0.138 | 1.2 | 0.086 | 0.102 | 1.2 |
| | 115-MP02E01 | SPRGFYGPGNALYDY | 29 | 0.060 | 0.060 | 1.0 | | | |
| | 126-MP02A03 | YDSSTFYPPTPARGIAD | 36 | 0.090 | 0.065 | 0.7 | | | |
| | 161-MP02C08 | GPYGDAAYRHGRIDS | 15 | 0.084 | 0.157 | 1.9 | | | |
| | 166-MP02H08 | DRNIKITADWSY | 8 | 0.085 | 0.105 | 1.2 | | | |
| | 168-MP02B09 | ARRGRAAVRSEGGYDF | 4 | 0.053 | 0.127 | 2.4 | | | |
| | 184-MP02E11 | PRSGRAGTRNQMDYEY | 22 | 0.059 | 0.074 | 1.3 | | | |
| | 189-MP02B12 | RMYSASTYYGDYDY | 25 | 0.053 | 0.066 | 1.3 | | | |
| | 193-MP02F12 | TDAVGWTTRWMTADFGF | 31 | 0.087 | 0.099 | 1.1 | | | |
| | 297-MP06A08 | ARDWFARNEYQYDY | 2 | 0.079 | 0.090 | 1.2 | | | |
| CD132 | 27-MP04G01 | GDGWSTYDY | 4 | 0.131 | 0.147 | 1.1 | 0.155 | 0.153 | 1.0 |
| | 28-MP04D02 | APTSFATTAYSGSNSYAY | 1 | 0.107 | 0.113 | 1.1 | 0.620 | 0.723 | 1.2 |
| | 29-MP04H02 | TFWIERATTPDIGQYAY | 7 | 0.179 | 0.260 | 1.4 | 0.683 | 0.534 | 0.8 |
| | 31-MP04E03 | DNPSTLATDYDN | 2 | NB | NB | NB | NB | NB | NB |
| | 32-MP04A08 | DSLPYGRPYYFQRSAGEYDY | 3 | 0.140 | NB | NC | HIGH | NB | NB |
| | 34-MP04A12 | APTSFATTAYSSSNSYAY | 1 | 0.117 | 0.112 | 1.0 | 0.132 | 0.158 | 1.2 |
| | 35-MP07G01 | GRYYNSAYDPSPGDFGS | 5 | NB | NB | NB | NB | NB | NB |
| | 36-MP07F02 | APTSFPTTAYSSSNSYAY | 1 | 0.117 | 0.121 | 1.0 | 0.145 | 0.222 | |
| | 37-MP07F09 | APTSFATTAYSSSNSYRY | 1 | 0.137 | 0.141 | 1.0 | 0.081 | 0.097 | 1.2 |
| | 38-MP07A11 | APTSFATTAYSSSNSYSY | 1 | 0.113 | 0.133 | 1.2 | 0.107 | 0.160 | 1.5 |

TABLE 3
Amino acid sequences of monovalent anti-CD25
(IL-2Rα), anti-CD122 (IL-2Rβ) and anti-CD132
(γc) VHHs. “ID” refers to
the SEQ ID NO as used herein.

| | | IL-2R | |
|---|---|---|---|
| ID | Name | subunit | Sequence |
| 1 | 1-MP01H01 | IL-2Rα | EVQLVESGGGLVQAGGSLRLSCAAS |
| | | | GRTFRTHNMGWFRRAPGKEREFVAA |
| | | | ISWNVDNTLYADSVKGRFTISRDNG |
| | | | RNMVYLQMNSLKPEDTAVYYCAADN |
| | | | IPLSSDVAATATEYDYWGQGTQVTV |
| | | | SS |
| | | | (SEQ ID NO: 1) |
| 2 | 2-MP01A02 | IL-2Rα | QVQLVESGGGLVQPGGSLRLSCATS |
| | | | GFTFRNNFMSWVRQAPGKGLEWVST |
| | | | ISYGGESTTYAESVKGRFTISRDNA |
| | | | KNTLYLQMNNLKPEDTAVYYCAKAT |
| | | | SYDSIRSGSRGQGTQVTVSS |
| | | | (SEQ ID NO: 2) |
| 3 | 3-MP01E03 | IL-2Rα | QVQLVESGGGLVQAGGSLRLSCTAS |
| | | | GRTLSSYSMAWFRQAADKGREFVTA |
| | | | ISSSGVVTHVLDSVKGRFTISRDNA |
| | | | KNTVYLQMNSLQPEDTALYFCAGTA |
| | | | SSYSTYEANYNYWGQGTLVTVSS |
| | | | (SEQ ID NO: 3) |
| 4 | 4-MP01F04 | IL-2Rα | QVQLVESGGGLVQAGGSLRLSCAAS |
| | | | GGTFRTRNMGWFRRAPGKEREFVAA |
| | | | VSWNVDNKLYADSVKGRFTISRDNG |
| | | | RNMVYLQMNSLKPEDTAVYYCAADN |
| | | | IPLSSDMRPTATEYDYWGQGTQVTV |
| | | | SS |
| | | | (SEQ ID NO: 4) |
| 5 | 5-MP01F05 | IL-2Rα | EVQLVESGGGLVQAGGSLRLSCAAS |
| | | | IRAFTTWSMAWFRQAPGKEREYVAR |
| | | | INVSGSVTYYADFVKGRFTISRDNA |
| | | | KKTMYLEMNNLKPEDTALYYCAADR |
| | | | TGVGTNDYNYWGQGTQVTVSS |
| | | | (SEQ ID NO: 5) |
| 6 | 6-MP01G05 | IL-2Rα | QVQLQESGGGLVQAGGSLRLSCAAS |
| | | | GRTFSSYAMGWFRQAPGKEREFVAV |
| | | | ISWNVDNTEYTDSVKGRFTISRDND |
| | | | KNMVYLQMNSLKPEDTAVYYCAADS |
| | | | IPLSSDMSPTATEYGYWGQGTQVTV |
| | | | SS |
| | | | (SEQ ID NO: 6) |
| 7 | 07-MP01G08 | IL-2Rα | EVQLVESGGGLVQAGGSLRLSCAAS |
| | | | GRAFSMYNMGWFRQAPGKEREFVAA |
| | | | TGWSGANTPYADSVKGRFTISRDNT |
| | | | QNTVYLQMNSLKPEDTATYYCAADS |
| | | | IRLRSDVTRIPLEYDYWGQGTQVTV |
| | | | SS |
| | | | (SEQ ID NO: 7) |
| 8 | 8-MP01C12 | IL-2Rα | QVQLVESGGGLVQPGGSLRLSCATS |
| | | | GFTFSNNFMSWVRQAPGKGLEFVST |
| | | | ISYGGESTTYAEAVKGRFTISRDNA |
| | | | KNTLYLQMNNLKPEDTAIYYCAKAT |
| | | | SYTSIRGAPRGQGTQVTVSS |
| | | | (SEQ ID NO: 8) |
| 9 | 9-MP05G01 | IL-2Rα | EVQLVESGGGLVQAGGSLRLSCAAS |
| | | | GGSIYTYNMGWFRQAPGKEREFVAG |
| | | | TLWSGGDSVYADFAKGRFTLSRENA |
| | | | KNTLYLQMNSLKPEDTATYYCAIDP |
| | | | LSLTSDWRVDELSSWGKGTLVTVSS |
| | | | (SEQ ID NO: 9) |
| 10 | 10-MP05F03 | IL-2Rα | QVQLVESGGGLVQAGGSLRLSCAAS |
| | | | GIPFDNYAMGWFRQAPGKEREFVAA |
| | | | RDLEGIITRYGDSVKGRFTISRGNA |
| | | | KNTVFLQMNSLKPEDTAVYYCAARD |
| | | | GGVVAGSRSSAQYNYWGQGTQVTVS |
| | | | S |
| | | | (SEQ ID NO: 10) |
| 11 | 11-MP05H04 | IL-2Rα | QLQLVESGGGLVQAGGSLRLSCAAS |
| | | | GLTFEGYAIGWFRQAPGKEREGVSY |
| | | | ITGSDGTTYYINSVKGRFTISSDNA |
| | | | KSTVYLQMNTLKPEDTAVYYCAVDR |
| | | | LGSQGRYASAWWRSGDMDLWGKGTQ |
| | | | VTVSS |
| | | | (SEQ ID NO: 11) |
| 12 | 12-MP05B12 | IL-2Rα | QVQLVESGGGLVQAGGSLRLSCAAS |
| | | | GFTFDGYAIGWFRQAPGKEREGVSY |
| | | | ITGSDGSTYYADSVKGRFTISSNNA |
| | | | KNTVYLHMNSLKPDDAAVYYCAIDR |
| | | | LGSRGAYVPIWWRSSDMDLWGKGTL |
| | | | VTVSS |
| | | | (SEQ ID NO: 12) |
| 13 | 13-MP05E12 | IL-2Rα | QVQLQESGGGLVQAGDSLRLSCAAS |
| | | | GFNFGWHAMGWFRQAPGKEREFVAT |
| | | | ITWTGRDTYYADSVRGRFTISKDNA |
| | | | KDTLFLQMNSLRPDDTGVYYCAKAR |
| | | | ERATWAYSEDDCDYWGQGTQVTVSS |
| | | | (SEQ ID NO: 13) |
| 14 | 14-MP02C03 | IL-2Rβ | EVQLVESGGGLVQTGGSLRLSCAAS |
| | | | GSQFINDVMGWYRQVPGKQRELVAD |
| | | | MDDTGSTEYADSVKGRFTILRDSVK |
| | | | NTAYLQMSNLKPEDTGVYYCKAGLW |
| | | | IKGRHFDYWGQGTQVTVSS |
| | | | (SEQ ID NO: 14) |
| 15 | 15-MP02E06 | IL-2Rβ | QVQLVESGGGSVQPGGSLRLSCAAS |
| | | | GFTFSNYAMSWVRQAPGKGLEWVAS |
| | | | ITGFGRGTDYADSVKGRFTISRDNA |
| | | | EDTLYLQMNSLKPEDTAVYYCAKYS |
| | | | SSTYYPPTPARGRDYRGQGTQVTVS |
| | | | S |
| | | | (SEQ ID NO: 15) |
| 16 | 16-MP02B08 | IL-2Rβ | EVQLVESGGGLVQAGGSLRLSCAAS |
| | | | GRAIENYPVGWFRQAPGKEREFVAA |
| | | | ITWISGSTLYADSVKGRFTISRDNA |
| | | | KNTVYLQMSSLKPEDTALYYCAAAL |
| | | | KTITRGQNDYSYWGQGTQVTVSS |
| | | | (SEQ ID NO: 16) |
| 17 | 17-MP02C09 | IL-2Rβ | QVQLQESGGGLVQAGGSLRLSCVAS |
| | | | GSVSSINGMAWYRQGADNQRVLVAA |
| | | | ISRVGNTAYGDSVKGRFTISRQNAR |
| | | | NTVYLQMNSLKPEDTAVYYCNADSW |
| | | | GGDDYWGQGTQVTVSS |
| | | | (SEQ ID NO: 17) |
| 18 | 18-MP03C03 | IL-2Rβ | QVQLVESGGGLVQPGGSLRLSCAIS |
| | | | GGTLDSYGIGWVRQAPGKQREGVSC |
| | | | MSRSDDRTYYADSVKGRFTISKDSA |
| | | | KNTVYLQMTSLKPEDTAVYYCAAVD |
| | | | AYGCSLVQPTTYDFWGLGTQVTVSS |
| | | | (SEQ ID NO: 18) |
| 19 | 19-MP03F08 | IL-2Rβ | EVQLVESGGGLVQTGGSLRLSCAAS |
| | | | GGTFSRDAMAWFRQVPGKEREFVAL |
| | | | ISWSGATTNYADSVKGRFAISRDNG |
| | | | KNTVYLQMNRLKPADTAIYYCAADR |
| | | | RPMGSRSYFEPTEYDDWGQGTQVTV |
| | | | SS |
| | | | (SEQ ID NO: 19) |
| 20 | 20-MP03F10 | IL-2Rβ | EVQLVESGGGLVQAGGSLRLSCAAS |
| | | | GRDFSSYAMGWFRQAPGKEREFVVA |
| | | | ITWTKRSTDFPDSVKGRFTISRDNA |
| | | | KNTVYLDMNSLKPEDTAVYYCASAR |
| | | | GLPVTPLGDIIYWGEGTLVTVSS |
| | | | (SEQ ID NO: 20) |
| 21 | 21-MP03A12 | IL-2Rβ | EVQLVESGGGLVQAGGSLRLSCAAS |
| | | | GRTFSINAMGWFRQAPGKEREFVAA |
| | | | ISRSGGSTVYVDGVKGRFTISRDNA |
| | | | KNTVYLQMNSLEPEDTAVYYCAATM |
| | | | AVGWTTRWRTADFDSWGQGTQVTVS |
| | | | S |
| | | | (SEQ ID NO: 21) |
| 22 | 22-MP03F12 | IL-2Rβ | EVQLVESGGGLVQAGGSLRLSCAAS |
| | | | GSIFSINAMAWFRQVPGMERELVAA |
| | | | ISRDGGASVYRDSVKGRFTISRDNS |
| | | | KNTVYLQMNTLKPEDTAIYVCAATR |
| | | | AIGWTARWITTDFDFWGQGTQVTVS |
| | | | S |
| | | | (SEQ ID NO: 22) |
| 23 | 23-MP06F03 | IL-2Rβ | QVQLVESGGGLVQAGGSLRLSCAVS |
| | | | GDVFVRYTMAWFRQAPGKEREFVAS |
| | | | VTDSGRTTDYVHSVKGRFTVSRDNA |
| | | | KNTVYLQMNNLKPEDTAVYYCAANT |
| | | | DYFQIKSLDANTWGQGTQVTVSS |
| | | | (SEQ ID NO: 23) |
| 24 | 24-MP06E05 | IL-2Rβ | QVQLVESGGELVQGGASLRLSCAAS |
| | | | GRTFSNANMAWFRQAPEKEREFVAL |
| | | | ITWSSGSTLYADSVKGRFTISRDNA |
| | | | RKMVYLQMNSLKPEDTAVYYCAADG |
| | | | PPYSGTYYRYDTYDYWGQGTQVTVS |
| | | | S |
| | | | (SEQ ID NO: 24) |
| 25 | 25-MP06F05 | IL-2Rβ | QVQLVESGGGLVQTGDSLRLSCAAS |
| | | | GRSLDTTYIAWFRQAPGKERDFLAY |
| | | | ISPRFSHTWYADSVKGRFTISRNIA |
| | | | KRTVDLEMNSLEPEDTAVYYCAARE |
| | | | HSGSTAWEHYDHWGQGTQVTVSS |
| | | | (SEQ ID NO: 25) |
| 26 | 26-MP06A07 | IL-2Rβ | QVQLQESGGGLVQAGGSLRLSCAAS |
| | | | GDVFVRYTMAWFRQAPGKEREFVAS |
| | | | VTDSGRTTEYVDSVKGRFTVSRDNA |
| | | | KNTAYLQMNNLKPEDTAIYYCAANT |
| | | | DYFQIRSLDLNTWGQGTQVTVSS |
| | | | (SEQ ID NO: 26) |
| 27 | 27-MP04G01 | IL-2Rγ | QVQLVESGGGLVQAGGSLTLSCAAP |
| | | | GRTFGTDVVGWFRQAPGKEREFVAS |
| | | | ISRSGDGIYYDDSVKGRFTISRNNA |
| | | | WNTVNLQMNSLKVEDTAVYYCAAGD |
| | | | GWSTYDYWGQGTQVTVSS |
| | | | (SEQ ID NO: 27) |
| 28 | 28-MP04D02 | IL-2Rγ | QVQLVESGGGLVQAGGSLRLSCAAS |
| | | | GRTLSRYAMGWFRQAPGKEREFVTA |
| | | | NSWGGDTYYADSVQGRFTFSRDNAK |
| | | | NTVYLQMNSLQPEDTAVYYCAAAPT |
| | | | SFATTAYSGSNSYAYWGQGTQVTVS |
| | | | S |
| | | | (SEQ ID NO: 28) |
| 29 | 29-MP04H02 | IL-2Rγ | QVQLVESGGGLVQAGGSLRLACVAS |
| | | | GLTFDNYYMGWFRQAPGKEREFVAG |
| | | | IIWNGDHTAYADSIKGRFTISRDNA |
| | | | KNTAYLRMNSLKPEDTAVYYCAATF |
| | | | WIERATTPDIGQYAYWGQGTQVTVS |
| | | | S |
| | | | (SEQ ID NO: 29) |
| 30 | 30-MP04C03 | IL-2Rγ | EVQLVESGGGWVQDGGSLRLSCALS |
| | | | GRTFVRGIMGWFRQAPGKEREFVAR |
| | | | IIWHINSTRYADSVKGRFTISRDSA |
| | | | KNTMYLQMDSLRPEDTAVYYCAARD |
| | | | RYGSGNSLSPSAYDYWGQGTQVTVS |
| | | | S |
| | | | (SEQ ID NO: 30) |
| 31 | 31-MP04E03 | IL-2Rγ | QVQLVESGGGLVQAGGSLRLSCTGY |
| | | | GGAFTGYALGWFRQAPGKEREFVAR |
| | | | INWSGSFTYYASSVKGRFTISRDNA |
| | | | KNTMYLQMNNLKPEDTAVYYCAADN |
| | | | PSTLATDYDNWGQGTQVTVSS |
| | | | (SEQ ID NO: 31) |
| 32 | 32-MP04A08 | IL-2Rγ | QVQLVESGGGLVQAGGSLRLSCAAS |
| | | | GRTFGSTAVGWFRQVPGKEREFVSA |
| | | | INRSGSATTYADSVKGRFTISRDNA |
| | | | KNTVYLQMNSLTPEDTGVYYCAADS |
| | | | LPYGRPYYFQRSAGEYDYWGQGTQV |
| | | | TVSS |
| | | | (SEQ ID NO: 32) |
| 33 | 33-MP04C09 | IL-2Rγ | QLQLVESGGGLVQAGGSLRLSCAAS |
| | | | GPTFSRVAVGWFRQAPGKEREFVAA |
| | | | VNRPATMTKYADSVKGRFTVSRDNA |
| | | | KNTVDLQMNSMKPEDTAVYYCAADS |
| | | | VPYGRPYYWQTSAGDYDYWGQGTQV |
| | | | TVSS |
| | | | (SEQ ID NO: 33) |
| 34 | 34-MP04A12 | IL-2Rγ | QVQLVESGGGLVQAGSSLRLSCAAS |
| | | | GRTLSRLAMGWFRQAPGKEREFVAV |
| | | | NSWGGDTFYADSVEGRFTYSRDNAK |
| | | | SAVYLQMNSLQPEDTAVYYCAAAPT |
| | | | SFATTAYSSSNSYAYWGQGAQVTVS |
| | | | S |
| | | | (SEQ ID NO: 34) |
| 35 | 35-MP07G01 | IL-2Rγ | QVQLQESGGGLVQGGGSLRLSCAAS |
| | | | GGIFSSYAMGWFRQAPGKEREFVAA |
| | | | ISRSGRSTNYADSVKGRFTISRDNA |
| | | | KSTVYLQMNSLKPEETAVYYCAAGR |
| | | | YYNSAYDPSPGDFGSWGHGTQVTVS |
| | | | S |
| | | | (SEQ ID NO: 35) |
| 36 | 36-MP07F02 | IL-2Rγ | QVQLVESGGGLVQAGGSLRLSCAAS |
| | | | GRTLSRYAMGWFRQAPGSEREFVAA |
| | | | SSWGGDTFYADSVEGRFTFSRDNAK |
| | | | NAVYLQMNSLQPEDTAAYYCAAAPT |
| | | | SFPTTAYSSSNSYAYWGQGTQVTVS |
| | | | S |
| | | | (SEQ ID NO: 36) |
| 37 | 37-MP07F09 | IL-2Rγ | QVQLVESGGGLVQAGGSLRLSCAAS |
| | | | GRTLSRYAMGWFRQAPGKEREYVAI |
| | | | DSWGGDTFYADSVEGRFTFSRDNAK |
| | | | NEVYLQMNSLQPEDTAVYYCAGAPT |
| | | | SFATTAYSSSNSYRYWGQGTQVTVS |
| | | | S |
| | | | (SEQ ID NO: 37) |
| 38 | 38-MP07A11 | IL-2Rγ | QVQLVESGGGLVQAGGSLRLSCAAS |
| | | | GRSLSRDAMGWFRQAPGKEREFVAV |
| | | | MSWGGDTFYTDSVEGRFTFSRDNAK |
| | | | NAVYLEMNDLQPEDTAVYYCAAAPT |
| | | | SFATTAYSSSNSYSYWGRGTQVTVS |
| | | | S |
| | | | (SEQ ID NO: 38) |

TABLE 4
Sequences for CDRs and frameworks. The first column refers to the SEQ ID NO of the complete immunoglobulin single
variable domain (ISV), i.e. FR1-CDR1-FR2-CDR2-FR3-CDR3-FR4. CDR1, CDR2, CDR3 were determined according to Kontermann,
12010 (Kontermann & Dübel, 2010 Springer, Antibody Engineering).

| SEQ | | SEQ | | SEQ | | SEQ | | SEQ | | SEQ | | SEQ | | SEQ | |
|---|---|---|---|---|---|---|---|---|---|---|---|---|---|---|---|
| ID | Name | ID | FR1 | ID | CDR1 | ID | FR2 | ID | CDR2 | ID | FR3 | ID | CDR3 | ID | FR4 |
| 1 | 1- | 1868 | EVQLVESGGGLVQAGG | 467 | THNMG | 1869 | WFRRAP | 468 | AISWNVD | 1870 | RFTISRDNGRNMVY | 469 | DNIPLSSDVAA | 1871 | WGQ [symbol] |
| | MP01H | | SLRLSCAASGRTFR | | | | GKEREF | | NTLYADSV | | LQMNSLKPEDTAVY | | TATEYDY | | QVTV [symbol] |
| | 01 | | | | | | VA | | KG | | YCAA | | | | |
| 2 | 2- | 1872 | QVQLVESGGGLVQPG | 470 | NNFMS | 1873 | WVRQAP | 471 | TISYGGES | 1874 | RFTISRDNAKNTLYL | 472 | ATSYDSIRSGS | 1875 | RGQGT |
| | MP01A | | GSLRLSCATSGFTFR | | | | GKGLEW | | TTYAESVK | | QMNNLKPEDTAVY | | | | QVTVSS |
| | 02 | | | | | | VS | | G | | YCAK | | | | |
| 3 | 3- | 1876 | QVQLVESGGGLVQAG | 473 | SYSMA | 1877 | WFRQAA | 474 | AISSSGVV | 1878 | RFTISRDNAKNTVY | 475 | TASSYSTYEA | 1879 | WGQGT |
| | MP01E | | GSLRLSCTASGRTLS | | | | DKGREF | | THVLDSVK | | LQMNSLOPEDTALY | | NYNY | | LVTVSS |
| | 03 | | | | | | VT | | G | | FCAG | | | | |
| 4 | 4- | 1880 | QVQLVESGGGLVQAG | 476 | TRNMG | 1881 | WFRRAP | 477 | AVSWNVD | 1882 | RFTISRDNGRNMVY | 478 | DNIPLSSDMR | 1883 | WGQGT |
| | MP01F | | GSLRLSCAASGGTFR | | | | GKEREF | | NKLYADS | | LQMNSLKPEDTAVY | | PTATEYDY | | QVTVSS |
| | 04 | | | | | | VA | | VKG | | YCAA | | | | |
| 5 | 5- | 1884 | EVQLVESGGGLVQAGG | 479 | TWSM | 1885 | WFRQAP | 480 | RINVSGSV | 1886 | RFTISRDNAKKTMY | 481 | DRTGVGTNDY | 1887 | WGQGT |
| | MP01F | | SLRLSCAASIRAFT | | A | | GKEREY | | TYYADFVK | | LEMNNLKPEDTALY | | NY | | QVTVSS |
| | 05 | | | | | | VA | | G | | YCAA | | | | |
| 6 | 6- | 1888 | QVQLQESGGGLVQAG | 482 | SYAMG | 1889 | WFRQAP | 483 | VISWNVDN | 1890 | RFTISRDNDKNMVY | 484 | DSIPLSSDMSP | 1891 | WGQGT |
| | MP01G | | GSLRLSCAASGRTFS | | | | GKEREF | | TEYTDSVK | | LQMNSLKPEDTAVY | | TATEYGY | | QVTVSS |
| | 05 | | | | | | VA | | G | | YCAA | | | | |
| 7 | 07- | 1892 | EVQLVESGGGLVQAGGS | 485 | MYNMG | 1893 | WFRQAP | 486 | ATGWSGA | 1894 | RFTISRDNTQNTVYL | 487 | DSIRLRSDVTRI | 1895 | WGQ [symbol] |
| | MP01G0 | | LRLSCAASGRAFS | | | | GKEREFV | | NTPYADSV | | QMNSLKPEDTATYY | | PLEYDY | | QVTV [symbol] |
| | 8 | | | | | | A | | KG | | CAA | | | | |
| 8 | 8- | 1896 | QVQLVESGGGLVQPG | 488 | NNFMS | 1897 | WVRQAP | 489 | TISYGGES | 1898 | RFTISRDNAKNTLYL | 490 | ATSYTSIRGAP | 1899 | RGQ [symbol] |
| | MP01C | | GSLRLSCATSGFTFS | | | | GKGLEFV | | TTYAEAVK | | QMNNLKPEDTAIYY | | | | QVTV [symbol] |
| | 12 | | | | | | S | | G | | CAK | | | | |
| 9 | 9- | 1900 | EVOLVESGGGLVQAGG | 491 | TYNMG | 1901 | WFRQAP | 492 | GTLWSGG | 1002 | RFTLSRENAKNTLY | 493 | DPLSLTSDWR | 1903 | WGKGT |
| | MP05G | | SLRLSCAASGGSIY | | | | GKEREF | | DSVYADF | | LQMNSLKPEDTATY | | VDELSS | | LVTVSS |
| | 01 | | | | | | VA | | AKG | | YCAI | | | | |
| 10 | 10- | 1904 | QVQLVESGGGLVQAG | 494 | NYAMG | 1905 | WFRQAP | 495 | ARDLEGIIT | 1906 | RFTISRGNAKNTVF | 496 | RDGGVVAGS | 1907 | WGQ [symbol] |
| | MP05F | | GSLRLSCAASGIPFD | | | | GKEREF | | RYGDSVK | | LQMNSLKPEDTAVY | | RSSAQYNY | | QVTV [symbol] |
| | 03 | | | | | | VA | | G | | YCAA | | | | |
| 11 | 11- | 1908 | QLQLVESGGGLVQAGG | 497 | GYAIG | 1909 | WFRQAP | 498 | YITGSDGT | 1910 | RFTISSDNAKSTVYL | 499 | DRLGSQGRYA | 1911 | WGKY |
| | MP05H | | SLRLSCAASGLTFE | | | | GKEREG | | TYYINSVK | | QMNTLKPEDTAVYY | | SAWWRSGDM | | QVTVSS |
| | 04 | | | | | | VS | | G | | CAV | | DL | | |
| 12 | 12- | 1912 | QVQLVESGGGLVQAG | 500 | GYAIG | 1913 | WFRQAP | 501 | YITGSDGS | 1914 | RFTISSNNAKNTVYL | 502 | DRLGSRGAYV | 1915 | WGKGT |
| | MP05B | | GSLRLSCAASGFTFD | | | | GKEREG | | TYYADSV | | HMNSLKPDDAAVY | | PIWWRSSDMD | | LVTVSS |
| | 12 | | | | | | VS | | KG | | YCAI | | L | | |
| 13 | 13- | 1916 | QVQLQESGGGLVQAGD | 503 | WHAM | 1917 | WFRQAP | 504 | TITWTGRD | 1918 | RFTISKDNAKDTLFLQ | 505 | ARERATWAYS | 1919 | WGQGT |
| | MP05E1 | | SLRLSCAASGFNFG | | G | | GKEREFV | | TYYADSVR | | MNSLRPDDTGVYYC | | EDDCDY | | QVTVSS |
| | 2 | | | | | | A | | G | | AK | | | | |
| 14 | 14- | 1920 | EVQLVESGGGLVQTGGS | 506 | NDVMG | 1921 | WYRQVP | 507 | DMDDTGST | 1922 | RFTILRDSVKNTAYL | 508 | GLWIKGRHFDY | 1923 | WGQGT |
| | MP02C0 | | LRLSCAASGSQFI | | | | GKQRELV | | EYADSVKG | | QMSNLKPEDTGVYY | | | | QVTVSS |
| | 3 | | | | | | A | | | | CKA | | | | |
| 15 | 15- | 1924 | QVQLVESGGGSVQPG | 509 | NYAMS | 1925 | WVRQAP | 510 | SITGFGRG | 1926 | RFTISRDNAEDTLYL | 511 | YSSSTYYPPT | 1927 | RGQGT |
| | MP02E | | GSLRLSCAASGFTFS | | | | GKGLEW | | TDYADSV | | QMNSLKPEDTAVYY | | PARGRDY | | QVTVSS |
| | 06 | | | | | | VA | | KG | | CAK | | | | |
| 16 | 16- | 1928 | EVQLVESGGGLVQAGG | 512 | NYPVG | 1929 | WFRQAP | 513 | AITWISGS | 1930 | RFTISRDNAKNTVY | 514 | ALKTITRGQN | 1931 | WGQ [symbol] |
| | MP02B | | SLRLSCAASGRAIE | | | | GKEREF | | TLYADSVK | | LQMSSLKPEDTALY | | DYSY | | QVTV [symbol] |
| | 08 | | | | | | VA | | G | | YCAA | | | | |
| 17 | 17- | 1932 | QVQLQESGGGLVQAG | 515 | INGMA | 1933 | WYRQGA | 516 | AISRVGNT | 1934 | RFTISRQNARNTVY | 517 | DSWGGDDY | 1935 | WGQ [symbol] |
| | MP02C | | GSLRLSCVASGSVSS | | | | DNQRVL | | AYGDSVK | | LQMNSLKPEDTAVY | | | | QVTV [symbol] |
| | 09 | | | | | | VA | | G | | YCNA | | | | |
| 18 | 18- | 1936 | QVQLVESGGGLVQPG | 518 | SYGIG | 1937 | WVRQAP | 519 | CMSRSDD | 1938 | RFTISKDSAKNTVYL | 520 | VDAYGCSLVQ | 1939 | WGL [symbol] |
| | MP03C | | GSLRLSCAISGGTLD | | | | GKQREG | | RTYYADS | | QMTSLKPEDTAVYY | | PTTYDF | | QVTVSS |
| | 03 | | | | | | VS | | VKG | | CAA | | | | |
| 19 | 19- | 1940 | EVQLVESGGGLVQTGG | 521 | RDAMA | 1941 | WFRQVP | 522 | LISWSGAT | 1942 | RFAISRDNGKNTVY | 523 | DRRPMGSRSY | 1943 | WGQGT |
| | MP03F | | SLRLSCAASGGTFS | | | | GKEREF | | TNYADSV | | LQMNRLKPADTAIY | | FEPTEYDD | | QVTVSS |
| | 08 | | | | | | VA | | KG | | YCAA | | | | |
| 20 | 20- | 1944 | EVOLVESGGGLVQAGG | 524 | SYAMG | 1945 | WFRQAP | 525 | AITWTKRS | 1946 | RFTISRDNAKNTVY | 526 | ARGLPVTPLG | 1947 | WGE [symbol] |
| | MP03F | | SLRLSCAASGRDFS | | | | GKEREF | | TDFPDSVK | | LDMNSLKPEDTAVY | | DIIY | | LVTV [symbol] |
| | 10 | | | | | | VV | | G | | YCAS | | | | |
| 21 | 21- | 1948 | EVQLVESGGGLVQAGGS | 527 | INAMG | 1949 | WFRQAP | 528 | AISRSGGS | 1950 | RFTISRDNAKNTVYL | 529 | TMAVGWTTRW | 1951 | WGQ [symbol] |
| | MP03A1 | | LRLSCAASGRTFS | | | | GKEREFV | | TVYVDGVK | | QMNSLEPEDTAVYY | | RTADFDS | | QVTVSS |
| | 2 | | | | | | A | | G | | CAA | | | | |
| 22 | 22- | 1952 | EVQLVESGGGLVQAGG | 530 | INAMA | 1953 | WFRQVP | 531 | AISRDGGA | 1954 | RFTISRDNSKNTVY | 532 | TRAIGWTARW | 1955 | WGQGT |
| | MP03F | | SLRLSCAASGSIFS | | | | GMEREL | | SVYRDSV | | LQMNTLKPEDTAIY | | ITTDFDF | | QVTVSS |
| | 12 | | | | | | VA | | KG | | VCAA | | | | |
| 23 | 23- | 1956 | QVQLVESGGGLVQAG | 533 | RYTMA | 1957 | WFRQAP | 534 | SVTDSGR | 1958 | RFTVSRDNAKNTVY | 535 | NTDYFQIKSLD | 1959 | WGQGT |
| | MP06F | | GSLRLSCAVSGDVFV | | | | GKEREF | | TTDYVHSV | | LQMNNLKPEDTAVY | | ANT | | QVTVSS |
| | 03 | | | | | | VA | | KG | | YCAA | | | | |
| 24 | 24- | 1960 | QVQLVESGGELVQGGA | 536 | NANMA | 1961 | WFRQAP | 537 | LITWSSGS | 1962 | RFTISRDNARKMVY | 538 | DGPPYSGTYY | 1963 | WGQGT |
| | MP06E | | SLRLSCAASGRTFS | | | | EKEREFV | | TLYADSVK | | LQMNSLKPEDTAVY | | RYDTYDY | | QVTVSS |
| | 05 | | | | | | A | | G | | YCAA | | | | |
| 25 | 25- | 1964 | QVQLVESGGGLVQTGDS | 539 | TTYIA | 1965 | WFRQAP | 540 | YISPRFSHT | 1966 | RFTISRNIAKRTVDLE | 541 | REHSGSTAWE | 1967 | WGQGT |
| | MP06F0 | | LRLSCAASGRSLD | | | | GKERDFL | | WYADSVK | | MNSLEPEDTAVYYC | | HYDH | | QVTVSS |
| | 5 | | | | | | A | | G | | AA | | | | |
| 26 | 26- | 1968 | QVQLQESGGGLVQAG | 542 | RYTMA | 1969 | WFRQAP | 543 | SVTDSGR | 1970 | RFTVSRDNAKNTAY | 544 | NTDYFQIRSLD | 1971 | WGQGT |
| | MP06A | | GSLRLSCAASGDVFV | | | | GKEREF | | TTEYVDSV | | LQMNNLKPEDTAIY | | LNT | | QVTVSS |
| | 07 | | | | | | VA | | KG | | YCAA | | | | |
| 27 | 27- | 1972 | QVQLVESGGGLVQAG | 545 | TDVVG | 1973 | WFRQAP | 546 | SISRSGDG | 1974 | RFTISRNNAWNTVN | 547 | GDGWSTYDY | 1975 | WGQ [symbol] |
| | MP04G | | GSLTLSCAAPGRTFG | | | | GKEREF | | IYYDDSVK | | LQMNSLKVEDTAVY | | | | QVTV [symbol] |
| | 01 | | | | | | VA | | G | | YCAA | | | | |
| 28 | 28- | 1976 | QVQLVESGGGLVQAG | 548 | RYAMG | 1977 | WFRQAP | 549 | ANSWGGD | 1978 | RFTFSRDNAKNTVY | 550 | APTSFATTAY | 1979 | WGQ [symbol] |
| | MP04D | | GSLRLSCAASGRTLS | | | | GKEREF | | TYYADSV | | LQMNSLQPEDTAVY | | SGSNSYAY | | QVT [symbol] |
| | 02 | | | | | | VT | | QG | | YCAA | | | | |
| 29 | 29- | 1980 | QVQLVESGGGLVQAG | 551 | NYYMG | 1981 | WFRQAP | 552 | GIIWNGDH | 1982 | RFTISRDNAKNTAY | 553 | TFWIERATTPD | 1983 | WGQGT |
| | MP04H | | GSLRLACVASGLTFD | | | | GKEREF | | TAYADSIK | | LRMNSLKPEDTAVY | | IGQYAY | | QVTVSS |
| | 02 | | | | | | VA | | G | | YCAA | | | | |
| 30 | 30- | 1984 | EVQLVESGGGWVQDGG | 554 | RGIMG | 1985 | WFRQAP | 555 | RIIWHINST | 1986 | FTISRDSAKNTMYLQ | 556 | RDRYGSGNSL | 1987 | WGQ [symbol] |
| | MP04C0 | | SLRLSCALSGRTFV | | | | GKEREFV | | RYADSVKG | | MDSLRPEDTAVYYC | | SPSAYDY | | QVTV [symbol] |
| | 3 | | | | | | A | | | | AA | | | | |
| 31 | 31- | 1988 | QVQLVESGGGLVQAG | 557 | GYALG | 1989 | WFRQAP | 558 | RINWSGSF | 1990 | RFTISRDNAKNTMY | 559 | DNPSTLATDY | 1991 | WGQ [symbol] |
| | MP04E | | GSLRLSCTGYGGAFT | | | | GKEREF | | TYYASSVK | | LQMNNLKPEDTAVY | | DN | | QVTVSS |
| | 03 | | | | | | VA | | G | | YCAA | | | | |
| 32 | 32- | 1992 | QVQLVESGGGLVQAG | 560 | STAVG | 1993 | WFRQVP | 561 | AINRSGSA | 1994 | RFTISRDNAKNTVY | 562 | DSLPYGRPYY | 1995 | WGQGT |
| | MP04A | | GSLRLSCAASGRTFG | | | | GKEREF | | TTYADSVK | | LQMNSLTPEDTGVY | | FQRSAGEYDY | | QVTVSS |
| | 08 | | | | | | VS | | G | | YCAA | | | | |
| 33 | 33- | 1996 | QLQLVESGGGLVQAGGS | 563 | RVAVG | 1997 | WFRQAP | 564 | AVNRPATM | 1998 | RFTVSRDNAKNTVDL | 565 | DSVPYGRPYY | 1999 | WGQGT |
| | MP04C0 | | LRLSCAASGPTFS | | | | GKEREFV | | TKYADSVK | | QMNSMKPEDTAVYY | | WQTSAGDYDY | | QVTVSS |
| | 9 | | | | | | A | | G | | CAA | | | | |
| 34 | 34- | 2000 | QVQLVESGGGLVQAGS | 566 | RLAMG | 2001 | WFRQAP | 567 | VNSWGGD | 2002 | RFTYSRDNAKSAVY | 568 | APTSFATTAY | 2003 | WGQGA |
| | MP04A | | SLRLSCAASGRTLS | | | | GKEREF | | TFYADSVE | | LQMNSLQPEDTAVY | | SSSNSYAY | | QVTVSS |
| | 12 | | | | | | VA | | G | | YCAA | | | | |
| 35 | 35- | 2004 | QVQLQESGGGLVQGG | 569 | SYAMG | 2005 | WFRQAP | 570 | AISRSGRS | 2006 | RFTISRDNAKSTVYL | 571 | GRYYNSAYDP | 2007 | WGHGT |
| | MP07G | | GSLRLSCAASGGIFS | | | | GKEREF | | TNYADSV | | QMNSLKPEETAVYY | | SPGDFGS | | QVTVSS |
| | 01 | | | | | | VA | | KG | | CAA | | | | |
| 36 | 36- | 2008 | QVQLVESGGGLVQAG | 570 | RYAMG | 2009 | WFRQAP | 573 | ASSWGGD | 2010 | RFTFSRDNAKNAVY | 574 | APTSFPTTAY | 2011 | WGQGT |
| | MP07F | | GSLRLSCAASGRTLS | | | | GSEREF | | TFYADSVE | | LQMNSLQPEDTAAY | | SSSNSYAY | | QVTVSS |
| | 02 | | | | | | VA | | G | | YCAA | | | | |
| 37 | 37- | 2012 | QVQLVESGGGLVQAG | 573 | RYAMG | 2013 | WFRQAP | 576 | IDSWGGD | 2014 | RFTFSRDNAKNEVY | 577 | APTSFATTAY | 2015 | WGQ [symbol] |
| | MP07F | | GSLRLSCAASGRTLS | | | | GKEREY | | TFYADSVE | | LQMNSLQPEDTAVY | | SSSNSYRY | | QVT [symbol] |
| | 09 | | | | | | VA | | G | | YCAG | | | | |
| 38 | 38- | 2016 | QVQLVESGGGLVQAG | 576 | RDAM | 2017 | WFRQAP | 579 | VMSWGGD | 2018 | RFTFSRDNAKNAVY | 580 | APTSFATTAY | 2019 | WGR [symbol] |
| | MP07A | | GSLRLSCAASGRSLS | | G | | GKEREF | | TFYTDSVE | | LEMNDLQPEDTAVY | | SSSNSYSY | | QVT [symbol] |
| | 11 | | | | | | VA | | G | | YCAA | | | | |

[symbol]

indicates data missing or illegible when filed

TABLE 5
Sequence of multispecific polypeptides.
“ID” refers to the SEQ ID NO as
used herein.

| ID | Name | Sequence |
|---|---|---|
| 249 | BsVHH-3 | QVQLVESGGGLVQAGGSLRLSCAASGRTFG |
| | (32x16) | STAVGWFRQVPGKEREFVSAINRSGSATTY |
| | SEQ ID | ADSVKGRFTISRDNAKNTVYLQMNSLTPED |
| | NO: 2020 | TGVYYCAADSLPYGRPYYFQRSAGEYDYWG |
| | | QGTQVTVSSGGGGSGGGGSGGGGSGSEVQL |
| | | VESGGGLVQAGGSLRLSCAASGRAIENYPV |
| | | GWFRQAPGKEREFVAAITWISGSTLYADSV |
| | | KGRFTISRDNAKNTVYLQMSSLKPEDTALY |
| | | YCAAALKTITRGQNDYSYWGQGTQVTVSSG |
| | | GGGSGGGGSGGGGSTVSSDKTHTCPPCPAP |
| | | EAAGGPSVFLFPPKPKDTLMISRTPEVTCV |
| | | VVDVSHEDPEVKFNWYVDGVEVHNAKTKPR |
| | | EEQYNSTYRVVSVLTVLHQDWLNGKEYKCK |
| | | VSNKALGAPIEKTISKAKGQPREPQVYTLP |
| | | PSRDELTKNQVSLTCLVKGFYPSDIAVEWE |
| | | SNGQPENNYKTTPPVLDSDGSFLLYSKLTV |
| | | DKSRWQQGNVFSCSVMHEALHNHYTQKSLS |
| | | LSPGK |
| 250 | BsVHH-4 | QVQLQESGGGLVQGGGSLRLSCAASGGIFS |
| | (35x16) | SYAMGWFRQAPGKEREFVAAISRSGRSTNY |
| | SEQ ID | ADSVKGRFTISRDNAKSTVYLQMNSLKPEE |
| | NO: 2021 | TAVYYCAAGRYYNSAYDPSPGDFGSWGHGT |
| | | QVTVSSGGGGSGGGGSGGGGSGSEVQLVES |
| | | GGGLVQAGGSLRLSCAASGRAIENYPVGWF |
| | | RQAPGKEREFVAAITWISGSTLYADSVKGR |
| | | FTISRDNAKNTVYLQMSSLKPEDTALYYCA |
| | | AALKTITRGQNDYSYWGQGTQVTVSSGGGG |
| | | SGGGGSGGGGSTVSSDKTHTCPPCPAPEAA |
| | | GGPSVFLEPPKPKDTLMISRTPEVTCVVDV |
| | | SHEDPEVKFNWYVDGVEVHNAKTKPREEQY |
| | | NSTYRVVSVLTVLHQDWLNGKEYKCKVSNK |
| | | ALGAPIEKTISKAKGQPREPQVYTLPPSRD |
| | | ELTKNQVSLTCLVKGFYPSDIAVEWESNGQ |
| | | PENNYKTTPPVLDSDGSFLLYSKLTVDKSR |
| | | WQQGNVFSCSVMHEALHNHYTQKSLSLSPG |
| | | K |
| 251 | BsVHH-5 | QVQLVESGGGLVQAGGSLRLSCAASGRTLS |
| | (36x16) | RYAMGWFRQAPGSEREFVAASSWGGDTFYA |
| | SEQ ID | DSVEGRFTFSRDNAKNAVYLQMNSLQPEDT |
| | NO: 2022 | AAYYCAAAPTSFPTTAYSSSNSYAYWGQGT |
| | | QVTVSSGGGGSGGGGSGGGGSGSEVQLVES |
| | | GGGLVQAGGSLRLSCAASGRAIENYPVGWF |
| | | RQAPGKEREFVAAITWISGSTLYADSVKGR |
| | | FTISRDNAKNTVYLQMSSLKPEDTALYYCA |
| | | AALKTITRGQNDYSYWGQGTQVTVSSGGGG |
| | | SGGGGSGGGGSTVSSDKTHTCPPCPAPEAA |
| | | GGPSVFLFPPKPKDTLMISRTPEVTCVVVD |
| | | VSHEDPEVKFNWYVDGVEVHNAKTKPREEQ |
| | | YNSTYRVVSVLTVLHQDWLNGKEYKCKVSN |
| | | KALGAPIEKTISKAKGQPREPQVYTLPPSR |
| | | DELTKNQVSLTCLVKGFYPSDIAVEWESNG |
| | | QPENNYKTTPPVLDSDGSFLLYSKLTVDKS |
| | | RWQQGNVFSCSVMHEALHNHYTQKSLSLSP |
| | | GK |
| 252 | BsVHH-6 | QVQLVESGGGLVQAGGSLTLSCAAPGRTFG |
| | (27x18) | TDVVGWFRQAPGKEREFVASISRSGDGIYY |
| | SEQ ID | DDSVKGRFTISRNNAWNTVNLQMNSLKVED |
| | NO: 2023 | TAVYYCAAGDGWSTYDYWGQGTQVTVSSGG |
| | | GGSGGGGSGGGGSGSQVQLVESGGGLVQPG |
| | | GSLRLSCAISGGTLDSYGIGWVRQAPGKQR |
| | | EGVSCMSRSDDRTYYADSVKGRFTISKDSA |
| | | KNTVYLQMTSLKPEDTAVYYCAAVDAYGCS |
| | | LVQPTTYDFWGLGTQVTVSSGGGGSGGGGS |
| | | GGGGSTVSSDKTHTCPPCPAPEAAGGPSVF |
| | | LFPPKPKDTLMISRTPEVTCVVVDVSHEDP |
| | | EVKFNWYVDGVEVHNAKTKPREEQYNSTYR |
| | | VVSVLTVLHQDWLNGKEYKCKVSNKALGAP |
| | | IEKTISKAKGQPREPQVYTLPPSRDELTKN |
| | | QVSLTCLVKGFYPSDIAVEWESNGQPENNY |
| | | KTTPPVLDSDGSFLLYSKLTVDKSRWQQGN |
| | | VFSCSVMHEALHNHYTQKSLSLSPGK |
| 253 | BsVHH-7 | QVQLVESGGGLVQAGGSLRLSCTGYGGAFT |
| | (31x18) | GYALGWFRQAPGKEREFVARINWSGSFTYY |
| | SEQ ID | ASSVKGRFTISRDNAKNTMYLQMNNLKPED |
| | NO: 2024 | TAVYYCAADNPSTLATDYDNWGQGTQVTVS |
| | | SGGGGSGGGGSGGGGSGSQVQLVESGGGLV |
| | | QPGGSLRLSCAISGGTLDSYGIGWVRQAPG |
| | | KQREGVSCMSRSDDRTYYADSVKGRFTISK |
| | | DSAKNTVYLQMTSLKPEDTAVYYCAAVDAY |
| | | GCSLVQPTTYDFWGLGTQVTVSSGGGGSGG |
| | | GGSGGGGSTVSSDKTHTCPPCPAPEAAGGP |
| | | SVFLFPPKPKDTLMISRTPEVTCVVVDVSH |
| | | EDPEVKFNWYVDGVEVHNAKTKPREEQYNS |
| | | TYRVVSVLTVLHQDWLNGKEYKCKVSNKAL |
| | | GAPIEKTISKAKGQPREPQVYTLPPSRDEL |
| | | TKNQVSLTCLVKGFYPSDIAVEWESNGQPE |
| | | NNYKTTPPVLDSDGSFLLYSKLTVDKSRWQ |
| | | QGNVFSCSVMHEALHNHYTQKSLSLSPGK |
| 254 | BsVHH-8 | QVQLVESGGGLVQAGGSLRLSCAASGRTFG |
| | (32x18) | STAVGWFRQVPGKEREFVSAINRSGSATTY |
| | SEQ ID | ADSVKGRFTISRDNAKNTVYLQMNSLTPED |
| | NO: 2025 | TGVYYCAADSLPYGRPYYFQRSAGEYDYWG |
| | | QGTQVTVSSGGGGSGGGGSGGGGSGSQVQL |
| | | VESGGGLVQPGGSLRLSCAISGGTLDSYGI |
| | | GWVRQAPGKQREGVSCMSRSDDRTYYADSV |
| | | KGRFTISKDSAKNTVYLQMTSLKPEDTAVY |
| | | YCAAVDAYGCSLVQPTTYDFWGLGTQVTVS |
| | | SGGGGSGGGGSGGGGSTVSSDKTHTCPPCP |
| | | APEAAGGPSVFLFPPKPKDTLMISRTPEVT |
| | | CVVVDVSHEDPEVKFNWYVDGVEVHNAKTK |
| | | PREEQYNSTYRVVSVLTVLHQDWLNGKEYK |
| | | CKVSNKALGAPIEKTISKAKGQPREPQVYT |
| | | LPPSRDELTKNQVSLTCLVKGFYPSDIAVE |
| | | WESNGQPENNYKTTPPVLDSDGSFLLYSKL |
| | | TVDKSRWQQGNVFSCSVMHEALHNHYTQKS |
| | | LSLSPGK |
| 255 | BsVHH-9 | QVQLQESGGGLVQGGGSLRLSCAASGGIFS |
| | (35x18) | SYAMGWFRQAPGKEREFVAAISRSGRSTNY |
| | SEQ ID | ADSVKGRFTISRDNAKSTVYLQMNSLKPEE |
| | NO: 2026 | TAVYYCAAGRYYNSAYDPSPGDFGSWGHGT |
| | | QVTVSSGGGGSGGGGSGGGGSGSQVQLVES |
| | | GGGLVQPGGSLRLSCAISGGTLDSYGIGWV |
| | | RQAPGKQREGVSCMSRSDDRTYYADSVKGR |
| | | FTISKDSAKNTVYLQMTSLKPEDTAVYYCA |
| | | AVDAYGCSLVQPTTYDFWGLGTQVTVSSGG |
| | | GGSGGGGSGGGGSTVSSDKTHTCPPCPAPE |
| | | AAGGPSVFLFPPKPKDTLMISRTPEVTCVV |
| | | VDVSHEDPEVKFNWYVDGVEVHNAKTKPRE |
| | | EQYNSTYRVVSVLTVLHQDWLNGKEYKCKV |
| | | SNKALGAPIEKTISKAKGQPREPQVYTLPP |
| | | SRDELTKNQVSLTCLVKGFYPSDIAVEWES |
| | | NGQPENNYKTTPPVLDSDGSFLLYSKLTVD |
| | | KSRWQQGNVFSCSVMHEALHNHYTQKSLSL |
| | | SPGK |
| 256 | BsVHH-10 | QVQLVESGGGLVQAGGSLRLSCAASGRTLS |
| | (36x18) | RYAMGWFRQAPGSEREFVAASSWGGDTFYA |
| | SEQ ID | DSVEGRFTFSRDNAKNAVYLQMNSLQPEDT |
| | NO: 2027 | AAYYCAAAPTSFPTTAYSSSNSYAYWGQGT |
| | | QVTVSSGGGGSGGGGSGGGGSGSQVQLVES |
| | | GGGLVQPGGSLRLSCAISGGTLDSYGIGWV |
| | | RQAPGKQREGVSCMSRSDDRTYYADSVKGR |
| | | FTISKDSAKNTVYLQMTSLKPEDTAVYYCA |
| | | AVDAYGCSLVQPTTYDFWGLGTQVTVSSGG |
| | | GGSGGGGSGGGGSTVSSDKTHTCPPCPAPE |
| | | AAGGPSVFLEPPKPKDTLMISRTPEVTCVV |
| | | VDVSHEDPEVKFNWYVDGVEVHNAKTKPRE |
| | | EQYNSTYRVVSVLTVLHQDWLNGKEYKCKV |
| | | SNKALGAPIEKTISKAKGQPREPQVYTLPP |
| | | SRDELTKNQVSLTCLVKGFYPSDIAVEWES |
| | | NGQPENNYKTTPPVLDSDGSFLLYSKLTVD |
| | | KSRWQQGNVFSCSVMHEALHNHYTQKSLSL |
| | | SPGK |
| 257 | BsVHH-11 | QVQLVESGGGLVQAGGSLTLSCAAPGRTFG |
| | (27x19) | TDVVGWFRQAPGKEREFVASISRSGDGIYY |
| | SEQ ID | DDSVKGRFTISRNNAWNTVNLQMNSLKVED |
| | NO: 2028 | TAVYYCAAGDGWSTYDYWGQGTQVTVSSGG |
| | | GGSGGGGSGGGGSGSEVQLVESGGGLVQTG |
| | | GSLRLSCAASGGTFSRDAMAWFRQVPGKER |
| | | EFVALISWSGATTNYADSVKGRFAISRDNG |
| | | KNTVYLQMNRLKPADTAIYYCAADRRPMGS |
| | | RSYFEPTEYDDWGQGTQVTVSSGGGGSGGG |
| | | GSGGGGSTVSSDKTHTCPPCPAPEAAGGPS |
| | | VFLFPPKPKDTLMISRTPEVTCVVVDVSHE |
| | | DPEVKFNWYVDGVEVHNAKTKPREEQYNST |
| | | YRVVSVLTVLHQDWLNGKEYKCKVSNKALG |
| | | APIEKTISKAKGQPREPQVYTLPPSRDELT |
| | | KNQVSLTCLVKGFYPSDIAVEWESNGQPEN |
| | | NYKTTPPVLDSDGSFLLYSKLTVDKSRWQQ |
| | | GNVFSCSVMHEALHNHYTQKSLSLSPGK |
| 258 | BsVHH-13 | QVQLVESGGGLVQAGGSLRLSCAASGRTFG |
| | (32x19) | STAVGWFRQVPGKEREFVSAINRSGSATTY |
| | SEQ ID | ADSVKGRFTISRDNAKNTVYLQMNSLTPED |
| | NO: 2029 | TGVYYCAADSLPYGRPYYFQRSAGEYDYWG |
| | | QGTQVTVSSGGGGSGGGGSGGGGSGSEVQL |
| | | VESGGGLVQTGGSLRLSCAASGGTFSRDAM |
| | | AWFRQVPGKEREFVALISWSGATTNYADSV |
| | | KGRFAISRDNGKNTVYLQMNRLKPADTAIY |
| | | YCAADRRPMGSRSYFEPTEYDDWGQGTQVT |
| | | VSSGGGGSGGGGSGGGGSTVSSDKTHTCPP |
| | | CPAPEAAGGPSVFLFPPKPKDTLMISRTPE |
| | | VTCVVVDVSHEDPEVKFNWYVDGVEVHNAK |
| | | TKPREEQYNSTYRVVSVLTVLHQDWLNGKE |
| | | YKCKVSNKALGAPIEKTISKAKGQPREPQV |
| | | YTLPPSRDELTKNQVSLTCLVKGFYPSDIA |
| | | VEWESNGQPENNYKTTPPVLDSDGSFLLYS |
| | | KLTVDKSRWQQGNVFSCSVMHEALHNHYTQ |
| | | KSLSLSPGK |
| 259 | BsVHH-15 | QVQLVESGGGLVQAGGSLRLSCAASGRTLS |
| | (36x19) | RYAMGWFRQAPGSEREFVAASSWGGDTFYA |
| | SEQ ID | DSVEGRFTFSRDNAKNAVYLQMNSLQPEDT |
| | NO: 2030 | AAYYCAAAPTSFPTTAYSSSNSYAYWGQGT |
| | | QVTVSSGGGGSGGGGSGGGGSGSEVQLVES |
| | | GGGLVQTGGSLRLSCAASGGTFSRDAMAWF |
| | | RQVPGKEREFVALISWSGATTNYADSVKGR |
| | | FAISRDNGKNTVYLQMNRLKPADTAIYYCA |
| | | ADRRPMGSRSYFEPTEYDDWGQGTQVTVSS |
| | | GGGGSGGGGSGGGGSTVSS |
| | | DKTHTCPPCPAPEAAGGPSVFLFPPKPKDT |
| | | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVYTLPPSRDELTKNQVSLTCLVK |
| | | GFYPSDIAVEWESNGQPENNYKTTPPVLDS |
| | | DGSFLLYSKLTVDKSRWQQGNVFSCSVMHE |
| | | ALHNHYTQKSLSLSPGK |
| 260 | BsVHH-16 | QVQLVESGGGLVQAGGSLTLSCAAPGRTFG |
| | (27x22) | TDVVGWFRQAPGKEREFVASISRSGDGIYY |
| | SEQ ID | DDSVKGRFTISRNNAWNTVNLQMNSLKVED |
| | NO: 2031 | TAVYYCAAGDGWSTYDYWGQGTQVTVSSGG |
| | | GGSGGGGSGGGGSGSEVQLVESGGGLVQAG |
| | | GSLRLSCAASGSIFSINAMAWFRQVPGMER |
| | | ELVAAISRDGGASVYRDSVKGRFTISRDNS |
| | | KNTVYLQMNTLKPEDTAIYVCAATRAIGWT |
| | | ARWITTDFDFWGQGTQVTVSSGGGGSGGGG |
| | | SGGGGSTVSS |
| | | DKTHTCPPCPAPEAAGGPSVFLEPPKPKDT |
| | | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVYTLPPSRDELTKNQVSLTCLVK |
| | | GFYPSDIAVEWESNGQPENNYKTTPPVLDS |
| | | DGSFLLYSKLTVDKSRWQQGNVFSCSVMHE |
| | | ALHNHYTQKSLSLSPGK |
| 261 | BsVHH-20 | QVQLVESGGGLVQAGGSLRLSCAASGRTLS |
| | (36x22) | RYAMGWFRQAPGSEREFVAASSWGGDTFYA |
| | SEQ ID | DSVEGRFTFSRDNAKNAVYLQMNSLQPEDT |
| | NO: 2032 | AAYYCAAAPTSFPTTAYSSSNSYAYWGQGT |
| | | QVTVSSGGGGSGGGGSGGGGSGSEVQLVES |
| | | GGGLVQAGGSLRLSCAASGSIFSINAMAWF |
| | | RQVPGMERELVAAISRDGGASVYRDSVKGR |
| | | FTISRDNSKNTVYLQMNTLKPEDTAIYVCA |
| | | ATRAIGWTARWITTDFDFWGQGTQVTVSSG |
| | | GGGSGGGGSGGGGSTVSSDKTHTCPPCPAP |
| | | EAAGGPSVFLFPPKPKDTLMISRTPEVTCV |
| | | VVDVSHEDPEVKFNWYVDGVEVHNAKTKPR |
| | | EEQYNSTYRVVSVLTVLHQDWLNGKEYKCK |
| | | VSNKALGAPIEKTISKAKGQPREPQVYTLP |
| | | PSRDELTKNQVSLTCLVKGFYPSDIAVEWE |
| | | SNGQPENNYKTTPPVLDSDGSFLLYSKLTV |
| | | DKSRWQQGNVFSCSVMHEALHNHYTQKSLS |
| | | LSPGK |

TABLE 6
Sequences of VHH-hFc fusion proteins.
“ID” refers to the SEQ ID NO
as used herein.

| ID | Name | Sequence |
|---|---|---|
| 2033 | Linker | GGGGSGGGGSGGGGS |
| 2034 | hFc with LALA-PG | DKTHTCPPCPAPEAAGGPSVFLFPP |
| | and cFAE | KPKDTLMISRTPEVTCVVVDVSHED |
| | mutation | PEVKFNWYVDGVEVHNAKTKPREEQ |
| | F405L | YNSTYRVVSVLTVLHQDWLNGKEYK |
| | | CKVSNKALGAPIEKTISKAKGQPRE |
| | | PQVYTLPPSRDELTKNQVSLTCLVK |
| | | GFYPSDIAVEWESNGQPENNYKTTP |
| | | PVLDSDGSFLLYSKLTVDKSRWQQG |
| | | NVFSCSVMHEALHNHYTQKSLSLSP |
| | | GK |
| 2035 | hFc with LALA-PG | DKTHTCPPCPAPEAAGGPSVFLFPP |
| | and cFAE | KPKDTLMISRTPEVTCVVVDVSHED |
| | mutation | PEVKFNWYVDGVEVHNAKTKPREEQ |
| | K409R | YNSTYRVVSVLTVLHQDWLNGKEYK |
| | | CKVSNKALGAPIEKTISKAKGQPRE |
| | | PQVYTLPPSRDELTKNQVSLTCLVK |
| | | GFYPSDIAVEWESNGQPENNYKTTP |
| | | PVLDSDGSFFLYSRLTVDKSRWQQG |
| | | NVFSCSVMHEALHNHYTQKSLSLSP |
| | | GK |

TABLE 7

| | ascending | | | | | | | | |
|---|---|---|---|---|---|---|---|---|---|
| | numbering | | | PE binding | | P.E. Off-rate ranking SPR- | | | |
| | Patent | | | ELISA | | mouse and human IL-2Ra | | | |
| Clone | clone | | | hu | mo | Rmax- | hd(1/s)- | Rmax- | kd (1/s)- |
| nr. | ID | V ID | Clone ID | IL-2R | IL-2R | hu | hu | mo | mo |
| 8 | 1 | 1-MP01H01 | FJ1427_P035MP01H01 | 1.705 | 0.048 | 126.56 | 2.29E−02 | 8.31 | N/A |
| 9 | 2 | 2-MP01A02 | FJ1427 P035MP01A02 | 2.615 | 0.046 | 846.81 | 1.53E−03 | 9.3 | N/A |
| 21 | 3 | 3-MP01E03 | FJ1427_P035MP01E03 | 2.535 | 0.046 | 413.12 | 7.99E−04 | 9.42 | N/A |
| 30 | 4 | 4-MP01F04 | FJ1427_P035MP01F04 | 2.493 | 0.046 | 690.4 | 2.58E−04 | 18.31 | 2.56E−01 |
| 38 | 5 | 5-MP01F05 | FJ1427_P035MP01F05 | 2.620 | 0.046 | 312.1 | 4.20E−04 | 9.23 | N/A |
| 39 | 6 | 6-MP01G05 | FJ1427_P035MP01G05 | 1.794 | 0.046 | 122.65 | 2.06E−02 | 9.9 | N/A |
| 63 | 7 | 07-MP01G08 | FJ1427 P035MP01G08 | 2.573 | 0.046 | 193.76 | 1.84E−03 | 9.92 | N/A |
| 91 | 8 | 8-MP01C12 | FJ1427_P035MP01C12 | 2.531 | 0.045 | 818.61 | 3.83E−04 | 8.42 | N/A |
| 391 | 9 | 9-MP05G01 | FJ1427_P035MP05G01 | 2.353 | 0.047 | 569.9 | 1.69E−04 | 119.21 | 4.42E−02 |
| 406 | 10 | 10-MP05F03 | FJ1427_P035MP05F03 | 2.465 | 0.054 | 779.86 | 6.39E−04 | 328.6 | 1.05E−01 |
| 416 | 11 | 11-MP05H04 | FJ1427 P035MP05H04 | 2.221 | 0.163 | 201.79 | 1.20E−03 | 533.39 | 1.63E−03 |
| 474 | 12 | 12-MP05B12 | FJ1427 P035MP05B12 | 2.136 | 0.936 | 584.53 | 2.22E−04 | 566.73 | 5.47E−04 |
| 440 | 13 | 13-MP05E12 | FJ1427_P035MP05H07 | 2.34 | 0.048 | 329.08 | 7.42E−04 | 59.27 | 4.78E−02 |
| 1 | 39 | | FJ1427_P035MP01A01 | 2.852 | 0.046 | 860.46 | 1.12E−03 | 9.37 | N/A |
| 2 | 40 | | FJ1427_P035MP01B01 | 2.783 | 0.046 | 306.62 | 1.78E−03 | 9.5 | N/A |
| 7 | 41 | | FJ1427_P035MP01G01 | 2.524 | 0.053 | 911.04 | 4.14E−04 | 8.31 | N/A |
| 13 | 42 | | FJ1427 P035MP01E02 | 2.861 | 0.046 | 840.08 | 1.28E−03 | 8.36 | N/A |
| 14 | 43 | | FJ1427_P035MP01F02 | 2.495 | 0.046 | 250.32 | 3.91E−03 | 8.25 | N/A |
| 16 | 44 | | FJ1427 P035MP01H02 | 2.349 | 0.047 | 388.34 | 1.15E−03 | 8.76 | N/A |
| 19 | 45 | | FJ1427_P035MP01C03 | 2.840 | 0.045 | 856.2 | 1.44E−03 | 8.37 | N/A |
| 20 | 46 | | FJ1427_P035MP01D03 | 2.592 | 0.048 | 696.18 | 1.89E−03 | 8.44 | N/A |
| 22 | 47 | | FJ1427 P035MP01F03 | 2.866 | 0.045 | 789.36 | 3.27E−03 | 9.73 | N/A |
| 23 | 48 | | FJ1427_P035MP01G03 | 0.188 | 0.048 | 244.34 | 1.08E−01 | 10.1 | N/A |
| 28 | 49 | | FJ1427_P035MP01D04 | 2.758 | 0.045 | 776.57 | 2.95E−03 | 10.54 | N/A |
| 29 | 50 | | FJ1427_P035MP01E04 | 2.613 | 0.046 | 790.82 | 2.35E−03 | 10.12 | N/A |
| 31 | 51 | | FJ1427 P035MP01G04 | 2.786 | 0.047 | 329.97 | 1.07E−03 | 11.74 | N/A |
| 37 | 52 | | FJ1427_P035MP01E05 | 2.591 | 0.046 | 347.15 | 4.98E−04 | 11.74 | N/A |
| 40 | 53 | | FJ1427_P035MP01H05 | 2.650 | 0.046 | 254.26 | 2.76E−03 | 9.95 | N/A |
| 46 | 54 | | FJ1427 P035MP01F06 | 2.705 | 0.046 | 388.75 | 5.26E−04 | 9.5 | N/A |
| 53 | 55 | | FJ1427_P035MP01E07 | 2.731 | 0.046 | 725.28 | 1.59E−03 | 9.09 | N/A |
| 54 | 56 | | FJ1427 P035MP01F07 | 2.760 | 0.047 | 356.5 | 6.70E−04 | 17.9 | 4.48E−02 |
| 55 | 57 | | FJ1427_P035MP01G07 | 2.747 | 0.047 | 808.25 | 1.50E−03 | 10.2 | N/A |
| 56 | 58 | | FJ1427_P035MP01H07 | 2.140 | 0.049 | 158.58 | 1.12E−03 | 9.38 | N/A |
| 60 | 59 | | FJ1427_P035MP01D08 | 2.621 | 0.045 | 402.24 | 8.42E−04 | 14.13 | N/A |
| 62 | 60 | | FJ1427 P035MP01F08 | 2.709 | 0.046 | 756.35 | 1.62E−03 | 9.58 | N/A |
| 64 | 61 | | FJ1427 P035MP01H08 | 2.605 | 0.047 | i.s. | i.s. | i.s. | i.s. |
| 68 | 62 | | FJ1427_P035MP01D09 | 2.566 | 0.045 | 729.19 | 2.12E−03 | 7.9 | N/A |
| 77 | 63 | | FJ1427_P035MP01E10 | 2.784 | 0.046 | 816.2 | 1.19E−03 | 8.47 | N/A |
| 79 | 64 | | FJ1427_P035MP01G10 | 2.755 | 0.046 | 414.58 | 4.43E−04 | 8.33 | N/A |
| 80 | 65 | | FJ1427_P035MP01H10 | 2.471 | 0.057 | i.s. | i.s. | i.s. | i.s. |
| 81 | 66 | | FJ1427 P035MP01A11 | 2.809 | 0.046 | 327.6 | 7.78E−04 | 8.91 | N/A |
| 83 | 67 | | FJ1427 P035MP01C11 | 2.784 | 0.045 | 296.26 | 5.56E−04 | 18.03 | 3.51E−01 |
| 84 | 68 | | FJ1427_P035MP01D11 | 3.207 | 0.045 | 750.95 | 2.70E−03 | 8.62 | N/A |
| 85 | 69 | | FJ1427_P035MP01E11 | 2.685 | 0.046 | 832.34 | 7.04E−04 | 8.91 | N/A |
| 86 | 70 | | FJ1427_P035MP01F11 | 2.743 | 0.047 | 830.65 | 1.18E−03 | 8.47 | N/A |
| 87 | 71 | | FJ1427 P035MP01G11 | 2.733 | 0.051 | 876.95 | 1.23E−04 | 54.04 | 3.12E−01 |
| 88 | 72 | | FJ1427_P035MP01H11 | 2.617 | 0.047 | 342.09 | 7.25E−04 | 9.83 | N/A |
| 90 | 73 | | FJ1427_P035MP01B12 | 3.053 | 0.046 | 838.87 | 1.44E−03 | 9.18 | N/A |
| 93 | 74 | | FJ1427_P035MP01E12 | 2.693 | 0.052 | 800.76 | 1.41E−04 | 46.48 | 3.35E−01 |
| 94 | 75 | | FJ1427_P035MP01F12 | 2.668 | 0.048 | 787.83 | 1.10E−04 | 15.5 | 3.29E−01 |
| 386 | 76 | | FJ1427 P035MP05B01 | 2.257 | 0.044 | 715.2 | 8.35E−05 | 28.68 | 4.01E−02 |
| 392 | 77 | | FJ1427 P035MP05H01 | 2.62 | 0.048 | 777.28 | 1.41E−04 | 45.46 | 2.91E−01 |
| 400 | 78 | | FJ1427 P035MP05H02 | 2.292 | 0.048 | 703.96 | 1.41E−04 | 55.18 | 3.09E−01 |
| 402 | 79 | | FJ1427_P035MP05B03 | 2.419 | 0.044 | 742.5 | 1.31E−04 | 55.73 | 5.37E−03 |
| 405 | 80 | | FJ1427_P035MP05E03 | 2.48 | 0.05 | 782.11 | 7.72E−04 | 281.3 | 2.38E−02 |
| 411 | 81 | | FJ1427 P035MP05C04 | 1.826 | 0.079 | 780.27 | 9.05E−04 | 514.39 | 5.70E−03 |
| 412 | 82 | | FJ1427 P035MP05D04 | 1.913 | 0.045 | 189.94 | 2.24E−03 | 206.8 | 1.22E−02 |
| 413 | 83 | | FJ1427 P035MP05E04 | 2.385 | 0.046 | 368.07 | 1.58E−03 | 201.85 | 1.96E−01 |
| 417 | 84 | | FJ1427 P035MP05A05 | 1.863 | 0.052 | 155.83 | 3.41E−03 | 185.24 | 1.72E−02 |
| 418 | 85 | | FJ1427_P035MP05B05 | 2.155 | 0.045 | 247.13 | 1.03E−03 | 311.02 | 1.14E−02 |
| 424 | 86 | | FJ1427_P035MP05H05 | 2.508 | 0.048 | 278.7 | 5.91E−04 | 122.79 | 1.07E−01 |
| 427 | 87 | | FJ1427 P035MP05C06 | 2.427 | 0.071 | 283.43 | 6.59E−04 | 344.54 | 3.11E−03 |
| 428 | 88 | | FJ1427 P035MP05D06 | 2.51 | 0.054 | 298.56 | 6.77E−04 | 101.39 | 7.55E−02 |
| 430 | 89 | | FJ1427_P035MP05F06 | 2.507 | 0.048 | 251.48 | 6.70E−04 | 114.08 | 1.15E−01 |
| 433 | 90 | | FJ1427_P035MP05A07 | 2.434 | 0.053 | 828.7 | 1.04E−03 | 388.01 | 3.69E−02 |
| 434 | 91 | | FJ1427 P035MP05B07 | 1.768 | 0.047 | 275.63 | 1.95E−03 | 157.1 | 1.27E−02 |
| 435 | 92 | | FJ1427 P035MP05C07 | 1.734 | 0.07 | 780.36 | 1.41E−03 | 559.5 | 7.14E−03 |
| 442 | 93 | | FJ1427_P035MP05B08 | 1.878 | 0.057 | 287.84 | 2.53E−03 | 442.89 | 6.08E−03 |
| 445 | 94 | | FJ1427_P035MP05E08 | 2.325 | 0.087 | 836.98 | 2.14E−04 | 409.05 | 9.59E−03 |
| 447 | 95 | | FJ1427_P035MP05G08 | 2.389 | 0.38 | 279.18 | 9.08E−04 | 397.99 | 2.58E−03 |
| 448 | 96 | | FJ1427_P035MP05H08 | 2.167 | 0.05 | 186.2 | 2.78E−03 | 199.56 | 1.29E−02 |
| 451 | 97 | | FJ1427_P035MP05C09 | 0.366 | 0.059 | 212.3 | 6.09E−03 | 361.52 | 4.35E−03 |
| 456 | 98 | | FJ1427_P035MP05H09 | 2.262 | 0.154 | 264.1 | 1.05E−03 | 497.37 | 2.44E−03 |
| 457 | 99 | | FJ1427 P035MP05A10 | 2.181 | 0.24 | 324.06 | 1.20E−03 | 452.24 | 3.21E−03 |
| 459 | 100 | | FJ1427 P035MP05C10 | 0.63 | 0.071 | 239.18 | 4.06E−03 | 290.3 | 4.03E−03 |
| 460 | 101 | | FJ1427 P035MP05D10 | 2.089 | 0.107 | 349.75 | 1.08E−03 | 383.29 | 2.84E−03 |
| 461 | 102 | | FJ1427 P035MP05E10 | 2.266 | 0.718 | 144.76 | 1.69E−03 | 298.19 | 1.77E−03 |
| 467 | 103 | | FJ1427 P035MP05C11 | 2.141 | 0.632 | 182.57 | 4.40E−04 | 389.24 | 8.44E−04 |
| 468 | 104 | | FJ1427 P035MP05D11 | 2.544 | 0.045 | 308.43 | 6.62E−04 | 68.16 | 2.40E−02 |
| 469 | 105 | | FJ1427_P035MP05E11 | 2.346 | 0.049 | 532.77 | 1.00E−03 | 225.19 | 2.96E−02 |
| 470 | 106 | | FJ1427_P035MP05F11 | 2.271 | 0.046 | 705.9 | 1.73E−03 | 151.4 | 5.10E−02 |
| 475 | 107 | | FJ1427_P035MP05C12 | 2.402 | 0.045 | 160.88 | 8.74E−04 | 22.12 | 2.92E−02 |
| 476 | 108 | | FJ1427 P035MP05D12 | 2.386 | 0.066 | 430.66 | 2.35E−03 | 157.31 | 3.66E−02 |
| 477 | 109 | | FJ1427 P035MP05E12 | 2.151 | 0.047 | 614.28 | 2.73E−04 | 75.38 | 8.23E−02 |
| 478 | 110 | | FJ1427 P035MP05F12 | 2.185 | 0.113 | 197.87 | 4.69E−04 | 154.72 | 4.63E−03 |

| | ascend- | | | | | | |
|---|---|---|---|---|---|---|---|
| | ing | | | | | | |
| | number- | | | | | | |
| | ing | | | | | | CDR3 |
| Clone | Patent | | VH | | | | Family |
| nr. | clone ID | VH | ID | CDR1 | CDR2 | CDR3 | ID |
| 8 | 1 | EVQLVESGGGLVQAGGSLRLSCAASGRTFR | a12 | THNMG- | AISWNVDNTLYADSVKG- | DNIPLSSDVAATATEYDY- | 6 |
| | | THNMGWFRRAPGKEREFVAAISWNVDNTLY | | SEQ ID | SEQ ID | SEQ ID | |
| | | ADSVKGRFTISRDNGRNMVYLQMNSLKPED | | NO: 467 | NO: 468 | NO: 469 | |
| | | TAVYYCAADNIPLSSDVAATATEYDYWGQG | | | | | |
| | | TQVTVSS- | | | | | |
| | | SEQ ID | | | | | |
| | | NO: 1 | | | | | |
| 9 | 2 | QVQLVESGGGLVQPGGSLRLSCATSGFTFR | a80 | NNFMS- | TISYGGESTTYAESVKG- | ATSYDSIRSGS- | 2 |
| | | NNFMSWVRQAPGKGLEWVSTISYGGESTTY | | SEQ ID | SEQ ID | SEQ ID | |
| | | AESVKGRFTISRDNAKNTLYLQMNNLKPED | | NO: 470 | NO: 471 | NO: 472 | |
| | | TAVYYCAKATSYDSIRSGSRGQGTQVTVSS | | | | | |
| | | - | | | | | |
| | | SEQ ID | | | | | |
| | | NO: 2 | | | | | |
| 21 | 3 | QVQLVESGGGLVQAGGSLRLSCTASGRTLS | a74 | SYSMA- | AISSSGVVTHVLDSVKG- | TASSYSTYEANYNY- | 14 |
| | | SYSMAWFRQAADKGREFVTAISSSGVVTHV | | SEQ ID | SEQ ID | SEQ ID | |
| | | LDSVKGRFTISRDNAKNTVYLQMNSLQPED | | NO: 473 | NO: 474 | NO: 475 | |
| | | TALYFCAGTASSYSTYEANYNYWGQGTLVT | | | | | |
| | | VSS- | | | | | |
| | | SEQ ID | | | | | |
| | | NO: 3 | | | | | |
| 30 | 4 | QVQLVESGGGLVQAGGSLRLSCAASGGTFR | a60 | TRNMG- | AVSWNVDNKLYADSVKG- | DNIPLSSDMRPTATEYDY- | 6 |
| | | TRNMGWFRRAPGKEREFVAAVSWNVDNKLY | | SEQ ID | SEQ ID | SEQ ID | |
| | | ADSVKGRFTISRDNGRNMVYLQMNSLKPED | | NO: 476 | NO: 477 | NO: 478 | |
| | | TAVYYCAADNIPLSSDMRPTATEYDYWGQG | | | | | |
| | | TQVTVSS- | | | | | |
| | | SEQ ID | | | | | |
| | | NO: 4 | | | | | |
| 38 | 5 | EVQLVESGGGLVQAGGSLRLSCAASIRAFT | a15 | TWSMA- | RINVSGSVTYYADFVKG- | DRTGVGTNDYNY- | 9 |
| | | TWSMAWFRQAPGKEREYVARINVSGSVTYY | | SEQ ID | SEQ ID | SEQ ID | |
| | | ADFVKGRFTISRDNAKKTMYLEMNNLKPED | | NO: 479 | NO: 480 | NO: 481 | |
| | | TALYYCAADRTGVGTNDYNYWGQGTQVTVS | | | | | |
| | | S- | | | | | |
| | | SEQ ID | | | | | |
| | | NO: 5 | | | | | |
| 39 | 6 | QVQLQESGGGLVQAGGSLRLSCAASGRTFS | a49 | SYAMG- | VISWNVDNTEYTDSVKG- | DSIPLSSDMSPTATEYGY- | 10 |
| | | SYAMGWFRQAPGKEREFVAVISWNVDNTEY | | SEQ ID | SEQ ID | SEQ ID | |
| | | TDSVKGRFTISRDNDKNMVYLQMNSLKPED | | NO: 482 | NO: 483 | NO: 484 | |
| | | TAVYYCAADSIPLSSDMSPTATEYGYWGQG | | | | | |
| | | TQVTVSS- | | | | | |
| | | SEQ ID | | | | | |
| | | NO: 6 | | | | | |
| 63 | 7 | EVQLVESGGGLVQAGGSLRLSCAASGRAFS | a11 | MYNMG- | ATGWSGANTPYADSVKG- | DSIRLRSDVTRIPLEYDY- | 11 |
| | | MYNMGWFRQAPGKEREFVAATGWSGANTPY | | SEQ ID | SEQ ID | SEQ ID | |
| | | ADSVKGRFTISRDNTQNTVYLQMNSLKPED | | NO: 485 | NO: 486 | NO: 487 | |
| | | TATYYCAADSIRLRSDVTRIPLEYDYWGQG | | | | | |
| | | TQVTVSS- | | | | | |
| | | SEQ ID | | | | | |
| | | NO: 7 | | | | | |
| 91 | 8 | QVQLVESGGGLVQPGGSLRLSCATSGFTFS | a81 | NNFMS- | TISYGGESTTYAEAVKG- | ATSYTSIRGAP- | 3 |
| | | NNFMSWVRQAPGKGLEFVSTISYGGESTTY | | SEQ ID | SEQ ID | SEQ ID | |
| | | AEAVKGRFTISRDNAKNTLYLQMNNLKPED | | NO: 488 | NO: 489 | NO: 490 | |
| | | TAIYYCAKATSYTSIRGAPRGQGTQVTVSS | | | | | |
| | | - | | | | | |
| | | SEQ ID | | | | | |
| | | NO: 8 | | | | | |
| 391 | 9 | EVQLVESGGGLVQAGGSLRLSCAASGGSIY | a7 | TYNMG- | GTLWSGGDSVYADFAKG- | DPLSLTSDWRVDELSS- | 7 |
| | | TYNMGWFRQAPGKEREFVAGTLWSGGDSVY | | SEQ ID | SEQ ID | SEQ ID | |
| | | ADFAKGRFTLSRENAKNTLYLQMNSLKPED | | NO: 491 | NO: 492 | NO: 493 | |
| | | TATYYCAIDPLSLTSDWRVDELSSWGKGTL | | | | | |
| | | VTVSS- | | | | | |
| | | SEQ ID | | | | | |
| | | NO: 9 | | | | | |
| 406 | 10 | QVQLVESGGGLVQAGGSLRLSCAASGIPFD | a63 | NYAMG- | ARDLEGIITRYGDSVKG- | RDGGVVAGSRSSAQYNY- | 12 |
| | | NYAMGWFRQAPGKEREFVAARDLEGIITRY | | SEQ ID | SEQ ID | SEQ ID | |
| | | GDSVKGRFTISRGNAKNTVFLQMNSLKPED | | NO: 494 | NO: 495 | NO: 496 | |
| | | TAVYYCAARDGGVVAGSRSSAQYNYWGQGT | | | | | |
| | | QVTVSS- | | | | | |
| | | SEQ ID | | | | | |
| | | NO: 10 | | | | | |
| 416 | 11 | QLQLVESGGGLVQAGGSLRLSCAASGLTFE | a36 | GYAIG- | YITGSDGTTYYINSVKG- | DRLGSQGRYASAWWRSGDMDL- | 4 |
| | | GYAIGWFRQAPGKEREGVSYITGSDGTTYY | | SEQ ID | SEQ ID | SEQ ID | |
| | | INSVKGRFTISSDNAKSTVYLQMNTLKPED | | NO: 497 | NO: 498 | NO: 499 | |
| | | TAVYYCAVDRLGSQGRYASAWWRSGDMDLW | | | | | |
| | | GKGTQVTVSS- | | | | | |
| | | SEQ ID | | | | | |
| | | NO: 11 | | | | | |
| 474 | 12 | QVQLVESGGGLVQAGGSLRLSCAASGFTFD | a59 | GYAIG- | YITGSDGSTYYADSVKG- | DRLGSRGAYVPIWWRSSDMDL- | 4 |
| | | GYAIGWFRQAPGKEREGVSYITGSDGSTYY | | SEQ ID | SEQ ID | SEQ ID | |
| | | ADSVKGRFTISSNNAKNTVYLHMNSLKPDD | | NO: 500 | NO: 501 | NO: 502 | |
| | | AAVYYCAIDRLGSRGAYVPIWWRSSDMDLW | | | | | |
| | | GKGTLVTVSS- | | | | | |
| | | SEQ ID | | | | | |
| | | NO: 12 | | | | | |
| 440 | 13 | QVQLQESGGGLVQAGDSLRLSCAASGFNFG | a43 | WHAMG- | TITWTGRDTYYADSVRG- | ARERATWAYSEDDCDY- | 1 |
| | | WHAMGWFRQAPGKEREFVATITWTGRDTYY | | SEQ ID | SEQ ID | SEQ ID | |
| | | ADSVRGRFTISKDNAKDTLFLQMNSLRPDD | | NO: 503 | NO: 504 | NO: 505 | |
| | | TGVYYCAKARERATWAYSEDDCDYWGQGTQ | | | | | |
| | | VTVSS- | | | | | |
| | | SEQ ID | | | | | |
| | | NO: 13 | | | | | |
| 1 | 39 | EVQLVESGGGLVQAGDSLRLSCAASGRSFR | a3 | NYILA- | AISWLDGTLYADSVED- | DRGPVRPVLAPDVDY- | 8 |
| | | NYILAWYRQAPGKEREFLAAISWLDGTLYA | | SEQ ID | SEQ ID | SEQ ID | |
| | | DSVEDRFTISRDNPKNTVHLQMDNLKPEDT | | NO: 581 | NO: 582 | NO: 583 | |
| | | AVYYCAADRGPVRPVLAPDVDYWGQGTQVS | | | | | |
| | | VSS- | | | | | |
| | | SEQ ID | | | | | |
| | | NO: 39 | | | | | |
| 2 | 40 | EVQLVESGGGWVQAGDSLRLSCAASGRTFR | a23 | NYVMG- | VITSLGGTVYADSVAN- | DRGPARYISASDVDY- | 8 |
| | | NYVMGWFRQVPGKEREFLSVITSLGGTVYA | | SEQ ID | SEQ ID | SEQ ID | |
| | | DSVANRFTISRDNPKDTVYLQMDSLKPEDT | | NO: 584 | NO: 585 | NO: 586 | |
| | | AVYYCAADRGPARYISASDVDYWGQGTQVT | | | | | |
| | | ASS- | | | | | |
| | | SEQ ID | | | | | |
| | | NO: 40 | | | | | |
| 7 | 41 | QVQLQESGGGLVQAGGSLRLSCAASGRTFR | a48 | THNMG- | AISWNVDNTLYADSVKG- | DNIPLSSDVRATATEFGY- | 6 |
| | | THNMGWFRRAPGKEREFVAAISWNVDNTLY | | SEQ ID | SEQ ID | SEQ ID | |
| | | ADSVKGRFTISRDNDRNMVYLQMNSLKPED | | NO: 587 | NO: 588 | NO: 589 | |
| | | TAVYFCAADNIPLSSDVRATATEFGYWGQG | | | | | |
| | | TRVTVSS- | | | | | |
| | | SEQ ID | | | | | |
| | | NO: 41 | | | | | |
| 13 | 42 | QVQLVESGGGLVQAGDSLRLSCAASGRSFR | a56 | NYILA- | AISWLDGTLYADSVED- | DRGPVRPVLAPDVDY- | 8 |
| | | NYILAWYRQAPGKEREFLAAISWLDGTLYA | | SEQ ID | SEQ ID | SEQ ID | |
| | | DSVEDRFTISRDNPKNTVHLQMDNLKPEDT | | NO: 590 | NO: 591 | NO: 592 | |
| | | AVYYCAADRGPVRPVLAPDVDYWGQGTQVT | | | | | |
| | | VSS- | | | | | |
| | | SEQ ID | | | | | |
| | | NO: 42 | | | | | |
| 14 | 43 | QVQLQESGGGLVQAGGSLRLSCAASGRTFR | a47 | THNMG- | AISWNIDNKLYADSVKG- | DKIPLSSDVAATATEYDD- | 5 |
| | | THNMGWFRRAPGKEREFVAAISWNIDNKLY | | SEQ ID | SEQ ID | SEQ ID | |
| | | ADSVKGRSTISRDNGGNMVFLQMNSLKPED | | NO: 593 | NO: 594 | NO: 595 | |
| | | TAVYYCAADKIPLSSDVAATATEYDDWGQG | | | | | |
| | | TQVTVSS- | | | | | |
| | | SEQ ID | | | | | |
| | | NO: 43 | | | | | |
| 16 | 44 | QLQLVESGGGLVQAGDSLRLSCAASGRAFS | a28 | RYAVG- | AINSSGANTYVLDSVKG- | TSSSYSTYEANYNV- | 14 |
| | | RYAVGWFRMAPGKGREFVSAINSSGANTYV | | SEQ ID | SEQ ID | SEQ ID | |
| | | LDSVKGRFTISRDNDKNTAYLQMDSLQPED | | NO: 596 | NO: 597 | NO: 598 | |
| | | TAVYWCAATSSSYSTYEANYNVWGQGTQVT | | | | | |
| | | VSS- | | | | | |
| | | SEQ ID | | | | | |
| | | NO: 44 | | | | | |
| 19 | 45 | EVQLVESGGGLVQPGGSLRLSCATSGFTFR | a19 | NNFMS- | TISYGGESTTYAESVKG- | ATSYDSIRSGS- | 2 |
| | | NNFMSWVRQAPGKGLEWVSTISYGGESTTY | | SEQ ID | SEQ ID | SEQ ID | |
| | | AESVKGRFTISRDNAKNTLYLQMNNLKPED | | NO: 599 | NO: 600 | NO: 601 | |
| | | TAVYYCAKATSYDSIRSGSRGQGTQVTVSS | | | | | |
| | | - | | | | | |
| | | SEQ ID | | | | | |
| | | NO: 45 | | | | | |
| 20 | 46 | EVQLVESGGGLVQPGGSLRLSCATSGFTFR | a18 | NNFMS- | TISYGGESTTYAESVKG- | ATSYDSIRSGS- | 2 |
| | | NNFMSWVRQAPGEGLEWVSTISYGGESTTY | | SEQ ID | SEQ ID | SEQ ID | |
| | | AESVKGRFTISRDNAKNTLYLQMNNLKPED | | NO: 602 | NO: 603 | NO: 604 | |
| | | TAVYYCAKATSYDSIRSGSRGQGTQVTVSS | | | | | |
| | | - | | | | | |
| | | SEQ ID | | | | | |
| | | NO: 46 | | | | | |
| 22 | 47 | QVQLVESGGGLVQPGGSLRLSCAASGFTFS | a78 | NNFMS- | TISYGGESTTYAESVKG- | ATSYDSVRSGS- | 2 |
| | | NNFMSWVRQAPGKGLEWVSTISYGGESTTY | | SEQ ID | SEQ ID | SEQ ID | |
| | | AESVKGRFTISRDNAKNTLYLQMNNLKPED | | NO: 605 | NO: 606 | NO: 607 | |
| | | TAVYYCAKATSYDSVRSGSRGQGTQVTVSS | | | | | |
| | | - | | | | | |
| | | SEQ ID | | | | | |
| | | NO: 47 | | | | | |
| 23 | 48 | QVQLVESGGGLVQAGDSLRLSCAASGRSFR | a55 | NYILA- | AISWLDGTLYADSVED- | DQGPVRPVLAPDVDY- | 8 |
| | | NYILAWYRQAPGKEREFLAAISWLDGTLYA | | SEQ ID | SEQ ID | SEQ ID | |
| | | DSVEDRFTISRDNPKNTVHLQMDNLKPEDT | | NO: 608 | NO: 609 | NO: 610 | |
| | | AVYYCAADQGPVRPVLAPDVDYWGQGTQVT | | | | | |
| | | VSS- | | | | | |
| | | SEQ ID | | | | | |
| | | NO: 48 | | | | | |
| 28 | 49 | QVQLVESGGGWVQPGDSLRLSCAASGRSFR | a86 | NYVMG- | VITWLGGTLDADSVAD- | DRGPARYISASDVDY- | 8 |
| | | NYVMGWFRQVPGKEREFLSVITWLGGTLDA | | SEQ ID | SEQ ID | SEQ ID | |
| | | DSVADRFTISRDNSKNTVYLQMDSLKPEDT | | NO: 611 | NO: 612 | NO: 613 | |
| | | AVYYCAADRGPARYISASDVDYWGQGTQVT | | | | | |
| | | VSS- | | | | | |
| | | SEQ ID | | | | | |
| | | NO: 49 | | | | | |
| 29 | 50 | QVQLVESGGGLVQAGDSLRLSCAVSGRSFR | a58 | NYILA- | AISWLDGTFYANSVEG- | DRGPVRPVLAPDVDY- | 8 |
| | | NYILAWYRQAPGKEREFLAAISWLDGTFYA | | SEQ ID | SEQ ID | SEQ ID | |
| | | NSVEGRFTISRDNPKNTVHLQMDGLKPEDT | | NO: 614 | NO: 615 | NO: 616 | |
| | | AVYYCAADRGPVRPVLAPDVDYWGQGTQVT | | | | | |
| | | VAS- | | | | | |
| | | SEQ ID | | | | | |
| | | NO: 50 | | | | | |
| 31 | 51 | QVQLVESGGGLVQPGGSLRLSCAASGFTFS | a75 | NNFMS- | TISYFGDSTRYAESVKG- | ATSYDSIRSGS- | 2 |
| | | NNFMSWVRQAPGKGLEWVSTISYFGDSTRY | | SEQ ID | SEQ ID | SEQ ID | |
| | | AESVKGRFTISRDNEKNTLHLQMNNLKPED | | NO: 617 | NO: 618 | NO: 619 | |
| | | TAVYYCAKATSYDSIRSGSRGRGTQVTVSS | | | | | |
| | | - | | | | | |
| | | SEQ ID | | | | | |
| | | NO: 51 | | | | | |
| 37 | 52 | QVQLVESGGGLVQAGGSLRLSCAASGRTFR | a69 | THNMG- | VISWNVDNKLYADSVKG- | DNIPLSSDVRATATEFGY- | 6 |
| | | THNMGWFRRAPGEEREFVAVISWNVDNKLY | | SEQ ID | SEQ ID | SEQ ID | |
| | | ADSVKGRFTISRDNGRNMVYLQMNSLKPED | | NO: 620 | NO: 621 | NO: 622 | |
| | | TAVYYCAADNIPLSSDVRATATEFGYWGQG | | | | | |
| | | TQVTVSS- | | | | | |
| | | SEQ ID | | | | | |
| | | NO: 52 | | | | | |
| 40 | 53 | EVQLVESGGGLVQPGGSLRLSCAASGFTFS | a17 | NNFMS- | TISYFGDSTTYAESVKG- | ATSYDSIRSGS- | 2 |
| | | NNFMSWVRQAPGKGLEWVSTISYFGDSTTY | | SEQ ID | SEQ ID | SEQ ID | |
| | | AESVKGRFTISRDNAKNTLYLQMNNLKPED | | NO: 623 | NO: 624 | NO: 625 | |
| | | TAVYYCAKATSYDSIRSGSRGRGTQVTVSS | | | | | |
| | | - | | | | | |
| | | SEQ ID | | | | | |
| | | NO: 53 | | | | | |
| 46 | 54 | QLQLVESGGGLVQAGGSLRLSCAASGGAFS | a31 | RYAVG- | AIDLSGTNTHVLDSVKG- | TSSSYSDYEANYNI- | 14 |
| | | RYAVGWFRMAPGKGREFVSAIDLSGTNTHV | | SEQ ID | SEQ ID | SEQ ID | |
| | | LDSVKGRFTISRDNDKNTAYLQMDKLQPED | | NO: 626 | NO: 627 | NO: 628 | |
| | | TAVYWCAATSSSYSDYEANYNIWGQGTQVT | | | | | |
| | | VSS- | | | | | |
| | | SEQ ID | | | | | |
| | | NO: 54 | | | | | |
| 53 | 55 | QVQLQESGGGLVQAGDSLRLSCAASGRSFR | a46 | NYILA- | AISWLEGTLYADSVED- | DRGPVRPVLAPDVDY- | 8 |
| | | NYILAWYRQAPGKEREFLAAISWLEGTLYA | | SEQ ID | SEQ ID | SEQ ID | |
| | | DSVEDRFTISRDNPKNTVHLQMDSLKPEDT | | NO: 629 | NO: 630 | NO: 631 | |
| | | AVYYCAADRGPVRPVLAPDVDYWGQGTQVT | | | | | |
| | | VSS- | | | | | |
| | | SEQ ID | | | | | |
| | | NO: 55 | | | | | |
| 54 | 56 | QVQLVESGGGLVQAGGSLRLSCAATGLTFR | a71 | QHNMG- | AISWNVGSTYFADSVKG- | DNIPLASDMRATATAYGY- | 6 |
| | | QHNMGWFRRAPGKEREFVAAISWNVGSTYF | | SEQ ID | SEQ ID | SEQ ID | |
| | | ADSVKGRFTISRDNGKNMVYLQMNSLKPED | | NO: 632 | NO: 633 | NO: 634 | |
| | | TAVYYCAADNIPLASDMRATATAYGYWGQG | | | | | |
| | | TQVTVSS- | | | | | |
| | | SEQ ID | | | | | |
| | | NO: 56 | | | | | |
| 55 | 57 | QVQLVESGGGLVQTGDSLRLSCAASGRSFR | a82 | NYILA- | AISWLDGTLYADSVEG- | DRGPVRPVLAPDVDY- | 8 |
| | | NYILAWYRQAPGKEREFLAAISWLDGTLYA | | SEQ ID | SEQ ID | SEQ ID | |
| | | DSVEGRFTISRDNPKNTVHLQMDSLKPEDT | | NO: 635 | NO: 636 | NO: 637 | |
| | | AVYYCAADRGPVRPVLAPDVDYWGQGTQVT | | | | | |
| | | VSS- | | | | | |
| | | SEQ ID | | | | | |
| | | NO: 57 | | | | | |
| 56 | 58 | EVQLVESGGGLVQAGGSLRLSCAASGRTLS | a13 | GYAMA- | AISSSGIVTHVLDSVKG- | TASSYSDYEANYNY- | 14 |
| | | GYAMAWFRQPPDKGREFVTAISSSGIVTHV | | SEQ ID | SEQ ID | SEQ ID | |
| | | LDSVKGRFTISRDNAKNTVYLQMNSLQPED | | NO: 638 | NO: 639 | NO: 640 | |
| | | TALYFCAATASSYSDYEANYNYWGQGTLVT | | | | | |
| | | VSS- | | | | | |
| | | SEQ ID | | | | | |
| | | NO: 58 | | | | | |
| 60 | 59 | EVQLVESGGGLVQAGGSLRLSCAASGGTFR | a8 | TRNMG- | AVSWNVDNKLYAGSVKG- | DNIPLSSDMRPTATEYDY- | 6 |
| | | TRNMGWFRRAPGKEREFVAAVSWNVDNKLY | | SEQ ID | SEQ ID | SEQ ID | |
| | | AGSVKGRFTISRDNGRNMVYLQMNSLKPED | | NO: 641 | NO: 642 | NO: 643 | |
| | | TAVYYCAADNIPLSSDMRPTATEYDYWGQG | | | | | |
| | | TQVTVSS- | | | | | |
| | | SEQ ID | | | | | |
| | | NO: 59 | | | | | |
| 62 | 60 | EVQLVESGGGWVQPGDSLRLSCAASGRSFR | a24 | NYVMG- | VITSLGGTLDADSVAD- | DRGPARYISASDVDY- | 8 |
| | | NYVMGWFRQVPGKEREFLSVITSLGGTLDA | | SEQ ID | SEQ ID | SEQ ID | |
| | | DSVADRFTISRDNSKNTVYLQMDSLKPEDT | | NO: 644 | NO: 645 | NO: 646 | |
| | | AVYYCAADRGPARYISASDVDYWGQGTQVT | | | | | |
| | | VSS- | | | | | |
| | | SEQ ID | | | | | |
| | | NO: 60 | | | | | |
| 64 | 61 | EVQLVESGGRLVQSGGSLRLSCAASGRAFS | a25 | THNMG- | AISWNVDNKLYANSVKG- | DNIPLSSDMRATATEYDV- | 6 |
| | | THNMGWFRRAPGKEREFVAAISWNVDNKLY | | SEQ ID | SEQ ID | SEQ ID | |
| | | ANSVKGRFAISRDNGRNMVYLQMNSLKPED | | NO: 647 | NO: 648 | NO: 649 | |
| | | TAVYYCAADNIPLSSDMRATATEYDVWGQG | | | | | |
| | | TQVTVSS- | | | | | |
| | | SEQ ID | | | | | |
| | | NO: 61 | | | | | |
| 68 | 62 | QVQLVESGGGLVQPGGSLRLSCAASGFTFS | a77 | NNFMS- | TISYGGESTTYAESVKG- | ATSYDSIRSGS- | 2 |
| | | NNFMSWVRQAPGKGLEWVSTISYGGESTTY | | SEQ ID | SEQ ID | SEQ ID | |
| | | AESVKGRFTISRDNAKNTLYLQMNNLKPED | | NO: 650 | NO: 651 | NO: 652 | |
| | | TAVYYCAKATSYDSIRSGSRGQGTQVTVSS | | | | | |
| | | - | | | | | |
| | | SEQ ID | | | | | |
| | | NO: 62 | | | | | |
| 77 | 63 | QVQLVESGGGLVQAGDSLRLSCAASGRSFR | a57 | NYILA- | AISWLDGTLYADSVED- | DRGPVRPVLAPDVDY- | 8 |
| | | NYILAWYRQAPGKEREFLAAISWLDGTLYA | | SEQ ID | SEQ ID | SEQ ID | |
| | | DSVEDRFTISRDNPKNTVHLQMDSLKPEDT | | NO: 653 | NO: 654 | NO: 655 | |
| | | AVYYCAADRGPVRPVLAPDVDYWGQGTQVT | | | | | |
| | | VSS- | | | | | |
| | | SEQ ID | | | | | |
| | | NO: 63 | | | | | |
| 79 | 64 | EVQLVESGGGLVQAGGSLRLSCAASGGAFS | a6 | RYAVG- | AIDLSGTNTHVLDSVKG- | TSSSYSDYEANYNI- | 14 |
| | | RYAVGWFRMAPGKGREFVSAIDLSGTNTHV | | SEQ ID | SEQ ID | SEQ ID | |
| | | LDSVKGRFTISRDNDKNTAYLQMDKLQPED | | NO: 656 | NO: 657 | NO: 658 | |
| | | TAVYWCAATSSSYSDYEANYNIWGQGTQVT | | | | | |
| | | VSS- | | | | | |
| | | SEQ ID | | | | | |
| | | NO: 64 | | | | | |
| 80 | 65 | QVQLVESGGGLVETGGSLRLSCAASGITFN | a52 | DHTMA- | ARDWSDIITRYASSVKG- | REGGVVAGVRSGAPYDY- | 13 |
| | | DHTMAWFRQAPGKEREFVAARDWSDIITRY | | SEQ ID | SEQ ID | SEQ ID | |
| | | ASSVKGRFTISRDNAGNTGFLQMNSLRPED | | NO: 659 | NO: 660 | NO: 661 | |
| | | TAVYYCAAREGGVVAGVRSGAPYDYWGQGT | | | | | |
| | | QVTVSS- | | | | | |
| | | SEQ ID | | | | | |
| | | NO: 65 | | | | | |
| 81 | 66 | QVQLVESGGGLVQPGGSLRLSCAASGRTFE | a79 | THNMG- | VISWNVDSTEYTDSVKG- | DNIPLSSDMSPTAAEYGY- | 6 |
| | | THNMGWFRRAPGKEREFVAVISWNVDSTEY | | SEQ ID | SEQ ID | SEQ ID | |
| | | TDSVKGRFTISRDNDKNMVYLQMNSLKPED | | NO: 662 | NO: 663 | NO: 664 | |
| | | TGVYYCAADNIPLSSDMSPTAAEYGYWGQG | | | | | |
| | | TQVTVSS- | | | | | |
| | | SEQ ID | | | | | |
| | | NO: 66 | | | | | |
| 83 | 67 | QVQLVESGGGLVQAGGSLRLSCAASGGTFR | a61 | TRNMG- | AVSWNVDNKLYAGSVKG- | DNIPLSSDMRPTATEYDY- | 6 |
| | | TRNMGWFRRAPGKEREFVAAVSWNVDNKLY | | SEQ ID | SEQ ID | SEQ ID | |
| | | AGSVKGRFTISRDNGRNMVYLQMNSLKPED | | NO: 665 | NO: 666 | NO: 667 | |
| | | TAVYYCAADNIPLSSDMRPTATEYDYWGQG | | | | | |
| | | TQVTVSS- | | | | | |
| | | SEQ ID | | | | | |
| | | NO: 67 | | | | | |
| 84 | 68 | QLQLVESGGGLVQAGDSLRLSCAASGRSFR | a29 | NYIMA- | AISWLDGTLYADSVED- | DRGPVRPVLAPDVDY- | 8 |
| | | NYIMAWYRQAPGKEREFLAAISWLDGTLYA | | SEQ ID | SEQ ID | SEQ ID | |
| | | DSVEDRFTISRDNPKNTVHLQMDSLKPEDT | | NO: 668 | NO: 669 | NO: 670 | |
| | | AVYYCAADRGPVRPVLAPDVDYWGQGTQVT | | | | | |
| | | VSS- | | | | | |
| | | SEQ ID | | | | | |
| | | NO: 68 | | | | | |
| 85 | 69 | QVQLVESGGGLVQPGGSLRLSCAASGFTFS | a76 | NNFMS- | TISYFGDSTRYAESVKG- | ATSYDSIRSGS- | 2 |
| | | NNFMSWVRQAPGKGLEWVSTISYFGDSTRY | | SEQ ID | SEQ ID | SEQ ID | |
| | | AESVKGRFTISRDNEKNTLYLQMNNLKPED | | NO: 671 | NO: 672 | NO: 673 | |
| | | TAVYYCAKATSYDSIRSGSRGRGTQVTVSS | | | | | |
| | | - | | | | | |
| | | SEQ ID | | | | | |
| | | NO: 69 | | | | | |
| 86 | 70 | QVQLQESGGGLVQAGDSLRLSCAASGRSFR | a45 | NYILA- | AISWLDGTLYADSVED- | DRGPVRPVLAPDVDY- | 8 |
| | | NYILAWYRQAPGKEREFLAAISWLDGTLYA | | SEQ ID | SEQ ID | SEQ ID | |
| | | DSVEDRFTISRDNPKNTVHLQMDSLKPEDT | | NO: 674 | NO: 675 | NO: 676 | |
| | | AVYYCAADRGPVRPVLAPDVDYWGQGTQVT | | | | | |
| | | VSS- | | | | | |
| | | SEQ ID | | | | | |
| | | NO: 70 | | | | | |
| 87 | 71 | QVQLVESGGGLVQAGGSLRLSCAASGITFN | a66 | DHTMA- | ARDWSDIITRYASSVKG- | REGGVVAGVRSGAPYDY- | 13 |
| | | DHTMAWFRQAPGKEREFVAARDWSDIITRY | | SEQ ID | SEQ ID | SEQ ID | |
| | | ASSVKGRFTISRDNAGNTGFLQMNSLRPED | | NO: 677 | NO: 678 | NO: 679 | |
| | | TAVYYCAAREGGVVAGVRSGAPYDYWGRGT | | | | | |
| | | QVTVSS- | | | | | |
| | | SEQ ID | | | | | |
| | | NO: 71 | | | | | |
| 88 | 72 | QVQLVESGGGLVQAGGSLRLSCAASGRTFR | a70 | THNMG- | AISWNVDNTLYADSVKG- | DNIPLSSDVRATATEFGY- | 6 |
| | | THNMGWFRRAPGKEREFVAAISWNVDNTLY | | SEQ ID | SEQ ID | SEQ ID | |
| | | ADSVKGRFTISRDNDRNMVYLQMNSLKPED | | NO: 680 | NO: 681 | NO: 682 | |
| | | TAVYFCAADNIPLSSDVRATATEFGYWGQG | | | | | |
| | | TRVTVSS- | | | | | |
| | | SEQ ID | | | | | |
| | | NO: 72 | | | | | |
| 90 | 73 | QVQLQESGGGLVQAGDSLRLSCAASGRSFR | a44 | NYILA- | AISWLDGTFYANSVEG- | DRGPVRPVLAPDVDY- | 8 |
| | | NYILAWYRQAPGKEREFLAAISWLDGTFYA | | SEQ ID | SEQ ID | SEQ ID | |
| | | NSVEGRFTISRDNPKNTVHLQMDGLKPEDT | | NO: 683 | NO: 684 | NO: 685 | |
| | | AVYYCAADRGPVRPVLAPDVDYWGQGTQVT | | | | | |
| | | VAS- | | | | | |
| | | SEQ ID | | | | | |
| | | NO: 73 | | | | | |
| 93 | 74 | QVQLVESGGGLVQAGGSLRLSCAASGITFN | a65 | DHTMA- | ARDWSDIITRYASSVKG- | REGGVVAGVRSGAPYDY- | 13 |
| | | DHTMAWFRQAPGKEREFVAARDWSDIITRY | | SEQ ID | SEQ ID | SEQ ID | |
| | | ASSVKGRFTISRDNAGNTGFLQMNSLRPED | | NO: 686 | NO: 687 | NO: 688 | |
| | | TAVYYCAAREGGVVAGVRSGAPYDYWGQGT | | | | | |
| | | QVTVSS- | | | | | |
| | | SEQ ID | | | | | |
| | | NO: 74 | | | | | |
| 94 | 75 | EVQLVESGGGLVQAGGSLGLSCAASGITFN | a4 | DHTMA- | ARDWSDIITRYASSVKG- | REGGVVAGVRSGAPYDY- | 13 |
| | | DHTMAWFRQAPGKEREFVAARDWSDIITRY | | SEQ ID | SEQ ID | SEQ ID | |
| | | ASSVKGRFTISRDNAGNTGFLQMNSLRPED | | NO: 689 | NO: 690 | NO: 691 | |
| | | TAVYYCAAREGGVVAGVRSGAPYDYWGQGT | | | | | |
| | | QVTVSP- | | | | | |
| | | SEQ ID | | | | | |
| | | NO: 75 | | | | | |
| 386 | 76 | QVQLVESGGGLVQAGGSLRLSCAASGITFN | a64 | DHTMA- | ARDWSDIITRYASSVKG- | REGGVVAGVRSGAPYDY- | 13 |
| | | DHTMAWFRQAPGKEREFVAARDWSDIITRY | | SEQ ID | SEQ ID | SEQ ID | |
| | | ASSVKGRFTISRDNAGNTGFLQMNSLRPED | | NO: 692 | NO: 693 | NO: 694 | |
| | | TAVYYCAAREGGVVAGVRSGAPYDYWG* | | | | | |
| | | GTQVTVSS-SEQ ID | | | | | |
| | | NO: 76 | | | | | |
| 392 | 77 | EVQLVESGGGLVQAGGSLRLSCAASGITFN | a10 | DHTMA- | ARDWSDIITRYASSVKG- | REGGVVAGVRSGAPYDY- | 13 |
| | | DHTMAWFRQAPGKEREFVAARDWSDIITRY | | SEQ ID | SEQ ID | SEQ ID | |
| | | ASSVKGRFTISRDNAGNTGFLQMNSLRPED | | NO: 695 | NO: 696 | NO: 697 | |
| | | TAVYYCAAREGGVVAGVRSGAPYDYWGQGT | | | | | |
| | | QVTVSS- | | | | | |
| | | SEQ ID | | | | | |
| | | NO: 77 | | | | | |
| 400 | 78 | EVQLVESGGGLVQAGGSLRLSCAASGITFN | a9 | DHTMA- | ARDWSDIITRYAGSVKG- | REGGVVAGVRSGAPYDY- | 13 |
| | | DHTMAWFRQAPGKEREFVAARDWSDIITRY | | SEQ ID | SEQ ID | SEQ ID | |
| | | AGSVKGRFTISRDNAGNTGFLQMNSLRPED | | NO: 698 | NO: 699 | NO: 700 | |
| | | TAVYYCAAREGGVVAGVRSGAPYDYWGQGT | | | | | |
| | | QVTVSS- | | | | | |
| | | SEQ ID | | | | | |
| | | NO: 78 | | | | | |
| 402 | 79 | QVQLQESGGGLVQPGGSLRLSCAASGITFN | a50 | DHTMA- | ARDWSDIITRYASSVKG- | REGGVVAGVRSGAPYDY- | 13 |
| | | DHTMAWFRQAPGKEREFVAARDWSDIITRY | | SEQ ID | SEQ ID | SEQ ID | |
| | | ASSVKGRFTISRDNAGNTGFLQMNSLRPED | | NO: 701 | NO: 702 | NO: 703 | |
| | | TAVYYCAAREGGVVAGVRSGAPYDYWGQGT | | | | | |
| | | QVTVSS- | | | | | |
| | | SEQ ID | | | | | |
| | | NO: 79 | | | | | |
| 405 | 80 | EVQLVESGGGLVQAGGSLRLSCAASGRTLY | a14 | SSYGMA- | SISRYGTYTSYADSVKD- | YTYSGSFYSTVKTHRDEYQY- | 15 |
| | | SSYGMAWFRQAPGKERDFVGSISRYGTYTS | | SEQ ID | SEQ ID | SEQ ID | |
| | | YADSVKDRFTISRDNAKSTVYLQMNNTKPE | | NO: 704 | NO: 705 | NO: 706 | |
| | | DTAVYYCAAYTYSGSFYSTVKTHRDEYQYW | | | | | |
| | | GQGTQVTVSS- | | | | | |
| | | SEQ ID | | | | | |
| | | NO: 80 | | | | | |
| 411 | 81 | EVQLVESGGGSVQAGGSLRLSCEASGRTLF | a22 | SSYGMG- | AISRFGTYTSYADPVKD- | YTYSGSFYSTVKTHHDEYRY- | 15 |
| | | SSYGMGWFRQAPGKEREFVGAISRFGTYTS | | SEQ ID | SEQ ID | SEQ ID | |
| | | YADPVKDRFTISRDNAKSTVYLQMNNTKPE | | NO: 707 | NO: 708 | NO: 709 | |
| | | DTAVYYCAAYTYSGSFYSTVKTHHDEYRYW | | | | | |
| | | GQGTQVTVSS- | | | | | |
| | | SEQ ID | | | | | |
| | | NO: 81 | | | | | |
| 412 | 82 | QLQLVESGGGLVQAGGSLRLSCAASGLTFD | a35 | GYAIG- | YINGRDGSTFYADSVKG- | DRLGSRGAYVSSWWRSPDMDL- | 4 |
| | | GYAIGWFRQASGKEREGVSYINGRDGSTFY | | SEQ ID | SEQ ID | SEQ ID | |
| | | ADSVKGRFIISSDNAKNTVDLQMNSLNPDD | | NO: 710 | NO: 711 | NO: 712 | |
| | | AAVYYCAVDRLGSRGAYVSSWWRSPDMDLW | | | | | |
| | | GKGTLVTVSS- | | | | | |
| | | SEQ ID | | | | | |
| | | NO: 82 | | | | | |
| 413 | 83 | QVQLVESGGGLVQAGGSLRLSCAASGGTFR | a62 | TRNMG- | AVSWSVDNKLYAGSVKG- | DNIPLSSDMRPTATEFGY- | 6 |
| | | TRNMGWFRRAPGKEREFVAAVSWSVDNKLY | | SEQ ID | SEQ ID | SEQ ID | |
| | | AGSVKGRFTISRDNGRNMVYLQMNSLKPED | | NO: 713 | NO: 714 | NO: 715 | |
| | | TAVYYCAADNIPLSSDMRPTATEFGYWGQG | | | | | |
| | | TRVTVSS- | | | | | |
| | | SEQ ID | | | | | |
| | | NO: 83 | | | | | |
| 417 | 84 | QLQLVESGGGLVQAGGSLRLSCAASGLTFD | a33 | GYAIG- | YINGRDGSTFYADSVKG- | DRLGSRGAYVSTWWRPPDMDL- | 4 |
| | | GYAIGWFRQASGKEREGVSYINGRDGSTFY | | SEQ ID | SEQ ID | SEQ ID | |
| | | ADSVKGRFIISSDNAKNTVDLQMNSLKPDD | | NO: 716 | NO: 717 | NO: 718 | |
| | | AAVYYCAVDRLGSRGAYVSTWWRPPDMDLW | | | | | |
| | | GKGTLVTVSP- | | | | | |
| | | SEQ ID | | | | | |
| | | NO: 84 | | | | | |
| 418 | 85 | EVQLVESGGGLVQAGGSLRLSCAASGFTFD | a5 | GYAVG- | YITGSDGVTYYIDSVKG- | DRLGSQGRYASSWWRAGDMDF- | 4 |
| | | GYAVGWFRQAPGKEPEGVSYITGSDGVTYY | | SEQ ID | SEQ ID | SEQ ID | |
| | | IDSVKGRFTISSDNAKRTVYLQMNTLKPED | | NO: 719 | NO: 720 | NO: 721 | |
| | | TAVYYCAVDRLGSQGRYASSWWRAGDMDFW | | | | | |
| | | GKGTLVTVSS- | | | | | |
| | | SEQ ID | | | | | |
| | | NO: 85 | | | | | |
| 424 | 86 | QVQLVESGGGLVQAGDSLRLSCAASGFNFG | a54 | WHAMG- | TITWTGRDTYYADSVRG- | ARERATWAYSEDDCDY- | 1 |
| | | WHAMGWFRQAPGKEREFVATITWTGRDTYY | | SEQ ID | SEQ ID | SEQ ID | |
| | | ADSVRGRFTISKDNAKNTLFLQMSSLRPDD | | NO: 722 | NO: 723 | NO: 724 | |
| | | TGVYYCAKARERATWAYSEDDCDYWGQGTQ | | | | | |
| | | VTVSS- | | | | | |
| | | SEQ ID | | | | | |
| | | NO: 86 | | | | | |
| 427 | 87 | QVQLVESGGGLVQAGGSLRLSCAASGLTFD | a67 | GYAIG- | YINGRDGSTFYTDSVKG- | DRLGSRGAYVSSWWRSPDMDL- | 4 |
| | | GYAIGWFRQASGKEREGVSYINGRDGSTFY | | SEQ ID | SEQ ID | SEQ ID | |
| | | TDSVKGRFIISSDNAKNTVDLQMNSLKPDD | | NO: 725 | NO: 726 | NO: 727 | |
| | | AAVYYCAVDRLGSRGAYVSSWWRSPDMDLW | | | | | |
| | | GKGTLVTVSS- | | | | | |
| | | SEQ ID | | | | | |
| | | NO: 87 | | | | | |
| 428 | 88 | QVQLVESGGGLVQAGDSLRLSCAASGFNFG | a53 | WHAMG- | TITWTGRDTYYADSVRG- | ARERATWAYSEDDCDY- | 1 |
| | | WHAMGWFRQAPGKEREFVATITWTGRDTYY | | SEQ ID | SEQ ID | SEQ ID | |
| | | ADSVRGRFTISKDNAKDTLFLQMNSLRPDD | | NO: 728 | NO: 729 | NO: 730 | |
| | | TGVYYCAKARERATWAYSEDDCDYWGQGTQ | | | | | |
| | | VTVSS- | | | | | |
| | | SEQ ID | | | | | |
| | | NO: 88 | | | | | |
| 430 | 89 | EVQLVESGGGLVQAGDSLRLSCAASGFNFG | a1 | WHAMG- | TITWTGRDTYYADSVRG- | ARERATWAYSEDDCDY- | 1 |
| | | WHAMGWFRQAPGKEREFVATITWTGRDTYY | | SEQ ID | SEQ ID | SEQ ID | |
| | | ADSVRGRFTISKDNAKDTLFLQMNSLRPDD | | NO: 731 | NO: 732 | NO: 733 | |
| | | TGVYYCAKARERATWAYSEDDCDYWGQGTQ | | | | | |
| | | VTVSS- | | | | | |
| | | SEQ ID | | | | | |
| | | NO: 89 | | | | | |
| 433 | 90 | QVQLVESGGGLAQAGASLRLSCAASGRTLY | a51 | SSYGMA- | SISRYGTYTSYADSVKG- | YTYSGSFYSTVKTHRDEYQY- | 15 |
| | | SSYGMAWFRQAPGKERDFVGSISRYGTYTS | | SEQ ID | SEQ ID | SEQ ID | |
| | | YADSVKGRFTISRDNAKSTVYLQMNNTKPE | | NO: 734 | NO: 735 | NO: 736 | |
| | | DTAVYYCAAYTYSGSFYSTVKTHRDEYQYW | | | | | |
| | | GQGTQVTVSS- | | | | | |
| | | SEQ ID | | | | | |
| | | NO: 90 | | | | | |
| 434 | 91 | QLQLVESGGGLVQAGGSLRLSCAASGLTFD | a32 | GYAIG- | YINGRDGSTFYADSVKG- | DRLGSRGAYVSSWWRSPDMDL- | 4 |
| | | GYAIGWFRQASGKEREGVSYINGRDGSTFY | | SEQ ID | SEQ ID | SEQ ID | |
| | | ADSVKGRFIISSDNAKNTVDLQMNSLKPDD | | NO: 737 | NO: 738 | NO: 739 | |
| | | AAVYYCAVDRLGSRGAYVSSWWRSPDMDLW | | | | | |
| | | GKGTLVTVSS- | | | | | |
| | | SEQ ID | | | | | |
| | | NO: 91 | | | | | |
| 435 | 92 | QVQLVESGGGSVQAGGSLRLSCEASGRTLF | a85 | SSYGMG- | AISRFGTYTSYADPVKD- | YTYSGSFYSTVKTHHDEYRY- | 15 |
| | | SSYGMGWFRQAPGKEREFVGAISRFGTYTS | | SEQ ID | SEQ ID | SEQ ID | |
| | | YADPVKDRFTISRDNAKSTVYLQMNNTKPE | | NO: 740 | NO: 741 | NO: 742 | |
| | | DTAVYYCAAYTYSGSFYSTVKTHHDEYRYW | | | | | |
| | | GQGTQVTVSS- | | | | | |
| | | SEQ ID | | | | | |
| | | NO: 92 | | | | | |
| 442 | 93 | QLQLVESGGGLVQAGGSLRLSCVAHGLTFD | a40 | GYAIG- | YITGSDGSRYYADSVKG- | DRLGSRGAYVSAWWRSSDMDL- | 4 |
| | | GYAIGWFRQAPGKELEGVSYITGSDGSRYY | | SEQ ID | SEQ ID | SEQ ID | |
| | | ADSVKGRFTISSDVAKNTVYLHMNNLKPDD | | NO: 743 | NO: 744 | NO: 745 | |
| | | AARYYCAVDRLGSRGAYVSAWWRSSDMDLW | | | | | |
| | | GKGTLVTVSS- | | | | | |
| | | SEQ ID | | | | | |
| | | NO: 93 | | | | | |
| 445 | 94 | EVQLVESGGGSVQAGGSLRLSCEASGPTLF | a21 | SSYGMG- | AISRYGTYTSYADPVKD- | YTYSGSFYSTVKTHHDEYHY- | 15 |
| | | SSYGMGWFRQAPGKKEREFVGAISRYGTYT | | SEQ ID | SEQ ID | SEQ ID | |
| | | SYADPVKDRFTISRDNAKSTVYLQMNNTKP | | NO: 746 | NO: 747 | NO: 748 | |
| | | EDTAVYYCAAYTYSGSFYSTVKTHHDEYHY | | | | | |
| | | WGQGTQVTVSS- | | | | | |
| | | SEQ ID | | | | | |
| | | NO: 94 | | | | | |
| 447 | 95 | QLQLVESGGGLVQAGGSLRLSCTASGLTFE | a39 | GYAIG- | YITGSDASTYYIDSVKG- | DRLGSRGGYASSWWRSGDMDL- | 4 |
| | | GYAIGWFRQAPGKEREGVSYITGSDASTYY | | SEQ ID | SEQ ID | SEQ ID | |
| | | IDSVKGRFTISSDFAKSTVYLQMNNLKPED | | NO: 749 | NO: 750 | NO: 751 | |
| | | TAVYYCAVDRLGSRGGYASSWWRSGDMDLW | | | | | |
| | | GKGTLVTVSS- | | | | | |
| | | SEQ ID | | | | | |
| | | NO: 95 | | | | | |
| 448 | 96 | QLQLVESGGGLVQAGGSLRLSCAASGLTFD | a34 | GYAIG- | YINGRDGSTFYADSVKG- | DRLGSRGAYVSTWWRSPDMDL- | 4 |
| | | GYAIGWFRQASGKEREGVSYINGRDGSTFY | | SEQ ID | SEQ ID | SEQ ID | |
| | | ADSVKGRFIISSDNAKNTVDLQMNSLKPDD | | NO: 752 | NO: 753 | NO: 754 | |
| | | AAVYYCAVDRLGSRGAYVSTWWRSPDMDLW | | | | | |
| | | GKGTLVTVSS- | | | | | |
| | | SEQ ID | | | | | |
| | | NO: 96 | | | | | |
| 451 | 97 | QLQLVESGGGLVQAGGSLRLSCAASGFTFD | a30 | GYAIG- | YITGSDGSTYYADSVKG- | DRLGSRGAYVPIWWRSSDMDL- | 4 |
| | | GYAIGWFRQAPGKEREGVSYITGSDGSTYY | | SEQ ID | SEQ ID | SEQ ID | |
| | | ADSVKGRFTISSNNAKNTVYLHMNSLKPDD | | NO: 755 | NO: 756 | NO: 757 | |
| | | AAVYYCAIDRLGSRGAYVPIWWRSSDMDLW | | | | | |
| | | GKGTLVTVSS- | | | | | |
| | | SEQ ID | | | | | |
| | | NO: 97 | | | | | |
| 456 | 98 | QLQLVESGGDLVQPGGSLRLSCTASGLTFD | a26 | GYAIG- | YITGSNGSTYYIDSVKG- | DRLGSRGLYASSWWRSGDMDL- | 4 |
| | | GYAIGWFRQAPGKEREGVSYITGSNGSTYY | | SEQ ID | SEQ ID | SEQ ID | |
| | | IDSVKGRFTISSDIAKSTVYLQMNTLKPED | | NO: 758 | NO: 759 | NO: 760 | |
| | | TAVYYCAVDRLGSRGLYASSWWRSGDMDLW | | | | | |
| | | GKGTLVTVSS- | | | | | |
| | | SEQ ID | | | | | |
| | | NO: 98 | | | | | |
| 457 | 99 | QLQLVESGGGLVQAGGSLRLSCTASGLTFD | a38 | GYAIG- | YITGSDGSTYYIDSVKG- | DRLGSRGLYASSWWRSGDMDL- | 4 |
| | | GYAIGWFRQAPGKEREGVSYITGSDGSTYY | | SEQ ID | SEQ ID | SEQ ID | |
| | | IDSVKGRFTISSDIAKSTVYLQMNSLKPED | | NO: 761 | NO: 762 | NO: 763 | |
| | | TAVYYCAVDRLGSRGLYASSWWRSGDMDLW | | | | | |
| | | GKGTLVTVSS- | | | | | |
| | | SEQ ID | | | | | |
| | | NO: 99 | | | | | |
| 459 | 100 | QLQLVESGGGLVQTGGSLRLSCSTSGFTFD | a41 | GYAIG- | YITGSDGSTYYADSVKG- | DRLGSGGAYVPIWWRSSDMDL- | 4 |
| | | GYAIGWFRQAPGKEPEGVSYITGSDGSTYY | | SEQ ID | SEQ ID | SEQ ID | |
| | | ADSVKGRFTISSDNAKNTVYLHMNSLKPDD | | NO: 764 | NO: 765 | NO: 766 | |
| | | AAVYYCAIDRLGSGGAYVPIWWRSSDMDLW | | | | | |
| | | GKGTLVTVSS- | | | | | |
| | | SEQ ID | | | | | |
| | | NO: 100 | | | | | |
| 460 | 101 | QVQLVESGGGLVQAGGSLRLSCTASGLTFD | a72 | GYAIG- | YITGSDGSTYYRDSVKG- | DRLGSRGLYASSWWRSGDMDL- | 4 |
| | | GYAIGWFRQAPGKEREGVSYITGSDGSTYY | | SEQ ID | SEQ ID | SEQ ID | |
| | | RDSVKGRFTISSDNAKSTVYLQMNTLKPED | | NO: 767 | NO: 768 | NO: 769 | |
| | | TAVYYCAVDRLGSRGLYASSWWRSGDMDLW | | | | | |
| | | GKGTLVTVSS- | | | | | |
| | | SEQ ID | | | | | |
| | | NO: 101 | | | | | |
| 461 | 102 | EVQLVESGGGLVQAGGSLRLSCTASGLTFD | a16 | GYAIG- | YITGSDGSTYYIDSVKG- | DRLGSRGLYASSWWRSGDMDL- | 4 |
| | | GYAIGWFRQAPGKEREGVSYITGSDGSTYY | | SEQ ID | SEQ ID | SEQ ID | |
| | | IDSVKGRFTISSDIAKSTVYLQMNSLKPED | | NO: 770 | NO: 771 | NO: 772 | |
| | | TAVYYCAVDRLGSRGLYASSWWRSGDMDLW | | | | | |
| | | GKGTLVTVSS- | | | | | |
| | | SEQ ID | | | | | |
| | | NO: 102 | | | | | |
| 467 | 103 | QVQLVESGGGLVQAGGSLRLSCTASGLTFE | a73 | GYAIG- | YITGSDASTYYIDSVKG- | DRLGSRGGYASSWWRSGDMDL- | 4 |
| | | GYAIGWFRQAPGKEREGVSYITGSDASTYY | | SEQ ID | SEQ ID | SEQ ID | |
| | | IDSVKGRFTISSDFAKSTVYLQMNNLKPED | | NO: 773 | NO: 774 | NO: 775 | |
| | | TAVYYCAVDRLGSRGGYASSWWRSGDMDLW | | | | | |
| | | GKGTLVTVSS- | | | | | |
| | | SEQ ID | | | | | |
| | | NO: 103 | | | | | |
| 468 | 104 | QVQLVESGGGSVQAGGSLRLSCEASGPTLF | a84 | SSYGMG- | AISRYGTYTSYADPVKD- | YTYSGSFYSTVKTHHDEYHY- | 15 |
| | | SSYGMGWFRQAPGKKEREFVGAISRYGTYT | | SEQ ID | SEQ ID | SEQ ID | |
| | | SYADPVKDRLTISRGNAKSTVYLQMNNTKP | | NO: 776 | NO: 777 | NO: 778 | |
| | | EDTAVYYCAAYTYSGSFYSTVKTHHDEYHY | | | | | |
| | | WGQGTQVTVSS- | | | | | |
| | | SEQ ID | | | | | |
| | | NO: 104 | | | | | |
| 469 | 105 | EVQLVESGGGSVQAGGSLRLSCAASGRTLY | a20 | SSYGMA- | SISRYGTYTSYADSVKD- | YTYSGSFYSTVKTHRDEYQY- | 15 |
| | | SSYGMAWFRQAPGKERDFVGSISRYGTYTS | | SEQ ID | SEQ ID | SEQ ID | |
| | | YADSVKDRFTISRDNAKSTVYLQMNNTKPE | | NO: 779 | NO: 780 | NO: 781 | |
| | | DTAVYYCAAYTYSGSFYSTVKTHRDEYQYW | | | | | |
| | | GQGTQVTVSS- | | | | | |
| | | SEQ ID | | | | | |
| | | NO: 105 | | | | | |
| 470 | 106 | QVQLVESGGGSVQAGGSLRLSCAASGRTLY | a83 | SSYGMA- | SISRYGTYTSYADSVKD- | YTYSGSFYSAVKTHRDEYQY- | 15 |
| | | SSYGMAWFRQAPGKERDFVGSISRYGTYTS | | SEQ ID | SEQ ID | SEQ ID | |
| | | YADSVKDRFTISRDNAKSTVYLQMNNTKPE | | NO: 782 | NO: 783 | NO: 784 | |
| | | DTAVYYCAAYTYSGSFYSAVKTHRDEYQYW | | | | | |
| | | GQGTQVTVSS- | | | | | |
| | | SEQ ID | | | | | |
| | | NO: 106 | | | | | |
| 475 | 107 | EVQLVESGGGLVQAGDSLRLSCAASGFNFG | a2 | WHAMG- | TITWTGRDTYYADSVRG- | ARERATWAYSEDDCDY- | 1 |
| | | WHAMGWFRQAPGKEREFVATITWTGRDTYY | | SEQ ID | SEQ ID | SEQ ID | |
| | | ADSVRGRFTISKDNAKNTLFLQMSSLRPDD | | NO: 785 | NO: 786 | NO: 787 | |
| | | TGVYYCAKARERATWAYSEDDCDYWGQGTQ | | | | | |
| | | VTVSS- | | | | | |
| | | SEQ ID | | | | | |
| | | NO: 107 | | | | | |
| 476 | 108 | QLQLVESGGGSVQAGGSLRLSCAASGRTLY | a42 | SSYGMA- | SISRYGTYTSYADSVKD- | YTYSGSFYSTVKTHRDEYQY- | 15 |
| | | SSYGMAWFRQAPGKERDFVGSISRYGTYTS | | SEQ ID | SEQ ID | SEQ ID | |
| | | YADSVKDRFTISRDNAKSTVYLQMNNTKPE | | NO: 788 | NO: 789 | NO: 790 | |
| | | DTAVYYCAAYTYSGSFYSTVKTHRDEYQYW | | | | | |
| | | GQGTQVTVSS- | | | | | |
| | | SEQ ID | | | | | |
| | | NO: 108 | | | | | |
| 477 | 109 | QLQLVESGGGLVQAGDSLRLSCAASGFNFG | a27 | WHAMG- | TITWTGRDTYYADSVRG- | ARERATWAYSEDDCDY- | 1 |
| | | WHAMGWFRQAPGKEREFVATITWTGRDTYY | | SEQ ID | SEQ ID | SEQ ID | |
| | | ADSVRGRFTISKDNAKDTLFLQMNSLRPDD | | NO: 791 | NO: 792 | NO: 793 | |
| | | TGVYYCAKARERATWAYSEDDCDYWGQGTQ | | | | | |
| | | VTVSS- | | | | | |
| | | SEQ ID | | | | | |
| | | NO: 109 | | | | | |
| 478 | 110 | QLQLVESGGGLVQAGGSLRLSCLASGLTFD | a37 | GYAIG- | YITGSDGSSYYADSVKG- | DHLGSRGGYHSSWWRSSDMDL- | 4 |
| | | GYAIGWFRQAPGKELEGVSYITGSDGSSYY | | SEQ ID | SEQ ID | SEQ ID | |
| | | ADSVKGRFTISSDNAKNTVYLHMNSLKPDD | | NO: 794 | NO: 795 | NO: 796 | |
| | | AAVYYCAIDHLGSRGGYHSSWWRSSDMDLW | | | | | |
| | | GKGTLVTVSS- | | | | | |
| | | SEQ ID | | | | | |
| | | NO: 110 | | | | | |

TABLE 8

| | ascending | | | P.E. binding | | P.E. Off-rate ranking SPR- | | | |
|---|---|---|---|---|---|---|---|---|---|
| | numbering | | | ELISA | | mouse and human IL-2Ra | | | |
| Clone | Patent | | | hu | mo | Rmax- | hd(1/s)- | Rmax- | kd (1/s)- |
| nr. | clone ID | V ID | Clone ID | IL-2R | IL-2R | hu | hu | mo | mo |
| 115 | 14 | 14-MP02C03 | FJ1427 P035MP02C03 | 0.183 | 0.047 | 254.63 | 7.14E−04 | 3.85 | N/A |
| 141 | 15 | 15-MP02E06 | FJ1427 P035MP02E06 | 0.581 | 0.048 | 88.25 | 1.69E−03 | 3.25 | N/A |
| 154 | 16 | 16-MP02B08 | FJ1427_P035MP02B08 | 1.379 | 0.048 | 134.82 | 1.41E−04 | 2.5 | N/A |
| 163 | 17 | 17-MP02C09 | FJ1427_P035MP02C09 | 1.205 | 0.047 | 99.83 | 8.48E−04 | −4.22 | N/A |
| 211 | 18 | 18-MP03C03 | FJ1427_P035MP03C03 | 0.405 | 0.048 | 124.58 | 5.94E−04 | 13.86 | N/A |
| 254 | 19 | 19-MP03F08 | FJ1427_P035MP03F08 | 0.245 | 0.049 | 386.6 | 2.96E−04 | 5.01 | N/A |
| 270 | 20 | 20-MP03F10 | FJ1427_P035MP03F10 | 0.497 | 0.048 | 51.52 | 5.95E−04 | 6.86 | N/A |
| 281 | 21 | 21-MP03A12 | FJ1427 P035MP03A12 | 0.146 | 0.050 | 95.17 | 6.28E−03 | 7.01 | N/A |
| 286 | 22 | 22-MP03F12 | FJ1427_P035MP03F12 | 0.781 | 0.048 | 166.58 | 4.19E−04 | 4.04 | N/A |
| 502 | 23 | 23-MP06F03 | FJ1427_P035MP06F03 | 0.540 | 0.047 | 466.68 | 1.05E−03 | 28.66 | 1.93E−01 |
| 517 | 24 | 24-MP06E05 | FJ1427 P035MP06E05 | 0.355 | 0.046 | 315.21 | 4.71E−04 | 9.06 | N/A |
| 518 | 25 | 25-MP06F05 | FJ1427_P035MP06F05 | 0.159 | 0.047 | 200.28 | 8.36E−04 | 14.57 | N/A |
| 529 | 26 | 26-MP06A07 | FJ1427_P035MP06A07 | 1.027 | 0.048 | 451.44 | 3.63E−04 | 45.6 | 8.17E−03 |
| 97 | 111 | | FJ1427_P035MP02A01 | 0.311 | 0.048 | 363.33 | 2.30E−04 | 3.09 | N/A |
| 98 | 112 | | FJ1427_P035MP02B01 | 0.271 | 0.048 | 374.77 | 1.19E−04 | 7.83 | N/A |
| 99 | 113 | | FJ1427 P035MP02C01 | 0.700 | 0.047 | 377.07 | 7.89E−04 | 5.4 | N/A |
| 100 | 114 | | FJ1427 P035MP02D01 | 0.573 | 0.048 | 379.16 | 1.67E−04 | 6.12 | N/A |
| 101 | 115 | | FJ1427_P035MP02E01 | 1.111 | 0.049 | 392.8 | 4.50E−05 | 4.79 | N/A |
| 102 | 116 | | FJ1427_P035MP02F01 | 0.469 | 0.048 | 355.79 | 3.00E−04 | 4.93 | N/A |
| 103 | 117 | | FJ1427_P035MP02G01 | 0.169 | 0.049 | 314.66 | 3.68E−04 | 6.13 | N/A |
| 104 | 118 | | FJ1427_P035MP02H01 | 0.487 | 0.051 | 65.91 | 1.06E−03 | 4.83 | N/A |
| 105 | 119 | | FJ1427_P035MP02A02 | 0.223 | 0.049 | 77.21 | 2.62E−03 | 1.29 | N/A |
| 106 | 120 | | FJ1427_P035MP02B02 | 0.343 | 0.048 | 355.19 | 1.64E−04 | 4.82 | N/A |
| 107 | 121 | | FJ1427_P035MP02C02 | 0.401 | 0.048 | 268.38 | 5.70E−04 | 2.26 | N/A |
| 108 | 122 | | FJ1427 P035MP02D02 | 0.831 | 0.048 | 101.36 | 5.29E−04 | −0.13 | N/A |
| 109 | 123 | | FJ1427 P035MP02E02 | 0.254 | 0.049 | 324.42 | 2.69E−04 | 0.97 | N/A |
| 110 | 124 | | FJ1427 P035MP02F02 | 0.684 | 0.052 | 405.52 | 6.17E−04 | 7.09 | N/A |
| 111 | 125 | | FJ1427 P035MP02G02 | 0.177 | 0.049 | 349.11 | 1.12E−04 | 0.48 | N/A |
| 113 | 126 | | FJ1427_P035MP02A03 | 0.207 | 0.048 | 128.98 | 5.71E−04 | 1.39 | N/A |
| 114 | 127 | | FJ1427_P035MP02B03 | 0.383 | 0.048 | 335.78 | 2.15E−04 | −0.68 | N/A |
| 116 | 128 | | FJ1427_P035MP02D03 | 0.286 | 0.048 | 154.36 | 2.26E−04 | 0.65 | N/A |
| 117 | 129 | | FJ1427_P035MP02E03 | 0.242 | 0.049 | 302.37 | 1.69E−04 | 2.37 | N/A |
| 118 | 130 | | FJ1427_P035MP02F03 | 0.212 | 0.048 | 40.26 | 8.28E−04 | 2.77 | N/A |
| 119 | 131 | | FJ1427_P035MP02G03 | 0.197 | 0.049 | 25.37 | 2.93E−03 | −1.36 | N/A |
| 121 | 132 | | FJ1427 P035MP02A04 | 0.533 | 0.048 | 310.86 | 3.68E−04 | −0.8 | N/A |
| 122 | 133 | | FJ1427_P035MP02B04 | 0.375 | 0.047 | 77.27 | 4.49E−04 | 5.08 | N/A |
| 123 | 134 | | FJ1427_P035MP02C04 | 0.424 | 0.047 | 30.61 | 2.55E−03 | 2.91 | N/A |
| 124 | 135 | | FJ1427_P035MP02D04 | 0.447 | 0.048 | 356.6 | 3.58E−04 | 1.79 | N/A |
| 125 | 136 | | FJ1427_P035MP02E04 | 0.255 | 0.048 | 57.74 | 7.16E−04 | 4.96 | N/A |
| 126 | 137 | | FJ1427 P035MP02F04 | 0.516 | 0.048 | 332.62 | 3.43E−04 | 1.14 | N/A |
| 127 | 138 | | FJ1427_P035MP02G04 | 0.549 | 0.048 | 337.42 | 2.95E−04 | 0.95 | N/A |
| 128 | 139 | | FJ1427 P035MP02H04 | 0.376 | 0.050 | 77.91 | 5.10E−04 | −1.02 | N/A |
| 129 | 140 | | FJ1427_P035MP02A05 | 0.263 | 0.047 | 332.02 | 4.44E−04 | −1.23 | N/A |
| 130 | 141 | | FJ1427_P035MP02B05 | 0.553 | 0.048 | 343.63 | 2.92E−04 | 2.11 | N/A |
| 131 | 142 | | FJ1427 P035MP02C05 | 0.237 | 0.047 | 342.81 | 3.46E−04 | 3.8 | N/A |
| 132 | 143 | | FJ1427 P035MP02D05 | 0.118 | 0.049 | 103.1 | 2.79E−03 | 4.1 | N/A |
| 133 | 144 | | FJ1427_P035MP02E05 | 0.856 | 0.048 | 116.74 | 3.10E−04 | 3.63 | N/A |
| 134 | 145 | | FJ1427_P035MP02F05 | 0.206 | 0.048 | 355.08 | 3.34E−04 | 1.96 | N/A |
| 135 | 146 | | FJ1427 P035MP02G05 | 0.567 | 0.048 | 299.07 | 4.32E−04 | −1.49 | N/A |
| 136 | 147 | | FJ1427 P035MP02H05 | 0.192 | 0.049 | 277.67 | 2.68E−04 | −0.33 | N/A |
| 137 | 148 | | FJ1427_P035MP02A06 | 2.552 | 0.048 | 84.18 | 4.84E−04 | 0.61 | N/A |
| 138 | 149 | | FJ1427_P035MP02B06 | 0.125 | 0.047 | 29.02 | 4.60E−03 | 4.29 | N/A |
| 139 | 150 | | FJ1427 P035MP02C06 | 0.351 | 0.047 | 53 | 2.17E−03 | 3.82 | N/A |
| 140 | 151 | | FJ1427_P035MP02D06 | 0.298 | 0.047 | 345.4 | 3.74E−04 | 2 | N/A |
| 142 | 152 | | FJ1427_P035MP02F06 | 0.214 | 0.047 | 349.21 | 2.03E−04 | 2.53 | N/A |
| 145 | 153 | | FJ1427 P035MP02A07 | 0.791 | 0.049 | 423.04 | 2.11E−03 | 24.52 | 2.58E−01 |
| 146 | 154 | | FJ1427 P035MP02B07 | 0.380 | 0.046 | 140.58 | 3.36E−04 | 14.48 | N/A |
| 147 | 155 | | FJ1427_P035MP02C07 | 0.731 | 0.046 | 47.68 | 2.31E−03 | 10.64 | N/A |
| 148 | 156 | | FJ1427_P035MP02D07 | 0.747 | 0.046 | 64.76 | 9.82E−04 | 9.64 | N/A |
| 149 | 157 | | FJ1427_P035MP02E07 | 0.541 | 0.071 | 421.92 | 5.77E−04 | 23.81 | 3.28E−01 |
| 150 | 158 | | FJ1427 P035MP02F07 | 0.848 | 0.055 | 428.4 | 1.64E−03 | 25 | 3.77E−01 |
| 151 | 159 | | FJ1427_P035MP02G07 | 0.636 | 0.079 | 426.77 | 2.59E−04 | 46.84 | 1.29E−01 |
| 153 | 160 | | FJ1427_P035MP02A08 | 1.789 | 0.069 | 407.46 | 1.98E−03 | 5.67 | N/A |
| 155 | 161 | | FJ1427 P035MP02C08 | 0.254 | 0.047 | 71.07 | 3.33E−03 | −1.01 | N/A |
| 156 | 162 | | FJ1427 P035MP02D08 | 0.905 | 0.048 | 402.37 | 2.36E−03 | −0.73 | N/A |
| 157 | 163 | | FJ1427_P035MP02E08 | 0.450 | 0.058 | 398.11 | 8.46E−04 | 4.56 | N/A |
| 158 | 164 | | FJ1427_P035MP02F08 | 0.614 | 0.048 | 154.09 | 1.53E−03 | −2.21 | N/A |
| 159 | 165 | | FJ1427 P035MP02G08 | 0.441 | 0.055 | 419.38 | 1.78E−03 | 14.44 | N/A |
| 160 | 166 | | FJ1427_P035MP02H08 | 0.885 | 0.049 | 175.83 | 7.31E−04 | −1.64 | N/A |
| 161 | 167 | | FJ1427_P035MP02A09 | 1.299 | 0.053 | 431.17 | 6.17E−04 | 31.11 | 3.11E−01 |
| 162 | 168 | | FJ1427_P035MP02B09 | 0.811 | 0.047 | 148.47 | 2.20E−04 | −3.61 | N/A |
| 164 | 169 | | FJ1427 P035MP02D09 | 1.474 | 0.056 | 434.6 | 4.98E−04 | 11.47 | N/A |
| 166 | 170 | | FJ1427_P035MP02F09 | 1.468 | 0.051 | 422.26 | 1.07E−03 | 12.98 | N/A |
| 167 | 171 | | FJ1427_P035MP02G09 | 0.527 | 0.066 | 419.2 | 1.10E−03 | 4.38 | N/A |
| 168 | 172 | | FJ1427 P035MP02H09 | 1.510 | 0.049 | 172.49 | 1.41E−04 | −1.81 | N/A |
| 169 | 173 | | FJ1427 P035MP02A10 | 0.359 | 0.047 | 109.6 | 2.26E−03 | 0.77 | N/A |
| 170 | 174 | | FJ1427_P035MP02B10 | 0.476 | 0.047 | 152.72 | 2.84E−04 | 1.29 | N/A |
| 171 | 175 | | FJ1427_P035MP02C10 | 1.286 | 0.050 | 426.67 | 5.21E−04 | 9.46 | N/A |
| 172 | 176 | | FJ1427 P035MP02D10 | 0.557 | 0.047 | 96.27 | 1.09E−03 | 1.99 | N/A |
| 173 | 177 | | FJ1427_P035MP02E10 | 1.389 | 0.047 | 80.54 | 6.15E−04 | −3.94 | N/A |
| 174 | 178 | | FJ1427_P035MP02F10 | 0.595 | 0.051 | 400.6 | 7.88E−04 | 5.82 | N/A |
| 175 | 179 | | FJ1427_P035MP02G10 | 0.974 | 0.048 | 135.75 | 3.07E−04 | −3.41 | N/A |
| 176 | 180 | | FJ1427 P035MP02H10 | 0.435 | 0.051 | 406.01 | 7.57E−04 | 7.4 | N/A |
| 177 | 181 | | FJ1427_P035MP02A11 | 0.891 | 0.047 | 94.42 | 2.93E−04 | −1.82 | N/A |
| 179 | 182 | | FJ1427 P035MP02C11 | 1.570 | 0.060 | 440.23 | 4.33E−04 | 27.13 | 1.48E−01 |
| 180 | 183 | | FJ1427 P035MP02D11 | 0.463 | 0.047 | 102.96 | 8.33E−04 | −0.99 | N/A |
| 181 | 184 | | FJ1427 P035MP02E11 | 0.529 | 0.048 | 83.65 | 1.88E−03 | −0.86 | N/A |
| 182 | 185 | | FJ1427_P035MP02F11 | 0.330 | 0.047 | 71.84 | 6.86E−04 | −0.4 | N/A |
| 183 | 186 | | FJ1427_P035MP02G11 | 0.429 | 0.049 | 49.11 | 7.08E−04 | −0.23 | N/A |
| 184 | 187 | | FJ1427_P035MP02H11 | 0.795 | 0.049 | 49.64 | 7.73E−04 | −0.61 | N/A |
| 185 | 188 | | FJ1427 P035MP02A12 | 0.182 | 0.047 | 38.14 | 4.51E−03 | 4.65 | N/A |
| 186 | 189 | | FJ1427 P035MP02B12 | 1.255 | 0.048 | 350.63 | 7.16E−04 | 3.42 | N/A |
| 187 | 190 | | FJ1427_P035MP02C12 | 0.616 | 0.047 | 73.65 | 1.06E−03 | −2.68 | N/A |
| 188 | 191 | | FJ1427_P035MP02D12 | 0.658 | 0.046 | 50 | 1.44E−03 | −1.26 | N/A |
| 189 | 192 | | FJ1427_P035MP02E12 | 0.108 | 0.048 | 47.66 | 1.15E−02 | 10.71 | N/A |
| 190 | 193 | | FJ1427 P035MP02F12 | 0.628 | 0.048 | 82.48 | 2.94E−03 | 2.63 | N/A |
| 194 | 194 | | FJ1427 P035MP03B01 | 0.203 | 0.050 | 272.11 | 1.10E−03 | 11.6 | N/A |
| 196 | 195 | | FJ1427 P035MP03D01 | 0.307 | 0.048 | 165.84 | 1.27E−03 | 9.91 | N/A |
| 197 | 196 | | FJ1427 P035MP03E01 | 0.329 | 0.049 | 219.11 | 3.23E−04 | 11.43 | N/A |
| 198 | 197 | | FJ1427_P035MP03F01 | 0.221 | 0.049 | 32.03 | 4.47E−03 | 9.35 | N/A |
| 199 | 198 | | FJ1427_P035MP03G01 | 0.192 | 0.049 | 78.54 | 5.80E−04 | 9.43 | N/A |
| 200 | 199 | | FJ1427_P035MP03H01 | 0.327 | 0.051 | 352.35 | 3.65E−04 | 8.93 | N/A |
| 201 | 200 | | FJ1427_P035MP03A02 | 0.280 | 0.052 | 56.22 | 1.04E−03 | 7.41 | N/A |
| 202 | 201 | | FJ1427_P035MP03B02 | 0.529 | 0.068 | 19.84 | 2.41E−03 | 5.45 | N/A |
| 203 | 202 | | FJ1427 P035MP03C02 | 0.828 | 0.048 | 26.23 | 1.59E−03 | 8.29 | N/A |
| 204 | 203 | | FJ1427 P035MP03D02 | 0.116 | 0.048 | 345.41 | 7.96E−04 | 7.24 | N/A |
| 205 | 204 | | FJ1427_P035MP03E02 | 0.488 | 0.050 | 50.08 | 1.24E−03 | 6.16 | N/A |
| 206 | 205 | | FJ1427_P035MP03F02 | 0.113 | 0.049 | 38.07 | 1.91E−03 | 8.64 | N/A |
| 207 | 206 | | FJ1427_P035MP03G02 | 0.684 | 0.049 | 46.9 | 7.54E−04 | 7.03 | N/A |
| 209 | 207 | | FJ1427 P035MP03A03 | 0.296 | 0.050 | 341.4 | 2.48E−04 | 12.27 | N/A |
| 210 | 208 | | FJ1427_P035MP03B03 | 0.310 | 0.048 | 261.48 | 4.99E−04 | 15.53 | 3.21E−01 |
| 212 | 209 | | FJ1427_P035MP03D03 | 0.265 | 0.048 | 90.37 | 5.38E−04 | 10.94 | N/A |
| 213 | 210 | | FJ1427_P035MP03E03 | 0.281 | 0.048 | 53.86 | 1.36E−03 | 16.76 | 3.46E−01 |
| 214 | 211 | | FJ1427_P035MP03F03 | 0.107 | 0.048 | 347.43 | 3.61E−04 | 18.35 | 3.07E−01 |
| 215 | 212 | | FJ1427_P035MP03G03 | 0.137 | 0.050 | 105.4 | 1.14E−03 | 10.69 | N/A |
| 217 | 213 | | FJ1427_P035MP03A04 | 0.305 | 0.051 | 318.54 | 3.73E−04 | 7.23 | N/A |
| 218 | 214 | | FJ1427 P035MP03B04 | 0.186 | 0.048 | 286.39 | 4.30E−04 | 9.54 | N/A |
| 219 | 215 | | FJ1427 P035MP03C04 | 0.395 | 0.048 | 141.52 | 3.07E−04 | 8.91 | N/A |
| 220 | 216 | | FJ1427 P035MP03D04 | 0.447 | 0.048 | 34.63 | 1.60E−03 | 9.03 | N/A |
| 221 | 217 | | FJ1427_P035MP03E04 | 0.220 | 0.048 | 346.71 | 2.53E−04 | 10.26 | N/A |
| 222 | 218 | | FJ1427_P035MP03F04 | 0.153 | 0.049 | 258.53 | 4.02E−04 | 8.16 | N/A |
| 224 | 219 | | FJ1427_P035MP03H04 | 0.262 | 0.050 | 96.55 | 4.31E−04 | 8.43 | N/A |
| 226 | 220 | | FJ1427_P035MP03B05 | 0.401 | 0.050 | 400.47 | 8.98E−04 | 12.18 | N/A |
| 229 | 221 | | FJ1427 P035MP03E05 | 0.230 | 0.049 | 90.97 | 5.26E−04 | 6.31 | N/A |
| 231 | 222 | | FJ1427_P035MP03G05 | 0.914 | 0.049 | 94.07 | 3.51E−04 | 5.31 | N/A |
| 232 | 223 | | FJ1427 P035MP03H05 | 0.316 | 0.049 | 31.63 | 7.72E−04 | 5.66 | N/A |
| 233 | 224 | | FJ1427 P035MP03A06 | 0.393 | 0.047 | 351.15 | 1.95E−04 | 4.74 | N/A |
| 235 | 225 | | FJ1427 P035MP03C06 | 1.208 | 0.057 | 421.82 | 6.58E−04 | 20.02 | 4.33E−01 |
| 237 | 226 | | FJ1427_P035MP03E06 | 0.299 | 0.048 | 99.96 | 1.16E−03 | 7.58 | N/A |
| 238 | 227 | | FJ1427_P035MP03F06 | 0.434 | 0.048 | 359.12 | 2.86E−04 | 4.66 | N/A |
| 241 | 228 | | FJ1427_P035MP03A07 | 0.307 | 0.049 | 150 | 2.81E−04 | 4.97 | N/A |
| 243 | 229 | | FJ1427_P035MP03C07 | 0.372 | 0.047 | 52.24 | 5.33E−04 | 4.01 | N/A |
| 244 | 230 | | FJ1427 P035MP03D07 | 0.209 | 0.049 | 372.01 | 2.63E−03 | 6.21 | N/A |
| 245 | 231 | | FJ1427 P035MP03E07 | 0.211 | 0.057 | 399.68 | 1.68E−03 | 11.86 | N/A |
| 246 | 232 | | FJ1427 P035MP03F07 | 0.156 | 0.048 | 69.86 | 1.26E−03 | 3 | N/A |
| 247 | 233 | | FJ1427_P035MP03G07 | 0.201 | 0.048 | 55.87 | 2.86E−03 | 6.36 | N/A |
| 248 | 234 | | FJ1427_P035MP03H07 | 0.649 | 0.053 | 374.89 | 1.72E−03 | 8.41 | N/A |
| 250 | 235 | | FJ1427 P035MP03B08 | 1.124 | 0.052 | 413.11 | 6.40E−04 | 19.7 | 4.75E−01 |
| 251 | 236 | | FJ1427 P035MP03C08 | 0.972 | 0.054 | 407.61 | 7.57E−04 | 9.86 | N/A |
| 253 | 237 | | FJ1427_P035MP03E08 | 1.112 | 0.048 | 165.75 | 1.49E−04 | 2.97 | N/A |
| 255 | 238 | | FJ1427_P035MP03G08 | 1.260 | 0.053 | 419.28 | 5.50E−04 | 23.88 | 4.72E−01 |
| 257 | 239 | | FJ1427_P035MP03A09 | 0.208 | 0.049 | 370.9 | 4.80E−04 | 3.69 | N/A |
| 258 | 240 | | FJ1427_P035MP03B09 | 0.467 | 0.046 | 401.99 | 2.02E−03 | 9.85 | N/A |
| 260 | 241 | | FJ1427 P035MP03D09 | 0.313 | 0.059 | 397.85 | 1.61E−03 | 18.79 | 5.40E−01 |
| 262 | 242 | | FJ1427 P035MP03F09 | 0.621 | 0.048 | 190.2 | 3.47E−04 | 4.39 | N/A |
| 263 | 243 | | FJ1427_P035MP03G09 | 0.843 | 0.054 | 404.02 | 7.13E−04 | 13.59 | N/A |
| 264 | 244 | | FJ1427_P035MP03H09 | 0.166 | 0.052 | 67.9 | 1.84E−03 | 6.07 | N/A |
| 265 | 245 | | FJ1427_P035MP03A10 | 0.983 | 0.071 | 413.1 | 6.36E−04 | 15.94 | 5.06E−01 |
| 266 | 246 | | FJ1427 P035MP03B10 | 0.286 | 0.050 | 377.33 | 7.07E−04 | 7.72 | N/A |
| 267 | 247 | | FJ1427 P035MP03C10 | 0.596 | 0.047 | 128.12 | 4.39E−04 | 2.47 | N/A |
| 268 | 248 | | FJ1427_P035MP03D10 | 0.118 | 0.047 | 33.15 | 1.20E−03 | 4.14 | N/A |
| 269 | 249 | | FJ1427_P035MP03E10 | 0.398 | 0.048 | 33.99 | 2.07E−03 | 8.56 | N/A |
| 271 | 250 | | FJ1427_P035MP03G10 | 0.260 | 0.048 | 40.5 | 1.65E−03 | 4.73 | N/A |
| 272 | 251 | | FJ1427 P035MP03H10 | 0.578 | 0.049 | 48 | 1.09E−03 | 5.22 | N/A |
| 274 | 252 | | FJ1427 P035MP03B11 | 0.987 | 0.052 | 410.36 | 6.22E−04 | 13.53 | N/A |
| 275 | 253 | | FJ1427 P035MP03C11 | 1.306 | 0.059 | 411.83 | 5.14E−04 | 19.27 | 4.94E−01 |
| 276 | 254 | | FJ1427 P035MP03D11 | 0.118 | 0.049 | 94.76 | 1.02E−03 | 3.73 | N/A |
| 277 | 255 | | FJ1427_P035MP03E11 | 0.624 | 0.050 | 389.61 | 1.48E−03 | 14.78 | N/A |
| 278 | 256 | | FJ1427 P035MP03F11 | 0.474 | 0.048 | 118.25 | 2.77E−04 | 4.2 | N/A |
| 279 | 257 | | FJ1427_P035MP03G11 | 0.380 | 0.057 | 391.93 | 1.22E−03 | 16.07 | 4.30E−01 |
| 282 | 258 | | FJ1427 P035MP03B12 | 0.202 | 0.049 | 375.75 | 1.25E−03 | 7.3 | N/A |
| 283 | 259 | | FJ1427 P035MP03C12 | 0.321 | 0.047 | 386.64 | 1.40E−03 | 3.41 | N/A |
| 481 | 260 | | FJ1427_P035MP06A01 | 0.348 | 0.047 | 474.12 | 5.61E−04 | 23.32 | 2.86E−01 |
| 482 | 261 | | FJ1427_P035MP06B01 | 0.521 | 0.046 | 476.12 | 2.10E−03 | 23.68 | 4.27E−01 |
| 483 | 262 | | FJ1427_P035MP06C01 | 1.342 | 0.047 | 472.96 | 5.44E−04 | 38.33 | 1.77E−01 |
| 484 | 263 | | FJ1427 P035MP06D01 | 1.101 | 0.064 | 480.76 | 3.10E−04 | 57.08 | 1.43E−01 |
| 487 | 264 | | FJ1427 P035MP06G01 | 0.557 | 0.049 | 381.48 | 1.43E−04 | 8.51 | N/A |
| 488 | 265 | | FJ1427_P035MP06H01 | 0.720 | 0.048 | 462.91 | 1.01E−03 | 19.52 | 2.78E−01 |
| 490 | 266 | | FJ1427_P035MP06B02 | 0.435 | 0.048 | 452.14 | 8.53E−04 | 13.8 | N/A |
| 491 | 267 | | FJ1427 P035MP06C02 | 0.799 | 0.048 | 463.95 | 4.55E−04 | 23.13 | 2.17E−01 |
| 493 | 268 | | FJ1427_P035MP06E02 | 0.148 | 0.047 | 452.54 | 1.49E−03 | 23.64 | 3.27E−01 |
| 494 | 269 | | FJ1427_P035MP06F02 | 0.278 | 0.047 | 448.3 | 3.70E−04 | 25.59 | 1.42E−01 |
| 495 | 270 | | FJ1427_P035MP06G02 | 0.677 | 0.047 | 467.81 | 6.19E−04 | 19.76 | 1.86E−01 |
| 496 | 271 | | FJ1427_P035MP06H02 | 0.371 | 0.050 | 452.86 | 1.71E−04 | 34.32 | 8.89E−02 |
| 497 | 272 | | FJ1427_P035MP06A03 | 0.148 | 0.047 | 428.04 | 8.82E−04 | 16.47 | 3.37E−01 |
| 500 | 273 | | FJ1427_P035MP06D03 | 1.020 | 0.047 | 471.16 | 2.90E−04 | 60.68 | 1.49E−01 |
| 501 | 274 | | FJ1427 P035MP06E03 | 0.570 | 0.048 | 462.36 | 7.45E−04 | 37.48 | 2.04E−01 |
| 503 | 275 | | FJ1427_P035MP06G03 | 0.267 | 0.048 | 458.78 | 5.86E−04 | 34.77 | 2.69E−01 |
| 504 | 276 | | FJ1427_P035MP06H03 | 0.239 | 0.050 | 455.35 | 1.96E−04 | 49.62 | 1.25E−01 |
| 506 | 277 | | FJ1427_P035MP06B04 | 0.935 | 0.048 | 465.64 | 1.88E−04 | 54.68 | 1.33E−01 |
| 507 | 278 | | FJ1427_P035MP06C04 | 0.187 | 0.046 | 446.33 | 8.40E−04 | 29.68 | 2.58E−01 |
| 508 | 279 | | FJ1427_P035MP06D04 | 0.189 | 0.047 | 452.08 | 6.21E−04 | 30.89 | 3.20E−01 |
| 509 | 280 | | FJ1427_P035MP06E04 | 0.974 | 0.053 | 466.26 | 1.98E−04 | 66.48 | 1.28E−01 |
| 510 | 281 | | FJ1427 P035MP06F04 | 1.328 | 0.049 | 469.61 | 4.87E−04 | 52.43 | 2.12E−01 |
| 511 | 282 | | FJ1427_P035MP06G04 | 0.213 | 0.047 | 447.95 | 6.41E−04 | 25.85 | 2.45E−01 |
| 512 | 283 | | FJ1427_P035MP06H04 | 1.025 | 0.048 | 454.77 | 2.45E−04 | 36.08 | 1.16E−01 |
| 513 | 284 | | FJ1427_P035MP06A05 | 0.149 | 0.046 | 439.59 | 1.18E−03 | 22.03 | 4.61E−01 |
| 514 | 285 | | FJ1427_P035MP06B05 | 0.617 | 0.047 | 457.12 | 8.71E−04 | 30.53 | 2.37E−01 |
| 515 | 286 | | FJ1427_P035MP06C05 | 0.580 | 0.046 | 439.77 | 7.51E−04 | 15.43 | 2.23E−01 |
| 516 | 287 | | FJ1427_P035MP06D05 | 0.304 | 0.053 | 454.57 | 4.83E−04 | 42.58 | 1.86E−01 |
| 520 | 288 | | FJ1427_P035MP06H05 | 0.590 | 0.048 | 418.52 | 8.85E−04 | 25.16 | 1.81E−01 |
| 521 | 289 | | FJ1427_P035MP06A06 | 0.300 | 0.046 | 431.32 | 6.31E−04 | 28.53 | 2.23E−01 |
| 522 | 290 | | FJ1427 P035MP06B06 | 1.418 | 0.046 | 440.19 | 3.24E−04 | 30.17 | 1.21E−01 |
| 523 | 291 | | FJ1427_P035MP06C06 | 0.255 | 0.048 | 436.72 | 6.98E−04 | 19.74 | 2.05E−01 |
| 524 | 292 | | FJ1427_P035MP06D06 | 0.289 | 0.047 | 426.31 | 2.90E−04 | 22.59 | 1.27E−01 |
| 525 | 293 | | FJ1427_P035MP06E06 | 1.453 | 0.047 | 443.23 | 2.41E−04 | 36.18 | 8.34E−02 |
| 526 | 294 | | FJ1427_P035MP06F06 | 0.224 | 0.048 | 427.72 | 9.35E−04 | 19.34 | 1.47E−01 |
| 530 | 295 | | FJ1427_P035MP06B07 | 1.015 | 0.046 | 447.21 | 2.85E−04 | 39.69 | 1.14E−01 |
| 532 | 296 | | FJ1427_P035MP06D07 | 0.957 | 0.049 | 449.94 | 3.46E−04 | 51.29 | 1.70E−01 |
| 537 | 297 | | FJ1427 P035MP06A08 | 0.440 | 0.047 | 413.69 | 4.97E−04 | 2.11 | N/A |
| 539 | 298 | | FJ1427_P035MP06C08 | 0.297 | 0.048 | 430.14 | 3.35E−04 | 33.41 | 1.48E−01 |
| 540 | 299 | | FJ1427 P035MP06D08 | 0.836 | 0.048 | 434.79 | 2.09E−04 | 38.84 | 1.22E−01 |
| 541 | 300 | | FJ1427_P035MP06E08 | 0.359 | 0.047 | 419.89 | 2.67E−04 | 25.86 | 1.28E−01 |
| 542 | 301 | | FJ1427_P035MP06F08 | 0.343 | 0.048 | 428 | 2.74E−04 | 45.72 | 1.31E−01 |
| 543 | 302 | | FJ1427_P035MP06G08 | 0.255 | 0.048 | 429.73 | 2.86E−04 | 42.71 | 1.31E−01 |
| 544 | 303 | | FJ1427_P035MP06H08 | 0.313 | 0.048 | 428.17 | 3.49E−04 | 37.37 | 1.18E−01 |
| 545 | 304 | | FJ1427_P035MP06A09 | 0.192 | 0.046 | 413.96 | 9.47E−04 | 19.81 | 1.98E−01 |
| 546 | 305 | | FJ1427_P035MP06B09 | 0.329 | 0.066 | 424.36 | 2.25E−03 | 89.43 | 1.64E−02 |
| 547 | 306 | | FJ1427 P035MP06C09 | 0.690 | 0.046 | 436.33 | 1.01E−03 | 29.27 | 2.45E−01 |
| 550 | 307 | | FJ1427_P035MP06F09 | 0.564 | 0.046 | 383.12 | 1.47E−04 | 8.59 | N/A |
| 551 | 308 | | FJ1427 P035MP06G09 | 0.202 | 0.049 | 419.96 | 7.26E−04 | 34.74 | 1.58E−01 |
| 554 | 309 | | FJ1427_P035MP06B10 | 0.264 | 0.058 | 418.07 | 2.23E−03 | 80.94 | 6.01E−02 |
| 555 | 310 | | FJ1427_P035MP06C10 | 0.358 | 0.050 | 422.9 | 2.08E−04 | 53.92 | 1.55E−01 |
| 559 | 311 | | FJ1427_P035MP06G10 | 1.004 | 0.047 | 432.87 | 2.61E−04 | 48.85 | 1.42E−01 |
| 573 | 312 | | FJ1427 P035MP06E12 | 0.271 | 0.047 | 403.99 | 4.96E−04 | 19.27 | 1.56E−01 |

| | doorlopende | | | | | | |
|---|---|---|---|---|---|---|---|
| | nummering | | | | | | |
| | Patent | | | | | | CDR3 |
| Clone | clone | | VH | | | | Family |
| nr. | ID | VH | ID | CDR1 | CDR2 | CDR3 | ID |
| 115 | 14 | EVQLVESGGGLVQTGGSLRLSCAASGSQFI | b51 | NDVMG- | DMDDTGSTEYADSVKG- | GLWIKGRHFDY- | 14 |
| | | NDVMGWYRQVPGKQRELVADMDDTGSTEYA | | SEQ ID | SEQ ID | SEQ ID | |
| | | DSVKGRFTILRDSVKNTAYLQMSNLKPEDT | | NO: 505 | NO: 506 | NO: 507 | |
| | | GVYYCKAGLWIKGRHFDYWGQGTQVTVSS- | | | | | |
| | | SEQ ID | | | | | |
| | | NO: 14 | | | | | |
| 141 | 15 | QVQLVESGGGSVQPGGSLRLSCAASGFTFS | b176 | NYAMS- | SITGFGRGTDYADSVKG- | YSSSTYYPPTPARGRDY- | 38 |
| | | NYAMSWVRQAPGKGLEWVASITGFGRGTDY | | SEQ ID | SEQ ID | SEQ ID | |
| | | ADSVKGRFTISRDNAEDTLYLQMNSLKPED | | NO: 508 | NO: 509 | NO: 510 | |
| | | TAVYYCAKYSSSTYYPPTPARGRDYRGQGT | | | | | |
| | | QVTVSS- | | | | | |
| | | SEQ ID | | | | | |
| | | NO: 15 | | | | | |
| 154 | 16 | EVQLVESGGGLVQAGGSLRLSCAASGRAIE | b19 | NYPVG- | AITWISGSTLYADSVKG- | ALKTITRGQNDYSY- | 1 |
| | | NYPVGWFRQAPGKEREFVAAITWISGSTLY | | SEQ ID | SEQ ID | SEQ ID | |
| | | ADSVKGRFTISRDNAKNTVYLQMSSLKPED | | NO: 511 | NO: 512 | NO: 513 | |
| | | TALYYCAAALKTITRGQNDYSYWGQGTQVT | | | | | |
| | | VSS- | | | | | |
| | | SEQ ID | | | | | |
| | | NO: 16 | | | | | |
| 163 | 17 | QVQLQESGGGLVQAGGSLRLSCVASGSVSS | b81 | INGMA- | AISRVGNTAYGDSVKG- | DSWGGDDY- | 12 |
| | | INGMAWYRQGADNQRVLVAAISRVGNTAYG | | SEQ ID | SEQ ID | SEQ ID | |
| | | DSVKGRFTISRQNARNTVYLQMNSLKPEDT | | NO: 514 | NO: 515 | NO: 516 | |
| | | AVYYCNADSWGGDDYWGQGTQVTVSS- | | | | | |
| | | SEQ ID | | | | | |
| | | NO: 17 | | | | | |
| 211 | 18 | QVQLVESGGGLVQPGGSLRLSCAISGGTLD | b160 | SYGIG- | CMSRSDDRTYYADSVKG- | VDAYGCSLVQPTTYDF- | 34 |
| | | SYGIGWVRQAPGKQREGVSCMSRSDDRTYY | | SEQ ID | SEQ ID | SEQ ID | |
| | | ADSVKGRFTISKDSAKNTVYLQMTSLKPED | | NO: 517 | NO: 518 | NO: 519 | |
| | | TAVYYCAAVDAYGCSLVQPTTYDFWGLGTQ | | | | | |
| | | VTVSS- | | | | | |
| | | SEQ ID | | | | | |
| | | NO: 18 | | | | | |
| 254 | 19 | EVQLVESGGGLVQTGGSLRLSCAASGGTFS | b50 | RDAMA- | LISWSGATTNYADSVKG- | DRRPMGSRSYFEPTEYDD- | 9 |
| | | RDAMAWFRQVPGKEREFVALISWSGATTNY | | SEQ ID | SEQ ID | SEQ ID | |
| | | ADSVKGRFAISRDNGKNTVYLQMNRLKPAD | | NO: 520 | NO: 521 | NO: 522 | |
| | | TAIYYCAADRRPMGSRSYFEPTEYDDWGQG | | | | | |
| | | TQVTVSS- | | | | | |
| | | SEQ ID | | | | | |
| | | NO: 19 | | | | | |
| 270 | 20 | EVQLVESGGGLVQAGGSLRLSCAASGRDFS | b20 | SYAMG- | AITWTKRSTDFPDSVKG- | ARGLPVTPLGDIIY- | 3 |
| | | SYAMGWFRQAPGKEREFWVAITWTKRSTDF | | SEQ ID | SEQ ID | SEQ ID | |
| | | PDSVKGRFTISRDNAKNTVYLDMNSLKPED | | NO: 523 | NO: 524 | NO: 525 | |
| | | TAVYYCASARGLPVTPLGDIIYWGEGTLVT | | | | | |
| | | VSS- | | | | | |
| | | SEQ ID | | | | | |
| | | NO: 20 | | | | | |
| 281 | 21 | EVQLVESGGGLVQAGGSLRLSCAASGRTFS | b23 | INAMG- | AISRSGGSTVYVDGVKG- | TMAVGWTTRWRTADFDS- | 32 |
| | | INAMGWFRQAPGKEREFVAAISRSGGSTVY | | SEQ ID | SEQ ID | SEQ ID | |
| | | VDGVKGRFTISRDNAKNTVYLQMNSLEPED | | NO: 526 | NO: 527 | NO: 528 | |
| | | TAVYYCAATMAVGWTTRWRTADFDSWGQGT | | | | | |
| | | QVTVSS- | | | | | |
| | | SEQ ID | | | | | |
| | | NO: 21 | | | | | |
| 286 | 22 | EVQLVESGGGLVQAGGSLRLSCAASGSIFS | b30 | INAMA- | AISRDGGASVYRDSVKG- | TRAIGWTARWITTDFDF- | 33 |
| | | INAMAWFRQVPGMERELVAAISRDGGASVY | | SEQ ID | SEQ ID | SEQ ID | |
| | | RDSVKGRFTISRDNSKNTVYLQMNTLKPED | | NO: 529 | NO: 530 | NO: 531 | |
| | | TAIYVCAATRAIGWTARWITTDFDFWGQGT | | | | | |
| | | QVTVSS- | | | | | |
| | | SEQ ID | | | | | |
| | | NO: 22 | | | | | |
| 502 | 23 | QVQLVESGGGLVQAGGSLRLSCAVSGDVFV | b132 | RYTMA- | SVTDSGRTTDYVHSVKG- | NTDYFQIKSLDANT- | 20 |
| | | RYTMAWFRQAPGKEREFVASVTDSGRTTDY | | SEQ ID | SEQ ID | SEQ ID | |
| | | VHSVKGRFTVSRDNAKNTVYLQMNNLKPED | | NO: 532 | NO: 533 | NO: 534 | |
| | | TAVYYCAANTDYFQIKSLDANTWGQGTQVT | | | | | |
| | | VSS- | | | | | |
| | | SEQ ID | | | | | |
| | | NO: 23 | | | | | |
| 517 | 24 | QVQLVESGGELVQGGASLRLSCAASGRTFS | b86 | NANMA- | LITWSSGSTLYADSVKG- | DGPPYSGTYYRYDTYDY- | 5 |
| | | NANMAWFRQAPEKEREFVALITWSSGSTLY | | SEQ ID | SEQ ID | SEQ ID | |
| | | ADSVKGRFTISRDNARKMVYLQMNSLKPED | | NO: 535 | NO: 536 | NO: 537 | |
| | | TAVYYCAADGPPYSGTYYRYDTYDYWGQGT | | | | | |
| | | QVTVSS- | | | | | |
| | | SEQ ID | | | | | |
| | | NO: 24 | | | | | |
| 518 | 25 | QVQLVESGGGLVQTGDSLRLSCAASGRSLD | b162 | TTYIA- | YISPRFSHTWYADSVKG- | REHSGSTAWEHYDH- | 24 |
| | | TTYIAWFRQAPGKERDFLAYISPRFSHTWY | | SEQ ID | SEQ ID | SEQ ID | |
| | | ADSVKGRFTISRNIAKRTVDLEMNSLEPED | | NO: 538 | NO: 539 | NO: 540 | |
| | | TAVYYCAAREHSGSTAWEHYDHWGQGTQVT | | | | | |
| | | VSS- | | | | | |
| | | SEQ ID | | | | | |
| | | NO: 25 | | | | | |
| 529 | 26 | QVQLQESGGGLVQAGGSLRLSCAASGDVFV | b76 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 541 | NO: 542 | NO: 543 | |
| | | TAIYYCAANTDYFQIRSLDLNTWGQGTQVT | | | | | |
| | | VSS- | | | | | |
| | | SEQ ID | | | | | |
| | | NO: 26 | | | | | |
| 97 | 111 | QVQLVESGGGLVQTGGSLRLSCVVSGSHFI | b170 | SDVMG- | DMADGGSAKYGDSVKG- | GLWITGRHSDY- | 14 |
| | | SDVMGWYRQVPGKQRELVADMADGGSAKYG | | SEQ ID | SEQ ID | SEQ ID | |
| | | DSVKGRFTILRDSVKNTVYLQMSSLNPEDT | | NO: 798 | NO: 799 | NO: 800 | |
| | | GVYYCKAGLWITGRHSDYWGQGTQVTVSS- | | | | | |
| | | SEQ ID | | | | | |
| | | NO: 111 | | | | | |
| 98 | 112 | QVQLVESGGGLVQTGGSLRLSCAASGSQFI | b164 | NDVMG- | DMDDTGSTKYADSVKG- | GLWIKGRHFDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 801 | NO: 802 | NO: 803 | |
| | | GVYYCKAGLWIKGRHFDYWGQGTQVTVSS- | | | | | |
| | | SEQ ID | | | | | |
| | | NO: 112 | | | | | |
| 99 | 113 | QVQLVESGGGLVQPGGSLGLSCAASGFTFD | b151 | NYAMS- | SITGAGRGTHYADSVKG- | YSSSTYYPPTPARGTDY- | 38 |
| | | NYAMSWVRQAPGKGLEWVSSITGAGRGTHY | | SEQ ID | SEQ ID | SEQ ID | |
| | | ADSVKGRFTISRDNAKNTLYLQMNSLKPED | | NO: 804 | NO: 805 | NO: 806 | |
| | | TAVYYCAKYSSSTYYPPTPARGTDYRGPGT | | | | | |
| | | QVTVSS- | | | | | |
| | | SEQ ID | | | | | |
| | | NO: 113 | | | | | |
| 100 | 114 | QVQLQESGGGLVQTGGSLRLSCAASGSQFI | b84 | NDVMG- | DMDDTGSTKYADSVKG- | GLWIKGRHSDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 807 | NO: 808 | NO: 809 | |
| | | GVYYCKAGLWIKGRHSDYWGQGTQVTVSS- | | | | | |
| | | SEQ ID | | | | | |
| | | NO: 114 | | | | | |
| 101 | 115 | EVQLVESGGGLVQIGGSLRLSCAASGRTFS | b46 | SITMA- | GINFSGTRTFYADSVKG- | SPRGFYGPGNALYDY- | 29 |
| | | SITMAYFRQVPGKEREWVAGINFSGTRTFY | | SEQ ID | SEQ ID | SEQ ID | |
| | | ADSVKGRFLISRDDAKSTMYLQMNSLKPED | | NO: 810 | NO: 811 | NO: 812 | |
| | | TAVYYCAASPRGFYGPGNALYDYWGQGTQV | | | | | |
| | | TVSS- | | | | | |
| | | SEQ ID | | | | | |
| | | NO: 115 | | | | | |
| 102 | 116 | EVQLVESGGGLVQTGGSLRLSCAVSGSRFI | b57 | SDVMG- | DMADGGSAKYADSVKG- | GLWIAGRHSDY- | 14 |
| | | SDVMGWYRQVPGKQRELVADMADGGSAKYA | | SEQ ID | SEQ ID | SEQ ID | |
| | | DSVKGRFSILRDSVKNTVYLQMSSLKPEDT | | NO: 813 | NO: 814 | NO: 815 | |
| | | GIYYCKAGLWIAGRHSDYWGQGTQVTVSS- | | | | | |
| | | SEQ ID | | | | | |
| | | NO: 116 | | | | | |
| 103 | 117 | QVQLVESGGGLVQTGGSLRLSCAVSGSRFI | b168 | SDVMG- | DMADGGSAKYADSVKG- | GLWIAGRHSDY- | 14 |
| | | SDVMGWYRQVPGKQRELVADMADGGSAKYA | | SEQ ID | SEQ ID | SEQ ID | |
| | | DSVKGRFSILRDSAKNTVYLQMSSLKPEDT | | NO: 816 | NO: 817 | NO: 818 | |
| | | GIYYCKAGLWIAGRHSDYWGQGTQVTVSS- | | | | | |
| | | SEQ ID | | | | | |
| | | NO: 117 | | | | | |
| 104 | 118 | QVQLVESGGGLVQPGGSLGLSCAASGFTFD | b151 | NYAMS- | SITGAGRGTHYADSVKG- | YSSSTYYPPTPARGTDY- | 38 |
| | | NYAMSWVRQAPGKGLEWVSSITGAGRGTHY | | SEQ ID | SEQ ID | SEQ ID | |
| | | ADSVKGRFTISRDNAKNTLYLQMNSLKPED | | NO: 819 | NO: 820 | NO: 821 | |
| | | TAVYYCAKYSSSTYYPPTPARGTDYRGPGT | | | | | |
| | | QVTVSS- | | | | | |
| | | SEQ ID | | | | | |
| | | NO: 118 | | | | | |
| 105 | 119 | QVQLVESGGGLVQPGGSLRLSCAASGFTFD | b154 | NYAMT- | SITGAGRGTHYADSVKG- | YSSSTYYPTTPVRGTDY- | 38 |
| | | NYAMTWVRQAPGKGLEWVSSITGAGRGTHY | | SEQ ID | SEQ ID | SEQ ID | |
| | | ADSVKGRFTISRDNAKNTLYLQVNSLKPED | | NO: 822 | NO: 823 | NO: 824 | |
| | | TAVYYCARYSSSTYYPTTPVRGTDYRGQGT | | | | | |
| | | QVTVSS- | | | | | |
| | | SEQ ID | | | | | |
| | | NO: 119 | | | | | |
| 106 | 120 | EVQLVESGGTLVQTGGSLRLSCVVSGSHFI | b64 | SDVMG- | DMADGGSAKYGDSVKG- | GLWIKGRHSDY- | 14 |
| | | SDVMGWYRQVPGKQRELVADMADGGSAKYG | | SEQ ID | SEQ ID | SEQ ID | |
| | | DSVKGRFTIVRDSVKNTVYLQMSSLNPEDT | | NO: 825 | NO: 826 | NO: 827 | |
| | | GVYYCKAGLWIKGRHSDYWGQGTQVTVTS- | | | | | |
| | | SEQ ID | | | | | |
| | | NO: 120 | | | | | |
| 107 | 121 | QVQLVESGGGLVQPGGSLRLSCAASGSRFV | b159 | SDFMG- | DMADGGSARYGDSVKG- | GLWIAGRHFDY- | 14 |
| | | SDFMGWYRQVPGKQRELVADMADGGSARYG | | SEQ ID | SEQ ID | SEQ ID | |
| | | DSVKGRFTILRDNMNSTAFLQMSSLKPEDT | | NO: 828 | NO: 829 | NO: 830 | |
| | | GVYYCKAGLWIAGRHFDYWGQGTQVTVSS- | | | | | |
| | | SEQ ID | | | | | |
| | | NO: 121 | | | | | |
| 108 | 122 | QVQLVESGGGLVQVGGSLSVSCAASGRSFD | b175 | SITMA- | GINFSGSRTAYGDSVKG- | SPRGFYGPGHALYDY- | 29 |
| | | SITMAYFRQAPGNEREFIAGINFSGSRTAY | | SEQ ID | SEQ ID | SEQ ID | |
| | | GDSVKGRFTISRDNAKNTVFLQMNSLNPED | | NO: 831 | NO: 832 | NO: 833 | |
| | | TAVYYCAASPRGFYGPGHALYDYWGQGTQV | | | | | |
| | | TVSS- | | | | | |
| | | SEQ ID | | | | | |
| | | NO: 122 | | | | | |
| 109 | 123 | EVQLVESGGGLVQTGGSLRLSCAASGSQFI | b53 | NDVMG- | DMDDTGSTKYADSVKG- | GLWIKGRHSDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 834 | NO: 835 | NO: 836 | |
| | | GVYYCKAGLWIKGRHSDYWGQGTQVTVSS- | | | | | |
| | | SEQ ID | | | | | |
| | | NO: 123 | | | | | |
| 110 | 124 | EVQLVESGGGLVQAGGSLRLACALSGDVFV | b8 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 837 | NO: 838 | NO: 839 | |
| | | TAVYYCAANTDYFQIRSLDRNTWGQGTQVT | | | | | |
| | | VSS- | | | | | |
| | | SEQ ID | | | | | |
| | | NO: 124 | | | | | |
| 111 | 125 | QVQLVESGGGLVQPGGSLRLSCAASGSHFI | b158 | SDVMG- | DMADGGSVKYGDSVKG- | GLWITGRHFDY- | 14 |
| | | SDVMGWYRQVPGKQRELVADMADGGSVKYG | | SEQ ID | SEQ ID | SEQ ID | |
| | | DSVKGRFTILRDSVKNTVTLQMSSLNPEDT | | NO: 840 | NO: 841 | NO: 842 | |
| | | GVYYCKAGLWITGRHFDYWGQGIQVTVTS- | | | | | |
| | | SEQ ID | | | | | |
| | | NO: 125 | | | | | |
| 113 | 126 | QVQLVESGGGLVQPGGSLRLSCAASGFTFS | b157 | NYAMS- | SITGAGRGTYYAESVKG- | YDSSTFYPPTPARGIAD- | 36 |
| | | NYAMSWVRQAPGKGLEWVSSITGAGRGTYY | | SEQ ID | SEQ ID | SEQ ID | |
| | | AESVKGRFTISRDNDKNTLYLQMNSLKPED | | NO: 843 | NO: 844 | NO: 845 | |
| | | TAVYYCAKYDSSTFYPPTPARGIADRGQGT | | | | | |
| | | RVTVSS- | | | | | |
| | | SEQ ID | | | | | |
| | | NO: 126 | | | | | |
| 114 | 127 | QVQLVESGGGLVQTGGSLRLSCAASGSRFV | b166 | SDFMG- | DMADGGSARYGDSVKG- | GLWIAGRHFDY- | 14 |
| | | SDFMGWYRQVPGKQRELVADMADGGSARYG | | SEQ ID | SEQ ID | SEQ ID | |
| | | DSVKGRFTILRDSGNSTAYLQMSSLKPEDT | | NO: 846 | NO: 847 | NO: 848 | |
| | | GVYYCKAGLWIAGRHFDYWGQGTQVTVSS- | | | | | |
| | | SEQ ID | | | | | |
| | | NO: 127 | | | | | |
| 116 | 128 | QVQLVESGGGLVQAGGPLRLSCAASGGTFS | b91 | SYAMG- | ATTRGGGRTMYAVSTEG- | MTSYYSGTYYPDSSDFDI- | 18 |
| | | SYAMGWFRQAPGKEREFVAATTRGGGRTMY | | SEQ ID | SEQ ID | SEQ ID | |
| | | AVSTEGRFTCSRDNAKNTVYLLMNSLKPED | | NO: 849 | NO: 850 | NO: 851 | |
| | | TAVYYCAAMTSYYSGTYYPDSSDFDIWGQG | | | | | |
| | | TQVTVSS- | | | | | |
| | | SEQ ID | | | | | |
| | | NO: 128 | | | | | |
| 117 | 129 | EVQLVESGGGSVQTGGSLRLSCVVSGSHFI | b62 | SDVMG- | DMADGGSVKYGDSVKG- | GLWITGRHFDY- | 14 |
| | | SDVMGWYRQVPGKQRELVADMADGGSVKYG | | SEQ ID | SEQ ID | SEQ ID | |
| | | DSVKGRFTILRDSVKNTVTLQMSSLNPEDT | | NO: 852 | NO: 853 | NO: 854 | |
| | | GVYYCKAGLWITGRHFDYWGQGTQVTVTS- | | | | | |
| | | SEQ ID | | | | | |
| | | NO: 129 | | | | | |
| 118 | 130 | EVQLVESGGGLVQAGGSLRLSCAASGGTFS | b17 | SYAMG- | ATTRGGGRTMYAVSTEG- | MTSYYSGTYYPDSSDFDI- | 18 |
| | | SYAMGWFRQAPGKEREFVAATTRGGGRTMY | | SEQ ID | SEQ ID | SEQ ID | |
| | | AVSTEGRFTCSRDNAKNTVYLLMNSLKPED | | NO: 855 | NO: 856 | NO: 857 | |
| | | TAVYYCAAMTSYYSGTYYPDSSDFDIWGQG | | | | | |
| | | TQVTVSS- | | | | | |
| | | SEQ ID | | | | | |
| | | NO: 130 | | | | | |
| 119 | 131 | QVQLVESGGGLVQPGGSLGLSCAASGFTFS | b152 | NYAMS- | SITGFGRGTDYADSVKG- | YSSSTYYPPTPARGRDY- | 38 |
| | | NYAMSWVRQAPGKGLEWVASITGFGRGTDY | | SEQ ID | SEQ ID | SEQ ID | |
| | | ADSVKGRFTVSRDNAKNTLYLQMNSLKPED | | NO: 858 | NO: 859 | NO: 860 | |
| | | TAVYYCAKYSSSTYYPPTPARGRDYRGQGT | | | | | |
| | | QVTVSS- | | | | | |
| | | SEQ ID | | | | | |
| | | NO: 131 | | | | | |
| 121 | 132 | EVQLVESGGGLVQTGGSLRLSCTASGSQFI | b58 | NDVMG- | DMDDTGSTKYADSVNG- | GLWIKGRHSDY- | 14 |
| | | NDVMGWYRQVPGKQRELVADMDDTGSTKYA | | SEQ ID | SEQ ID | SEQ ID | |
| | | DSVNGRFTILRDSVKNTAYLQMSNLKPEDT | | NO: 861 | NO: 862 | NO: 863 | |
| | | GVYYCKAGLWIKGRHSDYWGQGTQVTVSS- | | | | | |
| | | SEQ ID | | | | | |
| | | NO: 132 | | | | | |
| 122 | 133 | EVQLVESGGGLVQAGGSLGLSCAASGGTFS | b7 | SYAMG- | ATTRGGGRTMYAVSTEG- | MTSYYSGTYYPDSSDFDI- | 18 |
| | | SYAMGWFRQAPGKEREFVAATTRGGGRTMY | | SEQ ID | SEQ ID | SEQ ID | |
| | | AVSTEGRFTCSRDNAKNTVYLQMNSLKPKD | | NO: 864 | NO: 865 | NO: 866 | |
| | | TAIYYCAAMTSYYSGTYYPDSSDFDIWGQG | | | | | |
| | | TQVTVSS- | | | | | |
| | | SEQ ID | | | | | |
| | | NO: 133 | | | | | |
| 123 | 134 | QVQLVESGGGLVQPGGSLELSCAASGFTFS | b150 | NYAMS- | SITGFGRGTDYADSVKG- | YSSSTYYPPTPARGRDY- | 38 |
| | | NYAMSWVRQAPGKGLEWVSSITGFGRGTDY | | SEQ ID | SEQ ID | SEQ ID | |
| | | ADSVKGRFTISRDNDKNTLYLQMNSLKPED | | NO: 867 | NO: 868 | NO: 869 | |
| | | TAVYYCARYSSSTYYPPTPARGRDYRGQGT | | | | | |
| | | QVTVSS- | | | | | |
| | | SEQ ID | | | | | |
| | | NO: 134 | | | | | |
| 124 | 135 | QVQLVESGGGLVQTGGSLRLSCAVSGSRFI | b169 | SDVMG- | DMADGGSAKYADSVKG- | GLWIAGRHSDY- | 14 |
| | | SDVMGWYRQVPGKQRELVADMADGGSAKYA | | SEQ ID | SEQ ID | SEQ ID | |
| | | DSVKGRFSILRDSVKNTVYLQMSSLKPEDT | | NO: 870 | NO: 871 | NO: 872 | |
| | | GIYYCKAGLWIAGRHSDYWGQGTQVTVSS- | | | | | |
| | | SEQ ID | | | | | |
| | | NO: 135 | | | | | |
| 125 | 136 | EVQLVESGGGLVQAGGSLRLSCAASGGTFS | b18 | SYAMG- | ATTRGGGRTMYAVSTEG- | MTSYYSGTYYPDSSDFDI- | 18 |
| | | SYAMGWFRQAPGKEREFVAATTRGGGRTMY | | SEQ ID | SEQ ID | SEQ ID | |
| | | AVSTEGRFTCSRDNAKNTVYLQMNSLKPKD | | NO: 873 | NO: 874 | NO: 875 | |
| | | TAIYYCAAMTSYYSGTYYPDSSDFDIWGQG | | | | | |
| | | TQVTVSS- | | | | | |
| | | SEQ ID | | | | | |
| | | NO: 136 | | | | | |
| 126 | 137 | EVQLVESGGGLVQTGGSLRLSCAASGSQFI | b52 | NDVMG- | DMDDTGSTKYADSVKG- | GLWIKGRHFDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 876 | NO: 877 | NO: 878 | |
| | | GVYYCKAGLWIKGRHFDYWGQGTQVTVSS- | | | | | |
| | | SEQ ID | | | | | |
| | | NO: 137 | | | | | |
| 127 | 138 | QVQLVESGGGLVQAGGSLRLSCAASGSQFI | b130 | NDVMG- | DMDDTGSTKYADSVKG- | GLWIKGRHFDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 879 | NO: 880 | NO: 881 | |
| | | GVYYCKAGLWIKGRHFDYWGQGTQVTVSS- | | | | | |
| | | SEQ ID | | | | | |
| | | NO: 138 | | | | | |
| 128 | 139 | QVQLVESGGGLVQAGDSLRLSCATSGLTFS | b90 | SYALG- | ATTRGGGRTMYAVSTEG- | MTSYYSGTYYPDSSDFDI- | 18 |
| | | SYALGWFRQAPGKKREFVAATTRGGGRTMY | | SEQ ID | SEQ ID | SEQ ID | |
| | | AVSTEGRFTCSRDNDKNTVYLLMNSLKPKD | | NO: 882 | NO: 883 | NO: 884 | |
| | | TAVYYCAAMTSYYSGTYYPDSSDFDIWGQG | | | | | |
| | | TQVTVSS- | | | | | |
| | | SEQ ID | | | | | |
| | | NO: 139 | | | | | |
| 129 | 140 | EVQLVESGGGLVQTGGSLRLSCAASGSQFI | b53 | NDVMG- | DMDDTGSTKYADSVKG- | GLWIKGRHSDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 885 | NO: 886 | NO: 887 | |
| | | GVYYCKAGLWIKGRHSDYWGQGTQVTVSS- | | | | | |
| | | SEQ ID | | | | | |
| | | NO: 140 | | | | | |
| 130 | 141 | EVQLVESGGGLVQTGGSLRLSCAASGSQFI | b52 | NDVMG- | DMDDTGSTKYADSVKG- | GLWIKGRHFDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 888 | NO: 889 | NO: 890 | |
| | | GVYYCKAGLWIKGRHFDYWGQGTQVTVSS- | | | | | |
| | | SEQ ID | | | | | |
| | | NO: 141 | | | | | |
| 131 | 142 | QVQLVESGGGLVQTGGSLRLSCAVSGSRFI | b169 | SDVMG- | DMADGGSAKYADSVKG- | GLWIAGRHSDY- | 14 |
| | | SDVMGWYRQVPGKQRELVADMADGGSAKYA | | SEQ ID | SEQ ID | SEQ ID | |
| | | DSVKGRFSILRDSVKNTVYLQMSSLKPEDT | | NO: 891 | NO: 892 | NO: 893 | |
| | | GIYYCKAGLWIAGRHSDYWGQGTQVTVSS- | | | | | |
| | | SEQ ID | | | | | |
| | | NO: 142 | | | | | |
| 132 | 143 | QVQLVESGGGLVQPGESLGLSCAASGFTFS | b149 | NYAMS- | SITGAGRGTDYADSVKG- | YSSGTYYPPTPVRGTDY- | 38 |
| | | NYAMSWVRQAPGKGLEWVSSITGAGRGTDY | | SEQ ID | SEQ ID | SEQ ID | |
| | | ADSVKGRFTISRDNAKNTLYLQMNSLKPED | | NO: 894 | NO: 895 | NO: 896 | |
| | | TAVYYCAKYSSGTYYPPTPVRGTDYRGQGT | | | | | |
| | | QVTVSS- | | | | | |
| | | SEQ ID | | | | | |
| | | NO: 143 | | | | | |
| 133 | 144 | EVQLVESGGGLVQIGGSLRLSCAASGRTFS | b45 | SITMA- | NFSGTRTFYADSVKG- | SPRGFYGPGNALYDY- | 29 |
| | | SITMAYFRQVPGKEHEWVAGINFSGTRTFY | | SEQ ID | SEQ ID | SEQ ID | |
| | | ADSVKGRFLISRDDAKSTMYLQMNSLKPED | | NO: 897 | NO: 898 | NO: 899 | |
| | | TAVYYCAASPRGFYGPGNALYDYWGQGTQV | | | | | |
| | | TVSS- | | | | | |
| | | SEQ ID | | | | | |
| | | NO: 144 | | | | | |
| 134 | 145 | EVQLVESGGGLVQTGGSLRLSCAVSGSRFI | b57 | SDVMG- | DMADGGSAKYADSVKG- | GLWIAGRHSDY- | 14 |
| | | SDVMGWYRQVPGKQRELVADMADGGSAKYA | | SEQ ID | SEQ ID | SEQ ID | |
| | | DSVKGRFSILRDSVKNTVYLQMSSLKPEDT | | NO: 900 | NO: 901 | NO: 902 | |
| | | GIYYCKAGLWIAGRHSDYWGQGTQVTVSS- | | | | | |
| | | SEQ ID | | | | | |
| | | NO: 145 | | | | | |
| 135 | 146 | EVQLVESGGGLVQTGGSLRLSCAASGSQFI | b54 | NDVTG- | DMDDTGSTKYADSVKG- | GLWIKGRHSDY- | 14 |
| | | NDVTGWYRQVPGKQRELVADMDDTGSTKYA | | SEQ ID | SEQ ID | SEQ ID | |
| | | DSVKGRFTILRDSVKNTAYLQMSNLKPEDT | | NO: 903 | NO: 904 | NO: 905 | |
| | | GVYYCKAGLWIKGRHSDYWGQGTQVTVSS- | | | | | |
| | | SEQ ID | | | | | |
| | | NO: 146 | | | | | |
| 136 | 147 | EVQLVESGGGSVQTGGSLRLSCVVSGSHFI | b61 | SDVMG- | DMADGGSAKYGDSVKG- | GLWITGRHSDY- | 14 |
| | | SDVMGWYRQVPGKQRELVADMADGGSAKYG | | SEQ ID | SEQ ID | SEQ ID | |
| | | DSVKGRFTILRDSVKNTVTLQMSSLNPEDT | | NO: 906 | NO: 907 | NO: 908 | |
| | | GVYYCKAGLWITGRHSDYWGQGTQVTVTS- | | | | | |
| | | SEQ ID | | | | | |
| | | NO: 147 | | | | | |
| 137 | 148 | QVQLQESGGGLVQAGGSLRLSCAASGGTFS | b78 | SYAMG- | ATTRGGGRTMYAVSTEG- | MTSYYSGTYYPDSSDFDI- | 18 |
| | | SYAMGWFRQAPGKEREFVAATTRGGGRTMY | | SEQ ID | SEQ ID | SEQ ID | |
| | | AVSTEGRFTCSRDNDKNTVYLLMNSLKPKD | | NO: 909 | NO: 910 | NO: 911 | |
| | | TAVYYCAAMTSYYSGTYYPDSSDFDIWGQG | | | | | |
| | | TQVTVSS- | | | | | |
| | | SEQ ID | | | | | |
| | | NO: 148 | | | | | |
| 138 | 149 | QVQLVESGGGLVQPGESLGLSCAASGFTFS | b149 | NYAMS- | SITGAGRGTDYADSVKG- | YSSGTYYPPTPVRGTDY- | 38 |
| | | NYAMSWVRQAPGKGLEWVSSITGAGRGTDY | | SEQ ID | SEQ ID | SEQ ID | |
| | | ADSVKGRFTISRDNAKNTLYLQMNSLKPED | | NO: 912 | NO: 913 | NO: 914 | |
| | | TAVYYCAKYSSGTYYPPTPVRGTDYRGQGT | | | | | |
| | | QVTVSS- | | | | | |
| | | SEQ ID | | | | | |
| | | NO: 149 | | | | | |
| 139 | 150 | QLQLVESGGGLVQPGGSLELSCAASGFTFS | b68 | NYAMS- | SITGFGRGTDYADSVKG- | YSSSTYYPPTPARGRDY- | 38 |
| | | NYAMSWVRQAPGKGLEWVSSITGFGRGTDY | | SEQ ID | SEQ ID | SEQ ID | |
| | | ADSVKGRFTISRDNAKNTLYLQMNSLKPED | | NO: 915 | NO: 916 | NO: 917 | |
| | | TAVYYCARYSSSTYYPPTPARGRDYRGQGT | | | | | |
| | | QVTVSS- | | | | | |
| | | SEQ ID | | | | | |
| | | NO: 150 | | | | | |
| 140 | 151 | EVQLVESGGGLVQTGGSLRLSCAASGSQFI | b53 | NDVMG- | DMDDTGSTKYADSVKG- | GLWIKGRHSDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 918 | NO: 919 | NO: 920 | |
| | | GVYYCKAGLWIKGRHSDYWGQGTQVTVSS- | | | | | |
| | | SEQ ID | | | | | |
| | | NO: 151 | | | | | |
| 142 | 152 | EVQLVESGGGLVQTGGSLRLSCAASGSRFI | b55 | SDFMG- | DMADGGSARYGDSVKG- | GLWIAGRHFDY- | 14 |
| | | SDFMGWYRQVPGKQRELVADMADGGSARYG | | SEQ ID | SEQ ID | SEQ ID | |
| | | DSVKGRFTILRESGNATAYLQMSSLKPEDT | | NO: 921 | NO: 922 | NO: 923 | |
| | | GVYYCKAGLWIAGRHFDYWGQGTQVTVSS- | | | | | |
| | | SEQ ID | | | | | |
| | | NO: 152 | | | | | |
| 145 | 153 | QVQLVESGGGLVQAGGSLRLSCAVSGDVFV | b137 | RYTMA- | TVTDSGRTTEYVDSVKG- | NTDYFRIRSLDANT- | 20 |
| | | RYTMAWFRQAPGKEREFVATVTDSGRTTEY | | SEQ ID | SEQ ID | SEQ ID | |
| | | VDSVKGRFTVSRDNAKNTVYLQMNNLKPED | | NO: 924 | NO: 925 | NO: 926 | |
| | | TAVYYCAANTDYFRIRSLDANTWGQGTQVT | | | | | |
| | | VSS- | | | | | |
| | | SEQ ID | | | | | |
| | | NO: 153 | | | | | |
| 146 | 154 | EVQLVESGGGLVEAGGSLRLSCAASGRAFN | b1 | NFPMG- | AITWISGSTLYADSVKG- | ALKTITRGQHDYTY- | 1 |
| | | NFPMGWFRQAPGKEREFVAAITWISGSTLY | | SEQ ID | SEQ ID | SEQ ID | |
| | | ADSVKGRFTISRDKSKNTVYLQMSGLKPED | | NO: 927 | NO: 928 | NO: 929 | |
| | | TALYYCAAALKTITRGQHDYTYWGQGTQVT | | | | | |
| | | VSS- | | | | | |
| | | SEQ ID | | | | | |
| | | NO: 154 | | | | | |
| 147 | 155 | QVQLVESGGGLVQAGGSLRLSCAASGRTFS | b120 | INTMG- | AISRSGDSTVYVDSVKG- | TVAVGWTTRWSTIDFDS- | 30 |
| | | INTMGWFRQAPGKEREFVAAISRSGDSTVY | | SEQ ID | SEQ ID | SEQ ID | |
| | | VDSVKGRFTISRDNAKNTVYLQMNTLKPED | | NO: 930 | NO: 931 | NO: 932 | |
| | | TAVYYCATTVAVGWTTRWSTIDFDSWGQGT | | | | | |
| | | QVTVSS- | | | | | |
| | | SEQ ID | | | | | |
| | | NO: 155 | | | | | |
| 148 | 156 | EVQLVESGGGLVQAGGSLRLSCAASGRSLS | b21 | TTEMG- | AITWSGRGTMYAESVE- | ARRGRAAVRSEGGYDF- | 4 |
| | | TTEMGWFRQAPGKEREFVAAITWSGRGTMY | | SEQ ID | SEQ ID | SEQ ID | |
| | | AESVEGRFAVSRENAKNTVYLQMNSLKPED | | NO: 933 | NO: 934 | NO: 935 | |
| | | TAIYYCAAARRGRAAVRSEGGYDFWGQGTQ | | | | | |
| | | VTVSS- | | | | | |
| | | SEQ ID | | | | | |
| | | NO: 156 | | | | | |
| 149 | 157 | QVQLVESGGGLVQAGGSLGLACALSGDVFV | b93 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 936 | NO: 937 | NO: 938 | |
| | | TAVYYCAANTDYFQIRSLDRNTWGQGTQVT | | | | | |
| | | VSS- | | | | | |
| | | SEQ ID | | | | | |
| | | NO: 157 | | | | | |
| 150 | 158 | QVQLVESGGGLVQAGGSLRLSCAASGDVFV | b101 | RYTMA- | SVTDSGRTTDYVDSVKG- | NTDYFQIRSLDANT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 939 | NO: 940 | NO: 941 | |
| | | TARYYCAANTDYFQIRSLDANTWGQGTQVT | | | | | |
| | | VSS- | | | | | |
| | | SEQ ID | | | | | |
| | | NO: 158 | | | | | |
| 151 | 159 | QVQLVESGGGLVQAGGSLRLSCAVSGDVFV | b135 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 942 | NO: 943 | NO: 944 | |
| | | TAVYYCAANTDYFQIRSLDLNTWGQGTQVT | | | | | |
| | | VSS- | | | | | |
| | | SEQ ID | | | | | |
| | | NO: 159 | | | | | |
| 153 | 160 | EVQLVESGGGLVQAGGSLRLSCTVSGDVFV | b36 | RYTMA- | TVTDSGRTTEYVDSVKG- | NTDYFRIRSLDANT- | 20 |
| | | RYTMAWFRQAPGKEREFVATVTDSGRTTEY | | SEQ ID | SEQ ID | SEQ ID | |
| | | VDSVKGRFTVSRDNAKNTVYLQMNNLKPED | | NO: 945 | NO: 946 | NO: 947 | |
| | | TAVYYCAANTDYFRIRSLDANTWGQGTQVT | | | | | |
| | | VSS- | | | | | |
| | | SEQ ID | | | | | |
| | | NO: 160 | | | | | |
| 155 | 161 | EVQLVESGGGLVQAGGSLRLSCAASGSTFS | b31 | RQPMY- | AITWSGRGTLYADSVEG- | GPYGDAAYRHGRIDS- | 15 |
| | | RQPMYWFRQAPGKEREFVAAITWSGRGTLY | | SEQ ID | SEQ ID | SEQ ID | |
| | | ADSVEGRFTISRDNAKNTVYLQMNSLRPDD | | NO: 948 | NO: 949 | NO: 950 | |
| | | TAAYYCAAGPYGDAAYRHGRIDSWGQGTQV | | | | | |
| | | TVSS- | | | | | |
| | | SEQ ID | | | | | |
| | | NO: 161 | | | | | |
| 156 | 162 | EVQLVESGGGLVQAGGSLRLSCVVSGDVFV | b40 | RYTMA- | SITDSGRTTEYVDSVKG- | NTDYFRIRSLDANT- | 20 |
| | | RYTMAWFRQAPGKEREFVISITDSGRTTEY | | SEQ ID | SEQ ID | SEQ ID | |
| | | VDSVKGRFTVSRDNAKNTVYLQMNNLKPED | | NO: 951 | NO: 952 | NO: 953 | |
| | | TAVYYCAANTDYFRIRSLDANTWGQGTQVT | | | | | |
| | | VSS- | | | | | |
| | | SEQ ID | | | | | |
| | | NO: 162 | | | | | |
| 157 | 163 | EVQLVESGGGLVQAGGSLRLSCVVSGDVFV | b39 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 954 | NO: 955 | NO: 956 | |
| | | TAVYYCAANTDYFQIRSVDNNAWGQGTQVT | | | | | |
| | | VSS- | | | | | |
| | | SEQ ID | | | | | |
| | | NO: 163 | | | | | |
| 158 | 164 | QVQLVESGGGLVQAGGSLRLSCAASGSIFS | b128 | LNTMG- | AISRSGGSATYGDSVKG- | SAAVGWTTRWSTIDFDS- | 28 |
| | | LNTMGWFRQAPGKEREFVAAISRSGGSATY | | SEQ ID | SEQ ID | SEQ ID | |
| | | GDSVKGRFTISTNNAKTTVYLQMNSLKPDD | | NO: 957 | NO: 958 | NO: 959 | |
| | | TAVYSCAASAAVGWTTRWSTIDFDSWGQGT | | | | | |
| | | QVTVSS- | | | | | |
| | | SEQ ID | | | | | |
| | | NO: 164 | | | | | |
| 159 | 165 | QVQLVESGGGLVQAGGSLRLSCTVSGDVFV | b141 | RYTMA- | SVTDSGRTTEYVHSVRG- | NTDYFRIRSLDANT- | 20 |
| | | RYTMAWFRQAPGKEREFVASVTDSGRTTEY | | SEQ ID | SEQ ID | SEQ ID | |
| | | VHSVRGRFTVSRDNAKNTVYLQMNNLKPED | | NO: 960 | NO: 961 | NO: 962 | |
| | | TAVYYCAANTDYFRIRSLDANTWGQGTQVT | | | | | |
| | | VSS- | | | | | |
| | | SEQ ID | | | | | |
| | | NO: 165 | | | | | |
| 160 | 166 | EVQLVESGGGLVQAGDSLRISCAASGRIFS | b3 | NYAMG- | THLTSTTHYADSVKG- | DRNIKITADWSY- | 8 |
| | | NYAMGWFRQAPGKETEFVAGITHLTSTTHY | | SEQ ID | SEQ ID | SEQ ID | |
| | | ADSVKGRFTISRDNAKNTMHLQMNNLKPED | | NO: 963 | NO: 964 | NO: 965 | |
| | | TALYYCAADRNIKITADWSYWGQGTQVTVA | | | | | |
| | | S- | | | | | |
| | | SEQ ID | | | | | |
| | | NO: 166 | | | | | |
| 161 | 167 | QVQLVESGGGLVQAGGSLRLSCAVSGDVFV | b133 | RYTMA- | SVTDSGRTTEYVDSAKG- | NTDYFQIRSLRANT- | 20 |
| | | RYTMAWFRQAPGKEREFVASVTDSGRTTEY | | SEQ ID | SEQ ID | SEQ ID | |
| | | VDSAKGRFTVSRDNAKNTVYLQMNSLKPED | | NO: 966 | NO: 967 | NO: 968 | |
| | | TAVYYCAANTDYFQIRSLRANTWGQGTQVT | | | | | |
| | | VSS- | | | | | |
| | | SEQ ID | | | | | |
| | | NO: 167 | | | | | |
| 162 | 168 | EVQLVESGGGLVQAGGSLRLSCAASGRTFS | b28 | TTEMG- | AITWTGRGTMYAESVEG- | ARRGRAAVRSEGGYDF- | 4 |
| | | TTEMGWFRQAPGKEREFVAAITWTGRGTMY | | SEQ ID | SEQ ID | SEQ ID | |
| | | AESVEGRFTISRENAKNMVYLQMNSLKPED | | NO: 969 | NO: 970 | NO: 971 | |
| | | TAIYYCAAARRGRAAVRSEGGYDFWGQGTQ | | | | | |
| | | VTVSS- | | | | | |
| | | SEQ ID | | | | | |
| | | NO: 168 | | | | | |
| 164 | 169 | QVQLVESGGGLVQAGGSLRLACALSGDVFV | b96 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 972 | NO: 973 | NO: 974 | |
| | | TAVYYCAANTDYFQIRSLDRNTWGQGTQVT | | | | | |
| | | VSS- | | | | | |
| | | SEQ ID | | | | | |
| | | NO: 169 | | | | | |
| 166 | 170 | EVQLVESGGGLVQAGGSLRLSCAASGDVFV | b14 | RYTMA- | SVTDSGRTTDYVDSVKG- | NTDYFQIRSLEFNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 975 | NO: 976 | NO: 977 | |
| | | TAVYYCAANTDYFQIRSLEFNTWGQGTQVT | | | | | |
| | | VSS- | | | | | |
| | | SEQ ID | | | | | |
| | | NO: 170 | | | | | |
| 167 | 171 | QVQLVESGGGLVQAGGSLRLSCTVSGDVFV | b140 | RYTMA- | LVTDSGRTTEYVDSVKG- | NTDYFRIRSLDANT- | 20 |
| | | RYTMAWFRQAPGKEREFVALVTDSGRTTEY | | SEQ ID | SEQ ID | SEQ ID | |
| | | VDSVKGRFTVSRDNAKNTVYLQMNNLKPED | | NO: 978 | NO: 979 | NO: 980 | |
| | | TAVYYCAANTDYFRIRSLDANTWGQGTQVT | | | | | |
| | | VSS- | | | | | |
| | | SEQ ID | | | | | |
| | | NO: 171 | | | | | |
| 168 | 172 | QLQLVESGGGLVQPGGSLRLSCAAAGRAIE | b69 | NYPVG- | AITWISGSTLYADSVKG- | ALKTITRGQNDYSY- | 1 |
| | | NYPVGWFRRAPGKEREFVAAITWISGSTLY | | SEQ ID | SEQ ID | SEQ ID | |
| | | ADSVKGRFTISRDNAKNTVYLQMSSLKPED | | NO: 981 | NO: 982 | NO: 983 | |
| | | TALYYCAAALKTITRGQNDYSYWGQGTQVT | | | | | |
| | | VSS- | | | | | |
| | | SEQ ID | | | | | |
| | | NO: 172 | | | | | |
| 169 | 173 | QLQLVESGGGLVQTGTSLRLSCAASGHTSR | b71 | INAMG- | AISRNGGSTVYVDSVKG- | TDAVGWTTRWMTADFGF- | 31 |
| | | INAMGWFRQFPGKEREFVAAISRNGGSTVY | | SEQ ID | SEQ ID | SEQ ID | |
| | | VDSVKGRFTISRDNAKSTVYLQMNSLKPED | | NO: 984 | NO: 985 | NO: 986 | |
| | | TAVYYCTATDAVGWTTRWMTADFGFWGQGT | | | | | |
| | | QVTVSS- | | | | | |
| | | SEQ ID | | | | | |
| | | NO: 173 | | | | | |
| 170 | 174 | QVQLVESGGGLVEAGGSLRLSCAASGRAFN | b88 | NFPMG- | AITWISGSTLYADSVKG- | ALKTITRGQHDYTY- | 1 |
| | | NFPMGWFRQAPGKEREFVAAITWISGSTLY | | SEQ ID | SEQ ID | SEQ ID | |
| | | ADSVKGRFTISRDKSKNTVYLQMSGLKPED | | NO: 987 | NO: 988 | NO: 989 | |
| | | TALYYCAAALKTITRGQHDYTYWGQGTQVT | | | | | |
| | | VSS- | | | | | |
| | | SEQ ID | | | | | |
| | | NO: 174 | | | | | |
| 171 | 175 | QVQLVESGGGLVQAGGSLRLACALSGDVFV | b96 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 990 | NO: 991 | NO: 992 | |
| | | TAVYYCAANTDYFQIRSLDRNTWGQGTQVT | | | | | |
| | | VSS- | | | | | |
| | | SEQ ID | | | | | |
| | | NO: 175 | | | | | |
| 172 | 176 | EVQLVESGGGLVQAGGSLRLSCAASGRTFS | b24 | SDAKA- | AITWAGRGTDYADSVKD- | NPTGVIGPGFRNSNRYDY- | 19 |
| | | SDAKAWFRQAPGKEREFVAAITWAGRGTDY | | SEQ ID | SEQ ID | SEQ ID | |
| | | ADSVKDRFTITRDNAKNMVYLQMSSLKPED | | NO: 993 | NO: 994 | NO: 995 | |
| | | TAVYYCALNPTGVIGPGFRNSNRYDYWGQG | | | | | |
| | | TQVTVAS- | | | | | |
| | | SEQ ID | | | | | |
| | | NO: 176 | | | | | |
| 173 | 177 | QVQLVESGGGLVQAGGSLRLSCAAAGRAIE | b98 | NYPVG- | AITWISGSTLYADSVKG- | ALKTITRGQNDYSY- | 1 |
| | | NYPVGWFRRAPGKEREFVAAITWISGSTLY | | SEQ ID | SEQ ID | SEQ ID | |
| | | ADSVKGRFTISRDNAKNTVYLQMSSLKPED | | NO: 996 | NO: 997 | NO: 998 | |
| | | TALYYCAAALKTITRGQNDYSYWGQGTQVT | | | | | |
| | | VSS- | | | | | |
| | | SEQ ID | | | | | |
| | | NO: 177 | | | | | |
| 174 | 178 | EVQLVESGGGLVQAGGSLRLSCVVSGDVFV | b39 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 999 | NO: 1000 | NO: 1001 | |
| | | TAVYYCAANTDYFQIRSVDNNAWGQGTQVT | | | | | |
| | | VSS- | | | | | |
| | | SEQ ID | | | | | |
| | | NO: 178 | | | | | |
| 175 | 179 | QVQLVESGGGLVQAGGSLRLSCAASGRTFS | b126 | TTEMG- | AITWTGRGTMYAESVEG- | ARRGRAAVRSEGGYDF- | 4 |
| | | TTEMGWFRQAPGKEREFVAAITWTGRGTMY | | SEQ ID | SEQ ID | SEQ ID | |
| | | AESVEGRFTISRENAKNMVYLQMNSLKPED | | NO: 1002 | NO: 1003 | NO: 1004 | |
| | | TAIYYCAAARRGRAAVRSEGGYDFWGQGTQ | | | | | |
| | | VTVSS- | | | | | |
| | | SEQ ID | | | | | |
| | | NO: 179 | | | | | |
| 176 | 180 | EVQLVESGGGLVQAGGSLRLSCAASGDVFV | b13 | RYTMA- | SVTDSGRTTDYVDSVKG- | NTDYFQIKSLDRNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1005 | NO: 1006 | NO: 1007 | |
| | | TAVYYCAANTDYFQIKSLDRNTWGQGTQVT | | | | | |
| | | VSS- | | | | | |
| | | SEQ ID | | | | | |
| | | NO: 180 | | | | | |
| 177 | 181 | EVQLVESGGGLVQAGGSLRLSCAASGRTFS | b26 | TTEMG- | AITWTGRGTMYAESVEG- | ARRGRAAVRSEGGYDF- | 4 |
| | | TTEMGWFRQAPGKEREFVAAITWTGRGTMY | | SEQ ID | SEQ ID | SEQ ID | |
| | | AESVEGRFTISRENAKNMVYLQMNSLKPED | | NO: 1008 | NO: 1009 | NO: 1010 | |
| | | TAIYYCAAARRGRAAVRSEGGYDFWGQGTQ | | | | | |
| | | ATVSS- | | | | | |
| | | SEQ ID | | | | | |
| | | NO: 181 | | | | | |
| 179 | 182 | QVQLVESGGGLVQAGGSLRLSCAVSGDVFV | b136 | RYTMA- | SVTDSGRTTEYVDSVKG- | NTDYFQIRSLRANT- | 20 |
| | | RYTMAWFRQAPGKEREFVASVTDSGRTTEY | | SEQ ID | SEQ ID | SEQ ID | |
| | | VDSVKGRFTVSRDNAKNTVYLQMNSLKPED | | NO: 1011 | NO: 1012 | NO: 1013 | |
| | | TAVYYCAANTDYFQIRSLRANTWGQGTQVT | | | | | |
| | | VSS- | | | | | |
| | | SEQ ID | | | | | |
| | | NO: 182 | | | | | |
| 180 | 183 | QVQLQESGGGLVQAGDSLRISCAASGRIFS | b72 | NYAMG- | THLTSTTHYADSVKG- | DRNIKITADWSY- | 8 |
| | | NYAMGWFRQAPGKETEFVAGITHLTSTTHY | | SEQ ID | SEQ ID | SEQ ID | |
| | | ADSVKGRFTISRDNAKNTMHLQMNNLKPED | | NO: 1014 | NO: 1015 | NO: 1016 | |
| | | TALYYCAADRNIKITADWSYWGQGTQVTVA | | | | | |
| | | S- | | | | | |
| | | SEQ ID | | | | | |
| | | NO: 183 | | | | | |
| 181 | 184 | QVQLQESGGGLVQAGGSLRLSCAASGRTFS | b79 | NYVMA- | AITQTGRGTFYADSVEG- | PRSGRAGTRNQMDYEY- | 22 |
| | | NYVMAWFRQAPGKEREFVGAITQTGRGTFY | | SEQ ID | SEQ ID | SEQ ID | |
| | | ADSVEGRFTISRINDKNTVYLQMNSLKPED | | NO: 1017 | NO: 1018 | NO: 1019 | |
| | | TAVYYCSAPRSGRAGTRNQMDYEYWGQGTQ | | | | | |
| | | VTVSS- | | | | | |
| | | SEQ ID | | | | | |
| | | NO: 184 | | | | | |
| 182 | 185 | EVQLVESGGGLVQAGGSLRLSCAASGRDFS | b20 | SYAMG- | AITWTKRSTDFPDSVKG- | ARGLPVTPLGDIIY- | 3 |
| | | SYAMGWFRQAPGKEREFWVAITWTKRSTDF | | SEQ ID | SEQ ID | SEQ ID | |
| | | PDSVKGRFTISRDNAKNTVYLDMNSLKPED | | NO: 1020 | NO: 1021 | NO: 1022 | |
| | | TAVYYCASARGLPVTPLGDIIYWGEGTLVT | | | | | |
| | | VSS- | | | | | |
| | | SEQ ID | | | | | |
| | | NO: 185 | | | | | |
| 183 | 186 | QLQLVESGGGLVQAGGSLRLSCAASGRDFS | b67 | SYAMG- | AITWTKRSTDFPDSVKG- | ARGLPVTPLGDIIY- | 3 |
| | | SYAMGWFRQAPGKEREFWAITWTKRSTDFP | | SEQ ID | SEQ ID | SEQ ID | |
| | | DSVKGRFTISRDNAKNTVYLDMNSLKPEDT | | NO: 1023 | NO: 1024 | NO: 1025 | |
| | | AVYYCASARGLPVTPLGDIIYWGEGTLVTV | | | | | |
| | | SS- | | | | | |
| | | SEQ ID | | | | | |
| | | NO: 186 | | | | | |
| 184 | 187 | EVQLVESGGGLVQAGGSLRLSCAASGRTFS | b27 | TTEMG- | AITWTGRGTMYAESVEG- | ARRGRAAVRSEGGYDF- | 4 |
| | | TTEMGWFRQAPGKEREFVAAITWTGRGTMY | | SEQ ID | SEQ ID | SEQ ID | |
| | | AESVEGRFTISRENAKNMVYLQMNSLKPED | | NO: 1026 | NO: 1027 | NO: 1028 | |
| | | TAIYYCAAARRGRAAVRSEGGYDFWGQGTQ | | | | | |
| | | VAVSS- | | | | | |
| | | SEQ ID | | | | | |
| | | NO: 187 | | | | | |
| 185 | 188 | QVQLQESGGGLVQAGGSLRLSCAVSGRSFR | b80 | NYVMG- | GITQFTSTTYYDDSVKG- | DRSIKMTADWAY- | 10 |
| | | NYVMGWFRQAPGREREIVAGITQFTSTTYY | | SEQ ID | SEQ ID | SEQ ID | |
| | | DDSVKGRFTISRDNAKNTVYLQMNSLKPED | | NO: 1029 | NO: 1030 | NO: 1031 | |
| | | TALYYCAADRSIKMTADWAYWGQGTQVTVS | | | | | |
| | | S- | | | | | |
| | | SEQ ID | | | | | |
| | | NO: 188 | | | | | |
| 186 | 189 | QVQLVESGGGLVQAGGSLRLSCAASGRADS | b115 | NAIMA- | AVTYSGMPTYQDDSVQG- | RMYSASTYYGDYDY- | 25 |
| | | NAIMAWFRQAPGKEREFIVAVTYSGMPTYQ | | SEQ ID | SEQ ID | SEQ ID | |
| | | DDSVQGRFTASRDNAKNTVYLQMNSLKPED | | NO: 1032 | NO: 1033 | NO: 1034 | |
| | | TAVYYCAARMYSASTYYGDYDYWGQGTQVT | | | | | |
| | | VSS- | | | | | |
| | | SEQ ID | | | | | |
| | | NO: 189 | | | | | |
| 187 | 190 | EVQLVESGGGSVQAGGSLRLSCAASGRTFS | b60 | DYTMG- | AITGTGHTTDMPDSVEG- | ARGLPVTPLGDIIY- | 3 |
| | | DYTMGWFRQAPGKEREFWVAITGTGHTTDM | | SEQ ID | SEQ ID | SEQ ID | |
| | | PDSVEGRFTISRDNAKNTIYLDMKNLKPGD | | NO: 1035 | NO: 1036 | NO: 1037 | |
| | | TAVYYCASARGLPVTPLGDIIYWGEGTLVT | | | | | |
| | | VSS- | | | | | |
| | | SEQ ID | | | | | |
| | | NO: 190 | | | | | |
| 188 | 191 | QVQLVESGGGLVQAGGSLTLSCAASGGAFS | b146 | SDAKA- | AITWAGRGTDYADSVKG- | NPRGVIGPAFRNSNHYTY- | 19 |
| | | SDAKAWFRQAPGKEREFVAAITWAGRGTDY | | SEQ ID | SEQ ID | SEQ ID | |
| | | ADSVKGRFTITRDNAKATVHLQMNSLKPED | | NO: 1038 | NO: 1039 | NO: 1040 | |
| | | TAVYYCALNPRGVIGPAFRNSNHYTYWGQG | | | | | |
| | | TQVTVSS- | | | | | |
| | | SEQ ID | | | | | |
| | | NO: 191 | | | | | |
| 189 | 192 | QVQLQESGGGLVQAGGSMRLSCAASGRTFS | b83 | NYVIA- | AITQTGRGTFYASSVEG- | PRSGRAGTRNQMDYEY- | 22 |
| | | NYVIAWFRQAPGKEREFVGAITQTGRGTFY | | SEQ ID | SEQ ID | SEQ ID | |
| | | ASSVEGRFTLSRLNDQSTVYLQMNSLNPED | | NO: 1041 | NO: 1042 | NO: 1043 | |
| | | TGVYYCSAPRSGRAGTRNQMDYEYWGQGTQ | | | | | |
| | | VTVSS- | | | | | |
| | | SEQ ID | | | | | |
| | | NO: 192 | | | | | |
| 190 | 193 | QLQLVESGGGLVQTGTSLRLSCAASGHTSR | b71 | INAMG- | AISRNGGSTVYVDSVKG- | TDAVGWTTRWMTADFGF- | 31 |
| | | INAMGWFRQFPGKEREFVAAISRNGGSTVY | | SEQ ID | SEQ ID | SEQ ID | |
| | | VDSVKGRFTISRDNAKSTVYLQMNSLKPED | | NO: 1044 | NO: 1045 | NO: 1046 | |
| | | TAVYYCTATDAVGWTTRWMTADFGFWGQGT | | | | | |
| | | QVTVSS- | | | | | |
| | | SEQ ID | | | | | |
| | | NO: 193 | | | | | |
| 194 | 194 | QVQLVESGGGLVQAGGSLRLSCAASGFTFS | b110 | NYAMS- | SITGFGRGTDYADSVKG- | YSSSTYYPPTPARGTDY- | 38 |
| | | NYAMSWVRQAPGKGLEWVSSITGFGRGTDY | | SEQ ID | SEQ ID | SEQ ID | |
| | | ADSVKGRFTISRDNAKNTLYLQMNSLKPED | | NO: 1047 | NO: 1048 | NO: 1049 | |
| | | TAVYYCARYSSSTYYPPTPARGTDYRGQGT | | | | | |
| | | QVTVSS- | | | | | |
| | | SEQ ID | | | | | |
| | | NO: 194 | | | | | |
| 196 | 195 | QVQLVESGGGLVQPGGSLGLSCAASGFTFD | b151 | NYAMS- | SITGAGRGTHYADSVKG- | YSSSTYYPPTPARGTDY- | 38 |
| | | NYAMSWVRQAPGKGLEWVSSITGAGRGTHY | | SEQ ID | SEQ ID | SEQ ID | |
| | | ADSVKGRFTISRDNAKNTLYLQMNSLKPED | | NO: 1050 | NO: 1051 | NO: 1052 | |
| | | TAVYYCAKYSSSTYYPPTPARGTDYRGPGT | | | | | |
| | | QVTVSS- | | | | | |
| | | SEQ ID | | | | | |
| | | NO: 195 | | | | | |
| 197 | 196 | QVQLVESGGGLVQAGGSLRLSCAASGGTFS | b114 | SYAMG- | ATTRGGGRTMYAVSTEG- | MTSYYSGTYYPDSSDFDI- | 18 |
| | | SYAMGWFRQAPGKEREFVAATTRGGGRTMY | | SEQ ID | SEQ ID | SEQ ID | |
| | | AVSTEGRFTCSRDNAKNTVYLLMNSLKPED | | NO: 1053 | NO: 1054 | NO: 1055 | |
| | | TAVYYCAAMTSYYSGTYYPDSSDFDIWGQG | | | | | |
| | | TQVTVSS- | | | | | |
| | | SEQ ID | | | | | |
| | | NO: 196 | | | | | |
| 198 | 197 | QVQLVESGGGLVQVGGSLRVSCAASGRTSG | b173 | DRNMG- | IITWSSGSTVYADSVKG- | DYYPYSGSWNIMDY- | 13 |
| | | DRNMGWFRQAPGKEREFVGIITWSSGSTVY | | SEQ ID | SEQ ID | SEQ ID | |
| | | ADSVKGRFTISRDNAKNTVYLDMSSLKPED | | NO: 1056 | NO: 1057 | NO: 1058 | |
| | | TAVYYCAADYYPYSGSWNIMDYWGKGTLVT | | | | | |
| | | VSS- | | | | | |
| | | SEQ ID | | | | | |
| | | NO: 197 | | | | | |
| 199 | 198 | EVQLVESGGGLVQAGGSLRLSCAASGGTFS | b16 | SYAMG- | ATTRGGGRTMYAVSTEG- | RTSYYSGTYYPASSDFDI- | 27 |
| | | SYAMGWFRQAPGKEREFVAATTRGGGRTMY | | SEQ ID | SEQ ID | SEQ ID | |
| | | AVSTEGRFTCSRDNAKNTVYLLMNRVKPED | | NO: 1059 | NO: 1060 | NO: 1061 | |
| | | TAVYYCAARTSYYSGTYYPASSDFDIWGQG | | | | | |
| | | TQVTVSS- | | | | | |
| | | SEQ ID | | | | | |
| | | NO: 198 | | | | | |
| 200 | 199 | QVQLVESGGGSVQTGGSLRLSCVVSGSHFI | b177 | SDVMG- | DMADGGSAKYADSVKG- | GLWITGRHSDY- | 14 |
| | | SDVMGWYRQVPGKQRELVADMADGGSAKYA | | SEQ ID | SEQ ID | SEQ ID | |
| | | DSVKGRFTILRDSVKNTVTLQMSSLNPEDT | | NO: 1062 | NO: 1063 | NO: 1064 | |
| | | GVYYCKAGLWITGRHSDYWGQGTQVTVTS- | | | | | |
| | | SEQ ID | | | | | |
| | | NO: 199 | | | | | |
| 201 | 200 | QVQLVESGGGLVQAGGSLRLSCAASGGTFS | b113 | SYAMG- | ATTRGGGRTMYAVSTEG- | MTSYYSGTYYPDSSDFDI- | 18 |
| | | SYAMGWFRQAPGKEREFVAATTRGGGRTMY | | SEQ ID | SEQ ID | SEQ ID | |
| | | AVSTEGRFTCSRDNAENTVYLQMNSLKPED | | NO: 1065 | NO: 1066 | NO: 1067 | |
| | | TAIYYCAAMTSYYSGTYYPDSSDFDIWGQG | | | | | |
| | | TQVTVSS- | | | | | |
| | | SEQ ID | | | | | |
| | | NO: 200 | | | | | |
| 202 | 201 | EVQLVESGGGLVQVGDSLRLSCAASGRTVG | b59 | SANMG- | VITWSTGSTVYADSVKG- | DVYPYSGNLNIMDY- | 13 |
| | | SANMGWFRQAPGKEREFVAVITWSTGSTVY | | SEQ ID | SEQ ID | SEQ ID | |
| | | ADSVKGRFTISRDNAKNTVYLDMSSLKPDD | | NO: 1068 | NO: 1069 | NO: 1070 | |
| | | TAVYYCAADVYPYSGNLNIMDYWGKGTLVT | | | | | |
| | | VSS- | | | | | |
| | | SEQ ID | | | | | |
| | | NO: 201 | | | | | |
| 203 | 202 | QVQLVESGGGLVQVGDSLRLSCAASGRTVG | b172 | SANMG- | VITWSTGSTVYADSVKG- | DVYPYSGNLNIMDY- | 13 |
| | | SANMGWFRQAPGKEREFVAVITWSTGSTVY | | SEQ ID | SEQ ID | SEQ ID | |
| | | ADSVKGRFTISRDNAKNTVYLDMSSLKPED | | NO: 1071 | NO: 1072 | NO: 1073 | |
| | | TAVYYCAADVYPYSGNLNIMDYWGKGTLVT | | | | | |
| | | VSS- | | | | | |
| | | SEQ ID | | | | | |
| | | NO: 202 | | | | | |
| 204 | 203 | QVQLQESGGGLVQTGGSLRLSCAVSGSRFI | b85 | SDVMG- | DMADGGSAKYADSVKG- | GLWLAGRHSDY- | 14 |
| | | SDVMGWYRQVPGKQRELVADMADGGSAKYA | | SEQ ID | SEQ ID | SEQ ID | |
| | | DSVKGRFSILRDSVKNTVYLQMSSLKPEDT | | NO: 1074 | NO: 1075 | NO: 1076 | |
| | | GIYYCKAGLWLAGRHSDYWGQGTQVTVSS- | | | | | |
| | | SEQ ID | | | | | |
| | | NO: 203 | | | | | |
| 205 | 204 | QVQLVESGGGLVQVGGSLSVSCAASGRSFD | b175 | SITMA- | GINFSGSRTAYGDSVKG- | SPRGFYGPGHALYDY- | 29 |
| | | SITMAYFRQAPGNEREFIAGINFSGSRTAY | | SEQ ID | SEQ ID | SEQ ID | |
| | | GDSVKGRFTISRDNAKNTVFLQMNSLNPED | | NO: 1077 | NO: 1078 | NO: 1079 | |
| | | TAVYYCAASPRGFYGPGHALYDYWGQGTQV | | | | | |
| | | TVSS- | | | | | |
| | | SEQ ID | | | | | |
| | | NO: 204 | | | | | |
| 206 | 205 | QVQLVESGGGLVQPGGSLRLSCAASGFTFS | b156 | NYAMS- | SITGAGRGTYYADSVKG- | YDSSTYYPPTPARGIAD- | 36 |
| | | NYAMSWVRQAPGKGLEWVSSITGAGRGTYY | | SEQ ID | SEQ ID | SEQ ID | |
| | | ADSVKGRFTISRDNDKNTLYLQMNSLKPED | | NO: 1080 | NO: 1081 | NO: 1082 | |
| | | TAVYYCAKYDSSTYYPPTPARGIADRGQGI | | | | | |
| | | QVTVSS- | | | | | |
| | | SEQ ID | | | | | |
| | | NO: 205 | | | | | |
| 207 | 206 | EVQLVESGGGLVQPGGSLRLSCAISGGTLD | b49 | SYGIG- | CMSRSDDRTFYADSVKG- | VDAYGCSLVQPTTYDF- | 34 |
| | | SYGIGWVRQAPGKQREGVSCMSRSDDRTFY | | SEQ ID | SEQ ID | SEQ ID | |
| | | ADSVKGRFTISKDNAKSTVYLQMTNLKPDD | | NO: 1083 | NO: 1084 | NO: 1085 | |
| | | TAVYYCAAVDAYGCSLVQPTTYDFWGLGTQ | | | | | |
| | | VTVSS- | | | | | |
| | | SEQ ID | | | | | |
| | | NO: 206 | | | | | |
| 209 | 207 | EVQLVESGGGSVQTGGSLRLSCVVSGSHFI | b62 | SDVMG- | DMADGGSVKYGDSVKG- | GLWITGRHFDY- | 14 |
| | | SDVMGWYRQVPGKQRELVADMADGGSVKYG | | SEQ ID | SEQ ID | SEQ ID | |
| | | DSVKGRFTILRDSVKNTVTLQMSSLNPEDT | | NO: 1086 | NO: 1087 | NO: 1088 | |
| | | GVYYCKAGLWITGRHFDYWGQGTQVTVTS- | | | | | |
| | | SEQ ID | | | | | |
| | | NO: 207 | | | | | |
| 210 | 208 | QLQLVESGGGLVQTGGSLRLSCAVSGSRFI | b70 | SDVMG- | DMADGGSAKYADSVKG- | GLWIAGRHSDY- | 14 |
| | | SDVMGWYRQVPGKQRELVADMADGGSAKYA | | SEQ ID | SEQ ID | SEQ ID | |
| | | DSVKGRFSILRDSVKNTVYLQMSSLKPEDT | | NO: 1089 | NO: 1090 | NO: 1091 | |
| | | GIYYCKAGLWIAGRHSDYWGQGTQVTVSS- | | | | | |
| | | SEQ ID | | | | | |
| | | NO: 208 | | | | | |
| 212 | 209 | QVQLVESGGGLVQVGDSLRLSCAASGRTVG | b171 | SANMG- | VITWSTGSTVYADSVKG- | DVYPYSGNLNIMDY- | 13 |
| | | SANMGWFRQAPGKEREFVAVITWSTGSTVY | | SEQ ID | SEQ ID | SEQ ID | |
| | | ADSVKGRFTISRDNAKNTVYLDLSSLKPED | | NO: 1092 | NO: 1093 | NO: 1094 | |
| | | TAVYYCAADVYPYSGNLNIMDYWGKGTQVT | | | | | |
| | | VSS- | | | | | |
| | | SEQ ID | | | | | |
| | | NO: 209 | | | | | |
| 213 | 210 | QVQLVESGGGLVQPGGSLRLSCAVSGGTLH | b161 | DYGIG- | CMSRSDDKTYYADYVKG- | VDAYGCSLVQPTTYDY- | 34 |
| | | DYGIGWIRQAPGKQREGVSCMSRSDDKTYY | | SEQ ID | SEQ ID | SEQ ID | |
| | | ADYVKGRFTISKDNAKNTVYLQMISLKPED | | NO: 1095 | NO: 1096 | NO: 1097 | |
| | | TAVYYCAAVDAYGCSLVQPTTYDYWGQGTQ | | | | | |
| | | VTVSS- | | | | | |
| | | SEQ ID | | | | | |
| | | NO: 210 | | | | | |
| 214 | 211 | QVQLVESGGTLVQTGGSLRLSCVVSGSHFI | b180 | SDVMG- | DMADGGSAKYGDSVKG- | GLWIKGRHSDY- | 14 |
| | | SDVMGWYRQVPGKQRELVADMADGGSAKYG | | SEQ ID | SEQ ID | SEQ ID | |
| | | DSVKGRFTIVRDSVKNTVYLQMSSLNPEDT | | NO: 1098 | NO: 1099 | NO: 1100 | |
| | | GVYYCKAGLWIKGRHSDYWGQGTQVTVTS- | | | | | |
| | | SEQ ID | | | | | |
| | | NO: 211 | | | | | |
| 215 | 212 | QVQLVESGGGLVQPGGSLGLSCAASGFTFD | b151 | NYAMS- | SITGAGRGTHYADSVKG- | YSSSTYYPPTPARGTDY- | 38 |
| | | NYAMSWVRQAPGKGLEWVSSITGAGRGTHY | | SEQ ID | SEQ ID | SEQ ID | |
| | | ADSVKGRFTISRDNAKNTLYLQMNSLKPED | | NO: 1101 | NO: 1102 | NO: 1103 | |
| | | TAVYYCAKYSSSTYYPPTPARGTDYRGPGT | | | | | |
| | | QVTVSS- | | | | | |
| | | SEQ ID | | | | | |
| | | NO: 212 | | | | | |
| 217 | 213 | EVQLVESGGGSVQTGGSLRLSCVVSGSHFI | b62 | SDVMG- | DMADGGSVKYGDSVKG- | GLWITGRHFDY- | 14 |
| | | SDVMGWYRQVPGKQRELVADMADGGSVKYG | | SEQ ID | SEQ ID | SEQ ID | |
| | | DSVKGRFTILRDSVKNTVTLQMSSLNPEDT | | NO: 1104 | NO: 1105 | NO: 1106 | |
| | | GVYYCKAGLWITGRHFDYWGQGTQVTVTS- | | | | | |
| | | SEQ ID | | | | | |
| | | NO: 213 | | | | | |
| 218 | 214 | QVQLVESGGGLVQTGGSLRLSCAASGSQFI | b165 | NDVMG- | DMDDTGSTKYADSVKG- | GLWIKGRHSDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 1107 | NO: 1108 | NO: 1109 | |
| | | GVYYCKAGLWIKGRHSDYWGQGTQVTVSS- | | | | | |
| | | SEQ ID | | | | | |
| | | NO: 214 | | | | | |
| 219 | 215 | EVQLVESGGGLVQAGASLELSCAASGGTFS | b2 | SYAMG- | ATTRGGGRTMYAVSTEG- | RTSYYDGTYYPASSDFDI- | 27 |
| | | SYAMGWFRQAPGKEREFVAATTRGGGRTMY | | SEQ ID | SEQ ID | SEQ ID | |
| | | AVSTEGRFSCSRDNAKNTVNLLIKNLKPED | | NO: 1110 | NO: 1111 | NO: 1112 | |
| | | TAVYYCAARTSYYDGTYYPASSDFDIWGQG | | | | | |
| | | TQVTVSS- | | | | | |
| | | SEQ ID | | | | | |
| | | NO: 215 | | | | | |
| 220 | 216 | QVQLVESGGGLVQPGGSLRLSCAASGFTFS | b155 | NYAMS- | SSITSAGRGTHYADTVKG- | YSSATYYPPTPAQGRDY- | 38 |
| | | NYAMSWVRQAPGKELEWVSSITSAGRGTHY | | SEQ ID | SEQ ID | SEQ ID | |
| | | ADTVKGRFTISRDNDENTLYLQMNSLEPED | | NO: 1113 | NO: 1114 | NO: 1115 | |
| | | TAVYYCAKYSSATYYPPTPAQGRDYRGQGT | | | | | |
| | | QVTVSS- | | | | | |
| | | SEQ ID | | | | | |
| | | NO: 216 | | | | | |
| 221 | 217 | QVQLVESGGGLVQTGGSLRLSCAVSGSHFI | b167 | SDVMG- | DMADGNSAKYADSVKG- | GLWIAGRHSDY- | 14 |
| | | SDVMGWYRQVPEKQRELVADMADGNSAKYA | | SEQ ID | SEQ ID | SEQ ID | |
| | | DSVKGRFSILRDSVKNTVYLQMSSLKPEDT | | NO: 1116 | NO: 1117 | NO: 1118 | |
| | | GIYYCKAGLWIAGRHSDYWGQGTQVTVSS- | | | | | |
| | | SEQ ID | | | | | |
| | | NO: 217 | | | | | |
| 222 | 218 | EVQLVESGGGLVQTGGSLRLSCAASGSQFI | b53 | NDVMG- | DMDDTGSTKYADSVKG- | GLWIKGRHSDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 1119 | NO: 1120 | NO: 1121 | |
| | | GVYYCKAGLWIKGRHSDYWGQGTQVTVSS- | | | | | |
| | | SEQ ID | | | | | |
| | | NO: 218 | | | | | |
| 224 | 219 | QVQLVESGGGLVQAGGSLALSCAASGGTFS | b92 | SYAMG- | ATTRGGGRTMYAVSTEG- | RTSYYDGTYYPASSDFDI- | 27 |
| | | SYAMGWFRQAPGKEREFVAATTRGGGRTMY | | SEQ ID | SEQ ID | SEQ ID | |
| | | AVSTEGRFSCSRDNAKNTVYLLIKNLKPED | | NO: 1122 | NO: 1123 | NO: 1124 | |
| | | TAVYYCAARTSYYDGTYYPASSDFDIWGQG | | | | | |
| | | TQVTVSS- | | | | | |
| | | SEQ ID | | | | | |
| | | NO: 219 | | | | | |
| 226 | 220 | EVQLVESGGGLVQAGGSLRLACALSGDVFV | b8 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1125 | NO: 1126 | NO: 1127 | |
| | | TAVYYCAANTDYFQIRSLDRNTWGQGTQVT | | | | | |
| | | VSS- | | | | | |
| | | SEQ ID | | | | | |
| | | NO: 220 | | | | | |
| 229 | 221 | EVQLVESGGGLVQAGGSLRLSCAVSGVIFW | b35 | SDVVG- | TINLDDTKHYGEVVKG- | RRGSEFY- | 26 |
| | | SDVVGWYRQASGKQRELVATINLDDTKHYG | | SEQ ID | SEQ ID | SEQ ID | |
| | | EVVKGRFAISRDSHKNAYYLQLNSLKPEDT | | NO: 1128 | NO: 1129 | NO: 1130 | |
| | | AVYYCNVRRGSEFYWGQGTQVTVSS- | | | | | |
| | | SEQ ID | | | | | |
| | | NO: 221 | | | | | |
| 231 | 222 | EVQLVESGGGLVQIGGSLRLSCAASGRTFS | b46 | SITMA- | GINFSGTRTFYADSVKG- | SPRGFYGPGNALYDY- | 29 |
| | | SITMAYFRQVPGKEREWVAGINFSGTRTFY | | SEQ ID | SEQ ID | SEQ ID | |
| | | ADSVKGRFLISRDDAKSTMYLQMNSLKPED | | NO: 1131 | NO: 1132 | NO: 1133 | |
| | | TAVYYCAASPRGFYGPGNALYDYWGQGTQV | | | | | |
| | | TVSS- | | | | | |
| | | SEQ ID | | | | | |
| | | NO: 222 | | | | | |
| 232 | 223 | QLQLVESGGGLVQAGGSLALSCAASGGTFS | b65 | SYAMG- | ATTRGGGRTMYAVSTEG- | RTSYYDGTYYPASSDFDI- | 27 |
| | | SYAMGWFRQAPGKEREFVAATTRGGGRTMY | | SEQ ID | SEQ ID | SEQ ID | |
| | | AVSTEGRFSCSRDNAKNTVYLLIKNLKPED | | NO: 1134 | NO: 1135 | NO: 1136 | |
| | | TAVYYCAARTSYYDGTYYPASSDFDIWGQG | | | | | |
| | | TQVTVSS- | | | | | |
| | | SEQ ID | | | | | |
| | | NO: 223 | | | | | |
| 233 | 224 | EVQLVESGGGLVQTGGSLRLSCAVSGSRFI | b56 | SDVMG- | DMADGGSAKYADSVKG- | GLWIAGRHSDY- | 14 |
| | | SDVMGWYRQVPGKARELVADMADGGSAKYA | | SEQ ID | SEQ ID | SEQ ID | |
| | | DSVKGRFSIVRDSGKSTMYLQMSSLKPEDT | | NO: 1137 | NO: 1138 | NO: 1139 | |
| | | GMYYCKAGLWIAGRHSDYWGQGTQVTVSS- | | | | | |
| | | SEQ ID | | | | | |
| | | NO: 224 | | | | | |
| 235 | 225 | QVQLVESGGGLVQAGGSLRLACALSGDVFV | b96 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1140 | NO: 1141 | NO: 1142 | |
| | | TAVYYCAANTDYFQIRSLDRNTWGQGTQVT | | | | | |
| | | VSS- | | | | | |
| | | SEQ ID | | | | | |
| | | NO: 225 | | | | | |
| 237 | 226 | QVQLVESGGGLVQAGRSLGLSCAASGFTFD | b148 | NYAMS- | SITGAGRGTHYADSVKG- | YSSSTYYPPTPARGTDY- | 38 |
| | | NYAMSWVRQAPGKGLEWVSSITGAGRGTHY | | SEQ ID | SEQ ID | SEQ ID | |
| | | ADSVKGRFTISRDNAKNTLYLQMNSLKPED | | NO: 1143 | NO: 1144 | NO: 1145 | |
| | | TAVYYCAKYSSSTYYPPTPARGTDYRGPGT | | | | | |
| | | QVTVSS- | | | | | |
| | | SEQ ID | | | | | |
| | | NO: 226 | | | | | |
| 238 | 227 | QVQLQESGGGLVQTGGSLRLSCAASGSQFI | b84 | NDVMG- | DMDDTGSTKYADSVKG- | GLWIKGRHSDY- | 14 |
| | | NDVMGWYRQVPGKQRELVADMDDTGSTKYA | | SEQ ID | SEQ ID | SEQ ID | |
| | | DSVKGRFTILRDSVKNTAYLQMSNLKPEDT | | NO: 1146 | NO: 1147 | NO: 1148 | |
| | | GVYYCKAGLWIKGRHSDYWGQGTQVTVSS- | | | | | |
| | | SEQ ID | | | | | |
| | | NO: 227 | | | | | |
| 241 | 228 | QVQLVESGGGLVEAGGSLRLSCAASGRAFN | b88 | NFPMG- | AITWISGSTLYADSVKG- | ALKTITRGQHDYTY- | 1 |
| | | NFPMGWFRQAPGKEREFVAAITWISGSTLY | | SEQ ID | SEQ ID | SEQ ID | |
| | | ADSVKGRFTISRDKSKNTVYLQMSGLKPED | | NO: 1149 | NO: 1150 | NO: 1151 | |
| | | TALYYCAAALKTITRGQHDYTYWGQGTQVT | | | | | |
| | | VSS- | | | | | |
| | | SEQ ID | | | | | |
| | | NO: 228 | | | | | |
| 243 | 229 | QVQLVESGGGLVQAGDSLRLSCAASGRSLS | b89 | TDCMG- | GITWGTGSTLYADSVEG- | DRYCYRGTVYRD- | 11 |
| | | TDCMGWVRQAPGKEREFVAGITWGTGSTLY | | SEQ ID | SEQ ID | SEQ ID | |
| | | ADSVEGRFTISKDNAKNTGYLQMSSLKPED | | NO: 1152 | NO: 1153 | NO: 1154 | |
| | | TAVYYCAVDRYCYRGTVYRDWGRGTQVTVS | | | | | |
| | | S- | | | | | |
| | | SEQ ID | | | | | |
| | | NO: 229 | | | | | |
| 244 | 230 | EVQLVESGGGLVQAGGSLRLSCAASGDVFV | b11 | RYTMA- | SVTDSGRTTDYGDSVKG- | NTDYFQIRSLDANT- | 20 |
| | | RYTMAWFRQAPGKEREFVASVTDSGRTTDY | | SEQ ID | SEQ ID | SEQ ID | |
| | | GDSVKGRFTVSRDNAKNTVYLQMNNLKPED | | NO: 1155 | NO: 1156 | NO: 1157 | |
| | | TAVYYCAANTDYFQIRSLDANTWGQGTQVT | | | | | |
| | | VSS- | | | | | |
| | | SEQ ID | | | | | |
| | | NO: 230 | | | | | |
| 245 | 231 | QVQLVESGGGLVQAGGSLRLSCAVSGDVFV | b137 | RYTMA- | TVTDSGRTTEYVDSVKG- | NTDYFRIRSLDANT- | 20 |
| | | RYTMAWFRQAPGKEREFVATVTDSGRTTEY | | SEQ ID | SEQ ID | SEQ ID | |
| | | VDSVKGRFTVSRDNAKNTVYLQMNNLKPED | | NO: 1158 | NO: 1159 | NO: 1160 | |
| | | TAVYYCAANTDYFRIRSLDANTWGQGTQVT | | | | | |
| | | VSS- | | | | | |
| | | SEQ ID | | | | | |
| | | NO: 231 | | | | | |
| 246 | 232 | QVQLVESGGGLVQAGGSLRLSCAASGRTFS | b124 | SYVMG- | AITGTRRTTDWPDSVKG- | ARGLPVPPLGDIIH- | 3 |
| | | SYVMGWFRQAPGKEREFWVAITGTRRTTDW | | SEQ ID | SEQ ID | SEQ ID | |
| | | PDSVKGRFTISRDNAQNTVYLDMNSLKPED | | NO: 1161 | NO: 1162 | NO: 1163 | |
| | | TAVYYCASARGLPVPPLGDIIHWGEGTLVT | | | | | |
| | | VSS- | | | | | |
| | | SEQ ID | | | | | |
| | | NO: 232 | | | | | |
| 247 | 233 | QVQLVESGGGLVQAGGSLRLSCAASGRTFS | b122 | SDAKG- | AITWAGRGTDYADSVKG- | NPEGVIGPYFRNSNRYNY- | 19 |
| | | SDAKGWFRQAPGKEREFVAAITWAGRGTDY | | SEQ ID | SEQ ID | SEQ ID | |
| | | ADSVKGRFTITRDNAKNMVYLQMSNLKPED | | NO: 1164 | NO: 1165 | NO: 1166 | |
| | | TAVYYCALNPEGVIGPYFRNSNRYNYWGQG | | | | | |
| | | TQVTVSS- | | | | | |
| | | SEQ ID | | | | | |
| | | NO: 233 | | | | | |
| 248 | 234 | EVQLVESGGGLVQAGGSLRLSCVVSGDVFV | b38 | RYTMA- | AVTDSGRTTEYVDSVKG- | NTDYFQIRSVDNNA- | 21 |
| | | RYTMAWFRQAPGKEREFVAAVTDSGRTTEY | | SEQ ID | SEQ ID | SEQ ID | |
| | | VDSVKGRFTVSRDNAKNTVYLQMNNLKPED | | NO: 1167 | NO: 1168 | NO: 1169 | |
| | | TAVYYCAANTDYFQIRSVDNNAWGQGTQVT | | | | | |
| | | VSS- | | | | | |
| | | SEQ ID | | | | | |
| | | NO: 234 | | | | | |
| 250 | 235 | QVQLVESGGGLVQAGGSLRLACALSGDVFV | b96 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1170 | NO: 1171 | NO: 1172 | |
| | | TAVYYCAANTDYFQIRSLDRNTWGQGTQVT | | | | | |
| | | VSS- | | | | | |
| | | SEQ ID | | | | | |
| | | NO: 235 | | | | | |
| 251 | 236 | EVQLVESGGGLVQAGGSLRLACALSGDVFV | b8 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1173 | NO: 1174 | NO: 1175 | |
| | | TAVYYCAANTDYFQIRSLDRNTWGQGTQVT | | | | | |
| | | VSS- | | | | | |
| | | SEQ ID | | | | | |
| | | NO: 236 | | | | | |
| 253 | 237 | QVQLVESGGGLVQAGGSLRLSCAAAGRAIE | b98 | RYTMA- | AITWISGSTLYADSVKG- | ALKTITRGQNDYSY- | 1 |
| | | NYPVGWFRRAPGKEREFVAAITWISGSTLY | | SEQ ID | SEQ ID | SEQ ID | |
| | | ADSVKGRFTISRDNAKNTVYLQMSSLKPED | | NO: 1176 | NO: 1177 | NO: 1178 | |
| | | TALYYCAAALKTITRGQNDYSYWGQGTQVT | | | | | |
| | | VSS- | | | | | |
| | | SEQ ID | | | | | |
| | | NO: 237 | | | | | |
| 255 | 238 | QVQLVESGGGLVQAGGSLRLACALSGDVFV | b97 | RYTMA- | SVTDSGRTTDYVASVQG- | NTDYFQIRSLDRNT- | 20 |
| | | RYTMAWFRQAPGKGREFVASVTDSGRTTDY | | SEQ ID | SEQ ID | SEQ ID | |
| | | VASVQGRFTVSRDNNKNTVYLQMNSLKPED | | NO: 1179 | NO: 1180 | NO: 1181 | |
| | | TAVYYCAANTDYFQIRSLDRNTWGQGTQVT | | | | | |
| | | VSS- | | | | | |
| | | SEQ ID | | | | | |
| | | NO: 238 | | | | | |
| 257 | 239 | QVQLVESGGGLVQAGGSLRLACAASGRTFS | b95 | RYTMA- | GISPSGGYAWHADSVKG- | DHPPYGAVASRSEYEYDD- | 6 |
| | | TLTMGWFRQAPGKEREFVAGISPSGGYAWH | | SEQ ID | SEQ ID | SEQ ID | |
| | | ADSVKGRFTIDRDNAKNTVYLQTRSLRPED | | NO: 1182 | NO: 1183 | NO: 1184 | |
| | | TAVYYCAADHPPYGAVASRSEYEYDDWGPG | | | | | |
| | | TRVTVSS- | | | | | |
| | | SEQ ID | | | | | |
| | | NO: 239 | | | | | |
| 258 | 240 | QVQLVESGGGLVQAGGSLRLSCVVSGDVFV | b145 | RYTMA- | SITDSGRTTEYVDSVKG- | NTDYFRIRSLDANT- | 20 |
| | | RYTMAWFRQAPGKEREFVISITDSGRTTEY | | SEQ ID | SEQ ID | SEQ ID | |
| | | VDSVKGRFTVSRDNAKNTVYLQMNNLKPED | | NO: 1185 | NO: 1186 | NO: 1187 | |
| | | TAVYYCAANTDYFRIRSLDANTWGQGTQVT | | | | | |
| | | VSS- | | | | | |
| | | SEQ ID | | | | | |
| | | NO: 240 | | | | | |
| 260 | 241 | QVQLVESGGGLVQAGGSLRLSCAVSGDVFV | b132 | RYTMA- | SVTDSGRTTDYVHSVKG- | NTDYFQIKSLDANT- | 20 |
| | | RYTMAWFRQAPGKEREFVASVTDSGRTTDY | | SEQ ID | SEQ ID | SEQ ID | |
| | | VHSVKGRFTVSRDNAKNTVYLQMNNLKPED | | NO: 1188 | NO: 1189 | NO: 1190 | |
| | | TAVYYCAANTDYFQIKSLDANTWGQGTQVT | | | | | |
| | | VSS- | | | | | |
| | | SEQ ID | | | | | |
| | | NO: 241 | | | | | |
| 262 | 242 | EVQLVESGGGLVQAGGSLTLSCAASGRAFS | b43 | RYTMA- | AITWLSGSTLYADSVEG- | ALKTITRGQNDYSY- | 1 |
| | | NYPVGWFRQAPGKEREFVAAITWLSGSTLY | | SEQ ID | SEQ ID | SEQ ID | |
| | | ADSVEGRFTISRDNAKNTVYLLMSSLKPED | | NO: 1191 | NO: 1192 | NO: 1193 | |
| | | TALYFCAAALKTITRGQNDYSYWGQGTQVT | | | | | |
| | | VSS- | | | | | |
| | | SEQ ID | | | | | |
| | | NO: 242 | | | | | |
| 263 | 243 | QVQLVESGGGLVQAGGSLRLSCVVSGDVFV | b144 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 1194 | NO: 1195 | NO: 1196 | |
| | | TAVYYCAANTDYFQIRSVDNNAWGQGTQVT | | | | | |
| | | VSS- | | | | | |
| | | SEQ ID | | | | | |
| | | NO: 243 | | | | | |
| 264 | 244 | QVQLVESGGGLVQAGGSLRLSCAASGSIFS | b127 | RYTMA- | AISRSGGSTAYVDSVKG- | TAAVGWTSRWITTDFDS- | 30 |
| | | INAMGWFRQAPGKEREFVAAISRSGGSTAY | | SEQ ID | SEQ ID | SEQ ID | |
| | | VDSVKGRFTISRDNSKNMVHLQMNSLKSED | | NO: 1197 | NO: 1198 | NO: 1199 | |
| | | EAVYYCAATAAVGWTSRWITTDFDSWGQGT | | | | | |
| | | QVTVSS- | | | | | |
| | | SEQ ID | | | | | |
| | | NO: 244 | | | | | |
| 265 | 245 | QVQLVESGGGLVQAGGSLRLACALSGDVFV | b96 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1200 | NO: 1201 | NO: 1202 | |
| | | TAVYYCAANTDYFQIRSLDRNTWGQGTQVT | | | | | |
| | | VSS- | | | | | |
| | | SEQ ID | | | | | |
| | | NO: 245 | | | | | |
| 266 | 246 | QVQLVESGGGLVQAGGSLRLSCVVSGDVFV | b144 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 1203 | NO: 1204 | NO: 1205 | |
| | | TAVYYCAANTDYFQIRSVDNNAWGQGTQVT | | | | | |
| | | VSS- | | | | | |
| | | SEQ ID | | | | | |
| | | NO: 246 | | | | | |
| 267 | 247 | QVQLVESGGGLVQAGGSLRLSCAASGRTFS | b126 | RYTMA- | AITWTGRGTMYAESVEG- | ARRGRAAVRSEGGYDF- | 4 |
| | | TTEMGWFRQAPGKEREFVAAITWTGRGTMY | | SEQ ID | SEQ ID | SEQ ID | |
| | | AESVEGRFTISRENAKNMVYLQMNSLKPED | | NO: 1206 | NO: 1207 | NO: 1208 | |
| | | TAIYYCAAARRGRAAVRSEGGYDFWGQGTQ | | | | | |
| | | VTVSS- | | | | | |
| | | SEQ ID | | | | | |
| | | NO: 247 | | | | | |
| 268 | 248 | QVQLQESGGGLVQAGDSLRLSCVVSGRDFS | b73 | RYTMA- | LITWSTGRVHYTDSVEG- | GRVGYTTNLHSYDY- | 16 |
| | | GYTMGWFRQPPGKEREFVALITWSTGRVHY | | SEQ ID | SEQ ID | SEQ ID | |
| | | TDSVEGRFTISRDSAKNTVYLQMNSLKPED | | NO: 1209 | NO: 1210 | NO: 1211 | |
| | | TAVYTCAAGRVGYTTNLHSYDYSGQGTQVI | | | | | |
| | | VSA- | | | | | |
| | | SEQ ID | | | | | |
| | | NO: 248 | | | | | |
| 269 | 249 | EVQLVESGGGLVQPGGSLRLSCAASGFTFR | b48 | RYTMA- | GITGEGLGTIYQDSVEG- | MASGTLFRDKPYEYTS- | 17 |
| | | NYAMSWVRQAPGKGLEWVGGITGEGLGTIY | | SEQ ID | SEQ ID | SEQ ID | |
| | | QDSVEGRFTISRNNAKNTLYLQMNSLRSED | | NO: 1212 | NO: 1213 | NO: 1214 | |
| | | TAVYYCTKMASGTLFRDKPYEYTSRGQGTQ | | | | | |
| | | VTVSS- | | | | | |
| | | SEQ ID | | | | | |
| | | NO: 249 | | | | | |
| 271 | 250 | QVQLVESGGGLVQAGGSLRLSCAASGRSYA | b117 | RYTMA- | AISWSASSTYYSESVKG- | RASPFVAGSYDPSDDPADYGS- | 23 |
| | | MGWFRQAPGKEREFVAAISWSASSTYYSES | | SEQ ID | SEQ ID | SEQ ID | |
| | | VKGRFTISRENAKNTVYLQMNSLKPEDTAV | | NO: 1215 | NO: 1216 | NO: 1217 | |
| | | YYCAVRASPFVAGSYDPSDDPADYGSWGQG | | | | | |
| | | TQVIVSS- | | | | | |
| | | SEQ ID | | | | | |
| | | NO: 250 | | | | | |
| 272 | 251 | QVQLVESGGGLVQAGGSLRLSCAASGRTFS | b126 | RYTMA- | AITWTGRGTMYAESVEG- | ARRGRAAVRSEGGYDF- | 4 |
| | | TTEMGWFRQAPGKEREFVAAITWTGRGTMY | | SEQ ID | SEQ ID | SEQ ID | |
| | | AESVEGRFTISRENAKNMVYLQMNSLKPED | | NO: 1218 | NO: 1219 | NO: 1220 | |
| | | TAIYYCAAARRGRAAVRSEGGYDFWGQGTQ | | | | | |
| | | VTVSS- | | | | | |
| | | SEQ ID | | | | | |
| | | NO: 251 | | | | | |
| 274 | 252 | EVQLVESGGGLVQAGGSLRLACALSGDVFV | b8 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1221 | NO: 1222 | NO: 1223 | |
| | | TAVYYCAANTDYFQIRSLDRNTWGQGTQVT | | | | | |
| | | VSS- | | | | | |
| | | SEQ ID | | | | | |
| | | NO: 252 | | | | | |
| 275 | 253 | QVQLVESGGGLVQAGGSLRLACALSGDVFV | b96 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1224 | NO: 1225 | NO: 1226 | |
| | | TAVYYCAANTDYFQIRSLDRNTWGQGTQVT | | | | | |
| | | VSS- | | | | | |
| | | SEQ ID | | | | | |
| | | NO: 253 | | | | | |
| 276 | 254 | QVQLVESGGGLVQAGGSLRLSCAASGRTFS | b125 | RYTMA- | AITWSSHSTLYADSVEG- | VRRGWAVVRTEGAYDF- | 35 |
| | | TTDVGWFRQAPGKEREFVAAITWSSHSTLY | | SEQ ID | SEQ ID | SEQ ID | |
| | | ADSVEGRFTVTRENAKNTVYLQMNSLKPED | | NO: 1227 | NO: 1228 | NO: 1229 | |
| | | TAVYYCAAVRRGWAVVRTEGAYDFWGQGTQ | | | | | |
| | | VTVSA- | | | | | |
| | | SEQ ID | | | | | |
| | | NO: 254 | | | | | |
| 277 | 255 | EVQLVESGGGLVQAGGSLRLSCVVSGDVFV | b37 | RYTMA- | AVTDSGRTTEYVDSVKG- | NTDYFQIRSVDNNA- | 21 |
| | | RYTMAWFRQAPGKEREFVAAVTDSGRTTEY | | SEQ ID | SEQ ID | SEQ ID | |
| | | VDSVKGRFTVSRDNAKNTVYLQMNNLEPED | | NO: 1230 | NO: 1231 | NO: 1232 | |
| | | TAVYYCAANTDYFQIRSVDNNAWGQGTQVT | | | | | |
| | | VSS- | | | | | |
| | | SEQ ID | | | | | |
| | | NO: 255 | | | | | |
| 278 | 256 | QVQLVESGGGLAQAGGSLRLSCAASGRTFS | b87 | RYTMA- | AITWSGRDTDYADSVKG- | YPQGTIGPYFRSSNHYDY- | 37 |
| | | QDAKAWFRQAPGKEREFVAAITWSGRDTDY | | SEQ ID | SEQ ID | SEQ ID | |
| | | ADSVKGRFTIARDNAKNTVYLQMDSLKPED | | NO: 1233 | NO: 1234 | NO: 1235 | |
| | | TAVYYCAVYPQGTIGPYFRSSNHYDYWGQG | | | | | |
| | | TQVTVSS- | | | | | |
| | | SEQ ID | | | | | |
| | | NO: 256 | | | | | |
| 279 | 257 | EVQLVESGGGLVQAGGSLRLSCAASGDVFV | b14 | RYTMA- | SVTDSGRTTDYVDSVKG- | NTDYFQIRSLEFNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1236 | NO: 1237 | NO: 1238 | |
| | | TAVYYCAANTDYFQIRSLEFNTWGQGTQVT | | | | | |
| | | VSS- | | | | | |
| | | SEQ ID | | | | | |
| | | NO: 257 | | | | | |
| 282 | 258 | EVQLVESGGGLVQAGGSLRLSCAASGDVFV | b10 | RYTMA- | AVTDSGRTADYVDSVKG- | NTDYFQIRSLDRNT- | 20 |
| | | RYTMAWFRQAPGKEREFVAAVTDSGRTADY | | SEQ ID | SEQ ID | SEQ ID | |
| | | VDSVKGRFTVSRDNAKNTVYLQMNNLKPED | | NO: 1239 | NO: 1240 | NO: 1241 | |
| | | TAVYYCAANTDYFQIRSLDRNTWGQGTQVT | | | | | |
| | | VSS- | | | | | |
| | | SEQ ID | | | | | |
| | | NO: 258 | | | | | |
| 283 | 259 | QVQLVESGGGLVQAGGSLRLSCAASGRAFS | b116 | RYTMA- | IISESGGIIDYGDSVKG- | ARDWFARTAYQYDY- | 2 |
| | | TYSMGWFRQTPGKEREFVAIISESGGIIDY | | SEQ ID | SEQ ID | SEQ ID | |
| | | GDSVKGRFTLSRDNAKNTVSLQMSSLQPED | | NO: 1242 | NO: 1243 | NO: 1244 | |
| | | TAVYYCAAARDWFARTAYQYDYWGQGTQVT | | | | | |
| | | VSA- | | | | | |
| | | SEQ ID | | | | | |
| | | NO: 259 | | | | | |
| 481 | 260 | QVQLVESGGGLVQAGGSLRLACALSGDVFV | b96 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1245 | NO: 1246 | NO: 1247 | |
| | | TAVYYCAANTDYFQIRSLDRNTWGQGTQVT | | | | | |
| | | VSS- | | | | | |
| | | SEQ ID | | | | | |
| | | NO: 260 | | | | | |
| 482 | 261 | QVQLVESGGGLVQAGGSLRLSCAASGDVFV | b103 | RYTMA- | SVTDSGRTTDYVHSVKG- | NTDYFQIRSLDANT- | 20 |
| | | RYTMAWFRQAPGKEREFVASVTDSGRTTDY | | SEQ ID | SEQ ID | SEQ ID | |
| | | VHSVKGRFTVSRDNAKNTVYLQMNNLKPED | | NO: 1248 | NO: 1249 | NO: 1250 | |
| | | TAVYYCAANTDYFQIRSLDANTWGQGTQVT | | | | | |
| | | VSS- | | | | | |
| | | SEQ ID | | | | | |
| | | NO: 261 | | | | | |
| 483 | 262 | QVQLVESGGGLVQAGGSLRLSCAASGDVFV | b100 | RYTMA- | SVTDSGRTTDYVDSVKG- | NTDYFQIKSLDLNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNALKPED | | NO: 1251 | NO: 1252 | NO: 1253 | |
| | | TAVYYCAANTDYFQIKSLDLNTWGQGTQVT | | | | | |
| | | VSS- | | | | | |
| | | SEQ ID | | | | | |
| | | NO: 262 | | | | | |
| 484 | 263 | QVQLVESGGRLVQAGGSLRLSCAASGDVFV | b178 | RYTMA- | SVTDSGRTTEYVDSVKG- | NTDYFQIRSLNLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1254 | NO: 1255 | NO: 1256 | |
| | | TAVYYCAANTDYFQIRSLNLNTWGQGTQVT | | | | | |
| | | VSS- | | | | | |
| | | SEQ ID | | | | | |
| | | NO: 263 | | | | | |
| 487 | 264 | EVQLVESGGGLVQAGDSLRLSCVVSGRGFS | b4 | RYTMA- | AITWSTGRTSYADSVKG- | DQVLWTTRPRDMRY- | 7 |
| | | TYTMGWFRQAPGKEREFVAAITWSTGRTSY | | SEQ ID | SEQ ID | SEQ ID | |
| | | ADSVKGRFTISRENAENTVYLQMNSLELED | | NO: 1257 | NO: 1258 | NO: 1259 | |
| | | TAVYYCATDQVLWTTRPRDMRYWGQGTQVT | | | | | |
| | | VSF- | | | | | |
| | | SEQ ID | | | | | |
| | | NO: 264 | | | | | |
| 488 | 265 | EVQLVESGGGLVQAGGSLRLSCAVSGDVFV | b33 | RYTMA- | SVTDSGRTTDYVHSVKG- | NTDYFQIKSLDANT- | 20 |
| | | RYTMAWFRQAPGKEREFVASVTDSGRTTDY | | SEQ ID | SEQ ID | SEQ ID | |
| | | VHSVKGRFTVSRDNAKNTVYLQMNNLKPED | | NO: 1260 | NO: 1261 | NO: 1262 | |
| | | TAVYYCAANTDYFQIKSLDANTWGQGTQVT | | | | | |
| | | VSS- | | | | | |
| | | SEQ ID | | | | | |
| | | NO: 265 | | | | | |
| 490 | 266 | QVQLVESGGGLVQAGGSLRLSCVVSGDVFV | b143 | RYTMA- | AVTDSGRTTEYVDSVKG- | NTDYFQIRSVDNNA- | 21 |
| | | RYTMAWFRQAPGKEREFVAAVTDSGRTTEY | | SEQ ID | SEQ ID | SEQ ID | |
| | | VDSVKGRFTVSRDNAKNTVYLQMNNLKPED | | NO: 1263 | NO: 1264 | NO: 1265 | |
| | | TAVYYCAANTDYFQIRSVDNNAWGQGTQVT | | | | | |
| | | VSS- | | | | | |
| | | SEQ ID | | | | | |
| | | NO: 266 | | | | | |
| 491 | 267 | QVQLVESGGGLVQAGGSLRLSCVVSGDVFV | b144 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 1266 | NO: 1267 | NO: 1268 | |
| | | TAVYYCAANTDYFQIRSVDNNAWGQGTQVT | | | | | |
| | | VSS- | | | | | |
| | | SEQ ID | | | | | |
| | | NO: 267 | | | | | |
| 493 | 268 | QVQLVESGGGLVQAGGSLRLSCAVSGDVFV | b137 | RYTMA- | TVTDSGRTTEYVDSVKG- | NTDYFRIRSLDANT- | 20 |
| | | RYTMAWFRQAPGKEREFVATVTDSGRTTEY | | SEQ ID | SEQ ID | SEQ ID | |
| | | VDSVKGRFTVSRDNAKNTVYLQMNNLKPED | | NO: 1269 | NO: 1270 | NO: 1271 | |
| | | TAVYYCAANTDYFRIRSLDANTWGQGTQVT | | | | | |
| | | VSS- | | | | | |
| | | SEQ ID | | | | | |
| | | NO: 268 | | | | | |
| 494 | 269 | QVQLQESGGGLVQAGGSLRLSCAASGDVFV | b74 | RYTMA- | SVTDSGRTTDYVDSVKG- | NTDYFQIKSLDLNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNALKPED | | NO: 1272 | NO: 1273 | NO: 1274 | |
| | | TAVYYCAANTDYFQIKSLDLNTWGQGTQVT | | | | | |
| | | VSS- | | | | | |
| | | SEQ ID | | | | | |
| | | NO: 269 | | | | | |
| 495 | 270 | EVQLVESGGGLVQAGGSLRLSCVVSGDVFV | b42 | RYTMA- | VVTDSGRTTEYVDSVKG- | NTDYFQIRSVDNNA- | 21 |
| | | RYTMAWFRQAPRKEREFVAVVTDSGRTTEY | | SEQ ID | SEQ ID | SEQ ID | |
| | | VDSVKGRFTVSRDNAKNTVYLQMNNLKPED | | NO: 1275 | NO: 1276 | NO: 1277 | |
| | | TAVYYCAANTDYFQIRSVDNNAWGQGTQVT | | | | | |
| | | VSS- | | | | | |
| | | SEQ ID | | | | | |
| | | NO: 270 | | | | | |
| 496 | 271 | QVQLVESGGGLVQAGGSLRLSCAVSGDVFV | b135 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1278 | NO: 1279 | NO: 1280 | |
| | | TAVYYCAANTDYFQIRSLDLNTWGQGTQVT | | | | | |
| | | VSS- | | | | | |
| | | SEQ ID | | | | | |
| | | NO: 271 | | | | | |
| 497 | 272 | EVQLVESGGGLVQAGGSLRLSCAASGDVFV | b14 | RYTMA- | SVTDSGRTTDYVDSVKG- | NTDYFQIRSLEFNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1281 | NO: 1282 | NO: 1283 | |
| | | TAVYYCAANTDYFQIRSLEFNTWGQGTQVT | | | | | |
| | | VSS- | | | | | |
| | | SEQ ID | | | | | |
| | | NO: 272 | | | | | |
| 500 | 273 | QVQLVESGGGLVQAGGSLRLSCAASGDVFV | b104 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1284 | NO: 1285 | NO: 1286 | |
| | | TAIYYCAANTDYFQIRSLDLNTWGQGTQVT | | | | | |
| | | VSS- | | | | | |
| | | SEQ ID | | | | | |
| | | NO: 273 | | | | | |
| 501 | 274 | EVQLVESGGGLVQAGGSLRLSCAASGDVFV | b12 | RYTMA- | SVTDSGRTTDYVDSVKG- | NTDYFQIKSLDRNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1287 | NO: 1288 | NO: 1289 | |
| | | TAVYYCAANTDYFQIKSLDRNTWGQGTQVT | | | | | |
| | | VSP- | | | | | |
| | | SEQ ID | | | | | |
| | | NO: 274 | | | | | |
| 503 | 275 | QVQLVESGGGLVQAGGSLRLSCVVSGDVFV | b144 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 1290 | NO: 1291 | NO: 1292 | |
| | | TAVYYCAANTDYFQIRSVDNNAWGQGTQVT | | | | | |
| | | VSS- | | | | | |
| | | SEQ ID | | | | | |
| | | NO: 275 | | | | | |
| 504 | 276 | QVQLVESGGGLVQAGGSLRLSCAVSGDVFV | b135 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1293 | NO: 1294 | NO: 1295 | |
| | | TAVYYCAANTDYFQIRSLDLNTWGQGTQVT | | | | | |
| | | VSS- | | | | | |
| | | SEQ ID | | | | | |
| | | NO: 276 | | | | | |
| 506 | 277 | QVQLVESGGGLVQAGGSLRLSCAVSGDVFV | b135 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1296 | NO: 1297 | NO: 1298 | |
| | | TAVYYCAANTDYFQIRSLDLNTWGQGTQVT | | | | | |
| | | VSS- | | | | | |
| | | SEQ ID | | | | | |
| | | NO: 277 | | | | | |
| 507 | 278 | QVQLQESGGGLVQAGGSLRLSCAASGDVFV | b75 | RYTMA- | SVTDSGRTTDYVDSVKG- | NTDYFQIRSLEFNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1299 | NO: 1300 | NO: 1301 | |
| | | TAVYYCAANTDYFQIRSLEFNTWGQGTQVT | | | | | |
| | | VSS- | | | | | |
| | | SEQ ID | | | | | |
| | | NO: 278 | | | | | |
| 508 | 279 | QVQLQESGGGLVQAGGSLRLSCVVSGDVFV | b82 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 1302 | NO: 1303 | NO: 1304 | |
| | | TAVYYCAANTDYFQIRSVDNNAWGQGTQVT | | | | | |
| | | VSS- | | | | | |
| | | SEQ ID | | | | | |
| | | NO: 279 | | | | | |
| 509 | 280 | QVQLVESGGGLVQAGGSLRLSCAVSGDVFV | b135 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1305 | NO: 1306 | NO: 1307 | |
| | | TAVYYCAANTDYFQIRSLDLNTWGQGTQVT | | | | | |
| | | VSS- | | | | | |
| | | SEQ ID | | | | | |
| | | NO: 280 | | | | | |
| 510 | 281 | QVQLVESGGGLVQAGGSLRLSCAVSGDVFV | b136 | RYTMA- | SVTDSGRTTEYVDSVKG- | NTDYFQIRSLRANT- | 20 |
| | | RYTMAWFRQAPGKEREFVASVTDSGRTTEY | | SEQ ID | SEQ ID | SEQ ID | |
| | | VDSVKGRFTVSRDNAKNTVYLQMNSLKPED | | NO: 1308 | NO: 1309 | NO: 1310 | |
| | | TAVYYCAANTDYFQIRSLRANTWGQGTQVT | | | | | |
| | | VSS- | | | | | |
| | | SEQ ID | | | | | |
| | | NO: 281 | | | | | |
| 511 | 282 | QVQLQESGGGLVQAGGSLRLSCVVSGDVFV | b82 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 1311 | NO: 1312 | NO: 1313 | |
| | | TAVYYCAANTDYFQIRSVDNNAWGQGTQVT | | | | | |
| | | VSS- | | | | | |
| | | SEQ ID | | | | | |
| | | NO: 282 | | | | | |
| 512 | 283 | EVQLVESGGGLVQAGGSLRLSCAVSGDVFV | b34 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1314 | NO: 1315 | NO: 1316 | |
| | | TAVYYCAANTDYFQIRSLDLNTWGQGTQVT | | | | | |
| | | VSS- | | | | | |
| | | SEQ ID | | | | | |
| | | NO: 283 | | | | | |
| 513 | 284 | QVQLVESGGGLVQAGGSLRLSCAVSGDVFV | b132 | RYTMA- | SVTDSGRTTDYVHSVKG- | NTDYFQIKSLDANT- | 20 |
| | | RYTMAWFRQAPGKEREFVASVTDSGRTTDY | | SEQ ID | SEQ ID | SEQ ID | |
| | | VHSVKGRFTVSRDNAKNTVYLQMNNLKPED | | NO: 1317 | NO: 1318 | NO: 1319 | |
| | | TAVYYCAANTDYFQIKSLDANTWGQGTQVT | | | | | |
| | | VSS- | | | | | |
| | | SEQ ID | | | | | |
| | | NO: 284 | | | | | |
| 514 | 285 | QVQLVESGGGLVQAGGSLRLSCAASGDVFV | b102 | RYTMA- | SVTDSGRTTDYVDSVKG- | NTDYFQIRSLEFNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1320 | NO: 1321 | NO: 1322 | |
| | | TAVYYCAANTDYFQIRSLEFNTWGQGTQVT | | | | | |
| | | VSS- | | | | | |
| | | SEQ ID | | | | | |
| | | NO: 285 | | | | | |
| 515 | 286 | EVQLVESGGGLVQAGGSLRLSCVVSGDVFV | b41 | RYTMA- | VVTDSGRTTEYVDSVKG- | NTDYFQIRSVDNNA- | 21 |
| | | RYTMAWFRQAPGKGREFVAVVTDSGRTTEY | | SEQ ID | SEQ ID | SEQ ID | |
| | | VDSVKGRFTVSRDNAKNTVYLQMNNLKPED | | NO: 1323 | NO: 1324 | NO: 1325 | |
| | | TAVYYCAANTDYFQIRSVDNNAWGQGTQVT | | | | | |
| | | VSS- | | | | | |
| | | SEQ ID | | | | | |
| | | NO: 286 | | | | | |
| 516 | 287 | QVQLVESGGGLVQAGGSLRLSCAVSGDVFV | b136 | RYTMA- | SVTDSGRTTEYVDSVKG- | NTDYFQIRSLRANT- | 20 |
| | | RYTMAWFRQAPGKEREFVASVTDSGRTTEY | | SEQ ID | SEQ ID | SEQ ID | |
| | | VDSVKGRFTVSRDNAKNTVYLQMNSLKPED | | NO: 1326 | NO: 1327 | NO: 1328 | |
| | | TAVYYCAANTDYFQIRSLRANTWGQGTQVT | | | | | |
| | | VSS- | | | | | |
| | | SEQ ID | | | | | |
| | | NO: 287 | | | | | |
| 520 | 288 | QVQLVESGGRLVQAGGSLRLSCVVSGDVFV | b179 | RYTMA- | VVTDSGRTTEYVGSVKG- | NTDYFQIRSVDNNA- | 21 |
| | | RYTMAWFRQAPGKEREFVAVVTDSGRTTEY | | SEQ ID | SEQ ID | SEQ ID | |
| | | VGSVKGRFTVSRDNAKNTVYLQMNNLKPED | | NO: 1329 | NO: 1330 | NO: 1331 | |
| | | TAVYYCAANTDYFQIRSVDNNAWGQGTQVT | | | | | |
| | | VSS- | | | | | |
| | | SEQ ID | | | | | |
| | | NO: 288 | | | | | |
| 521 | 289 | QLQLVESGGGLVQAGGSLRLACALSGDVFV | b66 | RYTMA- | SVTDSGRTTDYVASVQG- | NTDYFQIRSLDRNT- | 20 |
| | | RYTMAWFRQAPGKEREFVASVTDSGRTTDY | | SEQ ID | SEQ ID | SEQ ID | |
| | | VASVQGRFTVSRDNNKNTVYLQMNSLKPED | | NO: 1332 | NO: 1333 | NO: 1334 | |
| | | TAVYYCAANTDYFQIRSLDRNTWGQGTQVT | | | | | |
| | | VSS- | | | | | |
| | | SEQ ID | | | | | |
| | | NO: 289 | | | | | |
| 522 | 290 | EVQLVESGGGLVQAGGPLRLSCAVSGDVFV | b6 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1335 | NO: 1336 | NO: 1337 | |
| | | TAVYYCAANTDYFQIRSLDLNTWGQGTQVT | | | | | |
| | | VSS- | | | | | |
| | | SEQ ID | | | | | |
| | | NO: 290 | | | | | |
| 523 | 291 | QVQLQESGGGLVQAGGSLRLSCVVSGDVFV | b82 | RYTMA- | VVTDSGRTTEYVDSVKG- | NTDYFQIRSVDNNA- | 21 |
| | | RYTMAWFRQAPGKEREFVAVVTDSGRTTEY | | SEQ ID | SEQ ID | SEQ ID | |
| | | VDSVKGRFTVSRDNAKNTVYLQMNNLKPED | | NO: 1338 | NO: 1339 | NO: 1340 | |
| | | TAVYYCAANTDYFQIRSVDNNAWGQGTQVT | | | | | |
| | | VSS- | | | | | |
| | | SEQ ID | | | | | |
| | | NO: 291 | | | | | |
| 524 | 292 | EVQLVESGGGLVQAGGSLRLSCAVSGDVFV | b34 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1341 | NO: 1342 | NO: 1343 | |
| | | TAVYYCAANTDYFQIRSLDLNTWGQGTQVT | | | | | |
| | | VSS- | | | | | |
| | | SEQ ID | | | | | |
| | | NO: 292 | | | | | |
| 525 | 293 | EVQLVESGGGLVQAGGSLRLFCAVSGDVFV | b9 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKDTAYLQMNNLKPED | | NO: 1344 | NO: 1345 | NO: 1346 | |
| | | TAVYYCAANTDYFQIRSLDLNTWGQGTQVT | | | | | |
| | | VSS- | | | | | |
| | | SEQ ID | | | | | |
| | | NO: 293 | | | | | |
| 526 | 294 | QVQLVESGGGLVQAGGSLRLSCAASGDVFV | b102 | RYTMA- | SVTDSGRTTDYVDSVKG- | NTDYFQIRSLEFNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1347 | NO: 1348 | NO: 1349 | |
| | | TAVYYCAANTDYFQIRSLEFNTWGQGTQVT | | | | | |
| | | VSS- | | | | | |
| | | SEQ ID | | | | | |
| | | NO: 294 | | | | | |
| 530 | 295 | EVQLVESGGGLVQAGGSLRLSCAVSGDVFV | b34 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1350 | NO: 1351 | NO: 1352 | |
| | | TAVYYCAANTDYFQIRSLDLNTWGQGTQVT | | | | | |
| | | VSS- | | | | | |
| | | SEQ ID | | | | | |
| | | NO: 295 | | | | | |
| 532 | 296 | QVQLVESGGGLVQAGGSLRLSCAASGDVFV | b104 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1353 | NO: 1354 | NO: 1355 | |
| | | TAIYYCAANTDYFQIRSLDLNTWGQGTQVT | | | | | |
| | | VSS- | | | | | |
| | | SEQ ID | | | | | |
| | | NO: 296 | | | | | |
| 537 | 297 | EVQLVESGGGLVQAGESLRLSCAASGQYFS | b5 | RYTMA- | IISESGGIKEYVDSVKG- | ARDWFARNEYQYDY- | 2 |
| | | TYSMAWFRRTPGKEREFVTIISESGGIKEY | | SEQ ID | SEQ ID | SEQ ID | |
| | | VDSVKGRFTVSRDNAKNTVYLQMNSLKPED | | NO: 1356 | NO: 1357 | NO: 1358 | |
| | | TAVYYCAAARDWFARNEYQYDYWGQGTQVT | | | | | |
| | | VSS- | | | | | |
| | | SEQ ID | | | | | |
| | | NO: 297 | | | | | |
| 539 | 298 | QVQLVESGGGLVQAGGSLRLSCAASGDVFV | b104 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1359 | NO: 1360 | NO: 1361 | |
| | | TAIYYCAANTDYFQIRSLDLNTWGQGTQVT | | | | | |
| | | VSS- | | | | | |
| | | SEQ ID | | | | | |
| | | NO: 298 | | | | | |
| 540 | 299 | QVQLVESGGGLVQAGGSLRLSCAVSGDVFV | b134 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1362 | NO: 1363 | NO: 1364 | |
| | | TAVYYCAANTDYFQIRSLDLNTWGQGTQVT | | | | | |
| | | VSL- | | | | | |
| | | SEQ ID | | | | | |
| | | NO: 299 | | | | | |
| 541 | 300 | EVQLVESGGGLVQAGGSLRLSCAVSGDVFV | b34 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1365 | NO: 1366 | NO: 1367 | |
| | | TAVYYCAANTDYFQIRSLDLNTWGQGTQVT | | | | | |
| | | VSS- | | | | | |
| | | SEQ ID | | | | | |
| | | NO: 300 | | | | | |
| 542 | 301 | QVQLVESGGGLVQAGGSLRLSCAASGDVFV | b105 | RYTMA- | SVTDSGRTTEYVDSVKG- | NTDYFQIRSLNLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1368 | NO: 1369 | NO: 1370 | |
| | | TAVYYCAANTDYFQIRSLNLNTWGQGTQVT | | | | | |
| | | VSS- | | | | | |
| | | SEQ ID | | | | | |
| | | NO: 301 | | | | | |
| 543 | 302 | QVQLQESGGGLVQAGGSLRLSCAASGDVFV | b77 | RYTMA- | SVTDSGRTTEYVDSVKG- | NTDYFQIRSLNLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1371 | NO: 1372 | NO: 1373 | |
| | | TAVYYCAANTDYFQIRSLNLNTWGQGTQVT | | | | | |
| | | VSS- | | | | | |
| | | SEQ ID | | | | | |
| | | NO: 302 | | | | | |
| 544 | 303 | QVQLVESGGGLVQAGGSLRLSCAASGDVFV | b104 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1374 | NO: 1375 | NO: 1376 | |
| | | TAIYYCAANTDYFQIRSLDLNTWGQGTQVT | | | | | |
| | | VSS- | | | | | |
| | | SEQ ID | | | | | |
| | | NO: 303 | | | | | |
| 545 | 304 | EVQLVESGGGLVQAGGSLRLSCAASGDVFV | b13 | RYTMA- | SVTDSGRTTDYVDSVKG- | NTDYFQIKSLDRNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1377 | NO: 1378 | NO: 1379 | |
| | | TAVYYCAANTDYFQIKSLDRNTWGQGTQVT | | | | | |
| | | VSS- | | | | | |
| | | SEQ ID | | | | | |
| | | NO: 304 | | | | | |
| 546 | 305 | QVQLVESGGGLVQAGGSLRLSCAVSGDVLV | b138 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1380 | NO: 1381 | NO: 1382 | |
| | | TAVYYCAANTDYFQIRSLDLNTWGQGTQVT | | | | | |
| | | VSS- | | | | | |
| | | SEQ ID | | | | | |
| | | NO: 305 | | | | | |
| 547 | 306 | EVQLVESGGGLVQAGGSLRLSCAASGDVFV | b15 | RYTMA- | SVTDSGRTTEYVDSVKG- | NTDYFQIRSLRANT- | 20 |
| | | RYTMAWFRQTPGKEREFVASVTDSGRTTEY | | SEQ ID | SEQ ID | SEQ ID | |
| | | VDSVKGRFTVSRDNAKNTVYLQMNNLKPED | | NO: 1383 | NO: 1384 | NO: 1385 | |
| | | TAVYYCAANTDYFQIRSLRANTWGQGTQVT | | | | | |
| | | VSS- | | | | | |
| | | SEQ ID | | | | | |
| | | NO: 306 | | | | | |
| 550 | 307 | QVQLVESGGGLVQAGGSLTLSCAASGRAFS | b147 | RYTMA- | AITWLSGSTLYADSVEG- | ALKTITRGQNDYSY- | 1 |
| | | NYPVGWFRQAPGKEREFVAAITWLSGSTLY | | SEQ ID | SEQ ID | SEQ ID | |
| | | ADSVEGRFTISRDNAKNTVYLLMSSLKPED | | NO: 1386 | NO: 1387 | NO: 1388 | |
| | | TALYFCAAALKTITRGQNDYSYWGQGTQVT | | | | | |
| | | VSS- | | | | | |
| | | SEQ ID | | | | | |
| | | NO: 307 | | | | | |
| 551 | 308 | QVQLVESGGGLVQPGGSLRLSCAASGDVFV | b153 | RYTMA- | SVTDSGRTTDYVDSVKG- | NTDYFQIKSLDRNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNNLKPED | | NO: 1389 | NO: 1390 | NO: 1391 | |
| | | TAVYYCAANTDYFQIKSLDRNTWGQGTQVT | | | | | |
| | | VSS- | | | | | |
| | | SEQ ID | | | | | |
| | | NO: 308 | | | | | |
| 554 | 309 | QVQLVESGGGLVQAGGSLRLSCAASGDVLV | b108 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1392 | NO: 1393 | NO: 1394 | |
| | | TAIYYCAANTDYFQIRSLDLNTWGQGTQVT | | | | | |
| | | VSS- | | | | | |
| | | SEQ ID | | | | | |
| | | NO: 309 | | | | | |
| 555 | 310 | QVQLVESGGGLVQAGGSLRLSCAVSGDVFV | b135 | RYTMA- | SVTDSGRTTEYVDSVKG- | NTDYFQIRSLDLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1395 | NO: 1396 | NO: 1397 | |
| | | TAVYYCAANTDYFQIRSLDLNTWGQGTQVT | | | | | |
| | | VSS- | | | | | |
| | | SEQ ID | | | | | |
| | | NO: 310 | | | | | |
| 559 | 311 | QVQLVESGGGLVQAGGSLRLSCAASGDVFV | b105 | RYTMA- | SVTDSGRTTEYVDSVKG- | NTDYFQIRSLNLNT- | 20 |
| | | RYTMAWFRQAPGKEREFVASVTDSGRTTEY | | SEQ ID | SEQ ID | SEQ ID | |
| | | VDSVKGRFTVSRDNAKNTAYLQMNNLKPED | | NO: 1398 | NO: 1399 | NO: 1400 | |
| | | TAVYYCAANTDYFQIRSLNLNTWGQGTQVT | | | | | |
| | | VSS- | | | | | |
| | | SEQ ID | | | | | |
| | | NO: 311 | | | | | |
| 573 | 312 | QVQLVESGGGLVQAGGSLRLSCAASGDVFV | b100 | RYTMA- | SVTDSGRTTDYVDSVKG- | NTDYFQIKSLDLNT- | 20 |
| | | RYTMAWFRQAPGKEREFVASVTDSGRTTDY | | SEQ ID | SEQ ID | SEQ ID | |
| | | VDSVKGRFTVSRDNAKNTVYLQMNALKPED | | NO: 1401 | NO: 1402 | NO: 1403 | |
| | | TAVYYCAANTDYFQIKSLDLNTWGQGTQVT | | | | | |
| | | VSS- | | | | | |
| | | SEQ ID | | | | | |
| | | NO: 312 | | | | | |

TABLE 9

| | doorlopende | | | | | | | | |
|---|---|---|---|---|---|---|---|---|---|
| | nummering | | | PE binding | | | | | |
| Clone | Patent | | | ELISA | | P.E. Off-rate ranking SPR-mouse and human IL-2Ra | | | |
| nr. | clone ID | V ID | Clone ID | hu IL-2R | mo IL-2R | Rmax-hu | hd(1/s)-hu | Rmax-mo | kd (1/s)-mo |
| 295 | 27 | 27- | FJ1427_P035MP04G01 | 0.362 | 0.048 | 96.63 | 1.31E−03 | 9.52 | N/A |
| | | MP04G01 | | | | | | | |
| 300 | 28 | 28-MP04D02 | FJ1427_P035MP04D02 | 0.782 | 0.054 | 166.11 | 8.80E−03 | 30.94 | 3.19E−01 |
| 304 | 29 | 29-MP04H02 | FJ1427_P035MP04H02 | 0.157 | 0.050 | 104.01 | 6.25E−03 | 10.31 | N/A |
| 307 | 30 | 30-MP04C03 | FJ1427_P035MP04C03 | 1.488 | 0.048 | 442.69 | 8.44E−03 | 11.53 | N/A |
| 309 | 31 | 31-MP04E03 | FJ1427_P035MP04E03 | 0.706 | 0.495 | 169.31 | 8.43E−02 | 27.1 | 2.83E−01 |
| 345 | 32 | 32-MP04A08 | FJ1427_P035MP04A08 | 0.407 | 0.050 | 256.29 | 6.16E−02 | 9.53 | N/A |
| 355 | 33 | 33-MP04C09 | FJ1427_P035MP04C09 | 1.690 | 0.047 | 332.57 | 3.10E−02 | 10.13 | N/A |
| 377 | 34 | 34-MP04A12 | FJ1427_P035MP04A12 | 1.299 | 3.599 | 323.31 | 3.33E−04 | 342.33 | 8.09E−03 |
| 583 | 35 | 35- | FJ1427_P035MP07G01 | 0.134 | 0.045 | −1.24 | N/A | 5.31 | N/A |
| | | MP07G01 | | | | | | | |
| 590 | 36 | 36-MP07F02 | FJ1427_P035MP07F02 | 0.896 | 0.060 | 63.96 | 3.31E−03 | 112.27 | 7.47E−02 |
| 646 | 37 | 37-MP07F09 | FJ1427_P035MP07F09 | 1.806 | 1.487 | 97.14 | 1.26E−03 | 309.78 | 7.00E−03 |
| 657 | 38 | 38-MP07A11 | FJ1427_P035MP07A11 | 1.307 | 0.173 | 59.76 | 2.35E−03 | 215.77 | 1.49E−02 |
| 289 | 313 | | FJ1427_P035MP04A01 | 0.867 | 0.060 | 282.7 | 1.56E−03 | 78.22 | 1.32E−01 |
| 290 | 314 | | FJ1427_P035MP04B01 | 0.873 | 0.074 | 157.01 | 3.27E−03 | 53.81 | 1.96E−01 |
| 291 | 315 | | FJ1427_P035MP04C01 | 0.264 | 0.049 | 82.41 | 1.24E−03 | 8.6 | N/A |
| 292 | 316 | | FJ1427_P035MP04D01 | 1.024 | 0.066 | 278.79 | 1.82E−03 | 121.22 | 1.13E−01 |
| 293 | 317 | | FJ1427_P035MP04E01 | 1.078 | 0.067 | 188.27 | 2.66E−03 | 112.7 | 1.09E−01 |
| 294 | 318 | | FJ1427_P035MP04F01 | 1.656 | 0.085 | 195.73 | 2.68E−03 | 130.44 | 1.10E−01 |
| 296 | 319 | | FJ1427_P035MP04H01 | 1.665 | 0.109 | 264.66 | 2.06E−03 | 143.64 | 1.04E−01 |
| 297 | 320 | | FJ1427_P035MP04A02 | 1.450 | 0.085 | 177.95 | 3.06E−03 | 120.28 | 1.26E−01 |
| 298 | 321 | | FJ1427_P035MP04B02 | 1.969 | 0.048 | 409.02 | 2.85E−02 | 8.18 | N/A |
| 299 | 322 | | FJ1427_P035MP04C02 | 1.002 | 0.070 | 245.73 | 1.96E−03 | 113.3 | 1.26E−01 |
| 301 | 323 | | FJ1427_P035MP04E02 | 1.980 | 0.049 | 407.94 | 6.52E−03 | 9.4 | N/A |
| 302 | 324 | | FJ1427_P035MP04F02 | 0.196 | 0.049 | 64.5 | 2.60E−03 | 9.4 | N/A |
| 303 | 325 | | FJ1427_P035MP04G02 | 0.980 | 0.066 | 313.74 | 1.48E−03 | 107.94 | 1.14E−01 |
| 305 | 326 | | FJ1427_P035MP04A03 | 1.869 | 0.052 | 375.6 | 7.12E−03 | 9.12 | N/A |
| 306 | 327 | | FJ1427_P035MP04B03 | 1.373 | 0.048 | 373.15 | 4.21E−02 | 9.07 | N/A |
| 308 | 328 | | FJ1427_P035MP04D03 | 1.662 | 0.063 | 271.44 | 1.43E−03 | 51.2 | 1.68E−01 |
| 310 | 329 | | FJ1427_P035MP04F03 | 1.771 | 0.300 | 280.73 | 7.40E−04 | 192.73 | 6.10E−02 |
| 311 | 330 | | FJ1427_P035MP04G03 | 0.933 | 0.049 | 346.95 | 7.44E−03 | 9.04 | N/A |
| 312 | 331 | | FJ1427_P035MP04H03 | 1.254 | 0.442 | 159.47 | 1.00E−03 | 212.46 | 2.97E−02 |
| 313 | 332 | | FJ1427_P035MP04A04 | 1.307 | 0.050 | 370.31 | 3.78E−02 | 8.97 | N/A |
| 315 | 333 | | FJ1427_P035MP04C04 | 1.432 | 0.063 | 153.37 | 2.14E−03 | 67.05 | 1.51E−01 |
| 317 | 334 | | FJ1427_P035MP04E04 | 1.725 | 0.048 | 350.75 | 7.96E−03 | 9.48 | N/A |
| 319 | 335 | | FJ1427_P035MP04G04 | 1.672 | 0.276 | 270.25 | 1.22E−03 | 205.91 | 6.66E−02 |
| 322 | 336 | | FJ1427_P035MP04B05 | 1.489 | 0.063 | 139.55 | 2.92E−03 | 66.97 | 1.38E−01 |
| 323 | 337 | | FJ1427_P035MP04C05 | 1.810 | 0.047 | 376.56 | 7.75E−03 | 9.66 | N/A |
| 328 | 338 | | FJ1427_P035MP04H05 | 1.569 | 0.080 | 199.1 | 2.73E−03 | 127.82 | 1.08E−01 |
| 329 | 339 | | FJ1427_P035MP04A06 | 0.898 | 0.063 | 187.84 | 2.83E−03 | 118.49 | 1.13E−01 |
| 330 | 340 | | FJ1427_P035MP04B06 | 1.445 | 0.048 | 409.59 | 2.08E−02 | 11.37 | N/A |
| 331 | 341 | | FJ1427_P035MP04C06 | 0.797 | 0.061 | 222.95 | 2.01E−03 | 70.8 | 4.65E−02 |
| 332 | 342 | | FJ1427_P035MP04D06 | 2.099 | 0.637 | 181.62 | 1.45E−03 | 202.27 | 1.37E−02 |
| 334 | 343 | | FJ1427_P035MP04F06 | 1.409 | 0.077 | 146.16 | 2.98E−03 | 106.98 | 1.16E−01 |
| 337 | 344 | | FJ1427_P035MP04A07 | 1.898 | 0.049 | 404.72 | 1.96E−02 | 11.4 | N/A |
| 338 | 345 | | FJ1427_P035MP04B07 | 1.938 | 0.047 | 365.49 | 1.87E−02 | 10.21 | N/A |
| 341 | 346 | | FJ1427_P035MP04E07 | 0.919 | 0.079 | 298.94 | 1.89E−03 | 69.38 | 2.15E−01 |
| 342 | 347 | | FJ1427_P035MP04F07 | 0.789 | 0.064 | 195.88 | 2.49E−03 | 64.64 | 1.56E−01 |
| 343 | 348 | | FJ1427_P035MP04G07 | 0.863 | 0.049 | 364.63 | 9.10E−03 | 12.59 | N/A |
| 344 | 349 | | FJ1427_P035MP04H07 | 0.919 | 0.076 | 289.33 | 1.54E−03 | 132.57 | 1.01E−01 |
| 346 | 350 | | FJ1427_P035MP04B08 | 1.441 | 0.075 | 251 | 2.05E−03 | 127.43 | 1.13E−01 |
| 348 | 351 | | FJ1427_P035MP04D08 | 0.579 | 0.048 | 344.95 | 1.95E−02 | 11.46 | N/A |
| 349 | 352 | | FJ1427_P035MP04E08 | 1.228 | 0.078 | 261.72 | 1.97E−03 | 115.24 | 1.22E−01 |
| 351 | 353 | | FJ1427_P035MP04G08 | 1.836 | 0.049 | 390.91 | 8.92E−03 | 9.87 | N/A |
| 352 | 354 | | FJ1427_P035MP04H08 | 1.298 | 0.053 | 339.74 | 4.80E−02 | 10.92 | N/A |
| 353 | 355 | | FJ1427_P035MP04A09 | 1.231 | 0.049 | 279.47 | 5.76E−02 | 9.51 | N/A |
| 354 | 356 | | FJ1427_P035MP04B09 | 1.544 | 0.083 | 355.43 | 1.46E−03 | 134.71 | 1.22E−01 |
| 356 | 357 | | FJ1427_P035MP04D09 | 1.928 | 0.048 | 411.82 | 2.07E−02 | 13.61 | N/A |
| 357 | 358 | | FJ1427_P035MP04E09 | 0.652 | 0.070 | 278.01 | 1.62E−03 | 97.04 | 1.09E−01 |
| 358 | 359 | | FJ1427_P035MP04F09 | 1.774 | 0.049 | 394.46 | 7.95E−03 | 10.55 | N/A |
| 359 | 360 | | FJ1427_P035MP04G09 | 0.559 | 0.050 | 259.1 | 6.58E−02 | 11.22 | N/A |
| 360 | 361 | | FJ1427_P035MP04H09 | 1.871 | 0.050 | 385.95 | 2.22E−02 | 12.05 | N/A |
| 361 | 362 | | FJ1427_P035MP04A10 | 1.846 | 0.048 | 383.76 | 2.04E−02 | 10.63 | N/A |
| 362 | 363 | | FJ1427_P035MP04B10 | 1.474 | 0.047 | 348.83 | 7.49E−03 | 10.52 | N/A |
| 363 | 364 | | FJ1427_P035MP04C10 | 1.335 | 0.047 | 406.12 | 2.05E−02 | 10.49 | N/A |
| 364 | 365 | | FJ1427_P035MP04D10 | 1.263 | 0.077 | 364.55 | 1.39E−03 | 124.4 | 1.20E−01 |
| 366 | 366 | | FJ1427_P035MP04F10 | 0.751 | 0.048 | 306.49 | 1.47E−02 | 8.01 | N/A |
| 367 | 367 | | FJ1427_P035MP04G10 | 0.839 | 0.049 | 353.65 | 1.04E−02 | 11.45 | N/A |
| 368 | 368 | | FJ1427_P035MP04H10 | 1.804 | 0.049 | 406.1 | 9.98E−03 | 12.25 | N/A |
| 369 | 369 | | FJ1427_P035MP04A11 | 0.593 | 0.050 | 371.26 | 1.65E−02 | 12.01 | N/A |
| 370 | 370 | | FJ1427_P035MP04B11 | 0.750 | 0.047 | 343.62 | 1.03E−02 | 10.16 | N/A |
| 371 | 371 | | FJ1427_P035MP04C11 | 0.912 | 0.047 | 319.23 | 6.93E−02 | 10.94 | N/A |
| 372 | 372 | | FJ1427_P035MP04D11 | 1.411 | 0.048 | 295.74 | 4.01E−02 | 11.15 | N/A |
| 373 | 373 | | FJ1427_P035MP04E11 | 0.940 | 0.065 | 362.54 | 1.49E−03 | 113.67 | 1.17E−01 |
| 374 | 374 | | FJ1427_P035MP04F11 | 2.008 | 0.049 | 386.1 | 1.11E−02 | 10.84 | N/A |
| 375 | 375 | | FJ1427_P035MP04G11 | 1.396 | 0.048 | 356.15 | 1.16E−02 | 11.91 | N/A |
| 376 | 376 | | FJ1427_P035MP04H11 | 1.774 | 0.049 | 272.2 | 1.03E−02 | 13.42 | N/A |
| 378 | 377 | | FJ1427_P035MP04B12 | 0.936 | 0.074 | 98.96 | 3.44E−03 | 125.8 | 1.16E−01 |
| 379 | 378 | | FJ1427_P035MP04C12 | 1.684 | 0.048 | 372.74 | 7.32E−03 | 11.68 | N/A |
| 380 | 379 | | FJ1427_P035MP04D12 | 1.051 | 0.054 | 392.49 | 7.20E−03 | 9.89 | N/A |
| 381 | 380 | | FJ1427_P035MP04E12 | 1.333 | 0.051 | 403.48 | 6.88E−03 | 9.73 | N/A |
| 382 | 381 | | FJ1427_P035MP04F12 | 2.024 | 0.049 | 397.28 | 7.14E−03 | 9.91 | N/A |
| 577 | 382 | | FJ1427_P035MP07A01 | 0.478 | 0.045 | 191.09 | 1.51E−03 | 38.78 | 1.18E−01 |
| 578 | 383 | | FJ1427_P035MP07B01 | 0.518 | 0.049 | 165.79 | 2.35E−03 | 83.09 | 1.00E−01 |
| 579 | 384 | | FJ1427_P035MP07C01 | 0.945 | 0.923 | 129.88 | 1.13E−03 | 296.03 | 6.94E−03 |
| 580 | 385 | | FJ1427_P035MP07D01 | 1.191 | 0.051 | 108.22 | 2.94E−03 | 103.53 | 1.13E−01 |
| 581 | 386 | | FJ1427_P035MP07E01 | 0.830 | 0.139 | 73.58 | 1.56E−03 | 162.58 | 1.32E−02 |
| 582 | 387 | | FJ1427_P035MP07F01 | 0.556 | 0.047 | 102.03 | 2.97E−03 | 79.2 | 8.55E−02 |
| 584 | 388 | | FJ1427_P035MP07H01 | 0.818 | 0.133 | 135.33 | 1.25E−03 | 154.75 | 2.74E−02 |
| 585 | 389 | | FJ1427_P035MP07A02 | 0.829 | 0.063 | 96.66 | 1.76E−03 | 199.59 | 1.16E−02 |
| 586 | 390 | | FJ1427_P035MP07B02 | 1.324 | 0.061 | 71.34 | 2.32E−03 | 153.09 | 1.51E−02 |
| 587 | 391 | | FJ1427_P035MP07C02 | 0.480 | 0.044 | 386.82 | 1.86E−02 | 11.07 | N/A |
| 588 | 392 | | FJ1427_P035MP07D02 | 0.599 | 0.057 | 111.68 | 1.52E−03 | 199.85 | 1.27E−02 |
| 589 | 393 | | FJ1427_P035MP07E02 | 0.620 | 0.061 | 88.48 | 2.06E−03 | 212.67 | 1.32E−02 |
| 591 | 394 | | FJ1427_P035MP07G02 | 1.220 | 0.051 | 86.45 | 3.65E−03 | 80.49 | 1.00E−01 |
| 592 | 395 | | FJ1427_P035MP07H02 | 0.801 | 0.066 | 106.45 | 1.88E−03 | 215.31 | 1.23E−02 |
| 593 | 396 | | FJ1427_P035MP07A03 | 1.691 | 0.869 | 66.93 | 1.74E−03 | 216.69 | 7.95E−03 |
| 594 | 397 | | FJ1427_P035MP07B03 | 1.552 | 0.058 | 48.01 | 2.78E−03 | 81.25 | 1.32E−02 |
| 595 | 398 | | FJ1427_P035MP07C03 | 0.982 | 0.046 | 71.75 | 3.53E−03 | 48.29 | 1.14E−01 |
| 596 | 399 | | FJ1427_P035MP07D03 | 1.247 | 0.046 | 84.13 | 3.39E−03 | 58.38 | 1.07E−01 |
| 597 | 400 | | FJ1427_P035MP07E03 | 0.445 | 0.048 | 111.6 | 3.11E−03 | 92.04 | 9.66E−02 |
| 598 | 401 | | FJ1427_P035MP07F03 | 0.497 | 0.046 | 103.9 | 3.21E−03 | 69.3 | 1.05E−01 |
| 599 | 402 | | FJ1427_P035MP07G03 | 0.495 | 0.048 | 113.65 | 3.09E−03 | 76.89 | 9.90E−02 |
| 601 | 403 | | FJ1427_P035MP07A04 | 0.507 | 0.046 | 130.63 | 1.26E−03 | 44.97 | 1.49E−01 |
| 602 | 404 | | FJ1427_P035MP07B04 | 0.970 | 0.047 | 121.03 | 2.77E−03 | 54.41 | 1.14E−01 |
| 603 | 405 | | FJ1427_P035MP07C04 | 0.406 | 0.045 | 153.69 | 2.67E−03 | 94.18 | 9.28E−02 |
| 604 | 406 | | FJ1427_P035MP07D04 | 0.792 | 0.046 | 99.59 | 2.43E−03 | 44.76 | 1.61E−01 |
| 605 | 407 | | FJ1427_P035MP07E04 | 0.729 | 0.046 | 146.95 | 2.59E−03 | 76.19 | 1.03E−01 |
| 606 | 408 | | FJ1427_P035MP07F04 | 0.400 | 0.047 | 128.1 | 2.96E−03 | 53.51 | 1.15E−01 |
| 607 | 409 | | FJ1427_P035MP07G04 | 1.017 | 0.047 | 135.81 | 2.81E−03 | 50.92 | 1.12E−01 |
| 608 | 410 | | FJ1427_P035MP07H04 | 0.593 | 0.052 | 142.73 | 1.77E−03 | 97.39 | 8.23E−02 |
| 609 | 411 | | FJ1427_P035MP07A05 | 0.973 | 0.046 | 105.7 | 3.23E−03 | 48.45 | 1.27E−01 |
| 610 | 412 | | FJ1427_P035MP07B05 | 0.439 | 0.045 | 139.96 | 2.82E−03 | 83.18 | 1.10E−01 |
| 611 | 413 | | FJ1427_P035MP07C05 | 0.479 | 0.047 | 113.52 | 1.82E−03 | 139.59 | 1.28E−02 |
| 612 | 414 | | FJ1427_P035MP07D05 | 0.733 | 0.054 | 132.87 | 1.53E−03 | 159.41 | 6.09E−02 |
| 613 | 415 | | FJ1427_P035MP07E05 | 0.434 | 0.045 | 84.27 | 3.72E−03 | 52.18 | 1.20E−01 |
| 614 | 416 | | FJ1427_P035MP07F05 | 0.405 | 0.045 | 101.39 | 3.23E−03 | 53 | 1.16E−01 |
| 615 | 417 | | FJ1427_P035MP07G05 | 0.450 | 0.046 | 88.5 | 2.57E−03 | 32.76 | 1.91E−01 |
| 616 | 418 | | FJ1427_P035MP07H05 | 1.141 | 0.049 | 134.27 | 2.79E−03 | 97.11 | 1.18E−01 |
| 617 | 419 | | FJ1427_P035MP07A06 | 1.119 | 0.047 | 134.97 | 2.73E−03 | 69.04 | 1.09E−01 |
| 618 | 420 | | FJ1427_P035MP07B06 | 0.902 | 0.045 | 119.08 | 3.22E−03 | 51.58 | 1.12E−01 |
| 619 | 421 | | FJ1427_P035MP07C06 | 0.319 | 0.045 | 133.96 | 2.79E−03 | 60.36 | 1.11E−01 |
| 620 | 422 | | FJ1427_P035MP07D06 | 0.919 | 0.045 | 143.54 | 2.88E−03 | 58.87 | 1.26E−01 |
| 621 | 423 | | FJ1427_P035MP07E06 | 0.464 | 0.046 | 169.4 | 2.72E−03 | 99.58 | 1.02E−01 |
| 622 | 424 | | FJ1427_P035MP07F06 | 1.343 | 0.082 | 162.47 | 1.62E−03 | 239.55 | 1.17E−02 |
| 625 | 425 | | FJ1427_P035MP07A07 | 1.039 | 0.047 | 194.33 | 1.73E−03 | 66.11 | 9.71E−02 |
| 626 | 426 | | FJ1427_P035MP07B07 | 0.290 | 0.045 | 128.36 | 2.59E−03 | 57.11 | 3.47E−03 |
| 627 | 427 | | FJ1427_P035MP07C07 | 0.469 | 0.047 | 145.52 | 2.50E−03 | 64.9 | 1.05E−01 |
| 629 | 428 | | FJ1427_P035MP07E07 | 0.359 | 0.047 | 74.37 | 2.25E−03 | 60.14 | 9.27E−02 |
| 630 | 429 | | FJ1427_P035MP07F07 | 0.372 | 0.045 | 91.37 | 3.00E−03 | 47.67 | 1.33E−01 |
| 631 | 430 | | FJ1427_P035MP07G07 | 0.479 | 0.045 | 128.46 | 2.73E−03 | 90.68 | 1.06E−01 |
| 632 | 431 | | FJ1427_P035MP07H07 | 1.415 | 0.047 | 80.51 | 4.18E−03 | 21.68 | 3.31E−03 |
| 633 | 432 | | FJ1427_P035MP07A08 | 0.889 | 0.049 | 143.26 | 2.55E−03 | 80.83 | 1.06E−01 |
| 634 | 433 | | FJ1427_P035MP07B08 | 0.425 | 0.044 | 89.12 | 3.26E−03 | 32.8 | 1.44E−01 |
| 635 | 434 | | FJ1427_P035MP07C08 | 0.528 | 0.049 | 153.91 | 2.59E−03 | 92.76 | 1.08E−01 |
| 636 | 435 | | FJ1427_P035MP07D08 | 1.031 | 0.050 | 159.87 | 2.63E−03 | 118.36 | 9.91E−02 |
| 637 | 436 | | FJ1427_P035MP07E08 | 0.642 | 0.049 | 109.34 | 2.29E−03 | 73.2 | 1.30E−01 |
| 638 | 437 | | FJ1427_P035MP07F08 | 1.241 | 0.052 | 130.42 | 1.85E−03 | 92.37 | 1.00E−01 |
| 639 | 438 | | FJ1427_P035MP07G08 | 0.504 | 0.047 | 124.62 | 3.00E−03 | 87.05 | 1.05E−01 |
| 640 | 439 | | FJ1427_P035MP07H08 | 0.510 | 0.047 | 127.29 | 3.03E−03 | 78.98 | 1.00E−01 |
| 641 | 440 | | FJ1427_P035MP07A09 | 0.518 | 0.055 | 129.97 | 1.61E−03 | 181.98 | 1.19E−02 |
| 642 | 441 | | FJ1427_P035MP07B09 | 1.268 | 0.082 | 119.23 | 1.68E−03 | 213.73 | 1.27E−02 |
| 643 | 442 | | FJ1427_P035MP07C09 | 0.723 | 0.755 | 104.15 | 1.37E−03 | 338.19 | 7.03E−03 |
| 644 | 443 | | FJ1427_P035MP07D09 | 0.865 | 0.045 | 57.07 | 4.47E−03 | 45.2 | 1.26E−01 |
| 645 | 444 | | FJ1427_P035MP07E09 | 0.428 | 0.045 | 89.11 | 3.30E−03 | 74.19 | 1.08E−01 |
| 647 | 445 | | FJ1427_P035MP07G09 | 1.144 | 0.047 | 61.25 | 2.40E−03 | 75.21 | 1.01E−01 |
| 648 | 446 | | FJ1427_P035MP07H09 | 1.111 | 0.049 | 79.96 | 3.46E−03 | 100.7 | 1.35E−01 |
| 649 | 447 | | FJ1427_P035MP07A10 | 0.759 | 0.053 | 80 | 2.42E−03 | 103.16 | 7.67E−02 |
| 650 | 448 | | FJ1427_P035MP07B10 | 0.716 | 0.059 | 87.5 | 1.75E−03 | 138.94 | 5.01E−02 |
| 651 | 449 | | FJ1427_P035MP07C10 | 0.424 | 0.074 | 77.95 | 1.76E−03 | 229.75 | 1.32E−02 |
| 652 | 450 | | FJ1427_P035MP07D10 | 0.734 | 0.048 | 53.81 | 3.00E−03 | 68.81 | 1.25E−01 |
| 653 | 451 | | FJ1427_P035MP07E10 | 0.434 | 0.047 | 93.39 | 3.30E−03 | 85.46 | 1.07E−01 |
| 654 | 452 | | FJ1427_P035MP07F10 | 0.670 | 0.089 | 64.41 | 1.99E−03 | 164.14 | 1.21E−02 |
| 655 | 453 | | FJ1427_P035MP07G10 | 0.682 | 0.740 | 96.71 | 1.22E−03 | 325.7 | 6.82E−03 |
| 656 | 454 | | FJ1427_P035MP07H10 | 0.572 | 0.049 | 54.04 | 2.36E−03 | 126.54 | 1.37E−02 |
| 658 | 455 | | FJ1427_P035MP07B11 | 1.246 | 0.246 | 54.36 | 2.14E−03 | 229.6 | 1.14E−02 |
| 659 | 456 | | FJ1427_P035MP07C11 | 0.858 | 0.046 | 62.86 | 4.85E−03 | 63.59 | 1.68E−01 |
| 661 | 457 | | FJ1427_P035MP07E11 | 0.398 | 0.045 | 107.25 | 3.04E−03 | 88.26 | 1.09E−01 |
| 662 | 458 | | FJ1427_P035MP07F11 | 0.983 | 0.686 | 107.78 | 1.31E−03 | 321.3 | 7.57E−03 |
| 663 | 459 | | FJ1427_P035MP07G11 | 0.999 | 0.046 | 77.9 | 3.51E−03 | 75.72 | 9.90E−02 |
| 664 | 460 | | FJ1427_P035MP07H11 | 1.316 | 0.057 | 70.12 | 2.52E−03 | 101.05 | 6.60E−02 |
| 665 | 461 | | FJ1427_P035MP07A12 | 0.485 | 0.052 | 67.93 | 2.70E−03 | 92.95 | 7.48E−02 |
| 666 | 462 | | FJ1427_P035MP07B12 | 1.789 | 0.700 | 61.23 | 1.53E−03 | 197.54 | 7.66E−03 |
| 667 | 463 | | FJ1427_P035MP07C12 | 0.426 | 0.071 | 64.81 | 1.97E−03 | 190.43 | 1.46E−02 |
| 668 | 464 | | FJ1427_P035MP07D12 | 0.511 | 0.051 | 62.42 | 1.99E−03 | 141.1 | 1.22E−02 |
| 669 | 465 | | FJ1427_P035MP07E12 | 0.956 | 0.050 | 60.15 | 3.05E−03 | 84.92 | 8.83E−02 |
| 670 | 466 | | FJ1427_P035MP07F12 | 0.823 | 0.341 | 46.09 | 1.97E−03 | 193.35 | 7.92E−03 |

| | doorlopende | | | | | | |
|---|---|---|---|---|---|---|---|
| | nummering | | | | | | |
| Clone | Patent clone | Sequence analysis | | | | | CDR3 |
| nr. | ID | VH | VH ID | CDR1 | CDR2 | CDR3 | Family ID |
| 295 | 27 | QVQLVESGGGLVQAGGSLTLSCAAPGRTFGTDVVGWFRQAPGKE | 5_c | TDVVG-SEQ | SISRSGDGIYY | GDGWSTYDY-SEQ | 4 |
| | | REFVASISRSGDGIYYDDSVKGRFTISRNNAWNTVNLQMNSLKVED | | ID NO: 545 | DDSVKG-SEQ | ID NO: 547 | |
| | | TAVYYCAAGDGWSTYDYWGQGTQVTVSS-SEQ ID NO: 27 | | | ID NO: 546 | | |
| 300 | 28 | QVQLVESGGGLVQAGGSLRLSCAASGRTLSRYAMGWFRQAPGKE | 7_c | RYAMG- | ANSWGGDTY | APTSFATTAYSGSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSGSNSYAYWGQGTQVTVSS-SEQ ID | | 548 | SEQ ID NO: | 550 | |
| | | NO: 28 | | | 549 | | |
| 304 | 29 | QVQLVESGGGLVQAGGSLRLACVASGLTFDNYYMGWFRQAPGKE | 9_c | NYYMG- | GIIWNGDHTA | TFWIERATTPDIGQ | 7 |
| | | REFVAGIIWNGDHTAYADSIKGRFTISRDNAKNTAYLRMNSLKPEDT | | SEQ ID NO: | YADSIKG- | YAY-SEQ ID NO: | |
| | | AVYYCAATFWIERATTPDIGQYAYWGQGTQVTVSS-SEQ ID NO: 29 | | 551 | SEQ ID NO: | 553 | |
| | | | | | 552 | | |
| 307 | 30 | EVQLVESGGGWVQDGGSLRLSCALSGRTFVRGIMGWFRQAPGKE | 12_c | RGIMG-SEQ | RIIWHINSTRY | RDRYGSGNSLSPS | 6 |
| | | REFVARIIWHINSTRYADSVKGRFTISRDSAKNTMYLQMDSLRPEDT | | ID NO: 554 | ADSVKG-SEQ | AYDY-SEQ ID NO: | |
| | | AVYYCAARDRYGSGNSLSPSAYDYWGQGTQVTVSS-SEQ ID NO: | | | ID NO: 555 | 556 | |
| | | 30 | | | | | |
| 309 | 31 | QVQLVESGGGLVQAGGSLRLSCTGYGGAFTGYALGWFRQAPGKE | 14_c | GYALG-SEQ | RINWSGSFTY | DNPSTLATDYDN- | 2 |
| | | REFVARINWSGSFTYYASSVKGRFTISRDNAKNTMYLQMNNLKPED | | ID NO: 557 | YASSVKG- | SEQ ID NO: 559 | |
| | | TAVYYCAADNPSTLATDYDNWGQGTQVTVSS-SEQ ID NO: 31 | | | SEQ ID NO: | | |
| | | | | | 558 | | |
| 345 | 32 | QVQLVESGGGLVQAGGSLRLSCAASGRTFGSTAVGWFRQVPGKE | 31_c | STAVG-SEQ | AINRSGSATTY | DSLPYGRPYYFQRS | 3 |
| | | REFVSAINRSGSATTYADSVKGRFTISRDNAKNTVYLQMNSLTPEDT | | ID NO: 560 | ADSVKG-SEQ | AGEYDY-SEQ ID | |
| | | GVYYCAADSLPYGRPYYFQRSAGEYDYWGQGTQVTVSS-SEQ ID | | | ID NO: 561 | NO: 562 | |
| | | NO: 32 | | | | | |
| 355 | 33 | QLQLVESGGGLVQAGGSLRLSCAASGPTFSRVAVGWFRQAPGKER | 36_c | RVAVG-SEQ | AVNRPATMTK | DSVPYGRPYYWQT | 3 |
| | | EFVAAVNRPATMTKYADSVKGRFTVSRDNAKNTVDLQMNSMKPED | | ID NO: 563 | YADSVKG- | SAGDYDY-SEQ ID | |
| | | TAVYYCAADSVPYGRPYYWQTSAGDYDYWGQGTQVTVSS-SEQ | | | SEQ ID NO: | NO: 565 | |
| | | ID NO: 33 | | | 564 | | |
| 377 | 34 | QVQLVESGGGLVQAGSSLRLSCAASGRTLSRLAMGWFRQAPGKER | 53_c | RLAMG- | VNSWGGDTF | APTSFATTAYSSSN | 1 |
| | | EFVAVNSWGGDTFYADSVEGRFTYSRDNAKSAVYLQMNSLQPEDT | | SEQ ID NO: | YADSVEG- | SYAY-SEQ ID NO: | |
| | | AVYYCAAAPTSFATTAYSSSNSYAYWGQGAQVTVSS-SEQ ID NO: | | 566 | SEQ ID NO: | 568 | |
| | | 34 | | | 567 | | |
| 583 | 35 | QVQLQESGGGLVQGGGSLRLSCAASGGIFSSYAMGWFRQAPGKE | 61_c | SYAMG- | AISRSGRSTN | GRYYNSAYDPSPG | 5 |
| | | REFVAAISRSGRSTNYADSVKGRFTISRDNAKSTVYLQMNSLKPEET | | SEQ ID NO: | YADSVKG- | DFGS-SEQ ID NO: | |
| | | AVYYCAAGRYYNSAYDPSPGDFGSWGHGTQVTVSS-SEQ ID NO: | | 569 | SEQ ID NO: | 571 | |
| | | 35 | | | 570 | | |
| 590 | 36 | QVQLVESGGGLVQAGGSLRLSCAASGRTLSRYAMGWFRQAPGSE | 66_c | RYAMG- | ASSWGGDTF | APTSFPTTAYSSSN | 1 |
| | | REFVAASSWGGDTFYADSVEGRFTFSRDNAKNAVYLQMNSLQPED | | SEQ ID NO: | YADSVEG- | SYAY-SEQ ID NO: | |
| | | TAAYYCAAAPTSFPTTAYSSSNSYAYWGQGTQVTVSS-SEQ ID NO: | | 572 | SEQ ID NO: | 574 | |
| | | 36 | | | 573 | | |
| 646 | 37 | QVQLVESGGGLVQAGGSLRLSCAASGRTLSRYAMGWFRQAPGKE | 85_c | RYAMG- | IDSWGGDTFY | APTSFATTAYSSSN | 1 |
| | | REYVAIDSWGGDTFYADSVEGRFTFSRDNAKNEVYLQMNSLQPED | | SEQ ID NO: | ADSVEG-SEQ | SYRY-SEQ ID NO: | |
| | | TAVYYCAGAPTSFATTAYSSSNSYRYWGQGTQVTVSS-SEQ ID | | 575 | ID NO: 576 | 577 | |
| | | NO: 37 | | | | | |
| 657 | 38 | QVQLVESGGGLVQAGGSLRLSCAASGRSLSRDAMGWFRQAPGKE | 90_c | RDAMG- | VMSWGGDTF | APTSFATTAYSSSN | 1 |
| | | REFVAVMSWGGDTFYTDSVEGRFTFSRDNAKNAVYLEMNDLQPED | | SEQ ID NO: | YTDSVEG- | SYSY-SEQ ID NO: | |
| | | TAVYYCAAAPTSFATTAYSSSNSYSYWGRGTQVTVSS-SEQ ID NO: | | 578 | SEQ ID NO: | 580 | |
| | | 38 | | | 579 | | |
| 289 | 313 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1405 | SEQ ID NO: | 1407 | |
| | | 313 | | | 1406 | | |
| 290 | 314 | EVQLVESGGGLVQAGASLRLSCAASGRTLSRYAMGWFRQAPGKER | 2_c | RYAMG- | ASSWGGDTY | APTSFATTAYSSSN | 1 |
| | | EFVAASSWGGDTYYADSVEGRFTFSRDNAKNAVYLQMNSLQPEDT | | SEQ ID NO: | YADSVEG- | SYAY-SEQ ID NO: | |
| | | AVYYCAGAPTSFATTAYSSSNSYAYWGQGTQVTVSS-SEQ ID NO: | | 1408 | SEQ ID NO: | 1410 | |
| | | 314 | | | 1409 | | |
| 291 | 315 | QVQLVESGGGLVQAGGSLTLSCAASGRTFSTDVVGWFRQAPGKER | 3_c | TDVVG-SEQ | SISRSGDGIYY | GDGWSTYDY-SEQ | 4 |
| | | EFVASISRSGDGIYYDDSVKGRFTISRNNAWNTVNLQMNNLKVEDT | | ID NO: 1411 | DDSVKG-SEQ | ID NO: 1413 | |
| | | AVYYCAAGDGWSTYDYWGQGTQVTVSS-SEQ ID NO: 315 | | | ID NO: 1412 | | |
| 292 | 316 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1414 | SEQ ID NO: | 1416 | |
| | | 316 | | | 1415 | | |
| 293 | 317 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1417 | SEQ ID NO: | 1419 | |
| | | 317 | | | 1418 | | |
| 294 | 318 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1420 | SEQ ID NO: | 1422 | |
| | | 318 | | | 1421 | | |
| 296 | 319 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG-SEQ | SYAY-SEQ ID NO: | |
| | | TAVYYCAAAPTSFATTAYSSSNSYAYWGQGTQVTVSS-SEQ ID NO: | | 1423 | ID NO: 1424 | 1425 | |
| | | 319 | | | | | |
| 297 | 320 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1426 | SEQ ID NO: | 1428 | |
| | | 320 | | | 1427 | | |
| 298 | 321 | QVQLVESGGGWVQVGESLRLSCAPSGRTFVSGIMGWFRQTPGKE | 6_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1429 | TDSVKG-SEQ | AYDY-SEQ ID NO: | |
| | | AVYYCAARDRYGSGNSLSPSAYDYWGQGTQVTVSS-SEQ ID NO: | | | ID NO: 1430 | 1431 | |
| | | 321 | | | | | |
| 299 | 322 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1432 | SEQ ID NO: | 1434 | |
| | | 322 | | | 1433 | | |
| 301 | 323 | QVQLVESGGGWVQVGESLRLSCAPSGRTFVSGIMGWFRQTPGKE | 6_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1435 | TDSVKG-SEQ | AYDY-SEQ ID NO: | |
| | | AVYYCAARDRYGSGNSLSPSAYDYWGQGTQVTVSS-SEQ ID NO: | | | ID NO: 1436 | 1437 | |
| | | 323 | | | | | |
| 302 | 324 | QLQLVESGGGLVQAGGSLTLSCAASGRTFSRDVVGWFRQAPGKER | 8_c | RDVVG- | SISRSGDGTF | GDGWSTYDY-SEQ | 4 |
| | | EFVASISRSGDGTFYTDSVKGRFTISSRDNAKNTVLLQMNSLKVEDT | | SEQ ID NO: | YTDSVKG- | ID NO: 1440 | |
| | | AVYYCAAGDGWSTYDYWGQGTQVTVSS-SEQ ID NO: 324 | | 1438 | SEQ ID NO: | | |
| | | | | | 1439 | | |
| 303 | 325 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1441 | SEQ ID NO: | 1443 | |
| | | 325 | | | 1442 | | |
| 305 | 326 | EVQLVESGGGWVQVGESLRLSCAPSGRTFVSGIMGWFRQTPGKE | 10_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1444 | TDSVKG-SEQ | AYDY-SEQ ID NO: | |
| | | AVYYCAARDRYGSGNSLSPSAYDYWGQGTQVTVSS-SEQ ID NO: | | | D NO: 1445 | 1446 | |
| | | 326 | | | | | |
| 306 | 327 | QVQLQESGGGLVQAGESLRLSCAASGPTASRVAVAWFRQVPGKER | 11_c | RVAVA-SEQ | VVNRPGTMTK | DSVPYGRPYYFQTS | 3 |
| | | EFVAVVNRPGTMTKYADSVKGRFTISRDSAKNTVYLQMNSLKPEDT | | ID NO: 1447 | YADSVKG- | AGNYDY-SEQ ID | |
| | | AVYYCAADSVPYGRPYYFQTSAGNYDYWGQGTQVTVSS-SEQ ID | | | SEQ ID NO: | NO: 1449 | |
| | | NO: 327 | | | 1448 | | |
| 308 | 328 | EVQLVESGGGLVQAEGSLRLSCAASGRTLSRYAMGWFRQAPGKER | 13_c | RYAMG- | ANSWGGDTY | APTSFATTAYSSSN | 1 |
| | | EFVTANSWGGDTYYADSVQGRFTFSRDNAKNTVYLQMNSLQPEDT | | SEQ ID NO: | YADSVQG- | SYAY-SEQ ID NO: | |
| | | AVYYCAAAPTSFATTAYSSSNSYAYWGQGTQVTVSS-SEQ ID NO: | | 1450 | SEQ ID NO: | 1452 | |
| | | 328 | | | 1451 | | |
| 310 | 329 | QVQLVESGGGLVQAGGSLRLSCAASGRTLSRNAMGWFRQAPGKE | 15_c | RNAMG- | ADSWGGDTF | APTSFATTAYSSSN | 1 |
| | | REFVAADSWGGDTFYADSVEGRFTFSRDNAKNAVYLQMNSLQPED | | SEQ ID NO: | YADSVEG- | SYRY-SEQ ID NO: | |
| | | TAVYYCAGAPTSFATTAYSSSNSYRYWGQGTQVTVSS-SEQ ID | | 1453 | SEQ ID NO: | 1455 | |
| | | NO: 329 | | | 1454 | | |
| 311 | 330 | EVQLVESGGGWVQVGESLRLSCAPSGRTFVSGIMGWFRQAPGKE | 16_c | SGIMG-SEQ | RIILNNNSTRY | RDRYGSGNSLSPS | 6 |
| | | REFVARIILNNNSTRYTDSVKGRFTISRDSAKNTMYLQMDSLRPEDT | | ID NO: 1456 | TDSVKG-SEQ | AYDY-SEQ ID NO: | |
| | | AVYYCAARDRYGSGNSLSPSAYDYWGQGTQVTVSS-SEQ ID NO: | | | ID NO: 1457 | 1458 | |
| | | 330 | | | | | |
| 312 | 331 | QLQLVESGGGLVQAGGSLRLSCAASGRTLSRYAMGWFRQAPGKE | 17_c | RYAMG- | ADSWGGDTF | APTSFATTAYSSSN | 1 |
| | | REFVAADSWGGDTFYADSVEGRFTFSRDNAKNAAYLQMNSLQPED | | SEQ ID NO: | YADSVEG- | SYRY-SEQ ID NO: | |
| | | TATYYCAGAPTSFATTAYSSSNSYRYWGQGTQVTVSS-SEQ ID | | 1459 | SEQ ID NO: | 1461 | |
| | | NO: 331 | | | 1460 | | |
| 313 | 332 | QVQLVESGGGLVQAGESLRLSCAASGPTASRVAVAWFRQVPGKER | 18_c | RVAVA-SEQ | VVNRPGTMTK | DSVPYGRPYYFQTS | 3 |
| | | EFVAVVNRPGTMTKYADSVKGRFTISRDSAKNTVYLQMNSLKPEDT | | ID NO: 1462 | YADSVKG- | AGNYDY-SEQ ID | |
| | | AVYYCAADSVPYGRPYYFQTSAGNYDYWGQGTQVTVSS-SEQ ID | | | SEQ ID NO: | NO: 1464 | |
| | | NO: 332 | | | 1463 | | |
| 315 | 333 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1465 | SEQ ID NO: | 1467 | |
| | | 333 | | | 1466 | | |
| 317 | 334 | EVQLVESGGGWVQVGESLRLSCVPSGRTFVSGIMGWFRQAPGKE | 19_c | SGIMG-SEQ | RIILNSNSTRY | RDRYGSGNSLSPS | 6 |
| | | REFVARIILNSNSTRYTDSVKGRFTISRDSAKNTMYLQMNSLRPEDT | | ID NO: 1468 | TDSVKG-SEQ | AYDY-SEQ ID NO: | |
| | | AVYYCAARDRYGSGNSLSPSAYDYWGQGTQVTVSS-SEQ ID NO: | | | ID NO: 1469 | 1470 | |
| | | 334 | | | | | |
| 319 | 335 | QVQLVESGGGLVQAGGSLRLSCAASGRTLSRYAVGWFRQAPGKE | 20_c | RYAVG-SEQ | ANSWGGDTF | APTSFATTAYSSSN | 1 |
| | | REFVTANSWGGDTFYADSVQGRFTFSRDNAKNTVYLQMNSLLPED | | ID NO: 1471 | YADSVQG- | SYKV-SEQ ID NO: | |
| | | AAVYYCAAAPTSFATTAYSSSNSYKVWGQGTQVTVSS-SEQ ID NO: | | | SEQ ID NO: | 1473 | |
| | | 335 | | | 1472 | | |
| 322 | 336 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1474 | SEQ ID NO: | 1476 | |
| | | 336 | | | 1475 | | |
| 323 | 337 | QVQLVESGGGWVQVGESLRLSCAPSGRTFVSGIMGWFRQAPGKE | 22_c | SGIMG-SEQ | RIILNNNSTRY | RDRYGSGNSLSPS | 6 |
| | | REFVARIILNNNSTRYTDSVKGRFTISRDSAKDTMYLQMNTLRPEDT | | ID NO: 1477 | TDSVKG-SEQ | AYDY-SEQ ID NO: | |
| | | AVYYCAARDRYGSGNSLSPSAYDYWGQGTQVTVSS-SEQ ID NO: | | | ID NO: 1478 | 1479 | |
| | | 337 | | | | | |
| 328 | 338 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1480 | SEQ ID NO: | 1482 | |
| | | 338 | | | 1481 | | |
| 329 | 339 | QVQLVESGGGLVQPGGSLRLSCAASGRTLSRYAMGWFRQAPGKE | 23_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1483 | SEQ ID NO: | 1485 | |
| | | 339 | | | 1484 | | |
| 330 | 340 | QLQLVESGGGWVQVGESLRLSCVPSGRTFVSGIMGWFRQAPGKE | 24_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1486 | TDSVKG-SEQ | AYDY-SEQ ID NO: | |
| | | AVYYCAARDRYGSGNSLSPSAYDYWGQGTQVTVSS-SEQ ID NO: | | | ID NO: 1487 | 1488 | |
| | | 340 | | | | | |
| 331 | 341 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1489 | SEQ ID NO: | 1491 | |
| | | 341 | | | 1490 | | |
| 332 | 342 | QVQLVESGGGLVQAGGSLRLSCAASGRALSRLAMGWFRQAPGKE | 25_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1492 | ID NO: 1493 | 1494 | |
| | | 342 | | | | | |
| 334 | 343 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1495 | SEQ ID NO: | 1497 | |
| | | 343 | | | 1496 | | |
| 337 | 344 | QLQLVESGGGWVQVGESLRLSCAPSGRTFVSGIMGWFRQAPGKE | 26_c | SGIMG-SEQ | RIILNNNSTRYI | RDRYGSGNSLSPS | 6 |
| | | REFVARIILNNNSTRYIDSVKGRFTISRDSAKNTMYLQMDSLRPEDTA | | ID NO: 1498 | DSVKG-SEQ | AYDY-SEQ ID NO: | |
| | | VYYCAARDRYGSGNSLSPSAYDYWGQGTQVTVSS-SEQ ID NO: | | | ID NO: 1499 | 1500 | |
| | | 344 | | | | | |
| 338 | 345 | EVQLVESGGGWVQVGESLRLSCAPSGRTFVSGIMGWFRQTPGKE | 27_c | SGIMG-SEQ | RIILNNNSTRY | RDRYGSGNSLSPS | 6 |
| | | REFVARIILNNNSTRYTDSVKGRFTISRDSAKNTLYLQMNTLRPEDTA | | ID NO: 1501 | TDSVKG-SEQ | AYDY-SEQ ID NO: | |
| | | VYYCAARDRYGSGNSLSPSAYDYWGQGTQVTVSS-SEQ ID NO: | | | ID NO: 1502 | 1503 | |
| | | 345 | | | | | |
| 341 | 346 | QVQLVESGGGLVQAGGSLRLSCAASGRTLSRYAMGWFRQAPGKE | 28_c | RYAMG- | ATSWGGDTY | APTSFATTAYSSSN | 1 |
| | | RDFVAATSWGGDTYYADSVEGRFTFSRDNAKNAVYLQMNSLQPED | | SEQ ID NO: | YADSVEG- | SYAY-SEQ ID NO: | |
| | | TAVYYCAAAPTSFATTAYSSSNSYAYWGQGTQVTVSS-SEQ ID NO: | | 1504 | SEQ ID NO: | 1506 | |
| | | 346 | | | 1505 | | |
| 342 | 347 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1507 | SEQ ID NO: | 1509 | |
| | | 347 | | | 1508 | | |
| 343 | 348 | QVQLVESGGGWVQVGESLRLSCAPSGRTFVSGIMGWFRQAPGKE | 29_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1510 | TDSVKG-SEQ | AYDY-SEQ ID NO: | |
| | | AVYYCAARDRYGSGNSLSPSAYDYWGQGTQVTVSS-SEQ ID NO: | | | ID NO: 1511 | 1512 | |
| | | 348 | | | | | |
| 344 | 349 | QLQLVESGGGLVQAGGSLRLSCAASGRTLSRYAMGWFRQAPGKE | 30_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1513 | SEQ ID NO: | 1515 | |
| | | 349 | | | 1514 | | |
| 346 | 350 | QVQLVESGGGLVQAGGSLRLSCVASGRTLSRYAMGWFRQAPGKE | 32_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1516 | SEQ ID NO: | 1518 | |
| | | 350 | | | 1517 | | |
| 348 | 351 | QVQLVESGGGLVQAGESLRLSCAASGPTASRVAVAWFRQVPGKER | 18_c | RVAVA-SEQ | VVNRPGTMTK | DSVPYGRPYYFQTS | 3 |
| | | EFVAVVNRPGTMTKYADSVKGRFTISRDSAKNTVYLQMNSLKPEDT | | ID NO: 1519 | YADSVKG- | AGNYDY-SEQ ID | |
| | | AVYYCAADSVPYGRPYYFQTSAGNYDYWGQGTQVTVSS-SEQ ID | | | SEQ ID NO: | NO: 1521 | |
| | | NO: 351 | | | 1520 | | |
| 349 | 352 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1522 | SEQ ID NO: | 1524 | |
| | | 352 | | | 1523 | | |
| 351 | 353 | QVQLVESGGGWVQVGESLRLSCAPSGRTFVSGIMGWFRQAPGKE | 33_c | SGIMG-SEQ | RIILNNNSTRY | RDRYGSGNSLSPS | 6 |
| | | REFVARIILNNNSTRYTDSVKGRFTISRDSAKNTMYLQMDSLRPEDT | | ID NO: 1525 | TDSVKG-SEQ | AYDY-SEQ ID NO: | |
| | | AVYYCAARDRYGSGNSLSPSAYDYWGQGTQVTVSS-SEQ ID NO: | | | ID NO: 1526 | 1527 | |
| | | 353 | | | | | |
| 352 | 354 | EVQLVESGGGLVQAGESLRLSCAASGPTASRVAVAWFRQVPGKER | 34_c | RVAVA-SEQ | VVNRPGTMTK | DSVPYGRPYYFQTS | 3 |
| | | EFVAVVNRPGTMTKYADSVKGRFTISRDSAKNTVYLQMNSLKPEDT | | ID NO: 1528 | YADSVKG- | AGNYDY-SEQ ID | |
| | | AVYYCAADSVPYGRPYYFQTSAGNYDYWGQGTQVTVSS-SEQ ID | | | SEQ ID NO: | NO: 1530 | |
| | | NO: 354 | | | 1529 | | |
| 353 | 355 | QVQLVESGGGLVQAGGSLRLSCAASGRTFGAVAVGWFRQVPGKE | 35_c | AVAVG-SEQ | AINRNGSATK | DSLPYGRPYYFQTS | 3 |
| | | REFVAAINRNGSATKYADSAKGRFTISRDNAESPVYLQMNSLKPEDT | | ID NO: 1531 | YADSAKG- | AGEYDY-SEQ ID | |
| | | AIYYCAADSLPYGRPYYFQTSAGEYDYWGQGTQVTVSS-SEQ ID | | | SEQ ID NO: | NO: 1533 | |
| | | NO: 355 | | | 1532 | | |
| 354 | 356 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1534 | SEQ ID NO: | 1536 | |
| | | 356 | | | 1535 | | |
| 356 | 357 | QVQLVESGGGWVQVGESLRLSCAPSGRTFVRGIMGWFRQAPGKE | 37_c | RGIMG-SEQ | RIILNSNSTRYI | RDRYGSGNSLSPS | 6 |
| | | REFVARIILNSNSTRYIDSVKGRFTISRDSAKNTMYLQMNSLRPEDTA | | ID NO: 1537 | DSVKG-SEQ | AYDY-SEQ ID NO: | |
| | | VYYCAARDRYGSGNSLSPSAYDYWGQGTQVTVSS-SEQ ID NO: | | | ID NO: 1538 | 1539 | |
| | | 357 | | | | | |
| 357 | 358 | QVQLVESGGGLVQAGGSLRLSCAASGRTLSRYAMGWFRQAPGKE | 38_c | RYAMG- | ANSWGGDTY | APTSFATTAYSSSN | 1 |
| | | REFVTANSWGGDTYYADSVQGRSTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1540 | SEQ ID NO: | 1542 | |
| | | 358 | | | 1541 | | |
| 358 | 359 | QVQLVESGGGWVQVGESLRLSCAPSGRTFVRGIMGWFRQAPGKE | 39_c | RGIMG-SEQ | RIILNNNSTRY | RDRYGSGNSLSPS | 6 |
| | | REFVARIILNNNSTRYTDSVKGRFTISRDSAKNTMWLQMDSLRPEDT | | ID NO: 1543 | TDSVKG-SEQ | AYDY-SEQ ID NO: | |
| | | AVYYCAARDRYGSGNSLSPSAYDYWGQGTQVTVSS-SEQ ID NO: | | | ID NO: 1544 | 1545 | |
| | | 359 | | | | | |
| 359 | 360 | QVQLQESGGGLVQAGGSLRLSCAASGPTFSRVAVGWFRQVPGKE | 40_c | RVAVG-SEQ | AVNRPATMTK | DSVPYGRPYYFQTS | 3 |
| | | REFVAAVNRPATMTKYADSVKGRFTISRDNAKNTVDLQMNSMKPED | | ID NO: 1546 | YADSVKG- | AGEYDY-SEQ ID | |
| | | TAVYYCAADSVPYGRPYYFQTSAGEYDYWGQGTQVTVSS-SEQ ID | | | SEQ ID NO: | NO: 1548 | |
| | | NO: 360 | | | 1547 | | |
| 360 | 361 | EVQLVESGGGWVQVGESLRLSCAPSGRTFVSGIMGWFRQAPGKE | 41_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1549 | TDSVKG-SEQ | AYDY-SEQ ID NO: | |
| | | AVYYCAARDRYGSGNSLSPSAYDYWGQGTQVTVSS-SEQ ID NO: | | | ID NO: 1550 | 1551 | |
| | | 361 | | | | | |
| 361 | 362 | EVQLVESGGGWVQVGESLRLSCAPSGRTFVSGIMGWFRQAPGKE | 41_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1552 | TDSVKG-SEQ | AYDY-SEQ ID NO: | |
| | | AVYYCAARDRYGSGNSLSPSAYDYWGQGTQVTVSS-SEQ ID NO: | | | ID NO: 1553 | 1554 | |
| | | 362 | | | | | |
| 362 | 363 | EVQLVESGGGWVQVGESLRLSCAPSGRTFVSGIMGWFRQAPGKE | 42_c | SGIMG-SEQ | RIILNNNSTRYI | RDRYGSGNSLSPS | 6 |
| | | REFVARIILNNNSTRYIDSVKGRFTISRDSAKNTMYLQMDSLRPEDTA | | ID NO: 1555 | DSVKG-SEQ | AYDY-SEQ ID NO: | |
| | | VYYCAARDRYGSGNSLSPSAYDYWGQGTQVTVSL-SEQ ID NO: | | | ID NO: 1556 | 1557 | |
| | | 363 | | | | | |
| 363 | 364 | QVQLQESGGGWVQVGESLRLSCVPSGRTFVSGIMGWFRQAPGKE | 43_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1558 | TDSVKG-SEQ | AYDY-SEQ ID NO: | |
| | | AVYYCAARDRYGSGNSLSPSAYDYWGQGTQVTVSS-SEQ ID NO: | | | ID NO: 1559 | 1560 | |
| | | 364 | | | | | |
| 364 | 365 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1561 | SEQ ID NO: | 1563 | |
| | | 365 | | | 1562 | | |
| 366 | 366 | QLQLVESGGGWVQVGESLRLSCVPSGRTFVSGIMGWFRQAPGKE | 44_c | SGIMG-SEQ | RIISNNNSTRY | RDRYGSGNSLSPS | 6 |
| | | REFVARIISNNNSTRYTDSVKGRFTISRDSAKNTMYLQMNTLRPEDT | | ID NO: 1564 | TDSVKG-SEQ | AYDY-SEQ ID NO: | |
| | | AVYYCAARDRYGSGNSLSPSAYDYWGQGTQVTVSS-SEQ ID NO: | | | ID NO: 1565 | 1566 | |
| | | 366 | | | | | |
| 367 | 367 | QVQLVESGGGWVQVGESLRLSCAPSGRTFVSGIMGWFRQAPGKE | 45_c | SGIMG-SEQ | RIILNSNSTRY | RDRYGSGNSLSPS | 6 |
| | | REFVARIILNSNSTRYTDSAKGRFTISRDSAKNTMYLQMDSLRPEDT | | ID NO: 1567 | TDSAKG-SEQ | AYDY-SEQ ID NO: | |
| | | AVYYCAARDRYGSGNSLSPSAYDYWGQGTQVTVSS-SEQ ID NO: | | | ID NO: 1568 | 1569 | |
| | | 367 | | | | | |
| 368 | 368 | QVQLVESGGGLVQAGGSLRLSCAPSGRTFVSGIMGWFRQAPGKER | 46_c | SGIMG-SEQ | RIILNNNSTRYI | RDRYGSGNSLSPS | 6 |
| | | EFVARIILNNNSTRYIDSVKGRFTISRDSAKNTMYLQMDSLRPEDTAV | | ID NO: 1570 | DSVKG-SEQ | AYDY-SEQ ID NO: | |
| | | YYCAARDRYGSGNSLSPSAYDYWGQGTQVTVSS-SEQ ID NO: 368 | | | ID NO: 1571 | 1572 | |
| 369 | 369 | QVQLVESGGGLVQAGESLRLSCAASGPTASRVAVAWFRQVPGKER | 18_c | RVAVA-SEQ | VVNRPGTMTK | DSVPYGRPYYFQTS | 3 |
| | | EFVAVVNRPGTMTKYADSVKGRFTISRDSAKNTVYLQMNSLKPEDT | | ID NO: 1573 | YADSVKG- | AGNYDY-SEQ ID | |
| | | AVYYCAADSVPYGRPYYFQTSAGNYDYWGQGTQVTVSS-SEQ ID | | | SEQ ID NO: | NO: 1575 | |
| | | NO: 369 | | | 1574 | | |
| 370 | 370 | EVQLVESGGGWVQVGESLRLSCAPSGRTFVSGIMGWFRQAPGKE | 47_c | SGIMG-SEQ | RIILNNNSTRYI | RDRYGSGNSLSPS | 6 |
| | | REFVARIILNNNSTRYIDSVKGRFTISRDSAKNTMYLQMDSLRPEDTA | | ID NO: 1576 | DSVKG-SEQ | AYDY-SEQ ID NO: | |
| | | VYYCAARDRYGSGNSLSPSAYDYWGQGTQVTVSS-SEQ ID NO: | | | ID NO: 1577 | 1578 | |
| | | 370 | | | | | |
| 371 | 371 | QVQLVESGGGLVQHGGSLRLSCAASGPTFSRVAVGWFRQVPGKE | 48_c | RVAVG-SEQ | VVNRPGSMTK | DSVPYGRPYYFTTS | 3 |
| | | REFVAVVNRPGSMTKYADSVKGRFNISRDTAKNTVYLQMNSLKPED | | ID NO: 1579 | YADSVKG- | AGDYDY-SEQ ID | |
| | | TAVYYCAADSVPYGRPYYFTTSAGDYDYWGQGTQVTVSS-SEQ ID | | | SEQ ID NO: | NO: 1581 | |
| | | NO: 371 | | | 1580 | | |
| 372 | 372 | EVQLVESGGGLVQAGGSLRLSCAASGRTFGAVAVGWFRQVPGKE | 49_c | AVAVG-SEQ | AINRSGSATK | DSLPYGRPYYFQTS | 3 |
| | | REFVAAINRSGSATKYADSVKGRFTISRDNAESPVFLQMNSLKPEDT | | ID NO: 1582 | YADSVKG- | AGEYDY-SEQ ID | |
| | | AVYYCAADSLPYGRPYYFQTSAGEYDYWGQGTQVTVSS-SEQ ID | | | SEQ ID NO: | NO: 1584 | |
| | | NO: 372 | | | 1583 | | |
| 373 | 373 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1585 | SEQ ID NO: | 1587 | |
| | | 373 | | | 1586 | | |
| 374 | 374 | QVQLVESGGGWVQVGESLRLSCAPSGRTFVSGIMGWFRQTPGKE | 50_c | SGIMG-SEQ | RIILNNNSTRY | RDRYGSGNSLSPS | 6 |
| | | REFVARIILNNNSTRYTDSVKGRFTISRDSAKNTLYLQMNTLRPEDTA | | ID NO: 1588 | TDSVKG-SEQ | AYDY-SEQ ID NO: | |
| | | VYYCAARDRYGSGNSLSPSAYDYWGQGTQVTVSS-SEQ ID NO: | | | ID NO: 1589 | 1590 | |
| | | 374 | | | | | |
| 375 | 375 | QVQLVESGGGWVQVGESLRLSCAPSGRTFVSGIMGWFRQAPGEE | 51_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1591 | TDSVKG-SEQ | AYDY-SEQ ID NO: | |
| | | AVYYCAARDRYGSGNSLSPSAYDYWGQGTQVTVSS-SEQ ID NO: | | | ID NO: 1592 | 1593 | |
| | | 375 | | | | | |
| 376 | 376 | QVQLVESGGGWVQVGESPRLSCAPSGRTFVSGIMGWFRQTPGKE | 52_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1594 | TDSVKG-SEQ | AYDY-SEQ ID NO: | |
| | | AVYYCAARDRYGSGNSLSPSAYDYWGQGTQVTVSS-SEQ ID NO: | | | ID NO: 1595 | 1596 | |
| | | 376 | | | | | |
| 378 | 377 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1597 | SEQ ID NO: | 1599 | |
| | | 377 | | | 1598 | | |
| 379 | 378 | EVQLVESGGGWVQVGESLRLSCAPSGRTFVSGIMGWFRQAPGKE | 47_c | SGIMG-SEQ | RIILNNNSTRYI | RDRYGSGNSLSPS | 6 |
| | | REFVARIILNNNSTRYIDSVKGRFTISRDSAKNTMYLQMDSLRPEDTA | | ID NO: 1600 | DSVKG-SEQ | AYDY-SEQ ID NO: | |
| | | VYYCAARDRYGSGNSLSPSAYDYWGQGTQVTVSS-SEQ ID NO: | | | ID NO: 1601 | 1602 | |
| | | 378 | | | | | |
| 380 | 379 | QVQLVESGGGWVQVGKSLRLSCAPSGRTFVSGIMGWFRQAPGKE | 54_c | SGIMG-SEQ | RIILNNNSTRYI | RDRYGSGNSLSPS | 6 |
| | | REFVARIILNNNSTRYIDSVKGRFTISRDSAKNTMYLQMDSLRPEDTA | | ID NO: 1603 | DSVKG-SEQ | AYDY-SEQ ID NO: | |
| | | VYYCAARDRYGSGNSLSPSAYDYWGQGTQVTVSS-SEQ ID NO: | | | ID NO: 1604 | 1605 | |
| | | 379 | | | | | |
| 381 | 380 | QLQLVESGGGWVQVGESLRLSCAPSGRTFASGIMGWFRQAPGKE | 55_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1606 | TDSVKG-SEQ | AYDY-SEQ ID NO: | |
| | | AVYYCAARDRYGSGNSLSPSAYDYWGQGTQVTVSS-SEQ ID NO: | | | ID NO: 1607 | 1608 | |
| | | 380 | | | | | |
| 382 | 381 | QVQLQESGGGWVQVGESLRLSCVPSGRTFVSGIMGWFRQAPGKE | 43_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1609 | TDSVKG-SEQ | AYDY-SEQ ID NO: | |
| | | AVYYCAARDRYGSGNSLSPSAYDYWGQGTQVTVSS-SEQ ID NO: | | | ID NO: 1610 | 1611 | |
| | | 381 | | | | | |
| 577 | 382 | EVQLVESGGGLVQAGDSLRLSCAASGRTLSRYAMGWFRQAPGKE | 56_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1612 | SEQ ID NO: | 1614 | |
| | | 382 | | | 1613 | | |
| 578 | 383 | QVQLVESGGGLVQAGGSLRLSCAASGRTLSRYAMGWFRQAPGKE | 57_c | RYAMG- | ANSWGGDTY | APTSFATTAYSSSN | 1 |
| | | REFVTANSWGGDTYYADSVQGRFTFTRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1615 | SEQ ID NO: | 1617 | |
| | | 383 | | | 1616 | | |
| 579 | 384 | QVQLVESGGGLVQAGDSLRLSCVASGRTLSRCAMGWFRKAPGKE | 58_c | RCAMG- | ISSWGGDTFY | APTSFATTAYSSSN | 1 |
| | | REYVAISSWGGDTFYEDSVEGRFTFSRDNAKNTVYLQMNSLRPEDT | | SEQ ID NO: | EDSVEG-SEQ | SYAY-SEQ ID NO: | |
| | | AVYYCAAAPTSFATTAYSSSNSYAYWGQGTQVTVSS-SEQ ID NO: | | 1618 | ID NO: 1619 | 1620 | |
| | | 384 | | | | | |
| 580 | 385 | QVQLVESGGGLVQAGGSLRLSCAASGRTLSRYAMGWFRQAPGKE | 59_c | RYAMG- | ANSWGGDTY | APTSFATTAYSSSN | 1 |
| | | REFVAANSWGGDTYYTDSVEGRFTFSRDNAKNAVYLQMNSLQPGD | | SEQ ID NO: | YTDSVEG- | SYSY-SEQ ID NO: | |
| | | TAVYYCAAAPTSFATTAYSSSNSYSYWGQGTQVTVSS-SEQ ID NO: | | 1621 | SEQ ID NO: | 1623 | |
| | | 385 | | | 1622 | | |
| 581 | 386 | QVQLQESGGGLVQAGGSLRLSCAASGRTLSRYAMGWFRQAPGKE | 60_c | RYAMG- | CDSWGGDTF | APTSFATTAYSSSN | 1 |
| | | REFVACDSWGGDTFYADSVEGRFTFSRDNAKNATYLQMNSLQPED | | SEQ ID NO: | YADSVEG- | SYTV-SEQ ID NO: | |
| | | TAVYYCAGAPTSFATTAYSSSNSYTVWGQGTQVTVSL-SEQ ID NO: | | 1624 | SEQ ID NO: | 1626 | |
| | | 386 | | | 1625 | | |
| 582 | 387 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1627 | SEQ ID NO: | 1629 | |
| | | 387 | | | 1628 | | |
| 584 | 388 | QVQLVESGGGLVQAGGSLRLFCATSGRTLSRYAMGWFRQAPGRE | 62_c | RYAMG- | CDSWGGDTF | APTSFATTAYSSSN | 1 |
| | | REFVACDSWGGDTFYADSVKGRFTFSRDSAKNTVYLQMNSLQPED | | SEQ ID NO: | YADSVKG- | SYAY-SEQ ID NO: | |
| | | TAVYYCAAAPTSFATTAYSSSNSYAYWGQGTQVTVSS-SEQ ID NO: | | 1630 | SEQ ID NO: | 1632 | |
| | | 388 | | | 1631 | | |
| 585 | 389 | QVQLVESGGGLVQAGGSLRLSCAASGRALSRLAMGWFRQAPGKE | 63_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTASS-SEQ ID NO: | | 1633 | ID NO: 1634 | 1635 | |
| | | 389 | | | | | |
| 586 | 390 | EVQLVESGGGLVQAGGSLRLSCAASGRALSRLAMGWFRQAPGKE | 64_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1636 | ID NO: 1637 | 1638 | |
| | | 390 | | | | | |
| 587 | 391 | EVQLVESGGGWVQVGESLRLSCAPSGRTFVSGIMGWFRQTPGKE | 10_c | SGIMG-SEQ | RIILNNNSTRY | RDRYGSGNSLSPS | 6 |
| | | REFVARIILNNNSTRYTDSVKGRFTISRDSAKNTMYLQMNTLRPEDT | | ID NO: 1639 | TDSVKG-SEQ | AYDY-SEQ ID NO: | |
| | | AVYYCAARDRYGSGNSLSPSAYDYWGQGTQVTVSS-SEQ ID NO: | | | ID NO: 1640 | 1641 | |
| | | 391 | | | | | |
| 588 | 392 | QVQLQESGGGLVQAGGSLRLSCAASGRALSRLAMGWFRQAPGKE | 65_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1642 | ID NO: 1643 | 1644 | |
| | | 392 | | | | | |
| 589 | 393 | QVQLVESGGGLVQAGGSLRLSCAASGRALSRLAMGWFRQAPGKE | 25_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1645 | ID NO: 1646 | 1647 | |
| | | 393 | | | | | |
| 591 | 394 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1648 | SEQ ID NO: | 1650 | |
| | | 394 | | | 1649 | | |
| 592 | 395 | QVQLVESGGGLVQAGGSLRLSCAASGRALSRLAMGWFRQAPGKE | 25_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1651 | ID NO: 1652 | 1653 | |
| | | 395 | | | | | |
| 593 | 396 | EVQLVESGGGLVQAGGSLRLSCAASGRTLSRYAMGWFRQAPGKE | 67_c | RYAMG- | IDSWGGDTFY | APTSFATTAYSSSN | 1 |
| | | REYVAIDSWGGDTFYADSVEGRFTFSRDNAKNEVYLQMNSLQPED | | SEQ ID NO: | ADSVEG-SEQ | SYRY-SEQ ID NO: | |
| | | TAVYYCAGAPTSFATTAYSSSNSYRYWGQGTQVTVSS-SEQ ID | | 1654 | ID NO: 1655 | 1656 | |
| | | NO: 396 | | | | | |
| 594 | 397 | EVQLVESGGGLVQAGGSLRLSCAASGRALSRLAMGWFRQAPGKE | 64_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1657 | ID NO: 1658 | 1659 | |
| | | 397 | | | | | |
| 595 | 398 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1660 | SEQ ID NO: | 1662 | |
| | | 398 | | | 1661 | | |
| 596 | 399 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1663 | SEQ ID NO: | 1665 | |
| | | 399 | | | 1664 | | |
| 597 | 400 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1666 | SEQ ID NO: | 1668 | |
| | | 400 | | | 1667 | | |
| 598 | 401 | QVQLVESGGGLVQTGGSLRLSCAASGRTLSRYAMGWFRQAPGKE | 68_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1669 | SEQ ID NO: | 1671 | |
| | | 401 | | | 1670 | | |
| 599 | 402 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1672 | SEQ ID NO: | 1674 | |
| | | 402 | | | 1673 | | |
| 601 | 403 | QVQLVESGGGLVQAGGSLRLSCAASGRTLSRYAMGWFRQGPGKE | 69_c | RYAMG- | ADSWGGDTY | APTSFATTAYSSSN | 1 |
| | | REFVAADSWGGDTYYDDSVEGRFTFSRDITKNAVYLQMNSLQPED | | SEQ ID NO: | YDDSVEG- | SYAY-SEQ ID NO: | |
| | | TAVYYCAAAPTSFATTAYSSSNSYAYWGQGTQVTVSS-SEQ ID NO: | | 1675 | SEQ ID NO: | 1677 | |
| | | 403 | | | 1676 | | |
| 602 | 404 | EVQLVESGGGLVQAGGSLRLSCAASGRTLSRYAMGWFRQAPGKE | 1_c | RYAMG | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1678 | SEQ ID NO: | 1680 | |
| | | 404 | | | 1679 | | |
| 603 | 405 | QVQLVESGGGVVQAGGSLRLSCAASGRTLSRYAMGWFRQAPGKE | 70_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1681 | SEQ ID NO: | 1683 | |
| | | 405 | | | 1682 | | |
| 604 | 406 | EVQLVESGGGLVQAGGSLRLSCAASGRTLSRYAMGWFRQGPGKE | 71_c | RYAMG- | ADSWGGDTY | APTSFATTAYSSSN | 1 |
| | | REFVAADSWGGDTYYDDSVEGRFTFSRDITKNAVYLQMNSLQPED | | SEQ ID | YDDSVEG- | SYAY-SEQ ID NO: | |
| | | TAVYYCAAAPTSFATTAYSSSNSYAYWGQGTQVTVSS-SEQ ID NO: | | NO:1684 | SEQ ID NO: | 1686 | |
| | | 406 | | | 1685 | | |
| 605 | 407 | QVQLVESGGGLVQAGDSLRLSCEASGRTLSRYAMGWFRQAPGKE | 72_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1687 | SEQ ID NO: | 1689 | |
| | | 407 | | | 1688 | | |
| 606 | 408 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1690 | SEQ ID NO: | 1692 | |
| | | 408 | | | 1691 | | |
| 607 | 409 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1693 | SEQ ID NO: | 1695 | |
| | | 409 | | | 1694 | | |
| 608 | 410 | QVQLVESGGGLVQAGGSLRLSCAASGRTLSRYAMGWFRQAPGKE | 73_c | RYAMG- | AMSWGGDTF | APTSFATTAYSSSN | 1 |
| | | REFVAAMSWGGDTFYADSVEGRFTFSRDNAEIAVYLQMNSLQPED | | SEQ ID NO: | YADSVEG- | SYAY-SEQ ID NO: | |
| | | TAVYYCAAAPTSFATTAYSSSNSYAYWGQGTQVTVSH-SEQ ID NO: | | 1696 | SEQ ID NO: | 1698 | |
| | | 410 | | | 1697 | | |
| 609 | 411 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1699 | SEQ ID NO: | 1701 | |
| | | 411 | | | 1700 | | |
| 610 | 412 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1702 | SEQ ID NO: | 1704 | |
| | | 412 | | | 1703 | | |
| 611 | 413 | EVQLVESGGGLVQPGGSLRLSCAASGRALSRLAMGWFRQAPGKE | 74_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1705 | ID NO: 1706 | 1707 | |
| | | 413 | | | | | |
| 612 | 414 | QVQLVESGGGLVQAGGSLRLSCAASGRTLSRNAMGWFRQAPGKE | 15_c | RNAMG- | ADSWGGDTF | APTSFATTAYSSSN | 1 |
| | | REFVAADSWGGDTFYADSVEGRFTFSRDNAKNAVYLQMNSLQPED | | SEQ ID NO: | YADSVEG- | SYRY-SEQ ID NO: | |
| | | TAVYYCAGAPTSFATTAYSSSNSYRYWGQGTQVTVSS-SEQ ID | | 1708 | SEQ ID NO: | 1710 | |
| | | NO: 414 | | | 1709 | | |
| 613 | 415 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1711 | SEQ ID NO: | 1713 | |
| | | 415 | | | 1712 | | |
| 614 | 416 | EVQLVESGGGLVQAGGSLRLSCAASGRTLSRYAMGWFRQAPGRE | 75_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1714 | SEQ ID NO: | 1716 | |
| | | 416 | | | 1715 | | |
| 615 | 417 | QVQLVESGGGLVQAGGSLRLSCAASGRTLSRYAMGWFRQAPGKE | 76_c | RYAMG- | ADSWGGDTY | APTSFATTAYSSSN | 1 |
| | | REFVAADSWGGDTYYADSVEGRFTFSRDNANNAVYLQMNSLQPED | | SEQ ID NO: | YADSVEG- | SYAY-SEQ ID NO: | |
| | | TAVYYCAAAPTSFATTAYSSSNSYAYWGQGTQVTVSS-SEQ ID NO: | | 1717 | SEQ ID NO: | 1719 | |
| | | 417 | | | 1718 | | |
| 616 | 418 | QVQLVESGGGLVQAGGSLRLSCAASGRTLSRYAMGWFRQAPGKE | 59_c | RYAMG- | ANSWGGDTY | APTSFATTAYSSSN | 1 |
| | | REFVAANSWGGDTYYTDSVEGRFTFSRDNAKNAVYLQMNSLQPGD | | SEQ ID NO: | YTDSVEG- | SYSY-SEQ ID NO: | |
| | | TAVYYCAAAPTSFATTAYSSSNSYSYWGQGTQVTVSS-SEQ ID NO: | | 1720 | SEQ ID NO: | 1722 | |
| | | 418 | | | 1721 | | |
| 617 | 419 | QVQLVESGGGLVQPGGSLRLSCAASGRTLSRYAMGWFRQAPGKE | 23_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1723 | SEQ ID NO: | 1725 | |
| | | 419 | | | 1724 | | |
| 618 | 420 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1726 | SEQ ID NO: | 1728 | |
| | | 420 | | | 1727 | | |
| 619 | 421 | QVQLVESGGGLVQAGGSLRLSCAASGRTLSRYAMGWFRQAPGKE | 77_c | RYAMG- | ANSWGGDTY | APTSFATTAYSSSN | 1 |
| | | REFVTANSWGGDTYYADSVQGRFTFSRDNAENTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1729 | SEQ ID NO: | 1731 | |
| | | 421 | | | 1730 | | |
| 620 | 422 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1732 | SEQ ID NO: | 1734 | |
| | | 422 | | | 1733 | | |
| 621 | 423 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1735 | SEQ ID NO: | 1737 | |
| | | 423 | | | 1736 | | |
| 622 | 424 | QVQLVESGGGLVQAGGSLRLSCAASGRALSRLAMGWFRQAPGKE | 25_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1738 | ID NO: 1739 | 1740 | |
| | | 424 | | | | | |
| 625 | 425 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1741 | SEQ ID NO: | 1743 | |
| | | 425 | | | 1742 | | |
| 626 | 426 | QVQLVESGGGLVQAGGSLRLSCAASGRSLSRYAMGWFRQAPGKE | 78_c | RYAMG- | AISWGGDSFY | APTSFPTTAYSSSN | 1 |
| | | REFVAAISWGGDSFYTDSVEGRFTFSRDNAKNAVYLEMNDLQPEDT | | SEQ ID NO: | TDSVEG-SEQ | SYSY-SEQ ID NO: | |
| | | AVYYCAAAPTSFPTTAYSSSNSYSYWGRGTQVTVSS-SEQ ID NO: | | 1744 | ID NO: 1745 | 1746 | |
| | | 426 | | | | | |
| 627 | 427 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1747 | SEQ ID NO: | 1749 | |
| | | 427 | | | 1748 | | |
| 629 | 428 | EVQLVESGGGLVQAGGSLRLSCAASGRTLSRYAMGWFRQAPGSE | 79_c | RYAMG- | ASSWGGDTF | APTSFPTTAYSSSN | 1 |
| | | REFVAASSWGGDTFYADSVEGRFTFSRDNAKNAVYLQMNSLQPED | | SEQ ID NO: | YADSVEG- | SYAY-SEQ ID NO: | |
| | | TAAYYCAAAPTSFPTTAYSSSNSYAYWGQGTQVTVSS-SEQ ID NO: | | 1750 | SEQ ID NO: | 1752 | |
| | | 428 | | | 1751 | | |
| 630 | 429 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1753 | SEQ ID NO: | 1755 | |
| | | 429 | | | 1754 | | |
| 631 | 430 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1756 | SEQ ID NO: | 1758 | |
| | | 430 | | | 1757 | | |
| 632 | 431 | QVQLVESGGGLVQAGDSLRLSCAASGRTLSRYAMGWFRQAPGKE | 80_c | RYAMG- | ATSWGGDSY | APTSFATTAYSSSN | 1 |
| | | REFVAATSWGGDSYYADSVEGRFTFSRDNAKNAVYLQMNSLQPED | | SEQ ID NO: | YADSVEG- | SYAN-SEQ ID NO: | |
| | | TAVYYCAAAPTSFATTAYSSSNSYANWGQGTQVTVSS-SEQ ID NO: | | 1759 | SEQ ID NO: | 1761 | |
| | | 431 | | | 1760 | | |
| 633 | 432 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1762 | SEQ ID NO: | 1764 | |
| | | 432 | | | 1763 | | |
| 634 | 433 | EVQLVESGGGSVQPGGSLRLSCAASGRTLSRYAMGWFRQAPGKE | 81_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1765 | SEQ ID NO: | 1767 | |
| | | 433 | | | 1766 | | |
| 635 | 434 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1768 | SEQ ID NO: | 1770 | |
| | | 434 | | | 1769 | | |
| 636 | 435 | QLQLVESGGGLVQAGGSLRLSCAASGRTLSRYAMGWFRQAPGKE | 30_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1771 | SEQ ID NO: | 1773 | |
| | | 435 | | | 1772 | | |
| 637 | 436 | QVQLVESGGGLVQAGGSLRLSCAASGRTLSRYAMGWFRQAPGKE | 82_c | RYAMG- | ADSWGGDTY | APTSFATTAYSSSN | 1 |
| | | REFVAADSWGGDTYYDDSVEGRFTFSRDIAKNAVYLQMNSLQPED | | SEQ ID NO: | YDDSVEG- | SYAY-SEQ ID NO: | |
| | | TAVYYCAAAPTSFATTAYSSSNSYAYWGQGTQVTVSS-SEQ ID NO: | | 1774 | SEQ ID NO: | 1776 | |
| | | 436 | | | 1775 | | |
| 638 | 437 | QVQLVESGGGLVQAGGSLRLSCAASGRTLSRDAMGWFRQAPGKE | 83_c | RDAMG- | ADSWGGDTF | APTSFATTAYSSSN | 1 |
| | | REFVAADSWGGDTFYADSVEGRFTFSRDNAKNAVYLQMNSLQPED | | SEQ ID NO: | YADSVEG- | SYRY-SEQ ID NO: | |
| | | TAVYYCAGAPTSFATTAYSSSNSYRYWGQGTQVTVSS-SEQ ID | | 1777 | SEQ ID NO: | 1779 | |
| | | NO: 437 | | | 1778 | | |
| 639 | 438 | QVQLVESGGGLVQAGGSLRLSCAASGRTLSRYAMGWFRQAPGRE | 84_0 | RYAMG- | ANSWGGDTY | APTSFATTAYSSSN | 1 |
| | | REFVTANSWGGDTYYADSVQGRFTFSRDNAKNTVYLQMSSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1780 | SEQ ID NO: | 1782 | |
| | | 438 | | | 1781 | | |
| 640 | 439 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1783 | SEQ ID NO: | 1785 | |
| | | 439 | | | 1784 | | |
| 641 | 440 | QVQLVESGGGLVQAGGSLRLSCAASGRALSRLAMGWFRQAPGKE | 25_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1786 | ID NO: 1787 | 1788 | |
| | | 440 | | | | | |
| 642 | 441 | QVQLVESGGGLVQAGGSLRLSCAASGRALSRLAMGWFRQAPGKE | 25_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1789 | ID NO: 1790 | 1791 | |
| | | 441 | | | | | |
| 643 | 442 | QVQLVESGGGLVQAGGSLRLSCAASGRTLSRYAMGWFRQAPGKE | 85_c | RYAMG- | IDSWGGDTFY | APTSFATTAYSSSN | 1 |
| | | REYVAIDSWGGDTFYADSVEGRFTFSRDNAKNEVYLQMNSLQPED | | SEQ ID NO: | ADSVEG-SEQ | SYRY-SEQ ID NO: | |
| | | TAVYYCAGAPTSFATTAYSSSNSYRYWGQGTQVTVSS-SEQ ID | | 1792 | ID NO: 1793 | 1794 | |
| | | NO: 442 | | | | | |
| 644 | 443 | EVQLVESGGGLVQAGGSLRLSCAASGRTLSRYAMGWFRQAPGKE | 1_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1795 | SEQ ID NO: | 1797 | |
| | | 443 | | | 1796 | | |
| 645 | 444 | QVQLQESGGGLVQAGGSLRLSCAASGRTLSRYAMGWFRQAPGKE | 86_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1798 | SEQ ID NO: | 1800 | |
| | | 444 | | | 1799 | | |
| 647 | 445 | QVQLQESGGGLVQAGGSLRLSCAASGRTLSRVAMGWFRQAPGKE | 87_c | RVAMG- | ADSWGGDTF | APASFATTAYSSSN | 1 |
| | | REFVAADSWGGDTFYADSVEGRFTFSRDNAKNAVYLQMNSLQPED | | SEQ ID NO: | YADSVEG- | SYSY-SEQ ID NO: | |
| | | TAVYFCAGAPASFATTAYSSSNSYSYWGQGTQVTVSS-SEQ ID | | 1801 | SEQ ID NO: | 1803 | |
| | | NO: 445 | | | 1802 | | |
| 648 | 446 | QVQLVESGGGLVQAGGSLRLSCAASGRTLSRYAMGWFRQAPGKE | 88_c | RYAMG- | ANSWGGDTY | APTSFATTAYSSSN | 1 |
| | | REFVAANSWGGDTYYTDSVEGRFTFSRDNAKNAVYLQMNSLQPED | | SEQ ID NO: | YTDSVEG- | SYTY-SEQ ID NO: | |
| | | TAVYYCAAAPTSFATTAYSSSNSYTYWGQGTQVTVSS-SEQ ID NO: | | 1804 | SEQ ID NO: | 1806 | |
| | | 446 | | | 1805 | | |
| 649 | 447 | QVQLVESGGGLVQAGGSLRLSCAASGRTLSRYAMGWFRQAPGSE | 66_c | RYAMG- | ASSWGGDTF | APTSFPTTAYSSSN | 1 |
| | | REFVAASSWGGDTFYADSVEGRFTFSRDNAKNAVYLQMNSLQPED | | SEQ ID NO: | YADSVEG- | SYAY-SEQ ID NO: | |
| | | TAAYYCAAAPTSFPTTAYSSSNSYAYWGQGTQVTVSS-SEQ ID NO: | | 1807 | SEQ ID NO: | 1809 | |
| | | 447 | | | 1808 | | |
| 650 | 448 | QVQLVESGGGLVQAGGSLRLSCAASGRTLSRYAMGWFRQAPGKE | 89_c | RYAMG- | ADSWGGDTF | APTSFATTAYSSSN | 1 |
| | | REFVAADSWGGDTFYADSVEGRFTFSRDNAKNAAYLQMNSLQPED | | SEQ ID NO: | YADSVEG- | SYRY-SEQ ID NO: | |
| | | TATYYCAGAPTSFATTAYSSSNSYRYWGQGTQVTVSS-SEQ ID | | 1810 | SEQ ID NO: | 1812 | |
| | | NO: 448 | | | 1811 | | |
| 651 | 449 | QVQLVESGGGLVQAGGSLRLSCAASGRSLSRDAMGWFRQAPGKE | 90_c | RDAMG- | VMSWGGDTF | APTSFATTAYSSSN | 1 |
| | | REFVAVMSWGGDTFYTDSVEGRFTFSRDNAKNAVYLEMNDLQPED | | SEQ ID NO: | YTDSVEG- | SYSY-SEQ ID NO: | |
| | | TAVYYCAAAPTSFATTAYSSSNSYSYWGRGTQVTVSS-SEQ ID NO: | | 1813 | SEQ ID NO: | 1815 | |
| | | 449 | | | 1814 | | |
| 652 | 450 | QVQLVESGGGLVQAGGSLRLSCAASGRTLSRYAMGWFRQAPGKE | 82_c | RYAMG- | ADSWGGDTY | APTSFATTAYSSSN | 1 |
| | | REFVAADSWGGDTYYDDSVEGRFTFSRDIAKNAVYLQMNSLQPED | | SEQ ID NO: | YDDSVEG- | SYAY-SEQ ID NO: | |
| | | TAVYYCAAAPTSFATTAYSSSNSYAYWGQGTQVTVSS-SEQ ID NO: | | 1816 | SEQ ID NO: | 1818 | |
| | | 450 | | | 1817 | | |
| 653 | 451 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1819 | SEQ ID NO: | 1821 | |
| | | 451 | | | 1820 | | |
| 654 | 452 | QVQLQESGGGLVQAGGSLRLSCAASGRTLSRYAMGWFRQAPGKE | 60_c | RYAMG- | CDSWGGDTF | APTSFATTAYSSSN | 1 |
| | | REFVACDSWGGDTFYADSVEGRFTFSRDNAKNATYLQMNSLQPED | | SEQ ID NO: | YADSVEG- | SYTV-SEQ ID NO: | |
| | | TAVYYCAGAPTSFATTAYSSSNSYTVWGQGTQVTVSL-SEQ ID NO: | | 1822 | SEQ ID NO: | 1824 | |
| | | 452 | | | 1823 | | |
| 655 | 453 | QVQLVESGGGLVQAGGSLRLSCAASGRTLSRYAMGWFRQAPGKE | 85_c | RYAMG- | IDSWGGDTFY | APTSFATTAYSSSN | 1 |
| | | REYVAIDSWGGDTFYADSVEGRFTFSRDNAKNEVYLQMNSLQPED | | SEQ ID NO: | ADSVEG-SEQ | SYRY-SEQ ID NO: | |
| | | TAVYYCAGAPTSFATTAYSSSNSYRYWGQGTQVTVSS-SEQ ID | | 1825 | ID NO: 1826 | 1827 | |
| | | NO: 453 | | | | | |
| 656 | 454 | EVQLVESGGGLVQAGGSLRLSCAASGRALSRLAMGWFRQAPGKE | 64_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1828 | ID NO: 1829 | 1830 | |
| | | 454 | | | | | |
| 658 | 455 | QVQLVESGGGLVQAGGSLRLSCAASGRALSRLAMGWFRQAPGKE | 91_c | RLAMG- | ASSWGDDTFY | APTSFATTAYSSSN | 1 |
| | | REFVVASSWGDDTFYADSVEGRFTFSRDNAKSTVYLQMNSLQPGD | | SEQ ID NO: | ADSVEG-SEQ | SYAY-SEQ ID NO: | |
| | | TAVYYCAAAPTSFATTAYSSSNSYAYWGQGTQVTVPS-SEQ ID NO: | | 1831 | ID NO: 1832 | 1833 | |
| | | 455 | | | | | |
| 659 | 456 | QVQLVESGGGLVQAGASLRLSCAASGRTLSRYAMGWFRQAPGKE | 92_c | RYAMG- | ASSWGGDTY | APTSFATTAYSSSN | 1 |
| | | REFVAASSWGGDTYYADSVEGRFTFSRDNAKNAVYLQMNSLQPED | | SEQ ID NO: | YADSVEG- | SYAY-SEQ ID NO: | |
| | | TAVYYCAGAPTSFATTAYSSSNSYAYWGQGTQVTVSS-SEQ ID | | 1834 | SEQ ID NO: | 1836 | |
| | | NO: 456 | | | 1835 | | |
| 661 | 457 | QVQLVESGGGLVQAGGSLRLSCAASGRTLSRYAMGWFRQAPGKE | 4_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1837 | SEQ ID NO: | 1839 | |
| | | 457 | | | 1838 | | |
| 662 | 458 | QVQLVESGGGLVQAGGSLRLSCAASGRTLSRLAMGWFRQAPGKE | 93_c | RLAMG- | VNSWGGDTF | APTSFATTAYSSSN | 1 |
| | | REFVAVNSWGGDTFYADSVEGRFTYSRDNAKSAVYLQMNSLQPED | | SEQ ID NO: | YADSVEG- | SYAY-SEQ ID NO: | |
| | | TAVYYCAAAPTSFATTAYSSSNSYAYWGQGTQVTVSS-SEQ ID NO: | | 1840 | SEQ ID NO: | 1842 | |
| | | 458 | | | 1841 | | |
| 663 | 459 | QVQLQESGGGLVQAGGSLRLSCAASGRTLSRYAMGWFRQAPGKE | 86_c | RYAMG- | ANSWGGDTY | APTSFATTAYSSSN | 1 |
| | | REFVTANSWGGDTYYADSVQGRFTFSRDNAKNTVYLQMNSLQPED | | SEQ ID NO: | YADSVQG- | SYAY-SEQ ID NO: | |
| | | TAVYYCAAAPTSFATTAYSSSNSYAYWGQGTQVTVSS-SEQ ID NO: | | 1843 | SEQ ID NO: | 1845 | |
| | | 459 | | | 1844 | | |
| 664 | 460 | QVQLVESGGGLVQPGGSLRLSCAASGRTLSRYAMGWFRQAPGKD | 94_c | RYAMG- | ASSWGGDTF | APTSFATTAYSSSN | 1 |
| | | REFVAASSWGGDTFYADSVEGRFTFSRDNAENATYLQMNSLQPED | | SEQ ID NO: | YADSVEG- | SYGS-SEQ ID NO: | |
| | | TAVYYCAAAPTSFATTAYSSSNSYGSWGQGTQVTVSS-SEQ ID | | 1846 | SEQ ID NO: | 1848 | |
| | | NO: 460 | | | 1847 | | |
| 665 | 461 | QVQLQESGGGLVQAGGSLRLSCAASGRTLSRYAMGWFRQAPGSE | 95_c | RYAMG- | ASSWGGDTF | APTSFPTTAYSSSN | 1 |
| | | REFVAASSWGGDTFYADSVEGRFTFSRDNAKNAVYLQMNSLQPED | | SEQ ID NO: | YADSVEG- | SYAY-SEQ ID NO: | |
| | | TAAYYCAAAPTSFPTTAYSSSNSYAYWGQGTQVTVSS-SEQ ID NO: | | 1849 | SEQ ID NO: | 1851 | |
| | | 461 | | | 1850 | | |
| 666 | 462 | EVQLVESGGGLVQAGGSLRLSCAASGRTLSRYAMGWFRQAPGKE | 67_c | RYAMG- | IDSWGGDTFY | APTSFATTAYSSSN | 1 |
| | | REYVAIDSWGGDTFYADSVEGRFTFSRDNAKNEVYLQMNSLQPED | | SEQ ID NO: | ADSVEG-SEQ | SYRY-SEQ ID NO: | |
| | | TAVYYCAGAPTSFATTAYSSSNSYRYWGQGTQVTVSS-SEQ ID | | 1852 | ID NO: 1853 | 1854 | |
| | | NO: 462 | | | | | |
| 667 | 463 | QVQLVESGGGLVQAGGSLRLSCAASGRSLSRDAMGWFRQAPGKE | 90_c | RDAMG- | VMSWGGDTF | APTSFATTAYSSSN | 1 |
| | | REFVAVMSWGGDTFYTDSVEGRFTFSRDNAKNAVYLEMNDLQPED | | SEQ ID NO: | YTDSVEG- | SYSY-SEQ ID NO: | |
| | | TAVYYCAAAPTSFATTAYSSSNSYSYWGRGTQVTVSS-SEQ ID NO: | | 1855 | SEQ ID NO: | 1857 | |
| | | 463 | | | 1856 | | |
| 668 | 464 | QVQLVESGGGLVQAGGSLRLSCAASGRALSRLAMGWFRQAPGKE | 25_c | RLAMG- | ASSWGDDTFY | APTSFATTAYSSSN | 1 |
| | | REFVVASSWGDDTFYADSVKGRFTFSRDNAKNTVYLQMNSLQPGD | | SEQ ID NO: | ADSVKG-SEQ | SYAY-SEQ ID NO: | |
| | | TAVYYCAAAPTSFATTAYSSSNSYAYWGQGTQVTVSS-SEQ ID NO: | | 1858 | ID NO: 1859 | 1860 | |
| | | 464 | | | | | |
| 669 | 465 | QVQLVESGGGLVQAGGSLRLSCAASGRTLSRYAMGWFRQAPGKE | 96_c | RYAMG- | ANSWGGDTY | APTSFATTAYSSSN | 1 |
| | | REFVTANSWGGDTYYTDSVEGRFTFSRDNAKNAVYLQMNSLQPED | | SEQ ID NO: | YTDSVEG- | SYTY-SEQ ID NO: | |
| | | TAVYYCAAAPTSFATTAYSSSNSYTYWGQGTQVTVSS-SEQ ID NO: | | 1861 | SEQ ID NO: | 1863 | |
| | | 465 | | | 1862 | | |
| 670 | 466 | EVQLVESGGGLVQAGDSLRLSCVASGRTLSRCAMGWFRKAPGKER | 97_c | RCAMG- | ISSWGGDTFY | APTSFATTAYSSSN | 1 |
| | | EYVAISSWGGDTFYEDSVEGRFTFSRDNAKNTVYLQMNSLRPEDTA | | SEQ ID NO: | EDSVEG-SEQ | SYAY-SEQ ID NO: | |
| | | VYYCAAAPTSFATTAYSSSNSYAYWGQGTQVTVSS-SEQ ID NO: | | 1864 | ID NO: 1865 | 1866 | |
| | | 466 | | | | | |

TABLE 10

| | | | | | | Fold Max signal |
|---|---|---|---|---|---|---|
| | EC50 (nM) | | fold EC50 | Max signal (% pSTAT5+) | | (% pSTAT5+) |
| | Treg | NK | NK/Treg | Treg | NK | Treg/NK |
| IL-2 | 0.001183 | 0.3298 | 279 | 97 | 34 | 3 |
| DC0003 | 0.01177 | 2.592 | 220 | 95 | 20 | 5 |
| (TsVHH-48) | | | | | | |
| DC00039 | 0.0007609 | 0.03149 | 41 | 46 | 4 | 11 |
| DC00040 | 0.01579 | 0.7013 | 44 | 43 | 4 | 10 |
| DC00041 | <0.0001 | 0.1503 | >1503 | 94 | 17 | 5 |
| DC00042 | 0.0001058 | 0.7626 | 7208 | 94 | 21 | 5 |
| DC00043 | 0.000529 | 0.04251 | 80 | 54 | 3 | 16 |
| DC00044 | <0.0001 | 0.4415 | >4415 | 96 | 20 | 5 |
| DC00045 | 0.0001514 | 0.05733 | 379 | 88 | 7 | 12 |
| DC00046 | N/A | N/A | N/A | 25 | 6 | 4 |
| DC00047 | N/A | N/A | N/A | 0 | 0 | 1 |
| DC00048 | <0.0001 | 0.1762 | >1762 | 95 | 26 | 4 |
| DC00049 | 0.003361 | 0.1979 | 59 | 95 | 27 | 4 |
| DC00050 | 0.001344 | 0.0326 | 24 | 56 | 8 | 7 |
| DC00051 | <0.0001 | 0.1912 | >1912 | 96 | 33 | 3 |
| DC00052 | 0.004034 | 0.08119 | 20 | 79 | 9 | 8 |
| DC00053 | 0.0004882 | 0.05702 | 117 | 84 | 13 | 7 |
| DC00055 | <0.0001 | 0.8888 | >8888 | 97 | 34 | 3 |
| DC00057 | 0.002082 | 0.5485 | 263 | 89 | 19 | 5 |
| DC00058 | 0.002496 | 1.866 | 748 | 97 | 34 | 3 |
| DC00059 | 0.0002523 | 0.04008 | 159 | 86 | 14 | 6 |
| DC00060 | 0.003339 | 0.6452 | 193 | 56 | 8 | 7 |
| DC00061 | 0.004271 | 0.165 | 39 | 44 | 5 | 9 |
| DC00062 | <0.0001 | 0.03074 | >307.4 | 93 | 22 | 4 |
| DC00064 | <0.0001 | 0.1863 | >1863 | 94 | 38 | 2 |
| DC00066 | <0.0001 | 0.1782 | >1782 | 96 | 27 | 4 |
| DC00067 | <0.0001 | 0.2175 | >2175 | 95 | 39 | 2 |
| DC00068 | 0.001892 | 0.09357 | 49 | 83 | 14 | 6 |
| DC00069 | 0.001189 | 0.234 | 197 | 87 | 12 | 7 |
| DC00070 | 0.0004972 | 0.0538 | 108 | 80 | 11 | 7 |

TABLE 11

| | | | | | | Fold Max signal |
|---|---|---|---|---|---|---|
| | EC50 (nM) | | fold EC50 | Max signal (% pSTAT5+) | | (% pSTAT5+) |
| | Treg | NK | NK/Treg | Treg | NK | Treg/NK |
| IL-2 | 0.05135 | 0.399 | 46 | 82.8 | 18.2 | 5 |
| TsVHH-48 | 0.001948 | 1.135 | 583 | 64.1 | 8.26 | 8 |
| DC00060 | 0.002151 | 0.7691 | 358 | 19.8 | 2.49 | 8 |
| DC00068 | 0.0004247 | 0.00127 | 3 | 30 | 3.39 | 9 |
| DC00075 | <0.0001 | 0.003197 | >31.97 | 53.1 | 4.31 | 12 |
| DC00076 | <0.0001 | 2.423 | >24230 | 59.4 | 14 | 4 |
| DC00077 | <0.0001 | 0.0001907 | >1.907 | 58.4 | 7.96 | 7 |
| DC00078 | <0.0001 | 0.1948 | >1948 | 67.8 | 14.1 | 5 |
| DC00079 | <0.0001 | 0.09935 | >993.5 | 62.4 | 7.35 | 8 |
| DC00080 | <0.0001 | 0.006477 | >64.77 | 17.8 | 4.42 | 4 |
| DC00081 | <0.0001 | 0.194 | >1940 | 60.9 | 13.5 | 5 |
| DC00082 | 0.0008481 | 0.02268 | 27 | 4.57 | 2.11 | 2 |
| DC00083 | <0.0001 | 0.4958 | >4958 | 64.1 | 13 | 5 |
| DC00085 | 0.03127 | 0.6626 | 21 | 8.49 | 2.19 | 4 |
| DC00086 | 0.004405 | 0.187 | 42 | 44.1 | 4.9 | 9 |
| DC00087 | N/A | 0.009717 | N/A | 4.37 | 1.4 | 3 |
| DC00090 | <0.0001 | 0.5723 | >5723 | 23.5 | 2.67 | 9 |
| DC00091 | <0.0001 | 0.3069 | >3069 | 67.7 | 14.5 | 5 |
| DC00092 | <0.0001 | N/A | N/A | 26.7 | 4.82 | 6 |
| DC00093 | 0.7354 | 0.07519 | 0 | 66.7 | 11.4 | 6 |

TABLE 12

| | |
|---|---|
| DC00009 | QVQLVESGGGLVQAGGSLTLSCAAPGRTFGTDVVGWFRQAPGKEREFVASISRSGDGIYYDDSVKGRFTISRNNAWNTVNLQMNSLK |
| (SEQ ID NO: 2039) | VEDTAVYYCAAGDGWSTYDYWGQGTQVTVSSGGGGSGGGGGGGGSQVQLVESGGGLVQPGGSLRLSCATSGFTFRNNFMSWVR |
| | QAPGKGLEWVSTISYGGESTTYAESVKGRFTISRDNAKNTLYLQMNNLKPEDTAVYYCAKATSYDSIRSGSRGQGTQVTVSSDKTHTC |
| | PPCPAPEAAGGPSVFLFPPKPKDTLMISRTPEVTCVVVDVSHEDPEVKFNWYVDGVEVHNAKTKPREEQYNSTYRVVSVLTVLHQDW |
| | LNGKEYKCKVSNKALGAPIEKTISKAKGQPREPQVYTLPPSRDELTKNQVSLTCLVKGFYPSDIAVEWESNGQPENNYKTTPPVLDSD |
| | GSFFLYSRLTVDKSRWQQGNVFSCSVMHEALHNHYTQKSLSLSPGK |
| DC00010 | EVQLVESGGGLVQTGGSLRLSCAASGGTFSRDAMAWFRQVPGKEREFVALISWSGATTNYADSVKGRFAISRDNGKNTVYLQMNRL |
| (SEQ ID NO: 2040) | KPADTAIYYCAADRRPMGSRSYFEPTEYDDWGQGTQVTVSSGGGGSGGGGSGGGGSQVQLVESGGGLVQAGGSLTLSCAAPGRTFG |
| | TDVVGWFRQAPGKEREFVASISRSGDGIYYDDSVKGRFTISRNNAWNTVNLQMNSLKVEDTAVYYCAAGDGWSTYDYWGQGTQVT |
| | VSSGGGGSGGGGSGGGGSQVQLVESGGGLVQPGGSLRLSCATSGFTFRNNFMSWVRQAPGKGLEWVSTISYGGESTTYAESVKGRFT |
| | ISRDNAKNTLYLQMNNLKPEDTAVYYCAKATSYDSIRSGSRGQGTQVTVSSDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVSNKALGAPIEKTISKAKGQP |
| | REPQVYTLPPSRDELTKNQVSLTCLVKGFYPSDIAVEWESNGQPENNYKTTPPVLDSDGSFFLYSRLTVDKSRWQQGNVFSCSVMHEA |
| | LHNHYTQKSLSLSPGK |
| DC00011 | EVQLVESGGGLVQTGGSLRLSCAASGGTFSRDAMAWFRQVPGKEREFVALISWSGATTNYADSVKGRFAISRDNGKNTVYLQMNRL |
| (SEQ ID NO: 2041) | KPADTAIYYCAADRRPMGSRSYFEPTEYDDWGQGTQVTVSSGGGGSGGGGSGGGGSQVQLVESGGGLVQPGGSLRLSCATSGFTFRN |
| | NFMSWVRQAPGKGLEWVSTISYGGESTTYAESVKGRFTISRDNAKNTLYLQMNNLKPEDTAVYYCAKATSYDSIRSGSRGQGTQVTV |
| | SSGGGGSGGGGSGGGGSQVQLVESGGGLVQAGGSLTLSCAAPGRTFGTDVVGWFRQAPGKEREFVASISRSGDGIYYDDSVKGRFTIS |
| | RNNAWNTVNLQMNSLKVEDTAVYYCAAGDGWSTYDYWGQGTQVTVSSGGGGSGGGGSGGGGSDKTHTCPPCPAPEAAGGPSVFL |
| | FPPKPKDTLMISRTPEVTCVVVDVSHEDPEVKFNWYVDGVEVHNAKTKPREEQYNSTYRVVSVLTVLHQDWLNGKEYKCKVSNKAL |
| | GAPIEKTISKAKGQPREPQVYTLPPSRDELTKNQVSLTCLVKGFYPSDIAVEWESNGQPENNYKTTPPVLDSDGSFFLYSRLTVDKSRW |
| | QQGNVFSCSVMHEALHNHYTQKSLSLSPGK |
| DC00012 | QVQLVESGGGLVQPGGSLRLSCATSGFTFRNNFMSWVRQAPGKGLEWVSTISYGGESTTYAESVKGRFTISRDNAKNTLYLQMNNLK |
| (SEQ ID NO: 2042) | PEDTAVYYCAKATSYDSIRSGSRGQGTQVTVSSGGGGSGGGGSGGGGSQVQLVESGGGLVQAGGSLTLSCAAPGRTFGTDVVGWFR |
| | QAPGKEREFVASISRSGDGIYYDDSVKGRFTISRNNAWNTVNLQMNSLKVEDTAVYYCAAGDGWSTYDYWGQGTQVTVSSGGGGS |
| | GGGGSGGGGSDKTHTCPPCPAPEAAGGPSVFLFPPKPKDTLMISRTPEVTCVVVDVSHEDPEVKFNWYVDGVEVHNAKTKPREEQYN |
| | STYRVVSVLTVLHQDWLNGKEYKCKVSNKALGAPIEKTISKAKGQPREPQVYTLPPSRDELTKNQVSLTCLVKGFYPSDIAVEWESNG |
| | QPENNYKTTPPVLDSDGSFFLYSRLTVDKSRWQQGNVFSCSVMHEALHNHYTQKSLSLSPGK |
| DC00014 | EVQLVESGGGLVQTGGSLRLSCAASGGTFSRDAMAWFRQVPGKEREFVALISWSGATTNYADSVKGRFAISRDNGKNTVYLQMNRL |
| (SEQ ID NO: 2043) | KPADTAIYYCAADRRPMGSRSYFEPTEYDDWGQGTQVTVSSGGGGSGGGGSGGGGSQVQLVESGGGLVQAGGSLTLSCAAPGRTFG |
| | TDVVGWFRQAPGKEREFVASISRSGDGIYYDDSVKGRFTISRNNAWNTVNLQMNSLKVEDTAVYYCAAGDGWSTYDYWGQGTQVT |
| | VSSGGGGSGGGGSGGGGSDKTHTCPPCPAPEAAGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALGAPIEKTISKAKGQPREPQVYTLPPSRDELTKNQVSLTCLVKGFYPSDI |
| | AVEWESNGQPENNYKTTPPVLDSDGSFFLYSRLTVDKSRWQQGNVFSCSVMHEALHNHYTQKSLSLSPGK |
| DC00015 | QVQLVESGGGLVQPGGSLRLSCATSGFTFRNNFMSWVRQAPGKGLEWVSTISYGGESTTYAESVKGRFTISRDNAKNTLYLQMNNLK |
| (SEQ ID NO: 2044) | PEDTAVYYCAKATSYDSIRSGSRGQGTQVTVSSGGGGSGGGGSGGGGSQVQLVESGGGLVQAGGSLTLSCAAPGRTFGTDVVGWFR |
| | QAPGKEREFVASISRSGDGIYYDDSVKGRFTISRNNAWNTVNLQMNSLKVEDTAVYYCAAGDGWSTYDYWGQGTQVTVSSGGGGS |
| | GGGGSGGGGSEVQLVESGGGLVQTGGSLRLSCAASGGTFSRDAMAWFRQVPGKEREFVALISWSGATTNYADSVKGRFAISRDNGK |
| | NTVYLQMNRLKPADTAIYYCAADRRPMGSRSYFEPTEYDDWGQGTQVTVSSGGGGSGGGGSGGGGSDKTHTCPPCPAPEAAGGPSV |
| | FLFPPKPKDTLMISRTPEVTCVVVDVSHEDPEVKFNWYVDGVEVHNAKTKPREEQYNSTYRVVSVLTVLHQDWLNGKEYKCKVSNK |
| | ALGAPIEKTISKAKGQPREPQVYTLPPSRDELTKNQVSLTCLVKGFYPSDIAVEWESNGQPENNYKTTPPVLDSDGSFFLYSRLTVDKSR |
| | WQQGNVFSCSVMHEALHNHYTQKSLSLSPGK |
| DC00016 | QVQLVESGGGLVQAGGSLTLSCAAPGRTFGTDVVGWFRQAPGKEREFVASISRSGDGIYYDDSVKGRFTISRNNAWNTVNLQMNSLK |
| (SEQ ID NO: 2045) | VEDTAVYYCAAGDGWSTYDYWGQGTQVTVSSGGGGSGGGGSGGGGSEVQLVESGGGLVQTGGSLRLSCAASGGTFSRDAMAWFR |
| | QVPGKEREFVALISWSGATTNYADSVKGRFAISRDNGKNTVYLQMNRLKPADTAIYYCAADRRPMGSRSYFEPTEYDDWGQGTQVT |
| | VSSGGGGSGGGGSGGGGSDKTHTCPPCPAPEAAGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALGAPIEKTISKAKGQPREPQVYTLPPSRDELTKNQVSLTCLVKGFYPSDI |
| | AVEWESNGQPENNYKTTPPVLDSDGSFFLYSRLTVDKSRWQQGNVFSCSVMHEALHNHYTQKSLSLSPGK |
| DC00018 | QVQLVESGGGLVQPGGSLRLSCATSGFTFRNNFMSWVRQAPGKGLEWVSTISYGGESTTYAESVKGRFTISRDNAKNTLYLQMNNLK |
| (SEQ ID NO: 2046) | PEDTAVYYCAKATSYDSIRSGSRGQGTQVTVSSGGGGSGGGGSGGGGSQVQLVESGGGLVQPGGSLRLSCATSGFTFRNNFMSWVRQ |
| | APGKGLEWVSTISYGGESTTYAESVKGRFTISRDNAKNTLYLQMNNLKPEDTAVYYCAKATSYDSIRSGSRGQGTQVTVSSDKTHTCP |
| | PCPAPEAAGGPSVFLFPPKPKDTLMISRTPEVTCVVVDVSHEDPEVKFNWYVDGVEVHNAKTKPREEQYNSTYRVVSVLTVLHQDWL |
| | NGKEYKCKVSNKALGAPIEKTISKAKGQPREPQVYTLPPSRDELTKNQVSLTCLVKGFYPSDIAVEWESNGQPENNYKTTPPVLDSDGS |
| | FLLYSKLTVDKSRWQQGNVFSCSVMHEALHNHYTQKSLSLSPGK |
| DC00019 | QVQLVESGGGLVQPGGSLRLSCATSGFTFRNNFMSWVRQAPGKGLEWVSTISYGGESTTYAESVKGRFTISRDNAKNTLYLQMNNLK |
| (SEQ ID NO: 2047) | PEDTAVYYCAKATSYDSIRSGSRGQGTQVTVSSGGGGSGGGGSGGGGSEVQLVESGGGLVQTGGSLRLSCAASGGTFSRDAMAWFR |
| | QVPGKEREFVALISWSGATTNYADSVKGRFAISRDNGKNTVYLQMNRLKPADTAIYYCAADRRPMGSRSYFEPTEYDDWGQGTQVT |
| | VSSGGGGSGGGGSGGGGSQVQLVESGGGLVQAGGSLTLSCAAPGRTFGTDVVGWFRQAPGKEREFVASISRSGDGIYYDDSVKGRFT |
| | ISRNNAWNTVNLQMNSLKVEDTAVYYCAAGDGWSTYDYWGQGTQVTVSSGGGGSGGGGSGGGGSDKTHTCPPCPAPEAAGGPSVF |
| | LFPPKPKDTLMISRTPEVTCVVVDVSHEDPEVKFNWYVDGVEVHNAKTKPREEQYNSTYRVVSVLTVLHQDWLNGKEYKCKVSNKA |
| | LGAPIEKTISKAKGQPREPQVYTLPPSRDELTKNQVSLTCLVKGFYPSDIAVEWESNGQPENNYKTTPPVLDSDGSFFLYSRLTVDKSR |
| | WQQGNVFSCSVMHEALHNHYTQKSLSLSPGK |
| DC00020 | QVQLVESGGGLVQAGGSLTLSCAAPGRTFGTDVVGWFRQAPGKEREFVASISRSGDGIYYDDSVKGRFTISRNNAWNTVNLQMNSLK |
| (SEQ ID NO: 2048) | VEDTAVYYCAAGDGWSTYDYWGQGTQVTVSSGGGGSGGGGSGGGGSEVQLVESGGGLVQTGGSLRLSCAASGGTFSRDAMAWFR |
| | QVPGKEREFVALISWSGATTNYADSVKGRFAISRDNGKNTVYLQMNRLKPADTAIYYCAADRRPMGSRSYFEPTEYDDWGQGTQVT |
| | VSSGGGGSGGGGSGGGGSQVQLVESGGGLVQPGGSLRLSCATSGFTFRNNFMSWVRQAPGKGLEWVSTISYGGESTTYAESVKGRFT |
| | ISRDNAKNTLYLQMNNLKPEDTAVYYCAKATSYDSIRSGSRGQGTQVTVSSDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVSNKALGAPIEKTISKAKGQP |
| | REPQVYTLPPSRDELTKNQVSLTCLVKGFYPSDIAVEWESNGQPENNYKTTPPVLDSDGSFFLYSRLTVDKSRWQQGNVFSCSVMHEA |
| | LHNHYTQKSLSLSPGK |
| DC0021 | QVQLVESGGGLVQAGGSLTLSCAAPGRTFGTDVVGWFRQAPGKEREFVASISRSGDGIYYDDSVKGRFTISRNNAWNTVNLQMNSLK |
| (SEQ ID NO: 2049) | VEDTAVYYCAAGDGWSTYDYWGQGTQVTVSSGGGGSGGGGSGGGGSQVQLVESGGGLVQPGGSLRLSCATSGFTFRNNFMSWVR |
| | QAPGKGLEWVSTISYGGESTTYAESVKGRFTISRDNAKNTLYLQMNNLKPEDTAVYYCAKATSYDSIRSGSRGQGTQVTVSSGGGGS |
| | GGGGSGGGGSEVQLVESGGGLVQTGGSLRLSCAASGGTFSRDAMAWFRQVPGKEREFVALISWSGATTNYADSVKGRFAISRDNGK |
| | NTVYLQMNRLKPADTAIYYCAADRRPMGSRSYFEPTEYDDWGQGTQVTVSSGGGGSGGGGSGGGGSDKTHTCPPCPAPEAAGGPSV |
| | FLFPPKPKDTLMISRTPEVTCVVVDVSHEDPEVKFNWYVDGVEVHNAKTKPREEQYNSTYRVVSVLTVLHQDWLNGKEYKCKVSNK |
| | ALGAPIEKTISKAKGQPREPQVYTLPPSRDELTKNQVSLTCLVKGFYPSDIAVEWESNGQPENNYKTTPPVLDSDGSFFLYSRLTVDKSR |
| | WQQGNVFSCSVMHEALHNHYTQKSLSLSPGK |
| DC00024 | QVQLVESGGGLVQPGGSLRLSCATSGFTFRNNFMSWVRQAPGKGLEWVSTISYGGESTTYAESVKGRFTISRDNAKNTLYLQMNNLK |
| (SEQ ID NO: 2050) | PEDTAVYYCAKATSYDSIRSGSRGQGTQVTVSSGGGGSGGGGSGGGGSEVQLVESGGGLVQTGGSLRLSCAASGGTFSRDAMAWFR |
| | QVPGKEREFVALISWSGATTNYADSVKGRFAISRDNGKNTVYLQMNRLKPADTAIYYCAADRRPMGSRSYFEPTEYDDWGQGTQVT |
| | VSSGGGGSGGGGSGGGGSDKTHTCPPCPAPEAAGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALGAPIEKTISKAKGQPREPQVYTLPPSRDELTKNQVSLTCLVKGFYPSDI |
| | AVEWESNGQPENNYKTTPPVLDSDGSFLLYSKLTVDKSRWQQGNVFSCSVMHEALHNHYTQKSLSLSPGK |
| DC00026 | EVQLVESGGGLVQTGGSLRLSCAASGGTFSRDAMAWFRQVPGKEREFVALISWSGATTNYADSVKGRFAISRDNGKNTVYLQMNRL |
| (SEQ ID NO: 2051) | KPADTAIYYCAADRRPMGSRSYFEPTEYDDWGQGTQVTVSSGGGGSGGGGSGGGGSQVQLVESGGGLVQPGGSLRLSCATSGFTFRN |
| | NFMSWVRQAPGKGLEWVSTISYGGESTTYAESVKGRFTISRDNAKNTLYLQMNNLKPEDTAVYYCAKATSYDSIRSGSRGQGTQVTV |
| | SSDKTHTCPPCPAPEAAGGPSVFLFPPKPKDTLMISRTPEVTCVVVDVSHEDPEVKFNWYVDGVEVHNAKTKPREEQYNSTYRVVSVL |
| | TVLHQDWLNGKEYKCKVSNKALGAPIEKTISKAKGQPREPQVYTLPPSRDELTKNQVSLTCLVKGFYPSDIAVEWESNGQPENNYKTT |
| | PPVLDSDGSFLLYSKLTVDKSRWQQGNVFSCSVMHEALHNHYTQKSLSLSPGK |
| DC00028 | QVQLVESGGGLVQPGGSLRLSCATSGFTFRNNFMSWVRQAPGKGLEWVSTISYGGESTTYAESVKGRFTISRDNAKNTLYLQMNNLK |
| (SEQ ID NO: 2052) | PEDTAVYYCAKATSYDSIRSGSRGQGTQVTVSSDKTHTCPPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALGAPIEKTISKAKGQPREPQVYTLPPSRDELTKNQ |
| | VSLTCLVKGFYPSDIAVEWESNGQPENNYKTTPPVLDSDGSFLLYSKLTVDKSRWQQGNVFSCSVMHEALHNHYTQKSLSLSPGK |
| DC00071 | QVQLVESGGGLVQAGGSLRLSCAASGIPFDNYAMGWFRQAPGKEREFVAARDLEGIITRYGDSVKGRFTISRGNAKNTVFLQMNSLK |
| (SEQ ID NO: 2053) | PEDTAVYYCAARDGGVVAGSRSSAQYNYWGQGTQVTVSSDKTHTCPPCPAPEAAGGPSVFLFPPKPKDTLMISRTPEVTCVVVDVSH |
| | EDPEVKFNWYVDGVEVHNAKTKPREEQYNSTYRVVSVLTVLHQDWLNGKEYKCKVSNKALGAPIEKTISKAKGQPREPQVYTLPPS |
| | RDELTKNQVSLTCLVKGFYPSDIAVEWESNGQPENNYKTTPPVLDSDGSFLLYSKLTVDKSRWQQGNVFSCSVMHEALHNHYTQKSL |
| | SLSPGK |
| DC00072 | QVQLVESGGGLVQAGGSLRLSCAASGGTFRTRNMGWFRRAPGKEREFVAAVSWNVDNKLYADSVKGRFTISRDNGRNMVYLQMNS |
| (SEQ ID NO: 2054) | LKPEDTAVYYCAADNIPLSSDMRPTATEYDYWGQGTQVTVSSDKTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQDWLNGKEYKCKVSNKALGAPIEKTISKAKGQPREPQVYTLP |
| | PSRDELTKNQVSLTCLVKGFYPSDIAVEWESNGQPENNYKTTPPVLDSDGSFLLYSKLTVDKSRWQQGNVFSCSVMHEALHNHYTQK |
| | SLSLSPGK |
| DC00073 | EVQLVESGGGLVQAGGSLRLSCAASGGSIYTYNMGWFRQAPGKEREFVAGTLWSGGDSVYADFAKGRFTLSRENAKNTLYLQMNSL |
| (SEQ ID NO: 2055) | KPEDTATYYCAIDPLSLTSDWRVDELSSWGKGTLVTVSSDKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGKEYKCKVSNKALGAPIEKTISKAKGQPREPQVYTLPPSRD |
| | ELTKNQVSLTCLVKGFYPSDIAVEWESNGQPENNYKTTPPVLDSDGSFLLYSKLTVDKSRWQQGNVFSCSVMHEALHNHYTQKSLSL |
| | SPGK |
| DC00074 | QLQLVESGGGLVQAGDSLRLSCAASGFNFGWHAMGWFRQAPGKEREFVATITWTGRDTYYADSVRGRFTISKDNAKDTLFLQMNSL |
| (SEQ ID NO: 2056) | RPDDTGVYYCAKARERATWAYSEDDCDYWGQGTQVTVSSDKTHTCPPCPAPEAAGGPSVFLFPPKPKDTLMISRTPEVTCVVVDVSH |
| | EDPEVKFNWYVDGVEVHNAKTKPREEQYNSTYRVVSVLTVLHQDWLNGKEYKCKVSNKALGAPIEKTISKAKGQPREPQVYTLPPS |
| | RDELTKNQVSLTCLVKGFYPSDIAVEWESNGQPENNYKTTPPVLDSDGSFLLYSKLTVDKSRWQQGNVFSCSVMHEALHNHYTQKSL |
| | SLSPGK |

1. A trispecific binding molecule comprising:
at least one single domain antigen-binding region specific for the IL-2Rα;
at least one single domain antigen-binding region specific for the IL-2Rβ; and
at least one single domain antigen-binding region specific for the γc.

2. The trispecific binding molecule of claim 1 which is a trispecific antibody comprising:
at least one VHH domain specific for the IL-2Rα;
at least one VHH domain specific for the IL-2Rβ; and
at least one VHH domain specific for the γc.

3. The trispecific binding molecule of claim 2, wherein:
(a) the trispecific antibody has higher affinity, avidity, selectivity, efficacy and/or potency for the IL-2Rα/IL-2Rβ/γc receptor complex compared to the IL-2Rβ/γc receptor complex;
(b) the trispecific antibody activates the IL-2Rα/IL-2Rβ/γc receptor complex resulting in phosphorylation of STAT5; and/or
(c) the trispecific antibody preferentially expands Treg cells

4. The trispecific binding molecule of claim 2, wherein the binding molecule is an antibody comprising two heavy chain polypeptides, but not a light chain.

5. The trispecific binding molecule of claim 2, wherein the binding molecule is an antibody comprising two heavy chains wherein:
(a) the antibody has four antigen-binding region;
(b) the antibody has four antigen-binding regions, with two antigen-binding regions on each heavy chain polypeptide;
(c) the antibody has four antigen binding sites, with one antigen-binding region on one heavy chain polypeptide and three antigen-binding regions on the other heavy chain polypeptide;
(d) the antibody has five antigen binding sites, with two antigen-binding regions on one heavy chain polypeptide and three antigen-binding regions on the other heavy chain polypeptide;
(e) the antibody has six antigen-binding regions;
(f) the antibody has six antigen-binding regions, with three antigen binding regions present on each heavy chain polypeptide;
(g) the antibody is symmetrical in the sense that each of the two heavy chain polypeptides is the same;
(h) the antibody is symmetrical in the sense that each of the two heavy chain polypeptides is the same, with each heavy chain comprising two antigen binding regions;
(i) the antibody is symmetrical in the sense that each of the two heavy chain polypeptides is the same, with each heavy chain comprising three antigen binding regions;
or
(j) the antibody is biparatopic comprising two different single domain binding regions that each bind a different epitope of the same IL-2R chain polypeptide.

6. The trispecific binding molecule of claim 2, wherein the binding molecule is an antibody comprising:
(a) an Fc region; or
(b) an Fc region, but no CH1 domain.

7. The trispecific binding molecule of claim 2, wherein the binding molecule is an antibody which is biparatopic for at least one of IL-2Rα, IL-2Rβ, and γc, preferably wherein it is biparatopic for at least IL-2Rα.

8. The trispecific binding molecule of claim 2, wherein the binding molecule is an antibody comprising:
(a) one of the antibody formats 1 to 19 shown in FIG. 12A or FIG. 12B or FIG. 12C either with or without the indicated specific Fc modifications; or
(b) one of the antibody formats 1 to 19 shown in FIG. 12A or FIG. 12B or FIG. 12C with the specific Fc region modifications shown.

9. The trispecific binding molecule of claim 2, where the binding molecule is an antibody comprising a heavy chain modification selected from one or more of:
a) N297A-Asn297Ala which confers Fc silencing;
b) LALA-Leu234Ala/Leu235Ala which decreases binding to Fc receptors;
c) LFLEPS-Leu234Phe/Leu235Glu/Pro331/Ser which decreases binding to Fc receptors;
d) PG-Pro329Gly which decreases binding to C1q;
e) LALA-PG-Leu234Ala/Leu235Ala/Pro329Gly which decreases binding to Fc receptors and C1q;
f) TM-Pro331Ser/Leu234Glu/Leu235Phe—which decreases binding to Fc receptors and C1q;
g) DA—Asp265Ala—which decreases binding to Fc receptors;
h) GRLR—Gly236Arg/Leu328Arg-which decreases binding to Fc receptors;
i) cFAE—K409R/F405L which promote heterodimer formation; and
j) M252Y/S254T/T256E (YTE) which helps extend the half-life of the antibody.

10. The trispecific binding molecule of claim 2, which comprises:
(a) a VHH domain selected from TABLE 3;
(b) a VHH domain specific for the IL-2Rα selected from TABLE 7;
(c) a VHH domain specific for the IL-2Rβ selected from TABLE 8;
(d) a VHH domain specific for the γc selected from TABLE 9;
(e) a VHH domain specific for the IL-2Rα selected from TABLE 3, a VHH domain specific for the IL-2Rβ selected from TABLE 3, and a VHH domain specific for the γc selected from TABLE 3; or
(f) a VHH domain specific for the IL-2Rα selected from TABLE 7, a VHH domain specific for the IL-2Rβ selected from TABLE 8, and a VHH domain specific for the γc selected from TABLE 9.

11. The trispecific binding molecule of claim 2 which comprises:
(a) a set of three CDRs from a VHH domain selected from TABLE 3;
(b) a set of three CDRs from a VHH domain specific for the IL-2Rα selected from TABLE 7;
(c) a set of three CDRs from a VHH domain specific for the IL-2Rβ selected from TABLE 8;
(d) a set of three CDRs from a VHH domain specific for the γc selected from TABLE 9;
(e) a set of three CDRs from a VHH domain specific for the IL-2Rα selected from TABLE 3, a set of three CDRs from a VHH domain specific for the IL-2Rβ selected from TABLE 3, and a set of three CDRs from a VHH domain specific for the γc selected from TABLE 3; or
(f) a set of three CDRs from a VHH domain specific for the IL-2Rα selected from TABLE 7, a set of three CDRs from a VHH domain specific for the IL-2Rβ selected from TABLE 8, and a set of three CDRs from a VHH domain specific for the γc selected from TABLE 9.

12. The trispecific binding molecule of claim 2 which comprises:
(a) a set of three VHH domains as set out in TABLE 5 or the CDRs for a set of three VHH domains as set out in TABLE 5;
(b) a set of the VHH domains from one of the polypeptides in TABLE 12, all of the CDR sets of one of the polypeptides set out in TABLE 12; or
(c) a set of the VHH domains from a pair of the polypeptides in TABLE 12, all of the CDR sets from a pair of the polypeptides set out in TABLE 12, wherein the pair of polypeptides is one of those defined by the pairwise combinations set out in FIG. 12C.

13. The trispecific binding molecule of claim 2, which comprises the geometry and VHH domain sequences, or variants thereof, of one of the antibodies shown in FIG. 12.

14. The trispecific binding molecule of claim 2, which comprises:
(a) the CDRs of one of the binding molecules shown in FIG. 12C or variant CDRs of those CDRs;
(b) the VHH domains of one of the binding molecules shown in FIG. 12 or variant VHH domains;
(c) what is set out in (a) or (b) wherein the CDRs or VHHs domains are in the same order as shown in FIG. 12C, with the binding molecule having the same format as shown in FIG. 12C, but not necessarily the same sequence apart from the CDRs or VHHs, optionally wherein the binding molecule comprises the constant region modifications shown in FIG. 12C;
(d) the CDR sets of one of the polypeptide sequences set out in Table 12 or variants thereof;
(e) the CDR sets from a pair of polypeptides sequences set out in Table 12 or variants thereof, wherein the pair of polypeptides are one of the pairs of polypeptides shown in FIG. 12C;
(f) the VHH domains of one of the polypeptide sequences set out in Table 12 or variants thereof;
(g) the VHH domains from a pair of polypeptides sequences set out in Table 12 or variants thereof, wherein the pair of polypeptides are one of the pairs of polypeptides shown in FIG. 12C;
(h) at least one of the polypeptide sequences set out in Table 12 or variants thereof; or
(i) a pair of the polypeptide sequences set out in Table 12 or variants thereof, wherein the pair of polypeptides are one of the pairs of polypeptides shown in FIG. 12C;

15. The trispecific binding molecule of claim 1 for use as a medicament.

16. The trispecific binding molecule of claim 1 for use in a method of treating or preventing an autoimmune disorder, or an inflammatory disorder,
preferably wherein:
(a) the disorder is graft versus host disease (GvHD), preferably where the antibody is for use in a method where it is administered prior to, at the same time, or after a transplant of a cell, tissue, or organ; or
(b) the disorder is one involving dysfunction or unwanted proliferation of leukocytes, preferably of T cells, more preferably of Teff cells; such disorders may present with an imbalance of Tregs compared to Teff cells;
(c) the disorder is selected from inflammatory bowel disease (such as ulcerative colitis, Crohn's disease, pouchitis or celiac disease), SLE, multiple sclerosis, type 1 diabetes, myasthenia gravis, pemphigus vulgaris, and bullous pemphigoid;
(d) the disorder is selected from SLE, GvHD, psoriasis, autoimmune hepatitis, ulcerative colitis, eczema;
(e) the disorder is one that can be treated by expansion of Tregs; or
(f) the disorder is one involving dysfunction or unwanted deficiency of leukocytes, preferably regulatory T cells; such disorders may present with an imbalance of Tregs compared to Teff cells;
(g) the disorder is one involving reduced numbers, dysfunction or unwanted proliferation of leukocytes, preferably of T cells, more preferably of T reg cells; or
(h) the disorder is one that can benefit from increasing numbers and/or function of Tregs.

17. A method of stimulating cell proliferation comprising contacting a target cell expressing the IL-2Rα/IL-2Rβ/γc receptor complex with a trispecific binding molecule according to claim 1.

18. A pharmaceutical composition comprising a trispecific binding molecule according to claim 1 and a pharmaceutically acceptable carrier.

19. A method of detecting the IL-2Rα/IL-2Rβ/γc receptor complex comprising contacting a test sample with a binding molecule according to claim 1 and detecting binding of the binding molecule to the IL-2Rα/IL-2Rβ/γc receptor complex
preferably wherein the binding molecule is labelled and the binding of the antibody to the IL-2Rα/IL-2Rβ/γc receptor complex is detected via the label.
